(19) United States
(12) Patent Application Publication
(10) Pub. No.: US 2024/0417737 A1
(43) Pub. Date: Dec. 19, 2024

(54) METHODS AND COMPOSITIONS FOR TREATING EPILEPSY

(71) Applicants: UNIQURE FRANCE, Illkirch-Graffenstaden (FR); REGENXBIO INC., Rockville, MD (US); INSERM (Institut National de la Santé et de la Recherche Médicale), Paris (FR); CENTRE NATIONAL DE LA RECHERCHE SCIENTIFIQUE, Paris (FR); UNIVERSITÉ DE BORDEAUX, Bordeaux (FR); UNIVERSITÉ D'AIX-MARSEILLE, Marseille (FR)

(72) Inventors: Chenxia HE, Paris (FR); Richard PORTER, Binningen (CH); Andrew MERCER, Rockville, MD (US); Olivier DANOS, Rockville, MD (US); April R. TEPE, Laurel, MD (US)

(21) Appl. No.: 18/289,921

(22) PCT Filed: May 16, 2022
(86) PCT No.: PCT/US2022/029452

§ 371 Date: Nov. 8, 2023

Related U.S. Application Data
(60) Provisional application No. 63/219,446, filed on Jul. 8, 2021.
(60) Provisional application No. 63/189,552, filed on May 17, 2021.

(51) Int. Cl. C12N 15/113; A61P 25/08; C12N 15/86

(52) U.S. Cl. CPC C12N 15/1138; A61P 25/08; C12N 15/86; C12N 2310/11; C12N 2310/141; C12N 2310/531; C12N 2750/14143

(57) ABSTRACT

Disclosed are methods and compositions relating to antisense therapy for treating epilepsy, such as a temporal lobe epilepsy, in a subject in need thereof by targeting Grik2 mRNA. In particular, the disclosure provides inhibitory RNA constructs capable of inhibiting expression of GluK2 protein and inhibiting epileptiform discharges in hyperexcitable neuronal circuits.

### SEQUENCE LISTING

The instant application contains a Sequence Listing which has been submitted electronically in ASCII format and is hereby incorporated by reference in its entirety. Said ASCII copy, created on Jan. 25, 2024, is named “51460-007003_Sequence_Listing_1_25_24_ST25” and is 297,397 bytes in size.

### FIELD OF THE DISCLOSURE

The disclosure is in the field of epilepsy. In particular, the disclosure relates to methods and compositions for treating an epilepsy, such as, e.g., temporal lobe epilepsy.

### BACKGROUND

Globally, an estimated 5 million people are diagnosed each year with epilepsy, a neurological disorder marked by seizures, or sudden recurrent episodes of sensory disturbance, loss of consciousness, or convulsions associated with abnormal electrical activity in the brain. A typical diagnosis of epilepsy arises when a patient experiences two or more unprovoked seizures. Causes of epilepsy include genetic abnormalities, prior brain infection, prenatal injuries, developmental disorders, and other neurological issues such as strokes or brain tumors, though approximately 50% of people who are diagnosed with epilepsy have no known cause for the development of the disorder.

Temporal lobe epilepsy (TLE) is the most common form of partial epilepsy in adults (30-40% of all forms of epilepsies). It is well established that the hippocampus plays a key role in the pathophysiology of TLE. In human patients and animal models of TLE, an aberrant rewiring of neuronal circuits occurs. One of the best examples of network reorganization (“reactive plasticity”) is the sprouting of recurrent mossy fibers (rMF) that establish novel pathophysiological glutamatergic synapses onto dentate granule cells (DGCs) in the hippocampus (Tauck and Nadler, 1985; Represa et al., 1989a, 1989b; Sutula et al., 1989; Gabriel et al., 2004) leading to a recurrent excitatory loop. rMF synapses operate through ectopic kainate receptors (KARs) (Epsztein et al., 2005; Artinian et al., 2011, 2015). KARs are tetrameric glutamate receptors assembled from GluK1-GluK5 subunits. In heterologous expression systems, GluK1, GluK2, and GluK3 may form homomeric receptors, while GluK4 and GluK5 form heteromeric receptors in conjunction with GluK1-3 subunits. Native KARs are widely distributed in the brain with high densities of receptors found in the hippocampus (Carta et al, 2016, EJN), a key structure involved in TLE. Prior studies by the present inventors have established that epileptic activities including interictal spikes and ictal discharges were markedly reduced in mice lacking the GluK2 KAR subunit. Moreover, epileptiform activities were strongly reduced following the use of pharmacological small molecule antagonists of GluK2/GluK5-containing KARs, which block ectopic synaptic KARs (Peret et al., 2014). These data support a hypothesis that KARs ectopically expressed at rMFs in DGCs play a major role in chronic seizures in TLE. Therefore, aberrant KARs expressed in DGCs and composed of GluK2/GluK5 are considered to represent a promising target for the treatment of pharmaco-resistant epilepsies such as TLE.

RNA interference (RNAi) strategies have been proposed for many disease targets. Successful application of RNAi-based therapies has been limited. RNAi therapeutics face multiple challenges, such as the need for repeat dosing and formulation challenges. However, available RNAi-based gene therapies for the treatment of intractable TLE are limited. Therefore, there exists an urgent need for new therapeutic modalities for the treatment of seizure disorders, such as, e.g., TLE (e.g., TLE refractory to treatment).

### SUMMARY OF THE DISCLOSURE

The disclosure provides compositions and methods for the treatment or prevention of an epilepsy, such as, e.g., a temporal lobe epilepsy (TLE), in a subject (e.g., a human) in need thereof. The disclosed methods include administration of a therapeutically effective amount of a polynucleotide (e.g., an inhibitory polynucleotide), such as, e.g., an antisense oligonucleotide (ASO), shRNA, siRNA, microRNA, or shmiRNA, that targets an mRNA encoded by a glutamate ionotropic receptor kainate type subunit 2 (Grik2) gene, or a nucleic acid vector encoding the same (e.g., a lentiviral vector or an adeno-associated viral (AAV) vector, such as, e.g., an AAV9 vector), to a subject diagnosed as having or at risk of developing an epilepsy. The disclosed polynucleotides exhibit improved loading into the RNA-induced silencing complex (RISC) protein in order to enhance RNA-interference-mediated degradation of the Grik2 transcript. The disclosure also features pharmaceutical compositions containing one or more of the disclosed inhibitory nucleic acid (e.g., RNA) agents and nucleic acid vectors encoding the same.

This disclosure is based, in part, on the surprising discovery that the inhibitory polynucleotides described herein exhibit a significantly higher guide to passenger strand ratio (G/P ratio), which supports a direct, substantial increase in the processing of the inhibitory polynucleotide and a subsequent improvement in the reduction of both the expression levels of Grik2 mRNA and the resulting GluK2 protein. A challenge of microRNA (miRNA) therapeutics is low processing efficiency of the transfected polynucleotides. Therefore, an improvement in G/P ratio can be correlated with an increase in production of mature miRNA molecules, and, concomitantly, an increase in the desired therapeutic effect(s) of the administered miRNA therapy.

In a first aspect, the disclosure features an isolated inhibitory polynucleotide(s) that specifically hybridize(s) to a Grik2 mRNA including a stem-loop region including a 5′ arm (5p), a loop region, and a 3′ arm (3p), wherein the stem-loop region includes a guide strand sequence and a passenger strand sequence, and the guide strand sequence and passenger strand sequence includes: (a) a uracil (U)-adenine (A) base pair or a U-guanine (G) base pair at the 5′ end of the guide strand, (b) a cytosine (C)-G pair at the 5′ end of the passenger strand, (c) a U at the 5′ end of the guide strand sequence, (d) a mismatch in a seed region between the guide strand and passenger strand sequences; and/or (c) a C-G base pair or U-A base pair to replace a U-G wobble at a junction of the stem region and the loop region of the polynucleotide.

In some embodiments, a) and c) improve guide strand sequence loading into an RNA-induced silencing complex (RISC) protein. In some embodiments, b) impairs passenger strand sequence loading into a RISC protein. In some embodiments, d) promotes decoupling of the passenger strand sequence from the guide strand sequence during RISC loading. In some embodiments, e) improves cleavage of the loop region from the stem region by Dicer. In some embodiments, the seed region of the guide strand sequence comprises nucleotides 2 through 7 of the guide strand sequence.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 2. In some embodiments, the guide strand sequence has the nucleic acid sequence of SEQ ID NO: 17. In some embodiments, the guide strand of SEQ ID NO: 17 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 17 shown in Table 3. In some embodiments, the passenger strand sequence has the nucleic acid sequence of SEQ ID NO: 32. In some embodiments, the passenger strand of SEQ ID NO: 32 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 32 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 3. In some embodiments, the guide strand sequence has the nucleic acid sequence of SEQ ID NO: 18. In some embodiments, the guide strand of SEQ ID NO: 18 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 18 shown in Table 3. In some embodiments, the passenger strand sequence has the nucleic acid sequence of SEQ ID NO: 33. In some embodiments, the passenger strand of SEQ ID NO: 33 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 33 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 4. In some embodiments, the guide strand sequence has the nucleic acid sequence of SEQ ID NO: 19. In some embodiments, the guide strand of SEQ ID NO: 19 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 19 shown in Table 3. In some embodiments, the passenger strand sequence has the nucleic acid sequence of SEQ ID NO: 34. In some embodiments, the passenger strand of SEQ ID NO: 34 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 34 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 5. In some embodiments, the guide strand sequence has the nucleic acid sequence of SEQ ID NO: 20. In some embodiments, the guide strand of SEQ ID NO: 20 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 20 shown in Table 3. In some embodiments, the passenger strand sequence has the nucleic acid sequence of SEQ ID NO: 35. In some embodiments, the passenger strand of SEQ ID NO: 35 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 35 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 6. In some embodiments, the guide strand sequence has the nucleic acid sequence of SEQ ID NO: 21. In some embodiments, the guide strand of SEQ ID NO: 21 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 21 shown in Table 3. In some embodiments, the passenger strand sequence has the nucleic acid sequence of SEQ ID NO: 36. In some embodiments, the passenger strand of SEQ ID NO: 36 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 36 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 7. In some embodiments, the guide strand sequence has the nucleic acid sequence of SEQ ID NO: 22. In some embodiments, the guide strand of SEQ ID NO: 22 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 22 shown in Table 3. In some embodiments, the passenger strand sequence has the nucleic acid sequence of SEQ ID NO: 37. In some embodiments, the passenger strand of SEQ ID NO: 37 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 37 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 8. In some embodiments, the guide strand sequence has the nucleic acid sequence of SEQ ID NO: 23. In some embodiments, the guide strand of SEQ ID NO: 23 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 23 shown in Table 3. In some embodiments, the passenger strand sequence has the nucleic acid sequence of SEQ ID NO: 38. In some embodiments, the passenger strand of SEQ ID NO: 38 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 38 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 9. In some embodiments, the guide strand sequence has the nucleic acid sequence of SEQ ID NO: 24. In some embodiments, the guide strand of SEQ ID NO: 23 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 23 shown in Table 3. In some embodiments, the passenger strand sequence has the nucleic acid sequence of SEQ ID NO: 39. In some embodiments, the passenger strand of SEQ ID NO: 39 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 39 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 10. In some embodiments, the guide strand sequence has the nucleic acid sequence of SEQ ID NO: 25. In some embodiments, the guide strand of SEQ ID NO: 25 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 25 shown in Table 3. In some embodiments, the passenger strand sequence has the nucleic acid sequence of SEQ ID NO: 40. In some embodiments, the passenger strand of SEQ ID NO: 40 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 40 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 11. In some embodiments, the guide strand sequence has the nucleic acid sequence of SEQ ID NO: 26. In some embodiments, the guide strand of SEQ ID NO: 4 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 26 shown in Table 3. In some embodiments, the passenger strand sequence has the nucleic acid sequence of SEQ ID NO: 41. In some embodiments, the passenger strand of SEQ ID NO: 41 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 41 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 12. In some embodiments, the guide strand sequence has the nucleic acid sequence of SEQ ID NO: 27. In some embodiments, the guide strand of SEQ ID NO: 27 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 27 shown in Table 3. In some embodiments, the passenger strand sequence has the nucleic acid sequence of SEQ ID NO: 42. In some embodiments, the passenger strand of SEQ ID NO: 42 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 42 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 13. In some embodiments, the guide strand sequence has the nucleic acid sequence of SEQ ID NO: 28. In some embodiments, the guide strand of SEQ ID NO: 28 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 28 shown in Table 3. In some embodiments, the passenger strand sequence has the nucleic acid sequence of SEQ ID NO: 43. In some embodiments, the passenger strand of SEQ ID NO: 43 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 43 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 14. In some embodiments, the guide strand sequence has the nucleic acid sequence of SEQ ID NO: 29. In some embodiments, the guide strand of SEQ ID NO: 29 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 29 shown in Table 3. In some embodiments, the passenger strand sequence has the nucleic acid sequence of SEQ ID NO: 44. In some embodiments, the passenger strand of SEQ ID NO: 44 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 44 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 15. In some embodiments, the guide strand sequence has the nucleic acid sequence of SEQ ID NO: 30. In some embodiments, the guide strand of SEQ ID NO: 30 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 30 shown in Table 3. In some embodiments, the passenger strand sequence has the nucleic acid sequence of SEQ ID NO: 45. In some embodiments, the passenger strand of SEQ ID NO: 45 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 45 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 226. In some embodiments, the guide strand sequence has the nucleic acid sequence of SEQ ID NO: 230. In some embodiments, the guide strand of SEQ ID NO: 230 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 230 shown in Table 3. In some embodiments, the passenger strand sequence has the nucleic acid sequence of SEQ ID NO: 234. In some embodiments, the passenger strand of SEQ ID NO: 234 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 234 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 227. In some embodiments, the guide strand sequence has the nucleic acid sequence of SEQ ID NO: 231. In some embodiments, the guide strand of SEQ ID NO: 231 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 231 shown in Table 3. In some embodiments, the passenger strand sequence has the nucleic acid sequence of SEQ ID NO: 235. In some embodiments, the passenger strand of SEQ ID NO: 235 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 235 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 228. In some embodiments, the guide strand sequence has the nucleic acid sequence of SEQ ID NO: 232. In some embodiments, the guide strand of SEQ ID NO: 232 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 232 shown in Table 3. In some embodiments, the passenger strand sequence has the nucleic acid sequence of SEQ ID NO: 236. In some embodiments, the passenger strand of SEQ ID NO: 236 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 236 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 229. In some embodiments, the guide strand sequence has the nucleic acid sequence of SEQ ID NO: 233. In some embodiments, the guide strand of SEQ ID NO: 233 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 233 shown in Table 3. In some embodiments, the passenger strand sequence has the nucleic acid sequence of SEQ ID NO: 237. In some embodiments, the passenger strand of SEQ ID NO: 237 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 237 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 238. In some embodiments, the guide strand sequence has the nucleic acid sequence of SEQ ID NO: 242. In some embodiments, the guide strand of SEQ ID NO: 242 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 242 shown in Table 3. In some embodiments, the passenger strand sequence has the nucleic acid sequence of SEQ ID NO: 246. In some embodiments, the passenger strand of SEQ ID NO: 246 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 246 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 239. In some embodiments, the guide strand sequence has the nucleic acid sequence of SEQ ID NO: 243. In some embodiments, the guide strand of SEQ ID NO: 243 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 243 shown in Table 3. In some embodiments, the passenger strand sequence has the nucleic acid sequence of SEQ ID NO: 247. In some embodiments, the passenger strand of SEQ ID NO: 247 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 247 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 240. In some embodiments, the guide strand sequence has the nucleic acid sequence of SEQ ID NO: 244. In some embodiments, the guide strand of SEQ ID NO: 244 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 244 shown in Table 3. In some embodiments, the passenger strand sequence has the nucleic acid sequence of SEQ ID NO: 248. In some embodiments, the passenger strand of SEQ ID NO: 248 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 248 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 241. In some embodiments, the guide strand sequence has the nucleic acid sequence of SEQ ID NO: 245. In some embodiments, the guide strand of SEQ ID NO: 245 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 245 shown in Table 3. In some embodiments, the passenger strand sequence has the nucleic acid sequence of SEQ ID NO: 249. In some embodiments, the passenger strand of SEQ ID NO: 249 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 249 shown in Table 3.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 47. In some embodiments, the guide strand sequence has the nucleic acid sequence of SEQ ID NO: 64. In some embodiments, the guide strand of SEQ ID NO: 64 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 64 shown in Table 5. In some embodiments, the passenger strand sequence has the nucleic acid sequence of SEQ ID NO: 81. In some embodiments, the passenger strand of SEQ ID NO: 81 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 81 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 48. In some embodiments, the guide strand sequence has the nucleic acid sequence of SEQ ID NO: 65. In some embodiments, the guide strand of SEQ ID NO: 65 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 65 shown in Table 5. In some embodiments, the passenger strand sequence has the nucleic acid sequence of SEQ ID NO: 82. In some embodiments, the passenger strand of SEQ ID NO: 82 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 82 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 49. In some embodiments, the guide strand sequence has the nucleic acid sequence of SEQ ID NO: 66. In some embodiments, the guide strand of SEQ ID NO: 66 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 66 shown in Table 5. In some embodiments, the passenger strand sequence has the nucleic acid sequence of SEQ ID NO: 83. In some embodiments, the passenger strand of SEQ ID NO: 83 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 83 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 50. In some embodiments, the guide strand sequence has the nucleic acid sequence of SEQ ID NO: 67. In some embodiments, the guide strand of SEQ ID NO: 67 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 67 shown in Table 5. In some embodiments, the passenger strand sequence has the nucleic acid sequence of SEQ ID NO: 84. In some embodiments, the passenger strand of SEQ ID NO: 84 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 84 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 51. In some embodiments, the guide strand sequence has the nucleic acid sequence of SEQ ID NO: 68. In some embodiments, the guide strand of SEQ ID NO: 68 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 68 shown in Table 5. In some embodiments, the passenger strand sequence has the nucleic acid sequence of SEQ ID NO: 85. In some embodiments, the passenger strand of SEQ ID NO: 85 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 85 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 52. In some embodiments, the guide strand sequence has the nucleic acid sequence of SEQ ID NO: 69. In some embodiments, the guide strand of SEQ ID NO: 69 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 69 shown in Table 5. In some embodiments, the passenger strand sequence has the nucleic acid sequence of SEQ ID NO: 86. In some embodiments, the passenger strand of SEQ ID NO: 86 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 86 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 53. In some embodiments, the guide strand sequence has the nucleic acid sequence of SEQ ID NO: 70. In some embodiments, the guide strand of SEQ ID NO: 70 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 70 shown in Table 5. In some embodiments, the passenger strand sequence has the nucleic acid sequence of SEQ ID NO: 87. In some embodiments, the passenger strand of SEQ ID NO: 87 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 87 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 54. In some embodiments, the guide strand sequence has the nucleic acid sequence of SEQ ID NO: 71. In some embodiments, the guide strand of SEQ ID NO: 71 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 71 shown in Table 5. In some embodiments, the passenger strand sequence has the nucleic acid sequence of SEQ ID NO: 88. In some embodiments, the passenger strand of SEQ ID NO: 88 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 88 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 55. In some embodiments, the guide strand sequence has the nucleic acid sequence of SEQ ID NO: 72. In some embodiments, the guide strand of SEQ ID NO: 72 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 72 shown in Table 5. In some embodiments, the passenger strand sequence has the nucleic acid sequence of SEQ ID NO: 89. In some embodiments, the passenger strand of SEQ ID NO: 89 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 89 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 56. In some embodiments, the guide strand sequence has the nucleic acid sequence of SEQ ID NO: 73. In some embodiments, the guide strand of SEQ ID NO: 73 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 73 shown in Table 5. In some embodiments, the passenger strand sequence has the nucleic acid sequence of SEQ ID NO: 90. In some embodiments, the passenger strand of SEQ ID NO: 90 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 90 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 57. In some embodiments, the guide strand sequence has the nucleic acid sequence of SEQ ID NO: 74. In some embodiments, the guide strand of SEQ ID NO: 74 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 74 shown in Table 5. In some embodiments, the passenger strand sequence has the nucleic acid sequence of SEQ ID NO: 91. In some embodiments, the passenger strand of SEQ ID NO: 91 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 91 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 58. In some embodiments, the guide strand sequence has the nucleic acid sequence of SEQ ID NO: 75. In some embodiments, the guide strand of SEQ ID NO: 75 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 75 shown in Table 5. In some embodiments, the passenger strand sequence has the nucleic acid sequence of SEQ ID NO: 92. In some embodiments, the passenger strand of SEQ ID NO: 92 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 92 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 59. In some embodiments, the guide strand sequence has the nucleic acid sequence of SEQ ID NO: 76. In some embodiments, the guide strand of SEQ ID NO: 76 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 76 shown in Table 5. In some embodiments, the passenger strand sequence has the nucleic acid sequence of SEQ ID NO: 93. In some embodiments, the passenger strand of SEQ ID NO: 93 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 93 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 60. In some embodiments, the guide strand sequence has the nucleic acid sequence of SEQ ID NO: 77. In some embodiments, the guide strand of SEQ ID NO: 77 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 77 shown in Table 5. In some embodiments, the passenger strand sequence has the nucleic acid sequence of SEQ ID NO: 94. In some embodiments, the passenger strand of SEQ ID NO: 94 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 94 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 61. In some embodiments, the guide strand sequence has the nucleic acid sequence of SEQ ID NO: 78. In some embodiments, the guide strand of SEQ ID NO: 78 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 78 shown in Table 5. In some embodiments, the passenger strand sequence has the nucleic acid sequence of SEQ ID NO: 95. In some embodiments, the passenger strand of SEQ ID NO: 95 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 95 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 62. In some embodiments, the guide strand sequence has the nucleic acid sequence of SEQ ID NO: 79. In some embodiments, the guide strand of SEQ ID NO: 79 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 79 shown in Table 5. In some embodiments, the passenger strand sequence has the nucleic acid sequence of SEQ ID NO: 96. In some embodiments, the passenger strand of SEQ ID NO: 96 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 96 shown in Table 5.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 98. In some embodiments, the guide strand sequence has the nucleic acid sequence of SEQ ID NO: 110. In some embodiments, the guide strand of SEQ ID NO: 110 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 110 shown in Table 7. In some embodiments, the passenger strand sequence has the nucleic acid sequence of SEQ ID NO: 122. In some embodiments, tho passenger strand of SEQ ID NO: 122 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 122 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 99. In some embodiments, the guide strand sequence has the nucleic acid sequence of SEQ ID NO: 111. In some embodiments, the guide strand of SEQ ID NO: 111 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 111 shown in Table 7. In some embodiments, the passenger strand sequence has the nucleic acid sequence of SEQ ID NO: 123. In some embodiments, the passenger strand of SEQ ID NO: 123 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 123 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 100. In some embodiments, the guide strand sequence has the nucleic acid sequence of SEQ ID NO: 112. In some embodiments, the guide strand of SEQ ID NO: 112 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 112 shown in Table 7. In some embodiments, the passenger strand sequence has the nucleic acid sequence of SEQ ID NO: 124. In some embodiments, the passenger strand of SEQ ID NO: 124 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 124 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 101. In some embodiments, the guide strand sequence has the nucleic acid sequence of SEQ ID NO: 113. In some embodiments, the guide strand of SEQ ID NO: 113 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 113 shown in Table 7. In some embodiments, the passenger strand sequence has the nucleic acid sequence of SEQ ID NO: 125. In some embodiments, the passenger strand of SEQ ID NO: 125 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 125 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 102. In some embodiments, the guide strand sequence has the nucleic acid sequence of SEQ ID NO: 114. In some embodiments, the guide strand of SEQ ID NO: 114 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 114 shown in Table 7. In some embodiments, the passenger strand sequence has the nucleic acid sequence of SEQ ID NO: 126. In some embodiments, the passenger strand of SEQ ID NO: 126 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 126 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 103. In some embodiments, the guide strand sequence has the nucleic acid sequence of SEQ ID NO: 115. In some embodiments, the guide strand of SEQ ID NO: 115 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 115 shown in Table 7. In some embodiments, the passenger strand sequence has the nucleic acid sequence of SEQ ID NO: 127. In some embodiments, the passenger strand of SEQ ID NO: 127 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 127 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 104. In some embodiments, the guide strand sequence has the nucleic acid sequence of SEQ ID NO: 116. In some embodiments, the guide strand of SEQ ID NO: 116 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 116 shown in Table 7. In some embodiments, the passenger strand sequence has the nucleic acid sequence of SEQ ID NO: 128. In some embodiments, the passenger strand of SEQ ID NO: 128 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 128 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 105. In some embodiments, the guide strand sequence has the nucleic acid sequence of SEQ ID NO: 117. In some embodiments, the guide strand of SEQ ID NO: 117 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 117 shown in Table 7. In some embodiments, the passenger strand sequence has the nucleic acid sequence of SEQ ID NO: 129. In some embodiments, the passenger strand of SEQ ID NO: 129 contains 1-7 (e.g., 1, 2, 3, 4, 5, 6, or 7) nucleotide alterations (e.g., substitution, deletion, insertion, or mismatch), wherein the alteration(s) does not involve any ono of the bolded nucleotides of SEQ ID NO: 129 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 106. In some embodiments, the guide strand sequence has the nucleic acid sequence of SEQ ID NO: 118. In some embodiments, the guide strand of SEQ ID NO: 118 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 118 shown in Table 7. In some embodiments, the passenger strand sequence has the nucleic acid sequence of SEQ ID NO: 130. In some embodiments, the passenger strand of SEQ ID NO: 130 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 130 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 107. In some embodiments, the guide strand sequence has the nucleic acid sequence of SEQ ID NO: 119. In some embodiments, the guide strand of SEQ ID NO: 119 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 119 shown in Table 7. In some embodiments, the passenger strand sequence has the nucleic acid sequence of SEQ ID NO: 131. In some embodiments, the passenger strand of SEQ ID NO: 131 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 131 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 108. In some embodiments, the guide strand sequence has the nucleic acid sequence of SEQ ID NO: 120. In some embodiments, the guide strand of SEQ ID NO: 120 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 120 shown in Table 7. In some embodiments, the passenger strand sequence has the nucleic acid sequence of SEQ ID NO: 132. In some embodiments, the passenger strand of SEQ ID NO: 132 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 132 shown in Table 7.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 134. In some embodiments, the guide strand sequence has the nucleic acid sequence of SEQ ID NO: 140. In some embodiments, the guide strand of SEQ ID NO: 140 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 140 shown in Table 9. In some embodiments, the passenger strand sequence has the nucleic acid sequence of SEQ ID NO: 146. In some embodiments, the passenger strand of SEQ ID NO: 146 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 146 shown in Table 9.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 135. In some embodiments, the guide strand sequence has the nucleic acid sequence of SEQ ID NO: 141. In some embodiments, the guide strand of SEQ ID NO: 141 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 141 shown in Table 9. In some embodiments, the passenger strand sequence has the nucleic acid sequence of SEQ ID NO: 147. In some embodiments, the passenger strand of SEQ ID NO: 147 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 147 shown in Table 9.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 136. In some embodiments, the guide strand sequence has the nucleic acid sequence of SEQ ID NO: 142. In some embodiments, the guide strand of SEQ ID NO: 142 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 142 shown in Table 9. In some embodiments, the passenger strand sequence has the nucleic acid sequence of SEQ ID NO: 148. In some embodiments, the passenger strand of SEQ ID NO: 148 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 148 shown in Table 9.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 137. In some embodiments, the guide strand sequence has the nucleic acid sequence of SEQ ID NO: 143. In some embodiments, the guide strand of SEQ ID NO: 143 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 143 shown in Table 9. In some embodiments, the passenger strand sequence has the nucleic acid sequence of SEQ ID NO: 149. In some embodiments, the passenger strand of SEQ ID NO: 149 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 149 shown in Table 9.

In some embodiments, the stem-loop region is a polynucleotide having at least 85% (e.g., at least 86%, 87%, 88%, 89%, 90%, 91%, 92%, 93%, 94%, 95%, 96%, 97%, 98%, 99%, or 100%) sequence identity to the nucleic acid sequence of SEQ ID NO: 138. In some embodiments, the guide strand sequence has the nucleic acid sequence of SEQ ID NO: 144. In some embodiments, the guide strand of SEQ ID NO: 144 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 144 shown in Table 9. In some embodiments, the passenger strand sequence has the nucleic acid sequence of SEQ ID NO: 150. In some embodiments, the passenger strand of SEQ ID NO: 150 contains 1-7 (e.g., 1, 2, 3, 4, 5, 6, or 7) nucleotide alterations (e.g., substitution, deletion, insertion, or mismatch), wherein the alteration(s) does not involve any one of the bolded nucleotides of SEQ ID NO: 150 shown in Table 9.

In some embodiments, the inhibitory polynucleotide comprises an antisense oligonucleotide (ASO). In some embodiments, the inhibitory polynucleotide comprises a short interfering RNA (siRNA), a short hairpin RNA (shRNA), a microRNA (miRNA), or a short hairpin-adapted miRNA (shmiRNA).

In some embodiments, the polynucleotide is between 19 to 21 nucleotides. In some embodiments, the polynucleotide is 19 nucleotides. In some embodiments, the polynucleotide is 20 nucleotides. In some embodiments, the polynucleotide is 21 nucleotides.

In some embodiments, the Grik2 mRNA is encoded by a nucleic acid sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to any one of SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171. SEQ ID NO: 172, SEQ ID NO: 173, or SEQ ID NO: 174. In some embodiments, the Grik2 mRNA is encoded by a nucleic acid sequence of SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, or SEQ ID NO: 174.

In some embodiments, the inhibitory polynucleotide is capable of reducing a level of GluK2 protein in a cell (as is discussed further in the disclosure). In some embodiments, the polynucleotide reduces a level of GluK2 protein in the cell by at least 10%, at least at least 15%, at least 20%, at least 25%, at least 30%, at least 35%, at least 40%, at least 45%, at least 50%, at least 55%, at least 60%, at least 65%, at least 70%, or at least 75%. In some embodiments, the coll is a neuron, such as a hippocampal neuron (e.g., a dentate granule cell (DGC) or a glutamatergic pyramidal neuron). In other embodiments, including those in which the cell is a neuron, the cell is a human cell.

In another aspect, the disclosure features a vector comprising the polynucleotide of any one of the foregoing aspects and embodiments. In some embodiments, the vector is replication-defective. In some embodiments, the vector is a mammalian, insect, bacterial, or viral vector. In some embodiments, the vector is an expression vector. In some embodiments, the viral vector is selected from the group consisting of an adeno-associated virus (AAV), retrovirus, adenovirus, parvovirus, coronavirus, negative strand RNA viruses, orthomyxovirus, rhabdovirus, paramyxovirus, positive strand RNA viruses, picornavirus, alphavirus, a double stranded DNA virus, herpesvirus, Epstein-Barr virus, cytomegalovirus, fowlpox virus, and canarypox virus. In some embodiments, the vector is an AAV vector. In some embodiments, the AAV vector is an AAV5, AAV9, or AAVrh10 vector.

In another aspect, the disclosure features an expression cassette comprising a polynucleotide that encodes or comprises a polynucleotide corresponding to a stem-loop sequence of the first aspect of the disclosure, such as a stem-loop region having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of any one of SEQ ID NOs: 1-15, 226-229, and 238-241. In some embodiments, the stem-loop region has at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 4. In some embodiments, the stem-loop region has the nucleic acid sequence of SEQ ID NO: 4. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 135. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 258. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 259. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 260. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 261. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 256. In some embodiments, the expression cassette comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 257.

In another aspect, the disclosure provides an expression cassette comprising a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of any one of SEQ ID NOs: 46-62.

In another aspect, the disclosure provides an expression cassette comprising a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of any one of SEQ ID NOs: 97-108.

In another aspect, the disclosure provides an expression cassette comprising a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of any one of SEQ ID NOs: 133-138. In some embodiments, the stem-loop sequence has at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of SEQ ID NO: 135. In some embodiments, the stem-loop sequence has the nucleic acid sequence of SEQ ID NO: 135.

In some embodiments, the expression cassette comprises a 5′ flanking region, a loop region, and a 3′ flanking region. In some embodiments, the 5′ flanking region comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 217, 220, or 223. In some embodiments, the 3′ flanking region comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 218, 221, or 224. In some embodiments, the 5′ flanking region comprises a 5′ spacer sequence and a 5′ flanking sequence. In some embodiments, the 3′ flanking region comprises a 3′ spacer sequence and a 3′ flanking sequence. In some embodiments, the loop region comprises a microRNA loop sequence that is a E-miR-30, miR-218-1, or E-miR-124-3 sequence. In some embodiments, the microRNA loop sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 219, 222, or 225. In some embodiments, the microRNA loop sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 222. In some embodiments, the microRNA loop sequence comprises a polynucleotide having the nucleic acid sequence of SEQ ID NO: 222.

In some embodiments, the expression cassette comprises a Synapsin (hSyn) promoter or Calcium/Calmodulin Dependent Protein Kinase II (CaMKII) promoter. In some embodiments, the expression cassette comprises a constitutive promoter containing cytomegalovirus enhancer (e.g., CAG or CBA), U6, H1, or 7SK promoter.

In another aspect, the disclosure provides an expression cassette comprising, from 5′ to 3′: (a) a first promoter sequence; (b) a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of any one of SEQ ID NOs: 1-15, 46-62, 97-108, 133-138, 226-229, or 238-241; (c) optionally, a second promoter sequence; and (d) a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to a nucleic acid sequence of any one of SEQ ID NOs: 1-15, 46-62, 97-108, 133-138, 226-229, or 238-241. In some embodiments, the expression cassette comprises, from 5′ to 3′; (a) a first promoter sequence; (b) a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 4; (c) optionally, a second promoter sequence; and (d) a polynucleotide comprising a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 135. In some embodiments, the expression cassette comprises, from 5′ to 3′; (a) a first promotor sequence; (b) a polynucleotide comprising a stem-loop sequence having the nucleic acid sequence of SEQ ID NO: 4; (c) optionally, a second promoter sequence; and (d) a polynucleotide comprising a stem-loop sequence having the nucleic acid sequence of SEQ ID NO: 135. In some embodiments, the expression cassette comprises a sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 258.

In some embodiments, the polynucleotide comprising a stem-loop sequence having a nucleic acid sequence of any one of SEQ ID NOs: 1-15, 46-62, 97-108, 133-138, 226-229, or 238-241 or a variant thereof having at least 85% (e.g., at least 86%, 87%, 88%, 89%, 90%, 91%, 92%, 93%, 94%, 95%, 96%, 97%, 98%, 99%, or more (e.g., 100%)) sequence identity thereto comprises a passenger sequence which is complementary or substantially complementary to a guide sequence, wherein the passenger sequence is located 5′ or 3′ relative to a guide sequence. In some embodiments, the polynucleotide comprising a stem-loop sequence having a nucleic acid sequence of any one of SEQ ID NOs: 1-15, 46-62, 97-108, 133-138, 226-229, or 238-241 or a variant thereof having at least 85% (e.g., at least 86%, 87%, 88%, 89%, 90%, 91%, 92%, 93%, 94%, 95%, 96%, 97%, 98%, 99%, or more (e.g., 100%)) sequence identity thereto comprises a 5′ flanking region located 5′ relative to a guide sequence. In some embodiments, the polynucleotide comprising a stem-loop sequence having a nucleic acid sequence of any one of SEQ ID NOs: 1-15, 46-62, 97-108, 133-138, 226-229, or 238-241 or a variant thereof having at least 85% (e.g., at least 86%, 87%, 88%, 89%, 90%, 91%, 92%, 93%, 94%, 95%, 96%, 97%, 98%, 99%, or more (e.g., 100%)) sequence identity thereto comprises a 3′ flanking region located 3′ relative to the guide sequence. In some embodiments, the polynucleotide comprising a stem-loop sequence having a nucleic acid sequence of any one of SEQ ID NOs: 1-15, 46-62, 97-108, 133-138, 226-229, or 238-241 or a variant thereof having at least 85% (e.g., at least 86%, 87%, 88%, 89%, 90%, 91%, 92%, 93%, 94%, 95%, 96%, 97%, 98%, 99%, or more (e.g., 100%)) sequence identity thereto comprises a loop region located between the guide sequence and the passenger sequence, wherein the loop region comprises a microRNA loop sequence.

In some embodiments, the first promoter and/or, optionally, the second promoter is selected from the group consisting of an hSyn promoter or CaMKII promoter. The first and/or second promoter could also be selected from a constitutive promoter containing cytomegalovirus enhancer (e.g., CAG or CBA), U6, H1, and 7SK promotor.

In some embodiments, the 5′ flanking region comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 217, 220, or 223. In some embodiments, the 3′ flanking region comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 218, 221, or 224. In some embodiments, the microRNA loop sequence is a E-miR-30, miR-218-1, or E-miR-124-3 sequence. In some embodiments, the microRNA loop sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 219, 222, or 225. In some embodiments, the microRNA loop sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 222. In some embodiments, the microRNA loop sequence comprises a polynucleotide having the nucleic acid sequence of SEQ ID NO: 222.

In some embodiments, the expression cassette comprises a 5′-inverted terminal repeat (ITR) sequence on the 5′ end of said expression cassette and a 3′-ITR sequence on the 3′ end of said expression cassette. In some embodiments, the 5′-ITR and 3′ ITR sequences are AAV2 5′-ITR and 3′ ITR sequences. In some embodiments, the 5′-ITR sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 208 or SEQ ID NO: 209. In some embodiments, the 5′-ITR sequence comprises a polynucleotide having the nucleic acid sequence of SEQ ID NO: 208 or SEQ ID NO: 209. In some embodiments, the 5′-ITR sequence comprises a polynucleotide having the nucleic acid sequence of SEQ ID NO: 208. In some embodiments, the 3′-ITR sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence SEQ ID NO: 210, SEQ ID NO: 211, or SEQ ID NO: 212. In some embodiments, the 3′-ITR sequence comprises a polynucleotide having the nucleic acid sequence of SEQ ID NO: 210, SEQ ID NO: 211, or SEQ ID NO: 212. In some embodiments, the 3′-ITR sequence comprises a polynucleotide having the nucleic acid sequence of SEQ ID NO: 212.

In some embodiments, the expression cassette further includes an enhancer sequence. In an embodiment, the enhancer sequence can be located in an expression cassette or vector disclosed herein to augment the activity of a promoter in the expression cassette or vector (e.g., the enhancer sequence can be located 5′ to the promoter sequence in an expression cassette or vector described herein). In some embodiments, the enhancer sequence includes a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 207. In some embodiments, the enhancer sequence includes a polynucleotide having the nucleic acid sequence of SEQ ID NO: 207

In some embodiments, the expression cassette further includes an intron sequence. In an embodiment, the intron sequence can be located in an expression cassette or vector to improve expression of an inhibitory polynucleotide (e.g., an ASO (e.g., an miRNA sequence; e.g., an intron can be placed between a promoter and a nucleic acid sequence of an inhibitory polynucleotide). In some embodiments, the intron sequence is located between two or more inhibitory polynucleotide sequences (e.g., two or more miRNA sequences) described herein. In some embodiments, the intron sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 205 or SEQ ID NO: 206. In some embodiments, the intron sequence comprises a polynucleotide having the nucleic acid sequence of SEQ ID NO: 205 or SEQ ID NO: 206.

In some embodiments, the expression cassette further includes one or more (e.g., two, three, four, or five) polyadenylation signal sequences (e.g., to improve nuclear export, translation, and stability of an inhibitory polynucleotide of an expression cassette or vector disclosed herein). The polyadenylation signal sequence can be located 3′ to the terminal inhibitor polynucleotide sequence (e.g., an ASO sequence, such as a miRNA sequence, disclosed herein) and/or 5′ to the 3′ ITR sequence. In some embodiments, the polyadenylation signal sequence is a rabbit beta-globin (RBG) polyadenylation signal. In some embodiments, the RBG polyadenylation signal comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 213, SEQ ID NO: 214, or SEQ ID NO: 215. In some embodiments, the RBG polyadenylation signal comprises a polynucleotide having the nucleic acid sequence of SEQ ID NO: 213, SEQ ID NO: 214, or SEQ ID NO: 215. In some embodiments, the polyadenylation signal sequence is a bovine growth hormone (BGH) polyadenylation signal sequence. In some embodiments, the BGH polyadenylation signal sequence comprises a polynucleotide having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of SEQ ID NO: 216. In some embodiments, the BGH polyadenylation signal sequence comprises a polynucleotide having the nucleic acid sequence of SEQ ID NO: 216.

In some embodiments, the expression cassette further comprises one or more (e.g., two, three, four, or five) stuffer sequences. In some embodiments, the one or more (e.g., two, three, four, or five) stuffer sequences are positioned at the 3′ end of the expression cassette (e.g., between the polyadenylation sequence and the 3′ ITR sequence). In some embodiments, the one or more (e.g., two, three, four, or five) stuffer sequences have at least 85% (e.g., at least 86%, 90%, 95%, 96%, 97%, 98%, 99%, or more (e.g., 100%)) sequence identity to the nucleic acid sequence of SEQ ID NO: 250. In some embodiments, the one or more (e.g., two, three, four, or five) stuffer sequences have at least 90% (e.g., at least 91%, 95%, 96%, 97%, 98%, 99%, or more (e.g., 100%)) sequence identity to the nucleic acid sequence of SEQ ID NO: 250. In some embodiments, the one or more (e.g., two, three, four, or five) stuffer sequences have at least 95% (e.g., at least 96%, 97%, 98%, 99%, or more (e.g., 100%)) sequence identity to the nucleic acid sequence of SEQ ID NO: 250. In some embodiments, the one or more (e.g., two, three, four, or five) stuffer sequences have at least 99% sequence identity to the nucleic acid sequence of SEQ ID NO: 250. In some embodiments, the one or more (e.g., two, three, four, or five) stuffer sequences have the nucleic acid sequence of SEQ ID NO: 250. In some embodiments, the one or more (e.g., two, three, four, or five) stuffer sequences have at least 85% (e.g., at least 86%, 90%, 95%, 96%, 97%, 98%, 99%, or more (e.g., 100%)) sequence identity to the nucleic acid sequence of SEQ ID NO: 251. In some embodiments, the one or more (e.g., two, three, four, or five) stuffer sequences have at least 90% (e.g., at least 91%, 95%, 96%, 97%, 98%, 99%, or more (e.g., 100%)) sequence identity to the nucleic acid sequence of SEQ ID NO: 251. In some embodiments, the one or more (e.g., two, three, four, or five) stuffer sequences have at least 95% (e.g., at least 96%, 97%, 98%, 99%, or more (e.g., 100%)) sequence identity to the nucleic acid sequence of SEQ ID NO: 251. In some embodiments, the one or more (e.g., two, three, four, or five) stuffer sequences have at least 99% sequence identity to the nucleic acid sequence of SEQ ID NO: 251. In some embodiments, the one or more (e.g., two, three, four, or five) stuffer sequences have the nucleic acid sequence of SEQ ID NO: 251.

In some embodiments, the expression cassette of any of the foregoing aspects and embodiments includes, from 5′ to 3′: (a) a 5′ ITR sequence; (b) optionally, an enhancer sequence; (c) a first promoter sequence; (d) optionally, an intron sequence; (c) a polynucleotide comprising a stem-loop sequence; (f) optionally, a second promoter sequence; (g) optionally, a polynucleotide comprising a stem-loop sequence; (h) a polyadenylation signal sequence, such as a RBG polyadenylation signal sequence; (i) one or more stuffer sequences; and (j) a 3′ ITR.

In some embodiments, the expression cassette of the foregoing aspects and embodiments includes at least 70% (e.g., at least 75%, 80%, 85%, 86%, 87%, 88%, 89%, 90%, 91%, 92%, 93%, 94%, 95%, 96%, 97%, 98%, 99%, or more (e.g., 100%)) sequence identity to the sequence of any one of SEQ ID NOs: 252-261. In some embodiments, the expression cassette of the foregoing aspects and embodiments includes at least 70% (e.g., at least 75%, 80%, 85%, 86%, 87%, 88%, 89%, 90%, 91%, 92%, 93%, 94%, 95%, 96%, 97%, 98%, 99%, or more (e.g., 100%)) sequence identity to the sequence of any one of SEQ ID NOs: 256 and 258-261. In some embodiments, the expression cassette of the foregoing aspects and embodiments includes at least 70% (e.g., at least 75%, 80%, 85%, 86%, 87%, 88%, 89%, 90%, 91%, 92%, 93%, 94%, 95%, 96%, 97%, 98%, 99%, or more (e.g., 100%)) sequence identity to the sequence of SEQ ID NO: 261. In some embodiments, the expression cassette has the nucleic acid sequence of SEQ ID NO: 256.

In some embodiments, the expression cassette of the foregoing aspects and embodiments is incorporated into the vector of the foregoing aspects and embodiments. In some embodiments, the vector is a replication-defective vector. In some embodiments, the vector is a mammalian, insect, bacterial, or viral vector. In some embodiments, the vector is an expression vector. In some embodiments, the viral vector is selected from the group consisting of an adeno-associated virus (AAV), retrovirus, adenovirus, parvovirus, coronavirus, negative strand RNA viruses, orthomyxovirus, rhabdovirus, paramyxovirus, positive strand RNA viruses, picornavirus, alphavirus, a double stranded DNA virus, herpesvirus, Epstein-Barr virus, cytomegalovirus, fowlpox virus, and canarypox virus. In some embodiments, the vector is an AAV vector. In some embodiments, the AAV vector is an AAV5, AAV9, or AAVrh10 vector.

In another aspect, the disclosure provides a method of inhibiting Grik2 expression in a cell, the method including contacting the cell with at least one polynucleotide of the foregoing aspects and embodiments, the vector of the foregoing aspect and embodiments, or the expression cassette of the foregoing aspects and embodiments.

In some embodiments, the polynucleotide specifically hybridizes to a Grik2 mRNA and inhibits or reduces the expression of Grik2 in the cell (as is discussed further in the disclosure). In some embodiments, the method reduces a level of Grik2 in the cell by at least 10%, at least 15%, at least 20%, at least 25%, at least 30%, at least 40%, at least 45%, at least 50%, at least 55%, at least 60%, at least 65%, at least 70%, or at least 75%. In some embodiments, the method reduces a level of GluK2 protein in the cell. In some embodiments, the method reduces a level of GluK2 protein in the cell by at least 10%, at least 15%, at least 20%, at least 25%, at least 30%, at least 35%, at least 40%, at least 45%, at least 50%, at least 55%, at least 60%, at least 65%, at least 70%, or at least 75%.

In some embodiments, the cell is a human cell. In some embodiments, the cell is a neuron (e.g., a human neuron). In some embodiments, the neuron is a hippocampal neuron (e.g., a human hippocampal neuron). In some embodiments, the hippocampal neuron is a DGC (e.g., a human DGC or pyramidal neuron). In some embodiments, the DGC includes recurrent mossy fiber axon. The cell may also be a neuronal cell derived from an induced pluripotent stem cell (iPSC), such as an iPSC-derived glutamatergic neuron that expresses Grik2.

In another aspect, the disclosure provides a method of treating or ameliorating a disorder in a subject in need thereof, the method including administering to the subject at least one polynucleotide of the foregoing aspects and embodiments, the vector of the foregoing aspects and embodiment, or the expression cassette of the foregoing aspects and embodiments.

In some embodiments, the disorder is an epilepsy. In some embodiments, the epilepsy is a temporal lobe epilepsy (TLE), chronic epilepsy, and/or a refractory epilepsy. In some embodiments, the epilepsy is a TLE. In some embodiments, the TLE is a lateral TLE (lTLE), such as unilateral TLE and/or bilateral TLE. In some embodiments, the TLE is a mesial TLE (mTLE).

In some embodiments, the subject is a human.

In another aspect, the disclosure provides a pharmaceutical composition including the polynucleotide of the foregoing aspects and embodiments, the vector of the foregoing aspects and embodiments, or the expression cassette of the foregoing aspects and embodiments, and a pharmaceutically acceptable carrier, diluent, or excipient.

In another aspect, the disclosure provides a kit including the pharmaceutical composition of the foregoing aspect and a package insert. In some embodiments, the package insert includes instructions for use of the pharmaceutical composition in the method of the foregoing aspects and embodiments.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments and are not intended to limit the claimed technology. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

In this application, unless otherwise clear from context, (i) the term “a” may be understood to mean “at least one”; (ii) the term “or” may be understood to mean “and/or”; and (iii) the terms “including” and “comprising” may be understood to encompass itemized components or steps whether presented by themselves or together with one or more additional components or steps.

The term “about” refers to an amount that is +10% of the recited value and may be±5% of the recited value or ±2% of the recited value.

The terms “3′ untranslated region” and “3′ UTR” refer to the region 3′ with respect to the stop codon of an mRNA molecule (e.g., a Grik2 mRNA). The 3′ UTR is not translated into protein, but includes regulatory sequences important for polyadenylation, localization, stabilization, and/or translation efficiency of an mRNA transcript. Regulatory sequences in the 3′ UTR may include enhancers, silencers, AU-rich elements, poly-A tails, terminators, and microRNA recognition sequences. The terms “3′ untranslated region” and “3′ UTR” may also refer to the corresponding regions of the gene encoding the mRNA molecule.

The term “5′ untranslated region” and “5′ UTR” refer to a region of an mRNA molecule (e.g., a Grik2 mRNA) that is 5′ with respect to the start codon. This region is important for the regulation of translation initiation. The 5′ UTR can be entirely untranslated or may have some of its regions translated in some organisms. The transcription start site marks the start of the 5′ UTR and ends one nucleotide before the start codon. In eukaryotes, the 5′ UTR includes a Kozak consensus sequence harboring the start codon. The 5′ UTR may include cis-acting regulatory elements also known as upstream open reading frames that are important for the regulation of translation. This region may also harbor upstream AUG codons and termination codons. Given its high GC content, the 5′ UTR may form secondary structures, such as hairpin loops that play a role in the regulation of translation.

The term “administration” refers to providing or giving a subject a therapeutic agent (e.g., an inhibitory polynucleotide that binds to and inhibits the expression of a Grik2 mRNA, or a vector encoding the same, as is disclosed herein), by any effective route. Exemplary routes of administration are described herein and below (e.g., intracerebroventricular injection, intrathecal injection, intraparenchymal injection, intravenous injection, and stereotactic injection).

The term “adeno-associated viral vector” or “AAV vector” refers to a vector derived from an adeno-associated virus serotype, including without limitation, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16. AAV vectors can have one or more of the AAV wild-type genes deleted in whole or part, e.g., the rep and/or cap genes, but retain functional flanking ITR sequences. Functional ITR sequences promote the rescue, replication, and packaging of the AAV virion. Thus, an AAV vector is defined heroin to include at least those sequences required in cis for replication and packaging (e.g., functional ITRs) of the virus. ITRs do not need to be the wild-type polynucleotide sequences and may be altered, e.g., by the insertion, deletion, or substitution of nucleotides, so long as the sequences provide for functional rescue, replication, and packaging. AAV expression vectors are constructed using known techniques to at least provide as operatively linked components in the direction of transcription, control elements including a transcriptional initiation region, the DNA of interest (e.g., a polynucleotide encoding an inhibitory RNA agent of the disclosure) and a transcriptional termination region.

The terms “adeno-associated virus inverted terminal repeats” and “AAV ITRs” refer to art-recognized regions flanking each end of the AAV genome which function together in cis as origins of DNA replication and as packaging signals for the virus. AAV ITRs, together with the AAV rep coding region, provide for the efficient excision and integration of a polynucleotide sequence interposed between two flanking ITRs into a mammalian genome. The polynucleotide sequences of AAV ITR regions are known. As used herein, an “AAV ITR” does not necessarily include the wild-type polynucleotide sequence, which may be altered, e.g., by the insertion, deletion or substitution of nucleotides. Additionally, the AAV ITR may be derived from any of several AAV serotypes, including without limitation AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16, among others. Furthermore, 5′ and 3′ ITRs which flank a selected polynucleotide sequence in an AAV vector need not be identical or derived from the same AAV serotype or isolate, so long as they function as intended, e.g., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV. RHM4-1, AAV.hu37, AAV.Anc80, AAV.Anc80L65, AAV.7m8, AAV.PHP.B, AAV. PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16, among others.

The terms “antisense oligonucleotide” and “ASO” refer to an inhibitory polynucleotide capable of hybridizing through complementary base-pairing with a target mRNA molecule (e.g., a Grik2 mRNA) and inhibiting its expression through mRNA destabilization and degradation, or inhibition of translation.

The term “cDNA” refers to a nucleic acid sequence that is a DNA equivalent of an mRNA sequence (i.e., having uridine substituted with thymidine). Generally, the terms cDNA and mRNA may be used interchangeably in reference to a particular gene (e.g., Grik2 gene) as one of skill in the art would understand that a cDNA sequence is the same as the mRNA sequence with the exception that uridines are read as thymidines. Furthermore, in instances where a reference is made to a DNA sequence encoding the antisense constructs disclosed herein or to an RNA transcript encoded by the same, unless indicated otherwise by context, the terms “DNA” and “RNA” may be used interchangeably to refer to the antisense sequences. Moreover, certain DNA sequences disclosed herein (e.g., those encoding Grik2 antisense sequences) may contain RNA nucleotides, in which case, the sequence as a whole can be referred to as a “DNA sequence” or an “RNA sequence.”

The term “coding sequence” corresponds to a nucleic acid sequence of an mRNA molecule that encodes a protein or a portion thereof. Relatedly, a “non-coding sequence” corresponds to a nucleic acid sequence of an mRNA molecule that does not encode a protein or a portion thereof. Non-limiting examples of non-coding sequences include 5′ and 3′ untranslated regions (UTRs), introns, polyA tail, promoters, enhancers, terminators, and other cis-regulatory sequences.

The term “complementary,” when used to describe a first nucleotide or nucleoside sequence in relation to a second nucleotide or nucleoside sequence, refers to the ability of a polynucleotide including the first nucleotide sequence to hybridize and form a duplex structure under certain conditions with the polynucleotide including the second nucleotide sequence. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. Methods of determining the set of conditions most appropriate for a test of complementarity of two sequences in accordance with the ultimate application of the hybridized nucleotides or nucleosides are well-known in the art.

“Complementary” sequences, as used herein, can also include, or be formed entirely from, non-Watson-Crick base pairs and/or base pairs formed from non-natural and alternative nucleotides, in so far as the above requirements with respect to their ability to hybridize are fulfilled. Such non-Watson-Crick base pairs include, but are not limited to, G: U Wobble or Hoogstein base pairing. Complementary sequences between a polynucleotide and a target sequence as described herein, include base-pairing of the polynucleotide including a first nucleotide sequence to a polynucleotide including a second nucleotide sequence over the entire length of one or both nucleotide sequences. Such sequences can be referred to as “fully complementary” with respect to each other herein. Where a first sequence is referred to as “substantially complementary” with respect to a second sequence herein, the two sequences can be fully complementary or they can form one or more, but generally no more than 10, 9, 8, 7, 6, 5, 4, 3, 2, or 1 mismatched base pairs upon hybridization for a duplex of up to 30 base pairs, while retaining the ability to hybridize under the conditions most relevant to their ultimate application, e.g., binding to and inhibiting the expression of an mRNA, such as a Grik2 mRNA. For example, a polynucleotide is complementary to at least a part of the mRNA of interest if the sequence is substantially complementary to a non-interrupted portion of the mRNA of interest.

The term “region of complementarity” refers to the region of the inhibitory polynucleotide that is substantially complementary to all or a portion of a gene, primary transcript, a sequence (e.g., a target sequence), or processed mRNA, so as to interfere with expression of the endogenous gene (e.g., Grik2). Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- and/or 3′-terminus of the inhibitory polynucleotide.

The terms “conservative amino acid substitution”, “conservative substitution,” and “conservative mutation,” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Th | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

The phrase “contacting a cell with an inhibitory polynucleotide,” such as an inhibitory polynucleotide disclosed herein, includes contacting a cell by any possible means. Contacting a cell with an inhibitory polynucleotide includes contacting a cell in vitro with the inhibitory polynucleotide or contacting a cell in vivo with the inhibitory polynucleotide. Contacting a cell with an inhibitory polynucleotide may also refer to contacting the cell with a nucleic acid vector encoding the inhibitory polynucleotide or a pharmaceutical composition containing the same. The contacting may be done directly or indirectly. Thus, for example, the inhibitory polynucleotide may be put into physical contact with the cell by the individual performing the method, or alternatively, the inhibitory polynucleotide agent may be put into a situation that will permit or cause it to subsequently come into contact with the cell. Contacting a cell in vitro may be done, for example, by incubating the cell with the inhibitory polynucleotide. Contacting a cell in vivo may be done, for example, by injecting the inhibitory polynucleotide into or near the tissue where the cell is located, or by injecting the inhibitory polynucleotide agent into another area, e.g., the bloodstream or the subcutaneous space, such that the agent will subsequently reach the tissue where the cell to be contacted is located. Combinations of in vitro and in vivo methods of contacting are also possible. For example, a cell may also be contacted in vitro with an inhibitory polynucleotide and subsequently transplanted into a subject.

Contacting a cell with an inhibitory polynucleotide includes “introducing” or “delivering the inhibitory polynucleotide into the cell” by facilitating or effecting uptake or absorption into the cell. Absorption or uptake of an inhibitory polynucleotide or a nucleic acid vector encoding the same can occur through unaided diffusive or active cellular processes, or by auxiliary agents or devices. Introducing an inhibitory polynucleotide into a cell may be in vitro and/or in vivo. For example, for in vivo introduction, inhibitory polynucleotides can be injected into a tissue site or administered systemically. In vitro introduction into a cell includes methods known in the art such as electroporation and lipofection. In another example, an inhibitory polynucleotide can be introduced into a cell by transduction, such as by way of a viral vector encoding the inhibitory polynucleotide. The viral vector may undergo cellular processing (e.g., cellular internalization, capsid shedding, transcription of the inhibitory polynucleotide, and processing by Drosha and Dicer) in order to express the encoded inhibitory polynucleotide. Further approaches are described herein below and/or are known in the art.

The terms “disrupt expression of,” “inhibit expression of,” or “reduce the expression of,” with respect to a gene (e.g., Grik2), refers to preventing or reducing the formation of a functional gene product (e.g., a GluK2 protein). A gene product is functional if it fulfills its normal (wild-type) function(s). Disruption of the gene prevents or reduces the expression of a functional protein encoded by the gene. The disrupted gene may be disrupted by, e.g., an interfering RNA molecule (e.g., an ASO), such as those described herein.

The terms “effective amount,” “therapeutically effective amount,” and a “sufficient amount” of composition, vector construct, or viral vector described herein refer to a quantity sufficient to, when administered to the subject, including a mammal, for example a human, effect beneficial or desired results, including clinical results. As such, an “effective amount” or synonym thereof depends upon the context in which it is being applied. For example, in the context of treating temporal lobe epilepsy (TLE), it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder and its severity, the identity of the subject (e.g., age, sex, weight), host being treated, and/or, in the case of an epilepsy, the size (e.g., brain volume) of the epileptic focus, and the like, but can nevertheless be determined by according to methods well-known in the art. Also, as used herein, a “therapeutically effective amount” of a composition, vector construct, or viral vector of the disclosure is an amount which results in a beneficial or desired result in a subject as compared to a control. As defined herein, a therapeutically effective amount of a composition, vector construct, viral vector, or cell of the disclosure may be readily determined by methods known in the art. Dosage regime may be adjusted to provide the optimum therapeutic response.

The term “epilepsy” refers to one or more neurological disorders that clinically present with recurrent epileptic seizures. Epilepsy can be classified according the electroclinical syndromes following the Classification and Terminology of the International League Against Epilepsy (ILAE; Berg et al., 2010). These syndromes can be categorized by age at onset, distinctive constellations (surgical syndromes), and structural-metabolic causes, such as: (A) age at onset: (i) neonatal period includes benign familial neonatal epilepsy (BFNE), early myoclonic encephalopathy (EME), Ohtahara syndrome; (ii) infancy period includes epilepsy of infancy with migrating focal seizures, West syndrome, myoclonic epilepsy in infancy (MEI), benign infantile epilepsy, benign familial infantile epilepsy, Dravet syndrome, myoclonic encephalopathy in nonprogressive disorders; (iii) childhood period includes febrile seizures plus (FS+), Panayiotopoulos syndrome, epilepsy with myoclonic atonic (previously astatic) seizures, benign epilepsy with centrotemporal spikes (BECTS), autosomal-dominant nocturnal frontal lobe epilepsy (ADNFLE), late onset childhood occipital epilepsy (Gastaut type), epilepsy with myoclonic absences, Lennox-Gastaut syndrome, epileptic encephalopathy with continuous spike-and-wave during sleep (CSWS), Landau-Kleffner syndrome (LKS), childhood absence epilepsy (CAE); (iv) adolescence-adult period includes juvenile absence epilepsy (JAE) juvenile myoclonic epilepsy (JME), epilepsy with generalized tonic-clonic seizures alone, progressive myoclonus epilepsies (PME), autosomal dominant epilepsy with auditory features (ADEAF), other familial temporal lobe epilepsies; (v) variable age onset includes familial focal epilepsy with variable foci (childhood to adult), reflex epilepsies; (B) distinctive constellations (surgical syndromes) include mesial temporal lobe epilepsy (MTLE), Rasmussen syndrome, gelastic seizures with hypothalamic hamartoma, hemiconvulsion-hemiplegia-epilepsy; (C) epilepsies attributed to and organized by structural-metabolic causes include malformations of cortical development (hemimegalencephaly, heterotopias, etc.), neurocutaneous syndromes (tuberous sclerosis complex and Sturge-Weber), tumor, infection, trauma, angioma, perinatal insults, and stroke. The term “refractory epilepsy” refers to an epilepsy which is refractory to pharmaceutical treatment; that is to say that current pharmaceutical treatment does not allow an effective treatment of patients' disease (see for example Dario J. Englot et al., 2013).

The term “exon” refers to a region within the coding region of a gone (e.g., a Grik2 gone), the nucleotide sequence of which determines the amino acid sequence of the corresponding protein. The term “exon” also refers to the corresponding region of the RNA transcribed from a gene. Exons are transcribed into pre-mRNA and may be included in the mature mRNA depending on the alternative splicing of the gene. Exons that are included in the mature mRNA following processing are translated into protein. The sequence of the exon determines the amino acid composition of the protein. Alternatively, exons that are included in the mature mRNA may be non-coding (e.g., exons that do not translate into protein).

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include mRNAs, which are modified by processes such as capping, polyadenylation, methylation, and editing, and proteins (e.g., GluK2) modified by, for example, methylation, acetylation, phosphorylation, ubiquitination, SUMOylation, ADP-ribosylation, myristoylation, and glycosylation.

The term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: a decrease or increase in the quantity or concentration of mRNA encoding a corresponding protein (as assessed, e.g., using RNA detection procedures described herein or known in the art, such as quantitative polymerase chain reaction (qPCR) and RNA seq techniques), a decrease or increase in the quantity or concentration of a corresponding protein (as assessed, e.g., using protein detection methods described herein or known in the art, such as enzyme-linked immunosorbent assays (ELISA), among others), and/or a decrease or increase in the activity of a corresponding protein (e.g., in the case of an ion channel, as assessed using electrophysiological methods described herein or known in the art) in a sample obtained from the subject.

The term “GluK2”, also known as “GluR6”, “GRIK2”, “MRT6”, “EAA4”, or “GluK6”, refers to the glutamate ionotropic receptor kainato type subunit 2 protein, as named in the currently used IUPHAR nomenclature (Collingridge, G. L., Olsen, R. W., Peters, J., Spedding, M., 2009. A nomenclature for ligand-gated ion channels. Neuropharmacology 56, 2-5). The terms “GluK2-containing KAR,” “GluK2 receptor,” “GluK2 protein,” and “GluK2 subunit” may be used interchangeably throughout and generally refer to the protein encoded by or expressed by a Grik2 gene.

The terms “guide strand” and “guide sequence” refer to a component of a stem-loop RNA structure (e.g., an shRNA or microRNA) positioned on either the 5′ or the 3′ stem-loop arm of the stem-loop structure, wherein the guide strand/sequence includes a Grik2 mRNA antisense sequence (e.g., any one of SEQ ID NOs: 16-30, 63-79, 109-120, 139-144, 230-233, and 242-245 or a variant thereof having at least 85% (e.g., at least 86%, 90%, 95%, 96%, 97%, 98%, 99%, or more (e.g., 100%)) sequence identity to the nucleic acid sequence of any one of SEQ ID NOs: 16-30, 63-79, 109-120, 139-144, 230-233, and 242-245) capable of binding to and inhibiting the expression of the Grik2 mRNA. The guide strand/sequence may also include additional sequences, such as, e.g., spacer or linker sequences. The guide sequence may be complementary or substantially complementary (e.g., having no more than 7, 6, 5, 4, 3, 2, or 1 mismatches) to a passenger strand/sequence of the stem-loop RNA structure.

The term “ionotropic glutamate receptors” include members of the NMDA (N-methyl-D-aspartate), AMPA (a-amino-3-hydroxy-5-methyl-4-isoxazoleproprionic acid) and kainate receptor (KAR) classes. Functional KARs can be assembled into tetrameric assemblies from the homomeric or heteromeric combination of five subunits named GluK1, GluK2, GluK3, GluK4 and GluK5 subunits (Reiner et al., 2012). The targets of the disclosure are, in some instances, KAR complexes composed of GluK2 and GluK5. Inhibiting the expression of Grik2 gene is sufficient to abolish GluK2/GluK5 kainate receptor function, given the observation that the GluK5 subunit by itself does not form functional homomeric channels.

An “inhibitor of expression” refers to an agent (e.g., an inhibitory RNA agent of the disclosure) that has a biological effect to inhibit or decrease the expression of a gene, e.g., the Grik2 gene. Inhibiting expression of a gene, e.g., the Grik2 gene, will typically result in a decrease or even abolition of the gene product (protein, e.g., GluK2 protein) in target cells or tissues, although various levels of inhibition may be achieved. Inhibiting or decreasing expression is typically referred to as knockdown.

The term “isolated polynucleotide” refers to an isolated molecule including two or more covalently linked nucleotides. Such covalently linked nucleotides may also be referred to as nucleic acid molecules. Generally, an “isolated” polynucleotide refers to a polynucleotide that is man-made, chemically synthesized, purified, and/or heterologous with respect to the nucleic acid sequence from which it is obtained.

The term “microRNA” refers to a short (e.g., typically ˜22 nucleotide) sequence of non-coding RNA that regulates mRNA translation and thus influences target protein abundance. Some microRNAs are transcribed from a single, monocistronic gene, while others are transcribed as part of polycistronic gene clusters. The structure of a microRNA may include 5′ and 3′ flanking sequences, hairpin sequences including stem and loop sequences. During processing within the cell, an immature microRNA is truncated by Drosha, which cleaves off the 5′ and 3′ flanking sequences. Subsequently, the microRNA molecule is translocated from the nucleus to the cytoplasm, where it undergoes cleavage of the loop region by Dicer. The biological action of microRNAs is exerted at the level of translational regulation through binding to regions of the mRNA molecule, typically the 3′ untranslated region, and leading to the cleavage, degradation, destabilization, and/or less efficient translation of the mRNA. Binding of the microRNA to its target is generally mediated by a short (e.g., 6-8 nucleotide) “seed region/sequence” within the hairpin sequence of the microRNA. Throughout the disclosure, the term siRNA may include its equivalent miRNA, such that the miRNA encompasses the same bases that have homology to the target (e.g., in the seed region) as its equivalent siRNA. As described herein, a microRNA may be a non-naturally occurring microRNA, such as a microRNA having one or more heterologous nucleic acid sequences.

The term “nucleotide” is defined as a modified or naturally occurring deoxyribonucleotide or ribonucleotide. Nucleotides typically include purines and pyrimidines, which include thymidine, cytidine, guanosine, adenosine and uridine. The term “inhibitory polynucleotide” as used herein is defined as an oligomer of the nucleotides defined above or modified nucleotides disclosed herein. The term “inhibitory polynucleotide” refers to a nucleic acid sequence, 3′-5′ or 5′-3′ oriented, which may be single- or double-stranded. The inhibitory polynucleotide used in the context of the disclosure may in particular be DNA or RNA. The term may also include an “inhibitory polynucleotide analog,” which refers to an inhibitory polynucleotide having, e.g., (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Inhibitory polynucleotide analogs support bases capable of hydrogen bonding by Watson-Crick base pairing to standard polynucleotide bases, where the analog backbone presents the bases in a manner to permit such hydrogen bonding in a sequence-specific fashion between the inhibitory polynucleotide analog molecule and bases in a standard polynucleotide {e.g., single-stranded RNA or single-stranded DNA). Particularly, analogs are those having a substantially uncharged, phosphorus containing backbone. A substantially uncharged, phosphorus containing backbone in an inhibitory polynucleotide analog is one in which a majority of the subunit linkages, e.g., between 50-100%, typically at least 60% to 100% or 75% or 80% of its linkages, are uncharged, and contain a single phosphorous atom. Furthermore, the term “inhibitory polynucleotide” can include an inhibitory polynucleotide sequence that is inverted relative to its normal orientation for transcription and so corresponds to an RNA or DNA sequence that is complementary to a target gene mRNA molecule expressed within the host cell. An antisense guide strand may be constructed in a number of different ways, provided that it is capable of interfering with the expression of a target gene. For example, the antisense guide strand can be constructed by reverse-complementing the coding region (or a portion thereof) of the target gone relative to its normal orientation for transcription to allow the transcription of its complement, (e.g., RNAs encoded by the antisense and sense gene may be complementary). The inhibitory polynucleotide need not have the same intron or exon pattern as the target gene, and noncoding segments of the target gene may be equally effective in achieving antisense suppression of target gene expression as coding segments such as an ASO. In some cases, the inhibitory RNA has the same exon pattern as the target gene.

The inhibitory polynucleotide may be of any length that permits targeting and hybridization to a Grik2 mRNA (e.g., the inhibitory polynucleotide is perfectly, or substantially complementary to at least a region of a Grik2 mRNA), and may range from about 10-50 base pairs in length, e.g., about 15-50 base pairs in length or about 18-50 base pairs in length, for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the disclosure.

The terms “passenger strand” and “passenger sequence” refer to a component of a stem-loop RNA structure (e.g., an shRNA or microRNA) positioned on either the 5′ or the 3′ stem-loop arm of the stem-loop structure that includes a sequence complementary or substantially complementary (e.g., having no more than 7, 6, 5, 4, 3, 2, or 1 mismatches to Grik2 mRNA antisense sequence (e.g., any one of SEQ ID NOs: 16-30, 63-79, 109-120, 139-144, 230-233, and 242-245 or a variant thereof having at least 85% (e.g., at least 85%, 90%, 95%, 96%, 97%, 98%, 99%, or more (e.g., 100%)) sequence identity to the nucleic acid sequence of any one of SEQ ID NO: 16-30, 63-79, 109-120, 139-144, 230-233, and 242-245). The passenger strand/sequence may also include additional sequences, such as, e.g., spacer or linker sequences. The passenger sequence may be complementary or substantially complementary to a guide strand/sequence of the stem-loop RNA structure.

The term “plasmid” refers to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids, which have a bacterial origin of replication, and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked. As used herein, “genes” refer to polynucleotides encoding proteins, microRNAs, siRNAs, shRNAs, shmiRNAs, and further containing one or more regulatory sequences (e.g., promoters, enhancers, introns, termination sequences, among others).

The term “promoter” refers to a recognition site on DNA that is bound by an RNA polymerase. The polymerase drives transcription of the polynucleotide. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of polynucleotides, vectors, and target cell types.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are well-known in the art, for example, using publicly available computer software such as BLAST, BLAST-2, or Megalign software. Using well-recognized and conventional methods, the appropriate parameters can be determined for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A. Regardless of the percent sequence identity between a candidate sequence and a reference polynucleotide or polypeptide sequence, the candidate sequence retains at least 20%, 30%, 40%, 50%, 60%, 70%, 75%, 80%, 85%, 90%, 95%, 97%, 99%, or 100% of the function (e.g., the ability to reduce a level of Grik2 mRNA, as defined herein, or a level of expression of GluK2 protein, as defined herein) of the reference polynucleotide or polypeptide sequence.

The term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

The term “pharmaceutical composition,” as used herein, represents a composition containing a compound (e.g., an inhibitory nucleic acid molecule (e.g., an RNA) or vector containing the same) described herein formulated with a pharmaceutically acceptable excipient, and in some instances may be manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup), topical administration (e.g., as a cream, gel, lotion, or ointment), intravenous administration (e.g., as a sterile solution free of particulate emboli and in a solvent system suitable for intravenous use), intrathecal injection, intracerebroventricular injections, intraparenchymal injection, or in any other pharmaceutically acceptable formulation.

A “pharmaceutically acceptable excipient,” refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The compounds (e.g., an inhibitory nucleic acid molecule (e.g., an RNA) and vectors containing the same) described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “regulatory sequence” includes promotors, enhancers and other expression control elements (e.g., polyadenylation signal sequences) that control the transcription or translation of a gene. Such regulatory sequences are described, for example, in Perdew et al., Regulation of Gene Expression (Humana Press, New York, NY, (2014)); incorporated herein by reference.

The terms “target” or “targeting” refers to the ability of an inhibitory nucleic acid molecule (e.g., an RNA), such as an inhibitory RNA agent described herein, to specifically bind through complementary base pairing to a Grik2 gene or mRNA encoding a GluK2 protein.

The terms “short interfering RNA” and “siRNA” refer to an inhibitory polynucleotide containing double stranded nucleic acid in which each strand comprises RNA, RNA analog(s) or RNA and DNA.

The siRNA molecule can include between 19 and 23 nucleotides (e.g., 21 nucleotides). The siRNA typically has 2 bp overhangs on the 3′ ends of each strand such that the duplex region in the siRNA comprises 17-21 nucleotides (e.g., 19 nucleotides). Typically, the antisense strand of the siRNA is sufficiently complementary with the target sequence of the target gene/RNA. siRNA molecules operate within the RNA interference pathway, leading to inhibition of mRNA expression by binding to a target mRNA (e.g., Grik2 mRNA) and degrading the mRNA through Dicer-mediated mRNA cleavage. Throughout the disclosure, the term siRNA is meant to include its equivalent miRNA, such that the miRNA encompasses the same bases that have homology to the target as its equivalent siRNA.

The terms “short hairpin RNA” and “shRNA” refer to an inhibitory polynucleotide containing single-stranded RNA of 50 to 100 nucleotides that forms a stem-loop structure in a cell, which contains a loop region of 5 to 30 nucleotides, and long complementary RNAs of 15 to 50 nucleotides at both sides of the loop region, which form a double-stranded stem by base pairing between the complementary RNA sequences; and, in some cases, an additional 1 to 500 nucleotides included before and after each complementary strand forming the stem. For example, shRNA generally requires specific sequences 3′ of the hairpin to terminate transcription by RNA polymerase. Such shRNAs generally bypass processing by Drosha due to their inclusion of short 5′ and 3′ flanking sequences. Other shRNAs, such as “shRNA-like microRNAs,” which are transcribed from RNA polymerase II, include longer 5′ and 3′ flanking sequences, and require processing in the nucleus by Drosha, after which the cleaved shRNA is exported from the nucleus to cytosol and further cleaved in the cytosol by Dicer. Like siRNA, shRNA binds to the target mRNA in a sequence specific manner, thereby cleaving and destroying the target mRNA, and thus suppressing expression of the target mRNA.

As used herein, the terms “specifically hybridizes” and “specifically binds” refer to a polynucleotide having a sufficient degree of complementarity between the polynucleotide and a target nucleic acid (e.g., a Grik2 mRNA) to induce a desired effect (e.g., reduction or inhibition of expression of GluK2 from a Grik2 mRNA), while exhibiting minimal or no effects on non-target nucleic acids. Specific hybridization or binding may occur under physiological conditions. For example, specific hybridization or binding occurs when the number of nucleobases in a polynucleotide (e.g., an antisense polynucleotide) that are complementary to the nucleobases at a corresponding target nucleic acid (e.g., an mRNA sequence) promotes annealing of the polynucleotide to the target nucleic acid but not to non-target nucleic acid (e.g., the complementarity corresponds to, e.g., a percent sequence identity of 80% or greater (e.g., 85%, 90%, 95%, 97%, 99%, or 100%) of a binding portion of a polynucleotide to the target nucleic acid). Those skilled in the art will understand that in such a situation, the nucleic acid sequence in the polynucleotide (e.g., an antisense oligomer) and the nucleic acid sequence in the target nucleic acid have a high degree of complementarity (e.g., at least about 80%, 85%, 90%, 95%, 97%, 99%, or 100% complementary, such as over a defined number of polynucleotides (e.g., about 7-22 nucleobases).

The terms “subject” and “patient” refer to an animal (e.g., a mammal, such as a human). A subject to be treated according to the methods described herein may be one who has been diagnosed with an epilepsy (e.g., TLE), or one at risk of developing this condition. Diagnosis may be performed by any method or technique known in the art. A subject to be treated according to the disclosure may have been subjected to standard tests or may have been identified, without examination, as one at risk due to the presence of one or more risk factors associated with the disease or condition.

The terms “temporal lobe epilepsy” or “TLE” refers to a chronic neurological condition characterized by chronic and recurrent seizures (epilepsy) which originate in the temporal lobe of the brain. This disease is different from acute seizures in naïve brain tissue since TLE is characterized by morpho-functional reorganization of neuronal networks and sprouting of recurrent mossy fibers from granule cells of the dentate gyrus of the hippocampus, whereas acute seizures in naïve tissue do not precipitate such circuit-specific reorganization. TLE may result from an emergence of an epileptogenic focus in one or both hemispheres of the brain.

The terms “transduction” and “transduce” refer to a method of introducing a nucleic acid material (e.g., a vector, such as a viral vector construct, or a part thereof) into a cell and subsequent expression of a polynucleotide encoded by the nucleic acid material (e.g., the vector construct or part thereof) in the cell.

The term “treatment” or “treat” refers to both prophylactic and preventive treatment as well as curative or disease modifying treatment, including treatment of a patient at risk of contracting the disease or suspected to have contracted the disease, as well as a patient who is ill or has been diagnosed as suffering from a disease or medical condition. Treatment also includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria (e.g., disease manifestation).

The term “vector” includes a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a prokaryotic or eukaryotic cell. Examples of such expression vectors are disclosed in, e.g., WO 1994/011026; incorporated herein by reference as it pertains to vectors suitable for the expression of a nucleic acid material of interest. Expression vectors suitable for use with the compositions and methods described herein contain a polynucleotide sequence as well as, e.g., additional sequence elements used for the expression of heterologous nucleic acid materials (e.g., an ASO) in a mammalian cell. Certain vectors that can be used for the expression of the inhibitory nucleic acid (e.g., RNA) agents described herein include plasmids that contain regulatory sequences, such as promoter and enhancer regions, which direct gene transcription. Other useful vectors for expression of inhibitory nucleic acid (e.g., RNA) agents disclosed herein contain polynucleotide sequences that enhance the rate of translation of these polynucleotides or improve the stability or nuclear export of the nucleic acid (e.g., RNA) that results from gene transcription. These sequence elements include, e.g., 5′ and 3′ untranslated regions, an IRES, and polyadenylation signal sequence site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin.

As used herein, the term “variant” refers to a polynucleotide, such as, e.g., an inhibitory polynucleotide sequence of the disclosure or a complement thereof (e.g., substantial or full complement thereof) which is obtained by rationally including one or more (e.g., 1, 2, 3, 4, 5, 6, or 7) nucleotide modifications (substitutions, insertions, deletions, or mismatches) to a starting sequence (e.g., a reference sequence). Such modifications may improve at least one characteristic (e.g., a biological function) of the polynucleotide (e.g., improved RISC loading or retention of a guide strand, reduced RISC loading or retention of the passenger strand, or increased ratio of guide-to-strand production, and improved inhibition of a target nucleic acid sequence).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1W are images of stem-loop structures that contain the Grik2 mRNA-targeting antisense sequence GI (SEQ ID NO: 16) or a variant thereof embedded in an endogenous (E)-miR-30 microRNA scaffold. The stem-loop structures contain, from 5′ to 3, a guide strand containing the GI antisense sequence or a rationally designed variant thereof (SEQ ID NOs: 17-30 230-233, and 242-245), an E-miR-30 loop sequence, and a passenger sequence (SEQ ID NO: 31) or a rationally designed variant thereof (SEQ ID NOs: 32-45, 234-237, and 246-249). The starting construct (Construct A) is shown in FIG. 1A. Changes relative to the starting construct are shown in FIGS. 1B-1W, respectively. Small black dots correspond to U-G wobble pairs. Large black dots with numerals correspond to design benchmarks described in Example 1. *Drosha and Dicer cleavage sites are based on most abundant species observed in small RNA sequencing data obtained from the starting construct A, which was delivered into induced pluripotent stem cell (iPSC)-derived glutamatergic neurons (GlutaNeurons).

FIGS. 2A-2Q are images of stem-loop structures that contain the Grik2 mRNA-targeting antisense sequence G9 (SEQ ID NO: 63) or a variant thereof embedded in an endogenous E-miR-124-3 microRNA scaffold. The stem-loop structures contain a guide strand containing the G9 antisense sequence or a rationally designed variant thereof (SEQ ID NOs: 64-79), an E-miR-124-3 loop sequence, and a passenger sequence (SEQ ID NO: 80) or a rationally designed variant thereof (SEQ ID NOs: 81-96]). The starting construct (Construct B) is shown in FIG. 2A. Changes relative to the starting construct are shown in FIGS. 2B-2Q, respectively. Constructs shown in FIGS. 2A-21 feature stem-loop structures containing, from 5′ to 3′, the passenger strand, loop sequence, and guide strand, whereas FIGS. 2J-2Q feature stem-loop structures containing, from 5′ to 3′, the guide strand, loop sequence, and passenger strand. Small black dots correspond to U-G wobble pairs. Large black dots with numerals correspond to design benchmarks described in Example 1. *Drosha and Dicer cleavage sites are based on most abundant species observed in small RNA sequencing data obtained from the starting construct B, which was delivered into GlutaNeurons.

FIGS. 3A-3L are images of stem-loop structures that contain the Grik2 mRNA-targeting antisense sequence MW (SEQ ID NO: 109) or a variant thereof embedded in an endogenous E-miR-124-3 microRNA scaffold. The stem-loop structures contain, from 5′ to 3′, a passenger sequence (SEQ ID NO: 121) or a rationally designed variant thereof (SEQ ID NOs: 122-132), an E-miR-124-3 loop sequence, and a guide strand containing the MW antisense sequence or a rationally designed variant thereof (SEQ ID NOs: 110-120). The starting construct (Construct C) is shown in FIG. 3A. Changes relative to the starting construct are shown in FIGS. 3B-3L, respectively. Small black dots correspond to U-G wobble pairs. Large black dots with numerals correspond to design benchmarks described in Example 1. *Drosha and Dicer cleavage sites are based on most abundant species observed in small RNA sequencing data obtained from the starting construct C, which was delivered into GlutaNeurons.

FIGS. 4A-4F are images of stem-loop structures that contain the Grik2 mRNA-targeting antisense sequence MW (SEQ ID NO: 139) or a variant thereof embedded in an endogenous E-miR-218-1 microRNA scaffold. The stem-loop structures contain, from 5′ to 3, a guide strand containing the MW antisense sequence or a rationally designed variant thereof (SEQ ID NOs: 140-144), an E-miR-218-1 loop sequence, and a passenger sequence (SEQ ID NO: 145) or a rationally designed variant thereof (SEQ ID NOs: 146-150). The starting construct (Construct D) is shown in FIG. 4A. Changes relative to the starting construct are shown in FIGS. 4B-4F, respectively. Small black dots correspond to U-G wobble pairs. Large black dots with numerals correspond to design benchmarks described in Example 1. *Drosha and Dicer cleavage sites are based on most abundant species observed in small RNA sequencing data obtained from the starting construct D, which was delivered into GlutaNeurons.

FIGS. 5A-5E are images of AAV expression constructs containing single-microRNA constructs of the disclosure. General construct architecture features from 5′ to 3′: AAV 5′ ITR, hSyn1 promoter sequence, a stem-loop sequence containing from 5′ to 3′: a 5′ microRNA flanking sequence, a 5′ stem-loop arm containing either a guide strand or a passenger strand sequence, a microRNA (E-miR) loop sequence, a 3′ stem-loop arm containing either a passenger strand or a guide strand sequence, and a 3′ flanking sequence; a polyadenylation sequence (RGB polyA), and an AAV 3′ ITR (FIG. 5A). FIG. 5B shows an AAV vector, Construct #102, containing the stem-loop sequence of Construct #3 (SEQ ID NO: 4). FIG. 5C shows an AAV vector, Construct #103, containing the stem-loop sequence of Construct #51 (SEQ ID NO: 135). FIG. 5D shows an AAV vector containing the stem-loop sequence of Construct #39 (SEQ ID NO: 98). FIG. 5E shows an AAV vector containing the stem-loop sequence of Construct #40 (SEQ ID NO: 99).

FIGS. 6A and 6B are images of AAV expression constructs containing concatemer constructs of the disclosure. FIG. 6A shows a dual-microRNA AAV vector, Construct #100, containing the stem-loop sequence of Construct #3 (SEQ ID NO: 4) and Construct #51 (SEQ ID NO: 135), in which Construct #3 is positioned 5′ relative to Construct #51. FIG. 6B shows a concatemer AAV vector containing the stem-loop sequence of Construct #3 (SEQ ID NO: 4) and Construct #51 (SEQ ID NO: 135), in which Construct #3 is positioned 3′ relative to Construct #51.

FIG. 7 is a graph depicting relative expression levels of human Grik2 mRNA, as quantified by RT-qPCR, in SH-SY5Y cells transfected as indicated in Example 3. n=4 for all groups. One-way ANOVA, Dunnett's multiple comparisons test (versus siNegative). ** p<0.001; Error bars: standard deviation. Key: RNAiMAX-transfection reagent only; siNegative-siRNA negative control; siPositive=siRNA positivo control; A, C, D=Constructs A, C, and D, respectively; #1, #2, #3, #4, #39, #40, #50, and #51=Constructs #1, #2, #3, #4, #39, #40, #50, and #51, respectively.

FIGS. 8A and 8B are graphs showing the expression of miRNA GI and MW and GLUK2 protein levels, respectively, in mouse cortical neurons (MCNs) after transduction with AAV vectors. FIG. 8A shows GI and MW quantification by stem-loop RT-qPCR. The y-axis indicates the number of molecules of GI or MW miRNA, per 10 μg of total RNA, expressed in cells transduced with the AAV vectors: from left to right, a RNA null vector (Ctrl), a dual-miRNA concatemer, Construct #100 (Seq ID: 256), containing the stem-loop sequence of Construct #3 (SEQ ID NO: 4) positioned 5′ relative to Construct #51 (SEQ ID NO: 135), a dual-miRNA concatemer, Construct #101 (SEQ ID: 257), containing the stem-loop sequence of Construct #51 positioned 5′ relative to Construct #3, a single construct containing just the GI sequence (SEQ ID NO: 252), and a single construct containing just the MW sequence (SEQ ID NO: 253). FIG. 8B shows GLUK2 protein levels quantified by immunoblot. The control wells were treated with AAV9.hSyn.GFP, RNA null control vector or non-treated. The figure shows the fold change of GLUK2/GLUK3 expression normalized to beta-actin vs. AAV9.hSyn.GFP control for each of the conditions. ** P<0.01.

FIG. 9 is a graph showing Grik2 mRNA expression quantified by RNA sequencing in iPSC-derived GlutaNeuron cells after transduction with either RNA null vector (Ctrl) or an AAV encoding a dual-miRNA concatemer, Construct #100 (SEQ ID NO: 256), containing the stem-loop sequence of Construct #3 (SEQ ID NO: 4) positioned 5′ relative to Construct #51 (SEQ ID NO: 135). TPM, transcripts per million. ** FDR (P adj)<0.01.

FIGS. 10A and 10B are graphs displaying epileptiform activity of adjacent human brain slices from two patients with temporal lobe epilepsy (TLE). The brain slices of one patient were recorded under hyperexcitable conditions, and the brain slices from the other patient were recorded under physiological conditions. FIG. 10A shows adjacent organotypic hippocampal slices from a TLE patient recorded in the presence of 4-AP/gabazine. The left side of the panel shows raw traces of an ictal event that was recorded after transduction by a control vector (AAV9.hSyn.GFP). The right side of the panel shows raw traces depicting epileptiform discharges following transduction with Construct #100 (AAV9.hSyn.Construct #3/Construct #51; SEQ ID NO: 256). Compared to control, Construct #100 markedly suppressed spontaneous seizures from the TLE hippocampus ex-vivo under hyperexcitable conditions. FIG. 10B shows neuronal excitability of organotypic hippocampal slices from another TLE patient; these slices were recorded under physiological conditions to record spontaneous seizure activity after transduction with the RNA null control and Construct #100. Compared to control, Construct #100 markedly suppressed spontaneous seizures from the TLE hippocampus ex-vivo in physiological buffer conditions.

FIGS. 11A-C are graphs depicting behavioral assessment of epileptic related phenotypes in the pilocarpine mouse model. Chronic epileptic mice were treated with either RNA null control vector (Ctrl) or Construct #100 (SEQ ID NO: 256), Construct #101 (SEQ ID NO: 257), a single construct containing just the GI sequence (SEQ ID NO: 252), and a single construct containing just the MW sequence (SEQ ID NO: 253) (n=5), all applied at 1E+9 GC/brain. * p<0.05, ** p<0.01, Mann-Whitney test. The concatemer vectors, Construct #100 and Construct #101, were effective in improving epileptic related phenotypes in the pilocarpine model in vivo. FIG. 11A shows the total distance covered by chronic epileptic mice during 10 minutes of exploration in an open field box. Epileptic mice are hyperactive and travel approximately twice the distance relative to non-epileptic mice. Accordingly, mice treated with the concatemer vectors behaved more like non-epileptic mice and traveled loss distance after treatment. FIG. 11B shows the average daily number of seizures in chronic epileptic mice treated with either RNA null control, the first concatemer, Construct #100, or the second concatemer, Construct #101. FIG. 11C shows behavioral scoring based on five animal behaviors (nesting, shaking, hairs, handling, and locomotion). The Y-axis represents the sum of scorings for the five behaviors. The control represents the epileptic mice treated with control vector. The mice treated with Construct #100 exhibited behavior that was similar to normal, non-epileptic mice.

FIGS. 12A and 12B are graphs of distance traveled or seizure activity in pilocarpine mice treated with either RNA null control vector (Ctrl) or Construct #100 (SEQ ID NO: 256). At the tested dose of 1E+10 GC/brain, Construct #100 was effective in reducing hyperlocomotion phenotype and seizure activity in the pilocarpine mouse model in vivo. FIG. 12A shows the total distance covered by chronic epileptic mice during 10 min exploration in an open field box. Chronic epileptic mice were treated with either control vector or Construct #100 applied at 1E+10 GC/brain. **** p<0.0001, Mann-Whitney test. FIG. 12B shows the average daily number of seizures in chronic epileptic mice one month after treatment with either control vector or Construct #100. ** p<0.01, Mann-Whitney test.

FIGS. 13A and 13B are graphs depicting the dose-dependent reduction of hyperlocomotion phenotype and seizures in pilocarpine mice treated with Construct #100. FIG. 13A shows the total distance covered during 10 min exploration in an open field box. Chronic epileptic mice were treated with either the RNA null control vector (Ctrl) or Construct #100, 1E+8/1E+9/1E+10 GC/brain). ** p<0.01, Mann-Whitney test. Historical locomotor activity of wild type mice (WT) was assessed in a separate experiment but shown here for comparison. FIG. 13B shows the average daily number of seizures in chronic epileptic mice after treatment with either control vector or Construct #100.

FIG. 14 is an image of a vector map that includes the inhibitory polynucleotide sequences of Construct #100. While Construct #100 includes a lac promoter sequence, an ampicillin resistance (AmpR) promoter sequence, and a kanamycin resistance (KanR) sequence, other promoter and antibiotic resistance sequences (e.g., a chloramphenicol resistance sequence) can be included as alternatives.

### DETAILED DESCRIPTION

Described herein are compositions and methods for the treatment of an epilepsy, such as, e.g., a temporal lobe epilepsy (TLE; e.g., TLE refractory to treatment), in a subject (such as a mammalian subject, for example, a human) using inhibitory polynucleotides (e.g., polynucleotides encoding inhibitory RNA agent) with modifications designed to affect (e.g., improve) RNA-induced silencing complex (RISC) loading and, e.g., to enhance production of an antisense guide strand and minimize production of passenger strand, thereby promoting greater knockdown of Grik2 mRNA and GluK2 expression and reducing the potential risk of off-target effects and toxicity induced by the passenger strand. For example, a therapeutically effective amount of an inhibitory RNA molecule (e.g., an antisense oligonucleotide (ASO), shRNA, siRNA, shmiRNA, or nucleic acid vector encoding the same, such as those described herein) that targets an mRNA encoded by the glutamate ionotropic receptor kainato typo subunit 2 (Grik2) gene can be administered, e.g., according to the methods described herein, to treat an epilepsy in a subject (e.g., a human) in need thereof. Also described herein are compositions containing nucleic acid vectors (e.g., viral vectors, such as, e.g., adeno-associated viral (AAV) vectors) encoding an inhibitory RNA agent targeting the Grik2 mRNA for the treatment of TLE.

### Grik2

Grik2 is a gene encoding an ionotropic glutamate receptor subunit, GluK2, that is activated by the endogenous agonist glutamate and can also be selectively activated by the agonist kainate. GluK2-containing kainato receptors (KARs), like other ionotropic glutamate receptors, exhibit fast ligand gating by glutamate, which acts by opening a cation channel pore permeable to sodium and potassium. KAR complexes can be assembled from several subunits as heteromeric or homomeric assemblies of KAR subunits. Such receptors feature an extracellular N-terminus and a large peptide loop that together form the ligand-binding domain and an intracellular C-terminus. The ionotropic glutamate receptor complex itself acts as a ligand-gated ion channel, and upon binding glutamate mediates the passage of charged ions across the neuronal membrane. Generally, KARs are multimeric assemblies of GluK1, 2 and/or 3 (previously named GluR5, GluR6 and GluR7, respectively), GluK4 (KA1) and GluK5 (KA2) subunits (Collingridge, Neuropharmacology. 2009 January; 56 (1): 2-5). The various combinations of subunits involved in a KAR complex are often determined by RNA splicing and/or RNA editing (e.g., conversion of adenosine to inosine by adenosine deaminases) of mRNA encoding a particular KAR subunit. Furthermore, such RNA modification may impact the properties of the receptor, such as, e.g., altering calcium permeability of the channel. Increased activity of kainate receptors is known to be epileptogenic. GluK2-containing KARs are suitable targets for modulation of ionotropic glutamate receptor activity and subsequently amelioration of symptoms related to epileptogenesis (Peret et al., 2014).

### Temporal Lobe Epilepsy

Epileptogenesis is a process that leads to the establishment of epilepsy and which may appear latent while cellular, molecular, and morphological changes loading to pathological neuronal network reorganization occur. TLE is characterized by two main types based on the anatomical origin of the epileptogenic focus. TLE originating from the mesial temporal lobe (e.g., hippocampus, parahippocampal gyrus, subiculum, and amygdala, among others) is named mesial TLE (mTLE), whereas TLE originating from the lateral temporal lobe (e.g., temporal neocortex) is referred to as lateral TLE (lTLE). Additional features characteristic of TLE may include neuronal cell death in the CA1, CA3, dentate hilus, and dentate gyrus (DG) regions of the hippocampus, reversal of the GABA reversal potential, granule cell (GC) dispersion in the DG, and sprouting of recurrent GC mossy fibers that leads to the formation of pathophysiological recurrent excitatory synapses onto dentate GCs (rMF-DGC synapses).

Various causal factors have been attributed to the etiology of TLE including mesial temporal sclerosis, traumatic brain injury, brain infections (e.g., encephalitis and meningitis), hypoxic brain injury, stroke, cerebral tumors, genetic syndromes, and febrile seizures. Because plasticity of the CNS depends on both the developmental state and brain region-specific susceptibility, not all subjects with brain injuries develop epilepsy. The hippocampus, including the DG, has been identified as a brain region particularly susceptible to damage that leads to TLE, and, in some instances, has been associated with treatment-resistant (i.e., refractory) epilepsy (Jarero-Basulto, J. J., et al. Pharmaceuticals, 2018, 11, 17; doi: 10.3390/ph11010017). An amplification of excitatory glutamatergic signaling may facilitate spontaneous seizures (Kuruba, et al. Epilepsy Behav. 2009, 14 (Suppl. 1), 65-73).

Without wishing to be bound by theory, aberrant rMF-DGC synapses, which operate via ectopic GluK2-containing KARs (Epsztein et al., 2005; Artinian et al., 2011, 2015) may play a key role in chronic seizures in TLE (Peret et al., 2014). For example, interictal spikes and ictal events (i.e., electrophysiological signatures of epileptiform brain activity) were reduced in transgenic mice lacking the GluK2 receptor subunit or in the presence of a pharmacological agent inhibiting GluK2/GluK5 receptors (Peret et al., 2014; Crépel and Mulle, 2015). While knockdown or silencing of GluK2 in transgenic animal models designed to test these theories is feasible, designing an inhibitor selective for the GluK2 subunit and safe for use in humans is challenging. The GluK subunits are structurally conserved and their DNA coding sequences share significant homologies. The complex gene expression pattern in the brain with respect to homomeric and heteromeric ionotropic and metabotropic glutamate receptors further complicates any therapeutic strategy. The methods and compositions disclosed herein are suitable for the treatment of a TLE (e.g., mTLE or lTLE) by targeting Grik2 mRNA and decreasing (e.g., knocking down) the expression of GluK2-containing KARs in neurons or astroglia, which promotes, e.g., a reduction in spontaneous epileptiform discharges in neuronal circuits (e.g., hippocampal circuits). As such, the compositions and methods described herein target the physiological cause of the disease and can be used for therapy.

Inhibitory Polynucleotides Targeting Grik2 mRNA

Clinical management of TLE is notoriously difficult, with at least one third of TLE patients being unable to have adequate control of debilitating seizures using available medications. These patients often experience recurrent epileptic seizures that are refractory to treatment. In such scenarios, TLE patients may resort to invasive and irreversible surgical resection of the epileptogenic focus in the temporal lobe, which can result in unwanted cognitive deficits. Thus, a substantial fraction of TLE patients are in need of novel therapeutic avenues for treating pharmaco-resistant TLE. The compositions and methods described herein provide the benefit of treating the underlying molecular pathophysiology that leads to the development and progression of TLE.

The compositions described herein, which are polynucleotides encoding inhibitory nucleic acid constructs (e.g., inhibitory RNA agents or nucleic acid vectors encoding the same) that target a Grik2 mRNA (e.g., any one of SEQ ID NOs: 164-174), can be administered according to the methods described herein to treat an epilepsy, such as TLE. The methods and compositions described herein can be used to treat a TLE patient having any type of TLE, such as, e.g., TLE with focal seizures, TLE with generalized seizures, mTLE, or lTLE. Furthermore, the presently disclosed methods and compositions may be used to treat TLE resulting from any etiology such as, e.g., mesial temporal sclerosis, traumatic brain injury, brain infections (e.g., encephalitis and meningitis), hypoxic brain injury, stroke, cerebral tumors, genetic syndromes, or febrile seizures. The compositions and methods described herein may also be administered as a preventative treatment to a subject at risk of developing TLE, e.g., a subject in the latent phase of TLE progression.

According to the methods and compositions disclosed herein, the inhibitory nucleic acid (e.g., an inhibitory RNA agent) may inhibit the expression of GluK2 by causing the degradation of Grik2 mRNA in a cell (e.g., a neuron, such as, e.g., a hippocampal neuron, such as, e.g., a hippocampal neuron of the dentate gyrus, such as, e.g., a dentate granule cell (DGC), or a glutamatergic pyramidal neuron), thereby preventing translation of the mRNA into a functional GluK2 protein.

The inhibitory nucleic acid molecules (e.g., inhibitory RNA agents) targeting the Grik2 mRNA disclosed herein may act to decrease the frequency of or completely inhibit the occurrence of epileptic brain activity (e.g., epileptiform discharges) in one or more brain regions. Such brain regions may include, but are not limited to the mesial temporal lobe, lateral temporal lobe, frontal lobe, or more specifically, hippocampus (e.g., DG, CA1, CA2, CA3, subiculum) or neocortex. Due to the aberrant expression of GluK2-containing KARs in rMF-DGCs of the DG, the occurrence of epileptic brain activity may be inhibited in the DG.

Accordingly, the disclosure provides methods and compositions for reducing epileptiform discharges in a CNS cell (e.g., a DGC) by contacting the cell with an effective amount of an inhibitory nucleic acid molecule (e.g., an inhibitory RNA agent) with at least 85% (e.g., at least 85%, 90%, 95%, 96%, 97%, 98%, 99%, or more (e.g., 100%)) sequence identity to any one of SEQ ID NOs: 1-19, 34-62, 97-108, 133-147, 226-229, and 238-241, or a nucleic acid vector encoding the same, such as a nucleic acid vector with at least 85% (e.g., at least 85%, 90%, 95%, 96%, 97%, 98%, 99%, or more (e.g., 100%)) sequence identity to SEQ ID NO: 256. In some embodiments, the nucleic acid molecule includes, from 5′ to 3′, a miR-30 guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% sequence identity to SEQ ID NO: 4, and a miR-30 passenger sequence having at least 85% identity to SEQ ID NO: 34. In some embodiments, the nucleic acid molecule includes, from 5′ to 3′, a miR-30 guide sequence having the nucleic acid sequence identity of SEQ ID NO: 19, a miR-30 stem-loop sequence having the nucleic acid sequence of SEQ ID NO: 4, and a miR-30 passenger sequence having the nucleic acid sequence of SEQ ID NO: 34. In some embodiments, the nucleic acid molecule includes, from 5′ to 3′, a nucleic acid sequence having at least 85% sequence identity to SEQ ID NO: 4. In some embodiments, the nucleic acid molecule includes, from 5′ to 3′, the nucleic acid sequence of SEQ ID NO: 4. In some embodiments, the nucleic acid molecule includes, from 5′ to 3′, a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% sequence identity to SEQ ID NO: 147. In some embodiments, the nucleic acid molecule includes, from 5′ to 3′, a miR-218-1 guide sequence having the nucleic acid sequence of SEQ ID NO: 141, a miR-218-1 stem-loop sequence having the nucleic acid sequence of SEQ ID NO: 135, and a miR-218-1 passenger sequence having the nucleic acid sequence of SEQ ID NO: 147. In some embodiments, the nucleic acid molecule includes, from 5′ to 3′, a nucleic acid sequence having at least 85% (e.g., at least 85%, 90%, 95%, 96%, 97%, 98%, 99%, or more (e.g., 100%)) sequence identity to SEQ ID NO: 135. In some embodiments, the nucleic acid molecule includes, from 5′ to 3′, the nucleic acid sequence of SEQ ID NO: 135.

In some embodiments, the nucleic acid molecule includes, from 5′ to 3′, (a) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; and (b), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147. In some embodiments, the nucleic acid molecule includes, from 5′ to 3′, (a) a miR-30 sequence guide sequence having the sequence of SEQ ID NO: 19, a miR-30 stem-loop sequence having the sequence of SEQ ID NO: 4, and a miR-30 passenger sequence having the sequence of SEQ ID NO: 34; and (b), a miR-218-1 guide sequence having the sequence of SEQ ID NO: 141, a miR-218-1 stem-loop sequence having the sequence of SEQ ID NO: 135, and a miR-218-1 passenger sequence having the sequence of SEQ ID NO: 147. In some embodiments, the nucleic acid molecule includes a nucleic acid sequence having at least 85% (e.g., at least 85%, 90%, 95%, 96%, 97%, 98%, 99%, or more (e.g., 100%)) sequence identity to SEQ ID NO: 258. In some embodiments, the nucleic acid molecule includes the nucleic acid sequence of SEQ ID NO: 258.

In some embodiments, the nucleic acid molecule includes, from 5′ to 3′, (a) a hSyn promoter sequence with at least 85% (e.g., at least 85%, 90%, 95%, 96%, 97%, 98%, 99%, or more (e.g., 100%)) sequence identity to any one of SEQ ID NOs: 194-198, (b) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% (e.g., at least 85%, 90%, 95%, 96%, 97%, 98%, 99%, or more (e.g., 100%)) sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; and (c), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147. In some embodiments, the nucleic acid molecule includes, from 5′ to 3′, (a) a hSyn promoter sequence with at least 85% (e.g., at least 85%, 90%, 95%, 96%, 97%, 98%, 99%, or more (e.g., 100%)) sequence identity to SEQ ID NO: 198, (b) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% (e.g., at least 85%, 90%, 95%, 96%, 97%, 98%, 99%, or more (e.g., 100%)) sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; and (c), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147. In some embodiments, the nucleic acid molecule includes, from 5′ to 3′, (a) a hSyn promoter sequence having the sequence of SEQ ID NO: 198, (b) a miR-30 sequence guide sequence having the sequence of SEQ ID NO: 19, a miR-30 stem-loop sequence having the sequence of SEQ ID NO: 4, and a miR-30 passenger sequence having the sequence of SEQ ID NO: 34; and (c), a miR-218-1 guide sequence having the sequence of SEQ ID NO: 141, a miR-218-1 stem-loop sequence having the sequence of SEQ ID NO: 135, and a miR-218-1 passenger sequence having the sequence of SEQ ID NO: 147. In some embodiments, the nucleic acid molecule includes a nucleic acid sequence having at least 85% (e.g., at least 85%, 90%, 95%, 96%, 97%, 98%, 99%, or more (e.g., 100%)) sequence identity to SEQ ID NO: 259. In some embodiments, the nucleic acid molecule includes the nucleic acid sequence of SEQ ID NO: 259.

In some embodiments of any of the following nucleic acid molecules described herein, the nucleic acid molecule may include a single promoter, which can control expression of one or more (e.g., two) miRNA sequences, or two promoters, each of which can control expression of a single miRNA construct. For example, in some embodiments, the nucleic acid molecule includes, from 5′ to 3′, (a) a promoter sequence; (b) a miRNA sequence, such as a miR-30 sequence including a miR-30 guide sequence, a miR-30 stem-loop sequence, and a miR-30 passenger sequence; (c) optionally, a second promoter sequence; and (d) a second miRNA sequence, such as a miR-218 sequence including a miR-218-1 guide sequence, a miR-218-1 stem-loop sequence, and a miR-218-1 passenger sequence. In some embodiments, the nucleic acid molecule includes, from 5′ to 3′, (a) a promoter sequence; (b) a miRNA sequence, such as a miR-30 sequence including a miR-30 guide sequence, a miR-30 stem-loop sequence, and a miR-30 passenger sequence; and (c) a second miRNA sequence, such as a miR-218 sequence including a miR-218-1 guide sequence, a miR-218-1 stem-loop sequence, and a miR-218-1 passenger sequence. In some embodiments, the nucleic acid molecule includes, from 5′ to 3′, (a) a promoter sequence; (b) a miRNA sequence, such as a miR-30 sequence including a miR-30 guide sequence, a miR-30 stem-loop sequence, and a miR-30 passenger sequence; (c) a second promoter sequence; and (d) a second miRNA sequence, such as a miR-218 sequence including a miR-218-1 guide sequence, a miR-218-1 stem-loop sequence, and a miR-218-1 passenger sequence.

In some embodiments, the nucleic acid molecule includes, from 5′ to 3′, (a) a hSyn promoter sequence with at least 85% (e.g., at least 85%, 90%, 95%, 96%, 97%, 98%, 99%, or more (e.g., 100%)) sequence identity to any one of SEQ ID NOs: 194-198, (b) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% (e.g., at least 85%, 90%, 95%, 96%, 97%, 98%, 99%, or more (e.g., 100%)) sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; (c), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147; and (d), a rabbit beta-globin (RBG) poly-adenylation (polyA) signal sequence having at least 85% (e.g., at least 85%, 90%, 95%, 96%, 97%, 98%, 99%, or more (e.g., 100%)) sequence identity to one or more (e.g., two, three, four, or five) of SEQ ID NOs: 213, 214, and 215. In some embodiments, the nucleic acid molecule includes, from 5′ to 3′, (a) a hSyn promoter sequence with at least 85% (e.g., at least 85%, 90%, 95%, 96%, 97%, 98%, 99%, or more (e.g., 100%)) sequence identity to SEQ ID NO: 198, (b) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% (e.g., at least 85%, 90%, 95%, 96%, 97%, 98%, 99%, or more (e.g., 100%)) sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; (c), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147; and (d), a RBG polyA signal sequence having at least 85% (e.g., at least 85%, 90%, 95%, 96%, 97%, 98%, 99%, or more (e.g., 100%)) sequence identity to one or more (e.g., two, three, four, or five) of SEQ ID NOs: 213, 214, and 215. In some embodiments, the nucleic acid molecule includes, from 5′ to 3′, (a) a hSyn promoter sequence having the sequence of SEQ ID NO: 198, (b) a miR-30 sequence guide sequence having the sequence of SEQ ID NO: 19, a miR-30 stem-loop sequence having the sequence of SEQ ID NO: 4, and a miR-30 passenger sequence having the sequence of SEQ ID NO: 34; (c), a miR-218-1 guide sequence having the sequence of SEQ ID NO: 141, a miR-218-1 stem-loop sequence having the sequence of SEQ ID NO: 135, and a miR-218-1 passenger sequence having the sequence of SEQ ID NO: 147; and (d), a RBG polyA signal sequence having the sequence of any one of SEQ ID NOs: 213, 214, and 215. In some embodiments, the nucleic acid molecule includes a nucleic acid sequence having at least 85% (e.g., at least 85%, 90%, 95%, 96%, 97%, 98%, 99%, or more (e.g., 100%)) sequence identity to SEQ ID NO: 260. In some embodiments, the nucleic acid molecule includes the nucleic acid sequence of SEQ ID NO: 260.

In some embodiments, the nucleic acid molecule includes, from 5′ to 3′, (a) a 5′ ITR sequence with at least 85% (e.g., at least 85%, 90%, 95%, 96%, 97%, 98%, 99%, or more (e.g., 100%)) sequence identity to SEQ ID NO: 208, (b) a hSyn promoter sequence with at least 85% (e.g., at least 85%, 90%, 95%, 96%, 97%, 98%, 99%, or more (e.g., 100%)) sequence identity to SEQ ID NO: 198, (c) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% (e.g., at least 85%, 90%, 95%, 96%, 97%, 98%, 99%, or more (e.g., 100%)) sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; (d), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147; (e), a RBG polyA signal sequence having at least 85% (e.g., at least 85%, 90%, 95%, 96%, 97%, 98%, 99%, or more (e.g., 100%)) sequence identity to one or more (e.g., two, three, four, or five) of SEQ ID NOs: 213, 214, and 215; and (f), a 3′ ITR sequence with at least 85% (e.g., at least 85%, 90%, 95%, 96%, 97%, 98%, 99%, or more (e.g., 100%)) sequence identity to SEQ ID NO: 212. In some embodiments, the nucleic acid molecule includes, from 5′ to 3′, (a) a 5′ ITR sequence having the sequence of SEQ ID NO: 208, (b) a hSyn promoter sequence having the sequence of SEQ ID NO: 198, (c) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence having the sequence of SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; (d), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence having the sequence of SEQ ID NO: 135, and a miR-218-1 passenger sequence having the sequence of SEQ ID NO: 147; (e), a RBG polyA signal sequence having at least 85% (e.g., at least 85%, 90%, 95%, 96%, 97%, 98%, 99%, or more (e.g., 100%)) sequence identity to one or more (e.g., two, three, four, or five) of SEQ ID NOs: 213, 214, and 215; and (f), a 3′ ITR sequence having the sequence of SEQ ID NO: 212. In some embodiments, the nucleic acid molecule includes a nucleic acid sequence having at least 85% (e.g., at least 85%, 90%, 95%, 96%, 97%, 98%, 99%, or more (e.g., 100%)) sequence identity to SEQ ID NO: 261. In some embodiments, the nucleic acid molecule includes the nucleic acid sequence of SEQ ID NO: 261.

In some embodiments, the nucleic acid molecule includes, from 5′ to 3′, (a) a 5′ ITR sequence with at least 85% (e.g., at least 85%, 90%, 95%, 96%, 97%, 98%, 99%, or more (e.g., 100%)) sequence identity to SEQ ID NO: 208, (b) a hSyn promoter sequence with at least 85% (e.g., at least 85%, 90%, 95%, 96%, 97%, 98%, 99%, or more (e.g., 100%)) sequence identity to SEQ ID NO: 198, (c) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence with at least 85% (e.g., at least 85%, 90%, 95%, 96%, 97%, 98%, 99%, or more (e.g., 100%)) sequence identity to SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; (d), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence with at least 85% (e.g., at least 85%, 90%, 95%, 96%, 97%, 98%, 99%, or more (e.g., 100%)) sequence identity to SEQ ID NO: 135, and a miR-218-1 passenger sequence with at least 85% (e.g., at least 85%, 90%, 95%, 96%, 97%, 98%, 99%, or more (e.g., 100%)) sequence identity to SEQ ID NO: 147; (e), a RBG polyA signal sequence having at least 85% (e.g., at least 85%, 90%, 95%, 96%, 97%, 98%, 99%, or more (e.g., 100%)) sequence identity to one or more (e.g., two, three, four, or five) of SEQ ID NOs: 213, 214, and 215; (f), a stuffer sequence with at least 85% (e.g., at least 85%, 90%, 95%, 96%, 97%, 98%, 99%, or more (e.g., 100%)) sequence identity to one or more (e.g., two, three, four, or five) of SEQ ID NOs: 250 and 251; and (g), a 3′ ITR sequence with at least 85% (e.g., at least 85%, 90%, 95%, 96%, 97%, 98%, 99%, or more (e.g., 100%)) sequence identity to SEQ ID NO: 212. In some embodiments, the nucleic acid molecule includes, from 5′ to 3′, (a) a 5′ ITR sequence having the sequence of SEQ ID NO: 208, (b) a hSyn promoter sequence having the sequence of SEQ ID NO: 198, (c) a miR-30 sequence guide sequence having at least 85% (e.g., at least 85%, 90%, 95%, 96%, 97%, 98%, 99%, or more (e.g., 100%)) sequence identity to SEQ ID NO: 19, a miR-30 stem-loop sequence having the sequence of SEQ ID NO: 4, and a miR-30 passenger sequence having at least 85% (e.g., at least 85%, 90%, 95%, 96%, 97%, 98%, 99%, or more (e.g., 100%)) identity to SEQ ID NO: 34; (d), a miR-218-1 guide sequence having at least 85% (e.g., at least 85%, 90%, 95%, 96%, 97%, 98%, 99%, or more (e.g., 100%)) sequence identity to SEQ ID NO: 141, a miR-218-1 stem-loop sequence having the sequence of SEQ ID NO: 135, and a miR-218-1 passenger sequence having the sequence of SEQ ID NO: 147; (e), a RBG polyA signal sequence having at least 85% (e.g., at least 85%, 90%, 95%, 96%, 97%, 98%, 99%, or more (e.g., 100%)) sequence identity to one or more (e.g., two, three, four, or five) of SEQ ID NOs: 213, 214, and 215; (f), a stuffer sequence having the sequence of one or more (e.g., two, three, four, or five) of SEQ ID NOs: 250 and 251; and (g), a 3′ ITR sequence having the sequence of SEQ ID NO: 212.

In some embodiments, the nucleic acid molecule is encoded in an expression cassette having at least 85% (e.g., at least 85%, 90%, 95%, 96%, 97%, 98%, 99%, or more (e.g., 100%)) sequence identity to the nucleic acid sequence of SEQ ID NO: 256. In some embodiments, the expression cassette has the nucleic acid sequence of SEQ ID NO: 256.

The inhibitory nucleic acid molecules (e.g., inhibitory RNA agents) of the disclosure may be a GluK2 inhibitor. In particular, the GluK2 inhibitor may be a Grik2 mRNA expression inhibitor. Inhibiting the expression of GluK2 may also inhibit the levels of GluK5 (Ruiz et al, J Neuroscience 2005). While not wishing to be bound to any theory, the disclosure is based on the principle that sufficient removal of GluK2 alone should remove all GluK2/GluK5 heteromers, since GluK5 subunits alone are not capable of forming homomeric assemblies.

According to the disclosed methods and compositions, the inhibitory nucleic acid molecules (e.g., inhibitory RNA agents) disclosed herein may have a length from 15 to 50 nucleotides (e.g., 15, 16, 17, 18, 19, 20, 21, 22, 23, 24, or 25, 30, 35, 40, 45, or up to 50 nucleotides). For example, the inhibitory nucleic acid molecules (e.g., inhibitory RNA agents) disclosed herein may have a length of 15 nucleotides. In another example, the inhibitory nucleic acid molecule (e.g., inhibitory RNA agent) has a length of 16 nucleotides. In another example, the inhibitory nucleic acid molecule (e.g., inhibitory RNA agent) has a length of 17 nucleotides. In another example, the inhibitory nucleic acid molecule (e.g., inhibitory RNA agent) has a length of 18 nucleotides. In another example, the inhibitory nucleic acid molecules (e.g., inhibitory RNA agent) has a length of 19 nucleotides. In another example, the inhibitory nucleic acid molecule (e.g., inhibitory RNA agent) has a length of 20 nucleotides. In another example, the inhibitory nucleic acid molecule (e.g., inhibitory RNA agent) has a length of 21 nucleotides. In another example, the inhibitory nucleic acid molecule (e.g., inhibitory RNA agent) has a length of 22 nucleotides. In another example, the inhibitory nucleic acid molecule (e.g., inhibitory RNA agent) has a length of 23 nucleotides. In another example, the inhibitory nucleic acid molecule (e.g., inhibitory RNA agent) has a length of 24 nucleotides. In another example, the inhibitory nucleic acid molecule (e.g., inhibitory RNA agent) has a length of 25 nucleotides. In another example, the inhibitory nucleic acid molecule (e.g., inhibitory RNA agent) has a length of 25-30 nucleotides. In another example, the inhibitory nucleic acid molecule (e.g., inhibitory RNA agent) has a length of 30-35 nucleotides. In another example, the inhibitory nucleic acid molecule (e.g., inhibitory RNA agent) has a length of 35-40 nucleotides. In another example, the inhibitory nucleic acid molecule (e.g., inhibitory RNA agent) has a length of 40-45 nucleotides. In another example, the inhibitory nucleic acid molecule (e.g., inhibitory RNA agent) has a length of 45-50 nucleotides.

The inhibitory RNA agents of the disclosure include a sequence that is at least substantially complementary or fully complementary to a region of the sequence of Grik2 mRNA (e.g., any one of SEQ ID NOs: 164-174) or variants thereof, said complementarity being sufficient to yield specific binding under intracellular conditions. In some embodiments, the inhibitory RNA agents include a sequence that is at least substantially complementary or fully complementary to a region of the sequence of Grik2 mRNA, such as SEQ ID NO: 164, or variants thereof having at least 85% sequence identity to SEQ ID NO: 164. For example, the disclosure contemplates an inhibitory RNA agent having an antisense sequence that is complementary to at least 7 (e.g., at least 7, 8, 9, 10, 11, 12, 13, 14, 15, 16, 17, 18, 19, 20, 21, 22, or more) consecutive nucleotides of one or more regions of a Grik2 mRNA. In a particular example, the inhibitory RNA agent has an antisense sequence that is complementary to 7 consecutive nucleotides of one or more regions of a Grik2 mRNA. In another example, the inhibitory RNA agent has an antisense sequence that is complementary to 8 consecutive nucleotides of one or more regions of a Grik2 mRNA. In another example, the inhibitory RNA agent has an antisense sequence that is complementary to 9 consecutive nucleotides of one or more regions of a Grik2 mRNA. In another example, the inhibitory RNA agent has an antisense sequence that is complementary to 10 consecutive nucleotides of one or more regions of a Grik2 mRNA. In another example, the inhibitory RNA agent has an antisense sequence that is complementary to 11 consecutive nucleotides of one or more regions of a Grik2 mRNA. In another example, the inhibitory RNA agent has an antisense sequence that is complementary to 12 consecutive nucleotides of one or more regions of a Grik2 mRNA. In another example, the inhibitory RNA agent has an antisense sequence that is complementary to 13 consecutive nucleotides of one or more regions of a Grik2 mRNA. In another example, the inhibitory RNA agent has an antisense sequence that is complementary to 14 consecutive nucleotides of one or more regions of a Grik2 mRNA. In another example, the inhibitory RNA agent has an antisense sequence that is complementary to 15 consecutive nucleotides of one or more regions of a Grik2 mRNA. In another example, the inhibitory RNA agent has an antisense sequence that is complementary to 16 consecutive nucleotides of one or more regions of a Grik2 mRNA. In another example, the inhibitory RNA agent has an antisense sequence that is complementary to 17 consecutive nucleotides of one or more regions of a Grik2 mRNA. In another example, the inhibitory RNA agent has an antisense sequence that is complementary to 18 consecutive nucleotides of one or more regions of a Grik2 mRNA. In another example, the inhibitory RNA agent has an antisense sequence that is complementary to 19 consecutive nucleotides of one or more regions of a Grik2 mRNA. In another example, the inhibitory RNA agent has an antisense sequence that is complementary to 20 consecutive nucleotides of one or more regions of a Grik2 mRNA. In another example, the inhibitory RNA agent has an antisense sequence that is complementary to 21 consecutive nucleotides of one or more regions of a Grik2 mRNA. In another example, the inhibitory RNA agent has an antisense sequence that is complementary to 22 consecutive nucleotides of one or more regions of a Grik2 mRNA. In yet another example, the inhibitory RNA agent has an antisense sequence that is 100% complementary to the nucleotides of one or more regions of a Grik2 mRNA.

The disclosure contemplates inhibitory RNA agents that, when bound to one or more regions of a Grik2 mRNA (e.g., any one of the regions of Grik2 mRNA described in SEQ ID NOs: 164-174), form a duplex structure with the Grik2 mRNA of between 7-22 (e.g., 7, 8, 9, 10, 11, 12, 13, 14, 15, 16, 17, 18, 19, 20, 21, or 22) nucleotides in length. In some embodiments, an inhibitory RNA agent of the disclosure may bind to a region of Grik2 mRNA within the sequence of SEQ ID NO: 164 and form a duplex structure with the Grik2 mRNA of between 7-22 (e.g., 7, 8, 9, 10, 11, 12, 13, 14, 15, 16, 17, 18, 19, 20, 21, or 22) nucleotides in length. For example, the duplex structure between the inhibitory RNA agent and the Grik2 mRNA may be 7 nucleotides in length. In another example, the duplex structure between the inhibitory RNA agent and the Grik2 mRNA may be 8 nucleotides in length. In another example, the duplex structure between the inhibitory RNA agent and the Grik2 mRNA may be 9 nucleotides in length. In another example, the duplex structure between the inhibitory RNA agent and the Grik2 mRNA may be 10 nucleotides in length. In another example, the duplex structure between the inhibitory RNA agent and the Grik2 mRNA may be 11 nucleotides in length. In another example, the duplex structure between the inhibitory RNA agent and the Grik2 mRNA may be 12 nucleotides in length. In another example, the duplex structure between the inhibitory RNA agent and the Grik2 mRNA may be 13 nucleotides in length. In another example, the duplex structure between the inhibitory RNA agent and the Grik2 mRNA may be 14 nucleotides in length. In another example, the duplex structure between the inhibitory RNA agent and the Grik2 mRNA may be 15 nucleotides in length. In another example, the duplex structure between the inhibitory RNA agent and the Grik2 mRNA may be 16 nucleotides in length. In another example, the duplex structure between the inhibitory RNA agent and the Grik2 mRNA may be 17 nucleotides in length. In another example, the duplex structure between the inhibitory RNA agent and the Grik2 mRNA may be 18 nucleotides in length. In another example, the duplex structure between the inhibitory RNA agent and the Grik2 mRNA may be 19 nucleotides in length. In another example, the duplex structure between the inhibitory RNA agent and the Grik2 mRNA may be 20 nucleotides in length. In another example, the duplex structure between the inhibitory RNA agent and the Grik2 mRNA may be 21 nucleotides in length. In yet another example, the duplex structure between the inhibitory RNA agent and the Grik2 mRNA may be 10 nucleotides in length.

According to the disclosed methods and compositions, the duplex structure formed by an inhibitory RNA agent (e.g., an agent having at least 85% (at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, and 238-241), such as the duplex structure formed by an inhibitory RNA agent having at least 85% sequence identity to the nucleic acid sequence of SEQ ID NO: 258, and one or more regions of a Grik2 mRNA may include at least one (e.g., at least 1, 2, 3, 4, 5, 6, 7, 8, 9, 10, 11, 12, 13, 14, or 15) mismatch. For example, the duplex structure may contain 1 mismatch. In another example, the duplex structure contains 2 mismatches. In another example, the duplex structure contains 3 mismatches. In another example, the duplex structure contains 4 mismatches. In another example, the duplex structure contains 5 mismatches. In another example, the duplex structure contains 6 mismatches. In another example, the duplex structure contains 7 mismatches. In another example, the duplex structure contains 8 mismatches. In another example, the duplex structure contains 9 mismatches. In another example, the duplex structure contains 10 mismatches. In another example, the duplex structure contains 11 mismatches. In another example, the duplex structure contains 12 mismatches. In another example, the duplex structure contains 13 mismatches. In another example, the duplex structure contains 14 mismatches. In yet another example, the duplex structure contains 15 mismatches.

Accordingly, an object of the disclosure relates to isolated, synthetic, or recombinant inhibitory nucleic acid molecules (e.g., inhibitory RNA agents) targeting Grik2 mRNA. The inhibitory RNA agent of the disclosure may be of any suitable type, including RNA or DNA inhibitory polynucleotides. Thus, the disclosed methods and compositions feature a Grik2 expression inhibitor that is an inhibitory RNA agent (e.g., siRNA, shRNA, miRNA, or shmiRNA). Inhibitory RNA agents, including antisense RNA molecules and antisense DNA molecules, may act to directly block the translation of Grik2 mRNA by binding thereto and preventing protein translation or increasing mRNA degradation, thereby decreasing the level and activity of GluK2 proteins. For example, inhibitory RNA agents having at least about 19 bases and complementarity to unique regions of the mRNA transcript sequence encoding GluK2 can be synthesized, e.g., by conventional techniques (e.g., techniques disclosed herein) and administered by, e.g., intravenous injection or infusion, among other routes described heroin, such as direct injection to a region of the brain. Methods for using antisense techniques for specifically alleviating gene expression of genes whose sequence is known are well known in the art (e.g., see U.S. Pat. Nos. 6,566,135; 6,566, 131; 6,365,354; 6,410,323; 6,107,091; 6,046,321; and 5,981,732, each of which is incorporated by reference herein in its entirety).

In a particular example, a Grik2 inhibitory RNA agent of the disclosure may be a short interfering RNA (siRNA). Grik2 gone expression can be reduced by contacting the subject or cell with a small double stranded RNA (dsRNA), or a vector encoding the same, thereby causing the production of a small double stranded RNA capable of specifically inhibiting Grik2 expression by degradation of mRNAs in a sequence-specific manner (e.g., by way of the RNA interference pathway). Methods for selecting an appropriate dsRNA or dsRNA-encoding vector are known in the art for genes whose sequence is known (e.g., see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836, each of which is incorporated by reference herein in its entirety).

The Grik2 inhibitory RNA agent of the disclosure may also be a short hairpin RNA (shRNA). An shRNA is a sequence of RNA that makes a tight hairpin turn that can be used to silence gone expression via RNA interference. shRNA is generally expressed using a vector introduced into target cells, wherein the vector often utilizes the ubiquitous U6 promoter to ensure that the shRNA is constitutively expressed. This vector is usually passed on to daughter cells, allowing the gene silencing to be maintained following cell division. The shRNA hairpin structure is cleaved by the cellular machinery into siRNA, which is then bound to the RNA-induced silencing complex (RISC). This complex binds to and cleaves mRNAs that match the siRNA sequence to which it is bound.

Additionally, the Grik2 expression inhibitor of the disclosure may be a microRNA (miRNA). miRNA has a general meaning in the art and refers, e.g., to microRNA molecules that are generally 21 to 22 nucleotides in length, even though lengths of 19 and up to 23 nucleotides have been reported, and can be used to suppress translation of targeted mRNAs. miRNAs are each processed from a longer precursor RNA molecule (“precursor miRNA”). Precursor miRNAs are transcribed from non-protein-encoding genes. The precursor miRNAs have two regions of complementarity that allow them to form a stem-loop- or fold-back-like structure, which is cleaved in animals by a ribonuclease III-like nuclease enzyme called Dicer. The processed miRNA is typically a portion of the stem containing a “seed sequence” (typically 6-8 nucleotides) that is fully or substantially complementary to a region of the target mRNA. The processed miRNA (also referred to as “mature miRNA”) becomes part of a large complex to downregulate (e.g., decrease translation or degrade mRNA) of a particular target gene.

Furthermore, the GluK2 inhibitor of the disclosure may be a miRNA-adapted shRNA (shmiRNA). shmiRNA agents refer to chimeric molecules that incorporate antisense sequences within the -5p or the -3p arm of a microRNA scaffold (e.g., a E-miR-30 scaffold) containing microRNA flanking and loop sequences. Compared to an shRNA, shmiRNA generally has a longer stem-loop structure based on microRNA-derived sequences, with the -5p and the -3p arm exhibiting full or substantial complementarity (e.g., mismatches, G: U wobbles). Owing to their longer sequences and processing requirements, shmiRNAs are generally expressed from a Pol II promoter. These constructs have also been shown to exhibit reduced toxicity as compared to shRNA-based agents.

Multiple miRNAs may be employed to knockdown Grik2 mRNA expression (and subsequently its gene product, GluK2). The miRNAs may be complementary to different target transcripts or different binding sites of a single target transcript. Polycistronic or multi-gene transcripts may also be utilized to enhance the efficiency of target gene knockdown. Multiple genes encoding the same miRNAs or different miRNAs may be regulated together in a single transcript, or as separate transcripts in a single vector cassette. miRNAs of the disclosure may be packaged into a vector, such as, e.g., a viral vector, including but not limited to recombinant adeno-associated viral (rAAV) vectors, lentiviral vectors, retroviral vectors and retrotransposon-based vector systems.

The inhibitory RNA that is complementary (e.g., substantially or fully complementary) to the sense target sequence of a Grik2 mRNA is generally encoded by a DNA sequence for the production of any of the foregoing inhibitors (e.g., siRNAs, shRNAs, miRNAs, or shmiRNAs). The DNA encoding a double-stranded RNA of interest can be incorporated into a gene cassette (e.g., an expression cassette in which transcription of the DNA is controlled by a promoter).

### Improving RISC Loading for Guide Sequences

A step in RNA interference is assembly of the microRNA guide strand into the RNA-induced silencing complex (RISC) protein complex that mediates target mRNA cleavage. microRNA is produced as a double-stranded duplex containing a guide strand hybridized through complementary base-pairing to a passenger strand. Assembly of the guide strand into the RISC complex is generally accompanied by degradation of the passenger strand. RISC assembly favors a microRNA strand having a 5′ end with a greater propensity to fray or to be liberated from the duplex. The constructs described herein are designed to favor guide selection and loading and to disfavor passenger selection by RISC by destabilizing base pairing at the 5′ end of the guide strand (e.g., by introducing a U-A pair or U-G wobble pair at or near the 5′ end of the guide strand) and tightening base pairing at the 5′ end of the passenger strand (e.g., by introducing a G-C pair at or near the 5′ end of the passenger strand). This strategy is attainable because mismatches between the guide strand and the target mRNA are well-tolerated if they occur at the first nucleotide or near the 3′ end (e.g., within the last four nucleotides) of the guide strand. Such a strategy not only improves on-target knock-down by the guide strand, but also reduces the off-target effects from passenger strand production or retention by the RISC protein complex.

Accordingly, the anti-Grik2 antisense molecules (e.g., microRNA, shRNA, siRNA, or shmiRNA) described herein include one or more modifications that improve RISC loading or retention of the guide strand and reduce RISC loading or retention of the passenger strand, increase guide-to-passenger strand ratio within the cell, and increase the level of knockdown of the target Grik2 mRNA.

Base-pairing instability at or near the 5′ end of the guide strand was increased in several of the constructs described herein in order to improve RISC loading or retention of the guide strand. For example, base-pairing instability is achieved by introducing a U-A pair or a U-G wobble pair at or near the 5′ end of the guide strand of several constructs.

RISC loading or retention of the passenger strand is reduced in several of the constructs described herein by introducing base-pairing instability at the 5′ end of the passenger strand. The base-pairing instability is introduced by adding a C-G pair at or near the 5′ end of the passenger strand.

Several constructs of the disclosure were also designed to enhance RISC loading or retention of the guide strand by introducing a 5′-terminal uracil in the guide strand. This 5′-terminal nucleotide is not involved in hybridization to the target mRNA (e.g., Grik2 mRNA) and is generally anchored in the phosphate-binding pocket of Argonaute RISC Catalytic Component 2 (Ago2) proteins.

RISC loading or retention of the passenger strand is reduced for several of the disclosed constructs by introducing one or more mismatches (e.g., 1, 2, 3, 4, 5, 6, 7, or more mismatches) in a seed region of the guide strand (corresponding to nucleotides 2-7 of the guide strand; g2-g7). This strategy is employed to promote the unwinding and unloading of the passenger strand during RISC loading. While extensive complementarity in the seed region (guide nucleotide 2-8, g2-g8) and the middle region of guide strand are crucial for Ago2-mediated mRNA cleavage, base-paring at the 3′ end is not required. In fact, mismatches at positions g18, g19, g20, g21 of the guide strand with the target mRNA were determined to attenuate the release of the guide strand from Ago2, an unloading activity mediated by target mRNA.

Dicer cleavage of a loop region from a stem-loop structure of an anti-Grik2 construct is improved for several of the constructs disclosed herein by tightening the base-pairing at the junction of the stem and loop regions by replacing a U-G wobble pair to a C-G pair. The Grik2 mRNA-targeting constructs of the disclosure leverage the aforementioned modifications to promote an increase in the ratio of guide to passenger strand production and to improve silencing of Grik2 for the treatment of a seizure disorder (e.g., TLE).

Thus, the inhibitory RNA molecules described herein may include a stem-loop sequence containing guide strand and passenger strand sequences rationally designed from the anti-Grik2 sequence GI (SEQ ID NO: 16) embedded in an E-miR-30 microRNA scaffold and sequences complementary thereto (see, e.g., Table 2 (e.g., SEQ ID NOs: 1-15, 226-229, and 238-241)), or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity thereto.

TABLE 2
Grik2-targeting constructs containing the antisense sequence
GI or a variant thereof

| | | | | SEQ |
|---|---|---|---|---|
| Antisense | microRNA | | | ID |
| sequence* | scaffold | Version | Stem-loop Sequence | NO |
| GI | E-miR-30 | Construct | gtttgaatgaggcttcagtactttacagaatcgttgcctgcacatctt | 1 |
| (GI; SEQ | | A | ggaaacacttgctgggattacttcttcaggttaacccaacagaag | |
| ID NO: 16) | | | gctAAagaaggtatattgctgttgacagtgagcgacGTCTC | |
| | | | GATATGGAGAACCCATGctgtgaagccacagatggg | |
| | | | catgggttttatatcgagacgctgcctactgcctcggaCttcaag | |
| | | | gggctactttaggagcaattatcttgtttactaaaactgaatacctt | |
| | | | gctatctctttgatacatttttacaaagctgaattaaaatggtataaa | |
| | | | tta | |
| GI | E-miR-30 | Construct | gtttgaatgaggcttcagtactttacagaatcgttgcctgcacatctt | 2 |
| | | #1 | ggaaacacttgctgggattacttcttcaggttaacccaacagaag | |
| | | | gctAAagaaggtatattgctgttgacagtgagcgacTTCTC | |
| | | | GATATGGAGAACCCAGGcCgtgaagccacagatgg | |
| | | | gcCtgggttttatatcgagaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 3 |
| | | #2 | ggaaacacttgctgggattacttcttcaggttaacccaacagaag | |
| | | | gctAAagaaggtatattgctgttgacagtgagcgacTTCTC | |
| | | | GATATGGAGAACCCAGGcCgtgaagccacagatgg | |
| | | | gcCtgggttttatatcgCgaAgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 4 |
| | | #3 | ggaaacacttgctgggattacttcttcaggttaacccaacagaag | |
| | | | gctAAagaaggtatattgctgttgacagtgagcgacTTCTC | |
| | | | GATATGGAGAACCCAGGcCgtgaagccacagatgg | |
| | | | gcCtgggttttatatcgagCAgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 5 |
| | | #4 | ggaaacacttgctgggattacttcttcaggttaacccaacagaag | |
| | | | gctAAagaaggtatattgctgttgacagtgagcgacTTCTC | |
| | | | GATATGGAGAACCCAGGcCatgaagccacagatgg | |
| | | | gcCtgggttttatatcgaTaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 6 |
| | | #5 | ggaaacacttgctgggattacttcttcaggttaacccaacagaag | |
| | | | gctAAagaaggtatattgctgttgacagtgagcgacTTCTC | |
| | | | GATATGGAGAACCCATGcCgtgaagccacagatgg | |
| | | | gcatgggttttatatcgCgaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 7 |
| | | #6 | ggaaacacttgctgggattacttcttcaggttaacccaacagaag | |
| | | | gctAAagaaggtatattgctgttgacagtgagcgacTTCTC | |
| | | | GATATGGAGAACCCATGcCgtgaagccacagatgg | |
| | | | gcatgggttttatatcgagaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 8 |
| | | #7 | ggaaacacttgctgggattacttcttcaggttaacccaacagaag | |
| | | | gctAAagaaggtatattgctgttgacagtgagcgacTTCTC | |
| | | | GATATGGAGAACCCAGGctgtgaagccacagatgg | |
| | | | gcCtgggttttatatcgagaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 9 |
| | | #8 | ggaaacacttgctgggattacttcttcaggttaacccaacagaag | |
| | | | gctAAagaaggtatattgctgttgacagtgagcgacTTCTC | |
| | | | GATATGGAGAACCCAGGctgtgaagccacagatgg | |
| | | | gcCtgggttttatatcgCgaAgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 10 |
| | | #9 | ggaaacacttgctgggattacttcttcaggttaacccaacagaag | |
| | | | gctAAagaaggtatattgctgttgacagtgagcgacTTCTC | |
| | | | GATATGGAGAACCCATGctgtgaagccacagatggg | |
| | | | catgggttttatatcgCgaAgctgcctactgcctcggaCttcaag | |
| | | | gggctactttaggagcaattatcttgtttactaaaactgaatacctt | |
| | | | gctatctctttgatacatttttacaaagctgaattaaaatggtataaa | |
| | | | tta | |
| GI | E-miR-30 | Construct | gtttgaatgaggcttcagtactttacagaatcgttgcctgcacatctt | 11 |
| | | #10 | ggaaacacttgctgggattacttcttcaggttaacccaacagaag | |
| | | | gctAAagaaggtatattgctgttgacagtgagcgacTTCTC | |
| | | | GATATGGAGAACCCATGctgtgaagccacagatggg | |
| | | | catgggttttatatcgagaAgctgcctactgcctcggaCttcaag | |
| | | | gggctactttaggagcaattatcttgtttactaaaactgaatacctt | |
| | | | gctatctctttgatacatttttacaaagctgaattaaaatggtataaa | |
| | | | tta | |
| GI | E-miR-30 | Construct | gtttgaatgaggcttcagtactttacagaatcgttgcctgcacatctt | 12 |
| | | #11 | ggaaacacttgctgggattacttcttcaggttaacccaacagaag | |
| | | | gctAAagaaggtatattgctgttgacagtgagcgacTTCTC | |
| | | | GATATGGAGAACCCAGGctgtgaagccacagatagg | |
| | | | gcCtgggttttatatcgCgaGgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 13 |
| | | #12 | ggaaacacttgctgggattacttcttcaggttaacccaacagaag | |
| | | | gctAAagaaggtatattgctgttgacagtgagcgacTTCTC | |
| | | | GATATGGAGAACCCAGGctgtgaagccacagatgg | |
| | | | gcCtgggttttatatcgagaGgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 14 |
| | | #13 | ggaaacacttgctgggattacttcttcaggttaacccaacagaag | |
| | | | gctAAagaaggtatattgctgttgacagtgagcgacTTCTC | |
| | | | GATATGGAGAACCCATGctgtgaagccacagatggg | |
| | | | catgggttttatatcgCgaGgctgcctactgcctcggaCttcaag | |
| | | | gggctactttaggagcaattatcttgtttactaaaactgaatacctt | |
| | | | gctatctctttgatacatttttacaaagctgaattaaaatggtataaa | |
| | | | tta | |
| GI | E-miR-30 | Construct | gtttgaatgaggcttcagtactttacagaatcgttgcctgcacatctt | 15 |
| | | #14 | ggaaacacttgctgggattacttcttcaggttaacccaacagaag | |
| | | | gctAAagaaggtatattgctgttgacagtgagcgacTTCTC | |
| | | | GATATGGAGAACCCATGctgtgaagccacagatggg | |
| | | | catgggttttatatcgagaGgctgcctactgcctcggaCttcaag | |
| | | | gggctactttaggagcaattatcttgtttactaaaactgaatacctt | |
| | | | gctatctctttgatacatttttacaaagctgaattaaaatggtataaa | |
| | | | tta | |
| GI | E-miR-30 | Construct | gtttgaatgaggcttcagtactttacagaatcgttgcctgcacatctt | 226 |
| | | #15 | ggaaacacttgctgggattacttcttcaggttaacccaacagaag | |
| | | | gctAAagaaggtatattgctgttgacagtgagcgacTTCTC | |
| | | | GATATGGAGAACCCAGGctgtgaagccacagatgA | |
| | | | gcCtgggttttatatcgagaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 227 |
| | | #16 | ggaaacacttgctgggattacttcttcaggttaacccaacagaag | |
| | | | gctAAagaaggtatattgctgttgacagtgagcgacTTCTC | |
| | | | GATATGGAGAACCCAGGctgtgaagccacagatgA | |
| | | | gcCtgggttttatatcgCgaAgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 228 |
| | | #17 | ggaaacacttgctgggattacttcttcaggttaacccaacagaag | |
| | | | gctAAagaaggtatattgctgttgacagtgagcgacTTCTC | |
| | | | GATATGGAGAACCCATGctgtgaagccacagatgA | |
| | | | gcatgggttttatatcgagaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 229 |
| | | #18 | ggaaacacttgctgggattacttcttcaggttaacccaacagaag | |
| | | | gctAAagaaggtatattgctgttgacagtgagcgacTTCTC | |
| | | | GATATGGAGAACCCATGctgtgaagccacagatgA | |
| | | | gcatgggttttatatcgagaAgctgcctactgcctcggaCttcaa | |
| | | | ggggctactttaggagcaattatcttgtttactaaaactgaatacct | |
| | | | tgctatctctttgatacatttttacaaagctgaattaaaatggtataa | |
| | | | atta | |
| GI | E-miR-30 | Construct | gtttgaatgaggcttcagtactttacagaatcgttgcctgcacatctt | 238 |
| | | #19 | ggaaacacttgctgggattacttcttcaggttaacccaacagaag | |
| | | | gctAAagaaggtatattgctgttgacagtgagcgacTTCTC | |
| | | | GATATGGAGAACCCAGGcugtgaagccacagatgg | |
| | | | gcCtgggttttatatcgagCAgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 239 |
| | | #20 | ggaaacacttgctgggattacttcttcaggttaacccaacagaag | |
| | | | gctAAagaaggtatattgctgttgacagtgagcgacTTCTC | |
| | | | GATATGGAGAACCCAGGcugtgaagccacagatgg | |
| | | | gcCtggattttatatcgagCGgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 240 |
| | | #21 | ggaaacacttgctgggattacttcttcaggttaacccaacagaag | |
| | | | gctAAagaaggtatattgctgttgacagtgagcgacTTCTC | |
| | | | GATATGGAGAACCCAGGcugtgaagccacagatgg | |
| | | | gcCtgggttttatatcgagGGgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |
| GI | E-miR-30 | Construct | gtttgaatgaggcttcagtactttacagaatcgttgcctgcacatctt | 241 |
| | | #22 | ggaaacacttgctgggattacttcttcaggttaacccaacagaag | |
| | | | gctAAagaaggtatattgctgttgacagtgagcgacTTCTC | |
| | | | GATATGGAGAACCCAGGcugtgaagccacagatgg | |
| | | | gcCtgggttttatatcgagGAgctgcctactgcctcggaCttca | |
| | | | aggggctactttaggagcaattatcttgtttactaaaactgaatac | |
| | | | cttgctatctctttgatacatttttacaaagctgaattaaaatggtata | |
| | | | aatta | |

### Table 2 Sequence key:

The term “Antisense sequence,” as used in Table 2, refers to the antisense sequence GI or a variant thereof having 1-7 (e.g., 1, 2, 3, 4, 5, 6, or 7) modifications (e.g., substitution, deletion, insertion, or mismatch).
single and double underlined characters: stem-loop sequence;
CAPITAL ITALIC CHARACTERS WITH SINGLE UNDERLINE: guide strand;
single underlined lower-case characters: E-miR-30 loop sequence;
double-underlined characters: passenger strand;
CAPITAL BOLD CHARACTERS: substituted nucleotides.

Accordingly, the Grik2-targeting antisense constructs of the disclosure may include guide (SEQ ID NOs: 16-30, 230-233, and 242-245) and passenger strand (SEQ ID NOs: 31-45, 234-237, and 246-249) pairs described in Table 3, below:

TABLE 3
Guide and passenger strand pairs rationally designed from GI sequence in a E-miR-30 scaffold

| | | | | SEQ | | SEQ |
|---|---|---|---|---|---|---|
| Antisense | microRNA | | | ID | | ID |
| sequence* | scaffold | Version | Guide sequence | NO | Passenger sequence | NO |
| GI | E-miR-30 | Construct | gtctcgatatggagaacccatg | 16 | catgggttttatatcgagac | 31 |
| | | A | | | | |
| GI | E-miR-30 | Construct | TtctcgatatggagaacccaGg | 17 | cCtgggttttatatcgagaA | 32 |
| | | #1 | | | | |
| GI | E-miR-30 | Construct | TtctcgatatggagaacccaGg | 18 | cCtgggttttatatcgCgaA | 33 |
| | | #2 | | | | |
| GI | E-miR-30 | Construct | TtctcgatatggagaacccaGg | 19 | cCtgggttttatatcgagCA | 34 |
| | | #3 | | | | |
| GI | E-miR-30 | Construct | TtctcgatatggagaacccaGg | 20 | cCtgggttttatatcgaTaA | 35 |
| | | #4 | | | | |
| GI | E-miR-30 | Construct | Ttctcgatatggagaacccatg | 21 | catgggttttatatcgCgaA | 36 |
| | | #5 | | | | |
| GI | E-miR-30 | Construct | Ttctcgatatggagaacccatg | 22 | catgggttttatatcgagaA | 37 |
| | | #6 | | | | |
| GI | E-miR-30 | Construct | TtctcgatatggagaacccaGg | 23 | cCtgggttttatatcgagaA | 38 |
| | | #7 | | | | |
| GI | E-miR-30 | Construct | TtctcgatatggagaacccaGg | 24 | cCtgggttttatatcgCgaA | 39 |
| | | #8 | | | | |
| GI | E-miR-30 | Construct | Ttctcgatatggagaacccatg | 25 | catgggttttatatcgCgaA | 40 |
| | | #9 | | | | |
| GI | E-miR-30 | Construct | Ttctcgatatggagaacccatg | 26 | catgggttttatatcgagaA | 41 |
| | | #10 | | | | |
| GI | E-miR-30 | Construct | TtctcgatatggagaacccaGg | 27 | cCtgggttttatatcgCgaG | 42 |
| | | #11 | | | | |
| GI | E-miR-30 | Construct | TtctcgatatggagaacccaGg | 28 | cCtgggttttatatcgagaG | 43 |
| | | #12 | | | | |
| GI | E-miR-30 | Construct | Ttctcgatatggagaacccatg | 29 | catgggttttatatcgCgaG | 44 |
| | | #13 | | | | |
| GI | E-miR-30 | Construct | Ttctcgatatggagaacccatg | 30 | catgggttttatatcgagaG | 45 |
| | | #14 | | | | |
| GI | E-miR-30 | Construct | TtctcgatatggagaacccaGg | 230 | AgcCtgggttttatatcgagaA | 234 |
| | | #15 | | | | |
| GI | E-miR-30 | Construct | TtctcgatatggagaacccaGg | 231 | AgcCtgggttttatatcgCgaA | 235 |
| | | #16 | | | | |
| GI | E-miR-30 | Construct | Ttctcgatatggagaacccatg | 232 | AgcatgggttttatatcgagaA | 236 |
| | | #17 | | | | |
| GI | E-miR-30 | Construct | Ttctcgatatggagaacccatg | 233 | AgcatgggttttatatcgCgaA | 237 |
| | | #18 | | | | |
| GI | E-miR-30 | Construct | TtctcgatatggagaacccaGg | 242 | cCtgggttttatatcgagCA | 246 |
| | | #19 | | | | |
| GI | E-miR-30 | Construct | TtctcgatatggagaacccaGg | 243 | cCtgggttttatatcgagCG | 247 |
| | | #20 | | | | |
| GI | E-miR-30 | Construct | TtctcgatatggagaacccaGg | 244 | cCtgggttttatatcgagGG | 248 |
| | | #21 | | | | |
| GI | E-miR-30 | Construct | TtctcgatatggagaacccaGg | 245 | cCtgggttttatatcgagGA | 249 |
| | | #22 | | | | |

### Table 3 Sequence Key:

The term “Antisense Sequence,” as used in Table 3, refers to the antisense sequence GI or a variant thereof having 1-7 (e.g., 1, 2, 3, 4, 5, 6, or 7) modifications (e.g., substitution, deletion, insertion, or mismatch). 5
CAPITAL BOLD CHARACTERS: Nucleotides in a modified guide or passenger strand sequence relative to Construct A.

Also disclosed herein are inhibitory RNA molecules that may include a stem-loop sequence containing guide strand and passenger strand sequences rationally designed from the anti-Grik2 sequence G9 (SEQ ID NO: 63) embedded in an E-miR-124-3 microRNA scaffold and sequences complementary thereto (see, e.g., Table 4 (e.g., SEQ ID NOs: 46-62)), or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity thereto.

TABLE 4
Grik2-targeting constructs containing the antisense sequence G9 or a variant thereof

| Antisense | microRNA | | | SEQ |
|---|---|---|---|---|
| sequence* | scaffold | Version | Stem-loop Sequence | ID NO |
| G9 (SEQ | E-miR-124- | Construct B | tctgccgcggaaaggggagaagtgtgggctcctccgagtcggggg | 46 |
| ID NO: 63) | 3 | | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctacaatgggca | |
| | | | ctagacatgggatttaatgtctatacaatCCCATAGCTAATGC | |
| | | | CTGTTTTAgagaggcgcctccgccgctcctttctcatggaaatg | |
| | | | gcccgcgagcccgtccggcccagcgcccctcccgcgggaggaag | |
| | | | gcgagcccggcccccggcggccattcgcgccgcggacaaatccg | |
| | | | gcgaacaatgcgcccgcccagagtgcggcccagctgccgggccg | |
| | | | gggatctggccgcgggacacaaaggggcccgcacgcctctggcgt | |
| G9 | E-miR-124- | Construct | tctgccgcggaaaggggagaagtgtgggctcctccgagtcggggg | 47 |
| | 3 | #23 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctGcaaCAggc | |
| | | | actagacatgggatttaatgtctatacaatTCCATAGCTAATG | |
| | | | CCTGTTGCAgagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| G9 | E-miR-124- | Construct | tctgccgcggaaaggggagaagtgtgggctcctccgagtcggggg | 48 |
| | 3 | #24 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctGcaacAggc | |
| | | | actagacatggatttaatgtctatacaatTCATAGCTAATGCC | |
| | | | TGTTGCAgagaggcgcctccgccgctcctttctcatggaaatgg | |
| | | | cccgcgagcccgtccggcccagcgcccctcccgcgggaggaagg | |
| | | | cgagcccggcccccggcggccattcgcgccgcggacaaatccgg | |
| | | | cgaacaatgcgcccgcccagagtgcggcccagctgccgggccgg | |
| | | | ggatctggccgcgggacacaaaggggcccgcacgcctctggcgt | |
| G9 | E-miR-124- | Construct | tctgccgcggaaaggggagaagtgtgggctcctccgagtcggggg | 49 |
| | 3 | #25 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctGcaatgggca | |
| | | | ctagacatgggatttaatgtctatacaatTCCATAGCTAATGC | |
| | | | CTGTTGCAgagaggcgcctccgccgctcctttctcatggaaatg | |
| | | | gcccgcgagcccgtccggcccagcgcccctcccgcgggaggaag | |
| | | | gcgagcccggcccccggcggccattcgcgccgoggacaaatccg | |
| | | | gcgaacaatgcgcccgcccagagtgcggcccagctgccgggccg | |
| | | | gggatctggccgcgggacacaaaggggcccgcacgcctctggcgt | |
| G9 | E-miR-124- | Construct | tctgccgcggaaaggggagaagtgtgggctcctccgagtcggggg | 50 |
| | 3 | #26 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctacaaCAggc | |
| | | | actagacatgggatttaatgtctatacaatTCCATAGCTAATG | |
| | | | CCTGTTGTAgagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| G9 | E-miR-124- | Construct | tctgccgcggaaaggggagaagtgtgggctcctccgagtcggggg | 51 |
| | 3 | #27 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctacaatgggca | |
| | | | ctagacatgggatttaatgtctatacaatTCCATAGCTAATGC | |
| | | | CTGTTGTAgagaggcgcctccgccgctcctttctcatggaaatg | |
| | | | gcccgcgagcccgtccggcccagcgcccctcccgcgggaggaag | |
| | | | gcgagcccggcccccggcggccattcgcgccgcggacaaatccg | |
| | | | gcgaacaatgcgcccgcccagagtgcggcccagctgccgggccg | |
| | | | gggatctggccgcgggacacaaaggggcccgcacgcctctggcgt | |
| G9 | E-miR-124- | Construct | tctgccgcggaaaggggagaagtgtgggctcctccgagtcggggg | 52 |
| | 3 | #28 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctGcaatgggca | |
| | | | ctagacatggatttaatgtctatacaatTCATAGCTAATGCCT | |
| | | | GTTGCAgagaggcgcctccgccgctcctttctcatggaaatggc | |
| | | | ccgcgagcccgtccggcccagcgcccctcccgcgggaggaaggc | |
| | | | gagcccggcccccggcggccattcgcgccgcggacaaatccggc | |
| | | | gaacaatgcgcccgcccagagtgcggcccagctgccgggccggg | |
| | | | gatctggccgcgggacacaaaggggcccgcacgcctctggcgt | |
| G9 | E-miR-124- | Construct | tctgccgcggaaaggggagaagtgtgggctcctccgagtcggggg | 53 |
| | 3 | #29 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctacaaCAggc | |
| | | | actagacatggatttaatgtctatacaatTCATAGCTAATGCC | |
| | | | TGTTGTAgagaggcgcctccgccgctcctttctcatggaaatgg | |
| | | | cccgcgagcccgtccggcccagcgcccctcccgcgggaggaagg | |
| | | | cgagcccggcccccggcggccattcgcgccgcggacaaatccgg | |
| | | | cgaacaatgcgcccgcccagagtgcggcccagctgccgggccgg | |
| | | | ggatctggccgcgggacacaaaggggcccgcacgcctctggcgt | |
| G9 | E-miR-124- | Construct | tctgccgcggaaaggggagaagtgtgggctcctccgagtcggggg | 54 |
| | 3 | #30 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctctacaatgggca | |
| | | | ctagacatggatttaatgtctatacaatTCATAGCTAATGCCT | |
| | | | GTTGTAgagaggcgcctccgccgctcctttctcatggaaatggcc | |
| | | | cgcgagcccgtccggcccagcgcccctcccgcgggaggaaggcg | |
| | | | agcccggcccccggcggccattcgcgccgcggacaaatccggcg | |
| | | | aacaatgcgcccgcccagagtgcggcccagctgccgggccgggg | |
| | | | atctggccgcgggacacaaaggggcccgcacgcctctggcgt | |
| G9 | E-miR-124- | Construct | tctgccgcggaaaggggagaagtgtgggctcctccgagtcggggg | 55 |
| | 3 | #31 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTCCCATA | |
| | | | GCTAATGCCTGTGCGAttaatgtctatacatcgcacaggc | |
| | | | atcagctatggtaagagaggcgcctccgccgctcctttctcatggaa | |
| | | | atggcccgcgagcccgtccggcccagcgcccctcccgcgggagg | |
| | | | aaggcgagcccggcccccggcggccattcgcgccgcggacaaat | |
| | | | ccggcgaacaatgcgcccgcccagagtgcggcccagctgccggg | |
| | | | ccggggatctggccgcgggacacaaaggggcccgcacgcctctg | |
| | | | gcgt | |
| G9 | E-miR-124- | Construct | tctgccgcggaaaggggagaagtgtgggctcctccgagtcggggg | 56 |
| | 3 | #32 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTTCCATA | |
| | | | GCTAATGCCTGTGCGAttaatgtctatacatcgcacaggc | |
| | | | atcagctatggtaagagaggcgcctccgccgctcctttctcatggaa | |
| | | | atggcccgcgagcccgtccggcccagcgcccctcccgcgggagg | |
| | | | aaggcgagcccggcccccggcggccattcgcgccgcggacaaat | |
| | | | ccggcgaacaatgcgcccgcccagagtgcggcccagctgccggg | |
| | | | ccggggatctggccgcgggacacaaaggggcccgcacgcctctg | |
| | | | gcgt | |
| G9 | E-miR-124- | Construct | tctgccgcggaaaggggagaagtgtgggctcctccgagtcggggg | 57 |
| | 3 | #33 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTCCCATA | |
| | | | GCTAATGCCTGTGCGAttaatgtctatacatcgcacaTTc | |
| | | | atcagctatggtaagagaggcgcctccgccgctcctttctcatggaa | |
| | | | atggcccgcgagcccgtccggcccagcgcccctcccgcgggagg | |
| | | | aaggcgagcccggcccccggggccattcgcgccgcggacaaat | |
| | | | ccggcgaacaatgcgcccgcccagagtgcggcccagctgccggg | |
| | | | ccggggatctggccgcgggacacaaaggggcccgcacgcctctg | |
| | | | gcgt | |
| G9 | E-miR-124- | Construct | tctgccgcggaaaggggagaagtgtgggctcctccgagtcggggg | 58 |
| | 3 | #34 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTTCCATA | |
| | | | GCTAATGCCTGTGCGAttaatgtctatacatcgcacaTTc | |
| | | | atcagctatggtaagagaggcgcctccgccgctcctttctcatggaa | |
| | | | atggcccgcgagcccgtccggcccagcgcccctcccgcgggagg | |
| | | | aaggcgagcccggcccccggcggccattcgcgccgcggacaaat | |
| | | | ccggcgaacaatgcgcccgcccagagtgcggcccagctgccggg | |
| | | | ccggggatctggccgcgggacacaaaggggcccgcacgcctctg | |
| | | | gcgt | |
| G9 | E-miR-124- | Construct | tctgccgcggaaaggggagaagtgtgggctcctccgagtcggggg | 59 |
| | 3 | #35 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTCCCATA | |
| | | | GCTAATGCCTGTTTTAttaatgtctatacataaaacaggcat | |
| | | | cagctatggtaagagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| G9 | E-miR-124- | Construct | tctgccgcggaaaggggagaagtgtgggctcctccgagtcggggg | 60 |
| | 3 | #36 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTTCCATA | |
| | | | GCTAATGCCTGTTTTAttaatgtctatacataaaacaggcat | |
| | | | cagctatggtaagagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| G9 | E-miR-124- | Construct | tctgccgcggaaaggggagaagtgtgggctcctccgagtcggggg | 61 |
| | 3 | #37 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTCCCATA | |
| | | | GCTAATGCCTGTGCGAttaatgtctatacatCGCacagg | |
| | | | catcagctCtggtaagagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |
| G9 | E-miR-124- | Construct | tctgccgcggaaaggggagaagtgtgggctcctccgagtcggggg | 62 |
| | 3 | #38 | cggactgggacagcacagtcggctgagcgcagcgcccccgccct | |
| | | | gcccgccacgcggcgaagacgcctgagcgttcgcgcccctcgggc | |
| | | | gaggaccccacgcaagcccgagccggtcccgaccctggccccga | |
| | | | cgctcgccgcccgccccagccctgagggcccctcTTTCCATA | |
| | | | GCTAATGCCTGTGCGAttaatgtctatacatCGCacagg | |
| | | | catcagctCtggtaagagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |

### Table 4 Sequence key:

The term “Antisense sequence,” as used in Table 4, refers to the antisense sequence G9 or a variant thereof having 1-7 (e.g., 1, 2, 3, 4, 5, 6, or 7) modifications (e.g., substitution, deletion, insertion, or mismatch).
single and double underlined characters: stem-loop sequence;
CAPITAL ITALIC CHARACTERS WITH SINGLE UNDERLINE: guide strand;
single underlined lower-case characters: E-miR-124-3 loop sequence;
double-underlined characters: passenger strand;
CAPITAL BOLD CHARACTERS: substituted nucleotides.

Accordingly, the Grik2-targeting antisense constructs of the disclosure may include guide (SEQ ID NOs: 63-79) and passenger strand (SEQ ID NOs: 80-96) pairs described in Table 5, below:

TABLE 5
Guide and passenger strand pairs rationally designed from G9 sequence in a miR-124 scaffold

| | | | | SEQ | | SEQ |
|---|---|---|---|---|---|---|
| Antisense | miRNA | | | ID | | ID |
| sequence* | scaffold | Version | Guide sequence | NO | Passenger sequence | NO |
| G9 | E-miR- | Construct | cccatagctaatgcctgtttta | 63 | tacaatgggcactagacatggg | 80 |
| | 124-3 | B | | | | |
| G9 | E-miR- | Construct | TccatagctaatgcctgttGCa | 64 | tGcaaCAggcactagacatggg | 81 |
| | 124-3 | #23 | | | | |
| G9 | E-miR- | Construct | TcatagctaatgcctgttGCa | 65 | tGcaaCAggcactagacatgg | 82 |
| | 124-3 | #24 | | | | |
| G9 | E-miR- | Construct | TccatagctaatgcctgttGCa | 66 | tGcaatgggcactagacatggg | 83 |
| | 124-3 | #25 | | | | |
| G9 | E-miR- | Construct | TccatagctaatgcctgttGta | 67 | tacaaCAggcactagacatggg | 84 |
| | 124-3 | #26 | | | | |
| G9 | E-miR- | Construct | TccatagctaatgcctgttGta | 68 | tacaatgggcactagacatggg | 85 |
| | 124-3 | #27 | | | | |
| G9 | E-miR- | Construct | TcatagctaatgcctgttGCa | 69 | tGcaatgggcactagacatgg | 86 |
| | 124-3 | #28 | | | | |
| G9 | E-miR- | Construct | TcatagctaatgcctgttGta | 70 | tacaaCAggcactagacatgg | 87 |
| | 124-3 | #29 | | | | |
| G9 | E-miR- | Construct | TcatagctaatgcctgttGta | 71 | tacaatgggcactagacatgg | 88 |
| | 124-3 | #30 | | | | |
| G9 | E-miR- | Construct | cccatagctaatgcctgtGCGa | 72 | tcgcacaggcatcagctatggtaa | 89 |
| | 124-3 | #31 | | | | |
| G9 | E-miR- | Construct | TccatagctaatgcctgtGCGa | 73 | tcgcacaggcatcagctatggtaa | 90 |
| | 124-3 | #32 | | | | |
| G9 | E-miR- | Construct | cccatagctaatgcctgtGCGa | 74 | tcgcacaTTcatcagctatggtaa | 91 |
| | 124-3 | #33 | | | | |
| G9 | E-miR- | Construct | TccatagctaatgcctgtGCGa | 75 | tcgcacaTTcatcagctatggtaa | 92 |
| | 124-3 | #34 | | | | |
| G9 | E-miR- | Construct | cccatagctaatgcctgtttta | 76 | taaaacaggcatcagctatggtaa | 93 |
| | 124-3 | #35 | | | | |
| G9 | E-miR- | Construct | Tccatagctaatgcctgtttta | 77 | taaaacaggcatcagctatggtaa | 94 |
| | 124-3 | #36 | | | | |
| G9 | E-miR- | Construct | cccatagctaatgcctgtGCGa | 78 | tCGCacaggcatcagctCtggtaa | 95 |
| | 124-3 | #37 | | | | |
| G9 | E-miR- | Construct | TccatagctaatgcctgtGCGa | 79 | tCGCacaggcatcagctCtggtaa | 96 |
| | 124-3 | #38 | | | | |

### Table 5 Sequence Key:

The term “Antisense sequence,” as used in Table 5, refers to the antisense sequence G9 or a variant thereof having 1-7 (e.g., 1, 2, 3, 4, 5, 6, or 7) modifications (e.g., substitution, deletion, insertion, or mismatch).
CAPITAL BOLD CHARACTERS: Nucleotides in a modified guide or passenger strand sequence relative to Construct B.

The inhibitory RNA molecules described herein may include a stem-loop sequence containing guide strand and passenger strand sequences rationally designed from the anti-Grik2 sequence MW (SEQ ID NO: 109) embedded in an E-miR-124-3 microRNA scaffold and sequences complementary thereto (see, e.g., Table 6 (e.g., SEQ ID NOs: 97-108)), or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity thereto.

TABLE 6
Grik2-targeting constructs containing the antisense sequence MW or a variant thereof

| Antisense | miRNA | | | SEQ |
|---|---|---|---|---|
| sequence* | scaffold | Version | Stem-loop Sequence | ID NO |
| MW (SEQ | E-miR- | Construct | tctgccgcggaaaggggagaagtgtgggctcctccgagtcggggg | 97 |
| ID NO: | 124-3 | C | cggactgggacagcacagtcggctgagcgcagcgcccccgccct | |
| 109) | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgacgtttatcta | |
| | | | caacactctgatttaatgtctatacaatCAGAGCATTGCAGA | |
| | | | TGGACTGCgagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| MW | E-miR- | Construct | tctgccgcggaaaggggagaagtgtgggctcctccgagtcggggg | 98 |
| | 124-3 | #39 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgCAgtCCat | |
| | | | ctacaacactctgatttaatgtctatacaatTAGAGCATTGCA | |
| | | | GATGGACTGCgagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |
| MW | E-miR- | Construct | tctgccgcggaaaggggagaagtgtgggctcctccgagtcggggg | 99 |
| | 124-3 | #40 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaGgttCatc | |
| | | | tacaacactTtAatttaatgtctatacaatTAGAGCATTGCAG | |
| | | | ATGGACCGCgagaggcgcctccgccgctcctttctcatggaaa | |
| | | | tggcccgcgagcccgtccggcccagcgcccctcccgcgggagga | |
| | | | aggcgagcccggcccccggcggccattcgcgccgcggacaaatc | |
| | | | cggcgaacaatgcgcccgcccagagtgcggcccagctgccgggc | |
| | | | cggggatctggccgcgggacacaaaggggcccgcacgcctctgg | |
| | | | cgt | |
| MW | E-miR- | Construct | tctgccgcggaaaggggagaagtgtgggctcctccgagtcggggg | 100 |
| | 124-3 | #41 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaGgtCCat | |
| | | | ctacaacactTtAatttaatgtctatacaatTAGAGCATTGCA | |
| | | | GATGGACCGCgagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |
| MW | E-miR- | Construct | tctgccgcggaaaggggagaagtgtgggctcctccgagtcggggg | 101 |
| | 124-3 | #42 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgCAgtttatct | |
| | | | acaacactctgatttaatgtctatacaatTAGAGCATTGCAGA | |
| | | | TGGACTGCgagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| MW | E-miR- | Construct | tctgccgcggaaaggggagaagtgtgggctcctccgagtcggggg | 102 |
| | 124-3 | #43 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaAgtCCat | |
| | | | ctacaacactctgatttaatgtctatacaatTAGAGCATTGCA | |
| | | | GATGGACTGCgagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |
| MW | E-miR- | Construct | tctgccgcggaaaggggagaagtgtgggctcctccgagtcggggg | 103 |
| | 124-3 | #44 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaAgtttatct | |
| | | | acaacactctgatttaatgtctatacaatTAGAGCATTGCAGA | |
| | | | TGGACTGCgagaggcgcctccgccgctcctttctcatggaaat | |
| | | | ggcccgcgagcccgtccggcccagcgcccctcccgcgggaggaa | |
| | | | ggcgagcccggcccccggcggccattcgcgccgcggacaaatcc | |
| | | | ggcgaacaatgcgcccgcccagagtgcggcccagctgccgggcc | |
| | | | ggggatctggccgcgggacacaaaggggcccgcacgcctctggc | |
| | | | gt | |
| MW | E-miR- | Construct | tctgccgcggaaaggggagaagtgtgggctcctccgagtcggggg | 104 |
| | 124-3 | #45 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaAgtttatct | |
| | | | acaacactctAatttaatgtctatacaatTAGAGCATTGCAG | |
| | | | ATGGACTGCgagaggcgcctccgccgctcctttctcatggaaa | |
| | | | tggcccgcgagcccgtccggcccagcgcccctcccgcgggagga | |
| | | | aggcgagcccggcccccggcggccattcgcgccgcggacaaatc | |
| | | | cggcgaacaatgcgcccgcccagagtgcggcccagctgccgggc | |
| | | | cggggatctggccgcgggacacaaaggggcccgcacgcctctgg | |
| | | | cgt | |
| MW | E-miR- | Construct | tctgccgcggaaaggggagaagtgtgggctcctccgagtcggggg | 105 |
| | 124-3 | #46 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaGgttCatc | |
| | | | tacaacactctAatttaatgtctatacaatTAGAGCATTGCAG | |
| | | | ATGGACCGCgagaggcgcctccgccgctcctttctcatggaaa | |
| | | | tggcccgcgagcccgtccggcccagcgcccctcccgcgggagga | |
| | | | aggcgagcccggcccccggcggccattcgcgccgcggacaaatc | |
| | | | cggcgaacaatgcgcccgcccagagtgcggcccagctgccgggc | |
| | | | cggggatctggccgcgggacacaaaggggcccgcacgcctctgg | |
| | | | cgt | |
| MW | E-miR- | Construct | tctgccgcggaaaggggagaagtgtgggctcctccgagtcggggg | 106 |
| | 124-3 | #47 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaGgtttatct | |
| | | | acaacactctAatttaatgtctatacaatTAGAGCATTGCAG | |
| | | | ATGGACCGCgagaggcgcctccgccgctcctttctcatggaaa | |
| | | | tggcccgcgagcccgtccggcccagcgcccctcccgcgggagga | |
| | | | aggcgagcccggcccccggcggccattcgcgccgcggacaaatc | |
| | | | cggcgaacaatgcgcccgcccagagtgcggcccagctgccgggc | |
| | | | cggggatctggccgcgggacacaaaggggcccgcacgcctctgg | |
| | | | cgt | |
| MW | E-miR- | Construct | tctgccgcggaaaggggagaagtgtgggctcctccgagtcggggg | 107 |
| | 124-3 | #48 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgaGgtCCat | |
| | | | ctacaacactctAatttaatgtctatacaatTAGAGCATTGCA | |
| | | | GATGGACCGCgagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |
| MW | E-miR- | Construct | tctgccgcggaaaggggagaagtgtgggctcctccgagtcggggg | 108 |
| | 124-3 | #49 | cggactgggacagcacagtcggctgagcgcagcgcccccgccct | |
| | | | gcccgccacgcggcgaagacgcctgagcgttcgcgcccctcggg | |
| | | | cgaggaccccacgcaagcccgagccggtcccgaccctggccccg | |
| | | | acgctcgccgcccgccccagccctgagggcccctcgCGgtCCat | |
| | | | ctacaacactTtAatttaatgtctatacaatTAGAGCATTGCA | |
| | | | GATGGACCGCgagaggcgcctccgccgctcctttctcatgga | |
| | | | aatggcccgcgagcccgtccggcccagcgcccctcccgcgggag | |
| | | | gaaggcgagcccggcccccggcggccattcgcgccgcggacaa | |
| | | | atccggcgaacaatgcgcccgcccagagtgcggcccagctgccg | |
| | | | ggccggggatctggccgcgggacacaaaggggcccgcacgcctc | |
| | | | tggcgt | |

### Table 6 Sequence key:

The term “Antisense Sequence,” as used in Table 6, refers to the antisense sequence MW or a variant thereof having 1-7 (e.g., 1, 2, 3, 4, 5, 6, or 7) modifications (e.g., substitution, deletion, Insertion, or mismatch).
single and double underlined characters: stem-loop sequence;
CAPITAL ITALIC CHARACTERS WITH SINGLE UNDERLINE: guide strand;
single underlined lower-case characters: E-miR-124-3 loop sequence;
double-underlined characters: passenger strand;
CAPITAL BOLD CHARACTERS: substituted nucleotides.

Accordingly, the Grik2-targeting antisense constructs of the disclosure may include guide (SEQ ID NOs: 109-120) and passenger strand (SEQ ID NOs: 121-132) pairs described in Table 7, below:

TABLE 7
Guide and passenger strand pairs rationally designed from MW
sequence in a miR-124 scaffold

| | | | | SEQ | | SEQ |
|---|---|---|---|---|---|---|
| Antisense | miRNA | | | ID | | ID |
| sequence* | scaffold | Version | Guide sequence | NO | Passenger sequence | NC |
| MW | E-miR- | Construct C | cagagcattgcagatggactgc | 109 | gacgtttatctacaacactctg | 121 |
| | 124-3 | | | | | |
| MW | E-miR- | Construct | Tagagcattgcagatggactgc | 110 | gCAgtCCatctacaacactctg | 122 |
| | 124-3 | #39 | | | | |
| MW | E-miR- | Construct | TagagcattgcagatggacCgc | 111 | gaGgttCatctacaacactTtA | 123 |
| | 124-3 | #40 | | | | |
| MW | E-miR- | Construct | TagagcattgcagatggacCgc | 112 | gaGgtCCatctacaacactTtA | 124 |
| | 124-3 | #41 | | | | |
| MW | E-miR- | Construct | Tagagcattgcagatggactgc | 113 | gCAgtttatctacaacactctg | 125 |
| | 124-3 | #42 | | | | |
| MW | E-miR- | Construct | Tagagcattgcagatggactgc | 114 | gaAgtCCatctacaacactctg | 126 |
| | 124-3 | #43 | | | | |
| MW | E-miR- | Construct | Tagagcattgcagatggactgc | 115 | gaAgtttatctacaacactctg | 127 |
| | 124-3 | #44 | | | | |
| MW | E-miR- | Construct | Tagagcattgcagatggactgc | 116 | gaAgtttatctacaacactctA | 128 |
| | 124-3 | #45 | | | | |
| MW | E-miR- | Construct | TagagcattgcagatggacCgc | 117 | gaGgttCatctacaacactctA | 129 |
| | 124-3 | #46 | | | | |
| MW | E-miR- | Construct | TagagcattgcagatggacCgc | 118 | gaGgtttatctacaacactctA | 130 |
| | 124-3 | #47 | | | | |
| MW | E-miR- | Construct | TagagcattgcagatggacCgc | 119 | gaGgtCCatctacaacactctA | 131 |
| | 124-3 | #48 | | | | |
| MW | E-miR- | Construct | TagagcattgcagatggacCgc | 120 | gCGgtCCatctacaacactTtA | 132 |
| | 124-3 | #49 | | | | |

### Table 7 Sequence Key:

The term “Antisense sequence,” as used in Table 7, refers to the antisense sequence MW or a variant thereof having 1-7 (e.g., 1, 2, 3, 4, 5, 6, or 7) modifications (e.g., substitution, deletion, insertion, or mismatch).
CAPITAL BOLD CHARACTERS: Nucleotides in a modified guide or passenger strand sequence relative to Construct C.

The inhibitory RNA molecules described herein may include a stem-loop sequence containing guide strand and passenger strand sequences rationally designed from the anti-Grik2 sequence MW (SEQ ID NO: 109) embedded in an E-miR-218 microRNA scaffold and sequences complementary thereto (see, e.g., Table 8 (e.g., SEQ ID NOs: 133-138)), or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity thereto.

TABLE 8
Grik2-targeting constructs containing the antisense sequence MW or a variant thereof

| Antisense | miRNA | | | SEQ |
|---|---|---|---|---|
| sequence* | scaffold | Version | Stem-loop Sequence | ID NO |
| MW | E-miR- | Construct | acgtttccagaacgtctgtagcttttctcctccttccctccattttcctcttggtcttacc | 133 |
| | 218-1 | D | tttggcctagtggttggtgtagtgataatgtagcgagattttctgCAGAGCAT | |
| | | | TGCAGATGGACTGggttgcgaggtatgagtaaacagtccatacgcaa | |
| | | | tgctccgtggaacgtcacgcagctttctacagcatgacaagctgctgaggctta | |
| | | | aatcaggattttcctgtctctttctacaaaatcaaaatgaaaaaagagggcttttta | |
| | | | ggcatctccgagattatgtg | |
| MW | E-miR- | Construct | acgtttccagaacgtctgtagcttttctcctccttccctccattttcctcttggtcttacc | 134 |
| | 218-1 | #50 | tttggcctagtggttggtgtagtgataatgtagcgagattttctgTAGAGCATT | |
| | | | GCAGATGGCCCGggttgcgaggtatgagtaaacGgGccatacgca | |
| | | | atgctccAtggaacgtcacgcagctttctacagcatgacaagctgctgaggctt | |
| | | | aaatcaggattttcctgtctctttctacaaaatcaaaatgaaaaaagagggcttttt | |
| | | | aggcatctccgagattatgtg | |
| MW | E-miR- | Construct | acgtttccagaacgtctgtagcttttctcctccttccctccattttcctcttggtcttacc | 135 |
| | 218-1 | #51 | tttggcctagtggttggtgtagtgataatgtagcgagattttctgTAGAGCATT | |
| | | | GCAGATGGACCGgattacgaggtatgagtaaacGgtccatacgcaat | |
| | | | gctccAtggaacgtcacgcagctttctacagcatgacaagctgctgaggctta | |
| | | | aatcaggattttcctgtctctttctacaaaatcaaaatgaaaaaagagggcttttta | |
| | | | ggcatctccgagattatgtg | |
| MW | E-miR- | Construct | acgtttccagaacgtctgtagcttttctcctccttccctccattttcctcttggtcttacc | 136 |
| | 218-1 | #52 | tttggcctagtggttggtgtagtgataatgtagcgagattttctgCAGAGCAT | |
| | | | TGCAGATGGCCCGggttgcgaggtatgagtaaacGgGccatacgc | |
| | | | aatgctccgtggaacgtcacgcagctttctacagcatgacaagctgctgaggct | |
| | | | taaatcaggattttcctgtctctttctacaaaatcaaaatgaaaaaagagggctttt | |
| | | | taggcatctccgagattatgtg | |
| MW | E-miR- | Construct | acgtttccagaacgtctgtagcttttctcctccttccctccattttcctcttggtcttacc | 137 |
| | 218-1 | #53 | tttggcctagtggttggtgtagtgataatgtagcgagattttctgCAGAGCAT | |
| | | | TGCAGATGGACCGggttgcgaggtatgagtaaacGgtccatacgca | |
| | | | atgctccgtggaacgtcacgcagctttctacagcatgacaagctgctgaggctt | |
| | | | aaatcaggattttcctgtctctttctacaaaatcaaaatgaaaaaagagggcttttt | |
| | | | aggcatctccgagattatgtg | |
| MW | E-miR- | Construct | acgtttccagaacgtctgtagcttttctcctccttccctccattttcctcttggtcttacc | 138 |
| | 218-1 | #54 | tttggcctagtggttggtgtagtgataatgtagcgagattttctgTAGAGCATT | |
| | | | GCAGATGGACTGggttgcgaggtatgagtaaacagtccatacgcaat | |
| | | | gctccAtggaacgtcacgcagctttctacagcatgacaagctgctgaggctta | |
| | | | aatcaggattttcctgtctctttctacaaaatcaaaatgaaaaaagagggcttttta | |
| | | | ggcatctccgagattatgtg | |

### Table 8 Sequence key:

The term “Antisense sequence,” as used in Table 8, refers to the antisense sequence MW or a variant thereof having 1-7 (e.g., 1, 2, 3, 4, 5, 6, or 7) modifications (e.g., substitution, deletion, insertion, or mismatch).
single and double underlined characters: stem-loop sequence;
CAPITAL ITALIC CHARACTERS WITH SINGLE UNDERLINE: guide strand;
single underlined lower-case characters: E-miR-218 loop sequence;
double-underlined characters: passenger strand;
CAPITAL BOLD CHARACTERS: substituted nucleotides.

Accordingly, the Grik2-targeting antisense constructs of the disclosure may include guide (SEQ ID NOs: 139-144) and passenger strand (SEQ ID NOs: 145-146) pairs described in Table 9, below:

TABLE 9
Guide and passenger strand pairs rationally designed from MW sequence in a miR-218
scaffold

| | | | | SEQ | | SEQ |
|---|---|---|---|---|---|---|
| Antisense | miRNA | | | ID | | ID |
| sequence* | scaffold | Version | Guide sequence | NO | Passenger sequence | NO |
| MW | E-miR- | Construct | cagagcattgcagatggactg | 139 | cagtccatacgcaatgctccg | 145 |
| | 218-1 | D | | | | |
| MW | E-miR- | Construct | TagagcattgcagatggCcCg | 140 | cGgGccatacgcaatgctccA | 146 |
| | 218-1 | #50 | | | | |
| MW | E-miR- | Construct | TagagcattgcagatggacCg | 141 | cGgtccatacgcaatgctccA | 147 |
| | 218-1 | #51 | | | | |
| MW | E-miR- | Construct | cagagcattgcagatggCcCg | 142 | cGgGccatacgcaatgctccg | 148 |
| | 218-1 | #52 | | | | |
| MW | E-miR- | Construct | cagagcattgcagatggacCg | 143 | cGgtccatacgcaatgctccg | 149 |
| | 218-1 | #53 | | | | |
| MW | E-miR- | Construct | Tagagcattgcagatggactg | 144 | cagtccatacgcaatgctccA | 150 |
| | 218-1 | #54 | | | | |

### Table 9 Sequence Key:

The term “Antisense sequence,” as used in Table 9, refers to the antisense sequence MW or a variant thereof having 1-7 (e.g., 1, 2, 3, 4, 5, 6, or 7) modifications (e.g., substitution, deletion, insertion, or mismatch).
CAPITAL BOLD CHARACTERS: Nucleotides in a modified guide or passenger strand sequence relative to Construct D.

The foregoing sequences are represented as DNA (i.e., cDNA) sequences that can be incorporated into a vector of the disclosure. These sequences may also be represented as corresponding RNA sequences that are synthesized from the vector within the cell. One skilled in the art would understand that the cDNA sequence is equivalent to the mRNA sequence, except for the substitution of uridines with thymidines, and can be used for the same purpose herein, i.e., the generation of a polynucleotide for inhibiting the expression of Grik2 mRNA. In the case of DNA vectors (e.g., AAV), the polynucleotide containing the antisense nucleic acid is a DNA sequence. In the case of RNA vectors, the transgene cassette incorporates the RNA equivalent of the antisense DNA sequences described herein.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 1. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 1. In another example, the inhibitory RNA may have at least 95% (e.g., at least 95%, 96%, 97%, 98%, 99%, or more (e.g., 100%)) sequence identity to the nucleic acid sequence of SEQ ID NO: 1. In a further example, the inhibitory RNA may have the nucleic acid sequence of SEQ ID NO: 1.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 2. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 2. In another example, the inhibitory RNA may have at least 95% (e.g., at least 95%, 96%, 97%, 98%, 99%, or more (e.g., 100%)) sequence identity to the nucleic acid sequence of SEQ ID NO: 2. In a further example, the inhibitory RNA may have the nucleic acid sequence of SEQ ID NO: 2.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 3. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 3. In another example, the inhibitory RNA may have at least 95% (e.g., at least 95%, 96%, 97%, 98%, 99%, or more (e.g., 100%)) sequence identity to the nucleic acid sequence of SEQ ID NO: 3. In a further example, the inhibitory RNA may have the nucleic acid sequence of SEQ ID NO: 3.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 4. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 4. In another example, the inhibitory RNA may have at least 95% (e.g., at least 95%, 96%, 97%, 98%, 99%, or more (e.g., 100%)) sequence identity to the nucleic acid sequence of SEQ ID NO: 4. In a further example, the inhibitory RNA may have the nucleic acid sequence of SEQ ID NO: 4.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 5. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 5. In another example, the inhibitory RNA may have at least 95% (e.g., at least 95%, 96%, 97%, 98%, 99%, or more (e.g., 100%)) sequence identity to the nucleic acid sequence of SEQ ID NO: 5. In a further example, the inhibitory RNA may have the nucleic acid sequence of SEQ ID NO: 5.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 6. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 6. In another example, the inhibitory RNA may have at least 95% (e.g., at least 95%, 96%, 97%, 98%, 99%, or more (e.g., 100%)) sequence identity to the nucleic acid sequence of SEQ ID NO: 6. In a further example, the inhibitory RNA may have the nucleic acid sequence of SEQ ID NO: 6.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 7. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 7. In another example, the inhibitory RNA may have at least 95% (e.g., at least 95%, 96%, 97%, 98%, 99%, or more (e.g., 100%)) sequence identity to the nucleic acid sequence of SEQ ID NO: 7. In a further example, the inhibitory RNA may have the nucleic acid sequence of SEQ ID NO: 7.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 8. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 8. In another example, the inhibitory RNA may have at least 95% (e.g., at least 95%, 96%, 97%, 98%, 99%, or more (e.g., 100%)) sequence identity to the nucleic acid sequence of SEQ ID NO: 8. In a further example, the inhibitory RNA may have the nucleic acid sequence of SEQ ID NO: 8.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 9. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 9. In another example, the inhibitory RNA may have at least 95% (e.g., at least 95%, 96%, 97%, 98%, 99%, or more (e.g., 100%)) sequence identity to the nucleic acid sequence of SEQ ID NO: 9. In a further example, the inhibitory RNA may have the nucleic acid sequence of SEQ ID NO: 9.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 10. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 10. In another example, the inhibitory RNA may have at least 95% (e.g., at least 95%, 96%, 97%, 98%, 99%, or more (e.g., 100%)) sequence identity to the nucleic acid sequence of SEQ ID NO: 10. In a further example, the inhibitory RNA may have the nucleic acid sequence of SEQ ID NO: 10.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 11. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 11. In another example, the inhibitory RNA may have at least 95% (e.g., at least 95%, 96%, 97%, 98%, 99%, or more (e.g., 100%)) sequence identity to the nucleic acid sequence of SEQ ID NO: 11. In a further example, the inhibitory RNA may have the nucleic acid sequence of SEQ ID NO: 11.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 12. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 12. In another example, the inhibitory RNA may have at least 95% (e.g., at least 95%, 96%, 97%, 98%, 99%, or more (e.g., 100%)) sequence identity to the nucleic acid sequence of SEQ ID NO: 12. In a further example, the inhibitory RNA may have the nucleic acid sequence of SEQ ID NO: 12.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 13. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 13. In another example, the inhibitory RNA may have at least 95% (e.g., at least 95%, 96%, 97%, 98%, 99%, or more (e.g., 100%)) sequence identity to the nucleic acid sequence of SEQ ID NO: 13. In a further example, the inhibitory RNA may have the nucleic acid sequence of SEQ ID NO: 13.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 14. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 14. In another example, the inhibitory RNA may have at least 95% (e.g., at least 95%, 96%, 97%, 98%, 99%, or more (e.g., 100%)) sequence identity to the nucleic acid sequence of SEQ ID NO: 14. In a further example, the inhibitory RNA may have the nucleic acid sequence of SEQ ID NO: 14.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 15. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 15. In another example, the inhibitory RNA may have at least 95% (e.g., at least 95%, 96%, 97%, 98%, 99%, or more (e.g., 100%)) sequence identity to the nucleic acid sequence of SEQ ID NO: 15. In a further example, the inhibitory RNA may have the nucleic acid sequence of SEQ ID NO: 15.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 226. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 226. In another example, the inhibitory RNA may have at least 95% (e.g., at least 95%, 96%, 97%, 98%, 99%, or more (e.g., 100%)) sequence identity to the nucleic acid sequence of SEQ ID NO: 226. In a further example, the inhibitory RNA may have the nucleic acid sequence of SEQ ID NO: 226.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 227. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 227. In another example, the inhibitory RNA may have at least 95% (e.g., at least 95%, 96%, 97%, 98%, 99%, or more (e.g., 100%)) sequence identity to the nucleic acid sequence of SEQ ID NO: 227. In a further example, the inhibitory RNA may have the nucleic acid sequence of SEQ ID NO: 227.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 228. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 228. In another example, the inhibitory RNA may have at least 95% (e.g., at least 95%, 96%, 97%, 98%, 99%, or more (e.g., 100%)) sequence identity to the nucleic acid sequence of SEQ ID NO: 228. In a further example, the inhibitory RNA may have the nucleic acid sequence of SEQ ID NO: 228.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 229. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 229. In another example, the inhibitory RNA may have at least 95% (e.g., at least 95%, 96%, 97%, 98%, 99%, or more (e.g., 100%)) sequence identity to the nucleic acid sequence of SEQ ID NO: 229. In a further example, the inhibitory RNA may have the nucleic acid sequence of SEQ ID NO: 229.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 238. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 238. In another example, the inhibitory RNA may have at least 95% (e.g., at least 95%, 96%, 97%, 98%, 99%, or more (e.g., 100%)) sequence identity to the nucleic acid sequence of SEQ ID NO: 238. In a further example, the inhibitory RNA may have the nucleic acid sequence of SEQ ID NO: 238.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 239. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 239. In another example, the inhibitory RNA may have at least 95% (e.g., at least 95%, 96%, 97%, 98%, 99%, or more (e.g., 100%)) sequence identity to the nucleic acid sequence of SEQ ID NO: 239. In a further example, the inhibitory RNA may have the nucleic acid sequence of SEQ ID NO: 239.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 240. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 240. In another example, the inhibitory RNA may have at least 95% (e.g., at least 95%, 96%, 97%, 98%, 99%, or more (e.g., 100%)) sequence identity to the nucleic acid sequence of SEQ ID NO: 240. In a further example, the inhibitory RNA may have the nucleic acid sequence of SEQ ID NO: 240.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 241. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 241. In another example, the inhibitory RNA may have at least 95% (e.g., at least 95%, 96%, 97%, 98%, 99%, or more (e.g., 100%)) sequence identity to the nucleic acid sequence of SEQ ID NO: 241. In a further example, the inhibitory RNA may have the nucleic acid sequence of SEQ ID NO: 241.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 46. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 46. In another example, the inhibitory RNA may have at least 95% (e.g., at least 95%, 96%, 97%, 98%, 99%, or more (e.g., 100%)) sequence identity to the nucleic acid sequence of SEQ ID NO: 46. In a further example, the inhibitory RNA may have the nucleic acid sequence of SEQ ID NO: 46.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 47. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 47. In another example, the inhibitory RNA may have at least 95% (e.g., at least 95%, 96%, 97%, 98%, 99%, or more (e.g., 100%)) sequence identity to the nucleic acid sequence of SEQ ID NO: 47. In a further example, the inhibitory RNA may have the nucleic acid sequence of SEQ ID NO: 47.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 48. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 48. In another example, the inhibitory RNA may have at least 95% (e.g., at least 95%, 96%, 97%, 98%, 99%, or more (e.g., 100%)) sequence identity to the nucleic acid sequence of SEQ ID NO: 48. In a further example, the inhibitory RNA may have the nucleic acid sequence of SEQ ID NO: 48.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 49. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 49. In another example, the inhibitory RNA may have at least 95% (e.g., at least 95%, 96%, 97%, 98%, 99%, or more (e.g., 100%)) sequence identity to the nucleic acid sequence of SEQ ID NO: 49. In a further example, the inhibitory RNA may have the nucleic acid sequence of SEQ ID NO: 49.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 50. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 50. In another example, the inhibitory RNA may have at least 95% (e.g., at least 95%, 96%, 97%, 98%, 99%, or more (e.g., 100%)) sequence identity to the nucleic acid sequence of SEQ ID NO: 50. In a further example, the inhibitory RNA may have the nucleic acid sequence of SEQ ID NO: 50.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 51. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 51. In another example, the inhibitory RNA may have at least 95% (e.g., at least 95%, 96%, 97%, 98%, 99%, or more (e.g., 100%)) sequence identity to the nucleic acid sequence of SEQ ID NO: 51. In a further example, the inhibitory RNA may have the nucleic acid sequence of SEQ ID NO: 51.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 52. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 52. In another example, the inhibitory RNA may have at least 95% (e.g., at least 95%, 96%, 97%, 98%, 99%, or more (e.g., 100%)) sequence identity to the nucleic acid sequence of SEQ ID NO: 52. In a further example, the inhibitory RNA may have the nucleic acid sequence of SEQ ID NO: 52.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 53. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 53. In another example, the inhibitory RNA may have at least 95% (e.g., at least 95%, 96%, 97%, 98%, 99%, or more (e.g., 100%)) sequence identity to the nucleic acid sequence of SEQ ID NO: 53. In a further example, the inhibitory RNA may have the nucleic acid sequence of SEQ ID NO: 53.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 54. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 54. In another example, the inhibitory RNA may have at least 95% (e.g., at least 95%, 96%, 97%, 98%, 99%, or more (e.g., 100%)) sequence identity to the nucleic acid sequence of SEQ ID NO: 54. In a further example, the inhibitory RNA may have the nucleic acid sequence of SEQ ID NO: 54.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 55. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 55. In another example, the inhibitory RNA may have at least 95% (e.g., at least 95%, 96%, 97%, 98%, 99%, or more (e.g., 100%)) sequence identity to the nucleic acid sequence of SEQ ID NO: 55. In a further example, the inhibitory RNA may have the nucleic acid sequence of SEQ ID NO: 55.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 56. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 56. In another example, the inhibitory RNA may have at least 95% (e.g., at least 95%, 96%, 97%, 98%, 99%, or more (e.g., 100%)) sequence identity to the nucleic acid sequence of SEQ ID NO: 56. In a further example, the inhibitory RNA may have the nucleic acid sequence of SEQ ID NO: 56.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 57. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 57. In another example, the inhibitory RNA may have at least 95% (e.g., at least 95%, 96%, 97%, 98%, 99%, or more (e.g., 100%)) sequence identity to the nucleic acid sequence of SEQ ID NO: 57. In a further example, the inhibitory RNA may have the nucleic acid sequence of SEQ ID NO: 57.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 58. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 58. In another example, the inhibitory RNA may have at least 95% (e.g., at least 95%, 96%, 97%, 98%, 99%, or more (e.g., 100%)) sequence identity to the nucleic acid sequence of SEQ ID NO: 58. In a further example, the inhibitory RNA may have the nucleic acid sequence of SEQ ID NO: 58.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 59. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 59. In another example, the inhibitory RNA may have at least 95% (e.g., at least 95%, 96%, 97%, 98%, 99%, or more (e.g., 100%)) sequence identity to the nucleic acid sequence of SEQ ID NO: 59. In a further example, the inhibitory RNA may have the nucleic acid sequence of SEQ ID NO: 59.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 60. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 60. In another example, the inhibitory RNA may have at least 95% (e.g., at least 95%, 96%, 97%, 98%, 99%, or more (e.g., 100%)) sequence identity to the nucleic acid sequence of SEQ ID NO: 60 In a further example, the inhibitory RNA may have the nucleic acid sequence of SEQ ID NO: 60.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 61. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 61. In another example, the inhibitory RNA may have at least 95% (e.g., at least 95%, 96%, 97%, 98%, 99%, or more (e.g., 100%)) sequence identity to the nucleic acid sequence of SEQ ID NO: 61. In a further example, the inhibitory RNA may have the nucleic acid sequence of SEQ ID NO: 61.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 62. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 62. In another example, the inhibitory RNA may have at least 95% (e.g., at least 95%, 96%, 97%, 98%, 99%, or more (e.g., 100%)) sequence identity to the nucleic acid sequence of SEQ ID NO: 62. In a further example, the inhibitory RNA may have the nucleic acid sequence of SEQ ID NO: 62.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 97. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 97. In another example, the inhibitory RNA may have at least 95% (e.g., at least 95%, 96%, 97%, 98%, 99%, or more (e.g., 100%)) sequence identity to the nucleic acid sequence of SEQ ID NO: 97. In a further example, the inhibitory RNA may have the nucleic acid sequence of SEQ ID NO: 97.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 98. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 98. In another example, the inhibitory RNA may have at least 95% (e.g., at least 95%, 96%, 97%, 98%, 99%, or more (e.g., 100%)) sequence identity to the nucleic acid sequence of SEQ ID NO: 98. In a further example, the inhibitory RNA may have the nucleic acid sequence of SEQ ID NO: 98.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 99. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 99. In another example, the inhibitory RNA may have at least 95% (e.g., at least 95%, 96%, 97%, 98%, 99%, or more (e.g., 100%)) sequence identity to the nucleic acid sequence of SEQ ID NO: 99. In a further example, the inhibitory RNA may have the nucleic acid sequence of SEQ ID NO: 99.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 100. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 100. In another example, the inhibitory RNA may have at least 95% (e.g., at least 95%, 96%, 97%, 98%, 99%, or more (e.g., 100%)) sequence identity to the nucleic acid sequence of SEQ ID NO: 100. In a further example, the inhibitory RNA may have the nucleic acid sequence of SEQ ID NO: 100.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 101. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 101. In another example, the inhibitory RNA may have at least 95% (e.g., at least 95%, 96%, 97%, 98%, 99%, or more (e.g., 100%)) sequence identity to the nucleic acid sequence of SEQ ID NO: 101. In a further example, the inhibitory RNA may have the nucleic acid sequence of SEQ ID NO: 101.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 102. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 102. In another example, the inhibitory RNA may have at least 95% (e.g., at least 95%, 96%, 97%, 98%, 99%, or more (e.g., 100%)) sequence identity to the nucleic acid sequence of SEQ ID NO: 102. In a further example, the inhibitory RNA may have the nucleic acid sequence of SEQ ID NO: 102.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 103. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 103. In another example, the inhibitory RNA may have at least 95% (e.g., at least 95%, 96%, 97%, 98%, 99%, or more (e.g., 100%)) sequence identity to the nucleic acid sequence of SEQ ID NO: 103. In a further example, the inhibitory RNA may have the nucleic acid sequence of SEQ ID NO: 103.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 104. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 104. In another example, the inhibitory RNA may have at least 95% (e.g., at least 95%, 96%, 97%, 98%, 99%, or more (e.g., 100%)) sequence identity to the nucleic acid sequence of SEQ ID NO: 104. In a further example, the inhibitory RNA may have the nucleic acid sequence of SEQ ID NO: 104.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 105. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 105. In another example, the inhibitory RNA may have at least 95% (e.g., at least 95%, 96%, 97%, 98%, 99%, or more (e.g., 100%)) sequence identity to the nucleic acid sequence of SEQ ID NO: 105. In a further example, the inhibitory RNA may have the nucleic acid sequence of SEQ ID NO: 105.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 106. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 106. In another example, the inhibitory RNA may have at least 95% (e.g., at least 95%, 96%, 97%, 98%, 99%, or more (e.g., 100%)) sequence identity to the nucleic acid sequence of SEQ ID NO: 106. In a further example, the inhibitory RNA may have the nucleic acid sequence of SEQ ID NO: 106.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 107. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 107. In another example, the inhibitory RNA may have at least 95% (e.g., at least 95%, 96%, 97%, 98%, 99%, or more (e.g., 100%)) sequence identity to the nucleic acid sequence of SEQ ID NO: 107. In a further example, the inhibitory RNA may have the nucleic acid sequence of SEQ ID NO: 107.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 108. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 108. In another example, the inhibitory RNA may have at least 95% (e.g., at least 95%, 96%, 97%, 98%, 99%, or more (e.g., 100%)) sequence identity to the nucleic acid sequence of SEQ ID NO: 108. In a further example, the inhibitory RNA may have the nucleic acid sequence of SEQ ID NO: 108.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 133. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 133. In another example, the inhibitory RNA may have at least 95% (e.g., at least 95%, 96%, 97%, 98%, 99%, or more (e.g., 100%)) sequence identity to the nucleic acid sequence of SEQ ID NO: 133. In a further example, the inhibitory RNA may have the nucleic acid sequence of SEQ ID NO: 133.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 134. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 134. In another example, the inhibitory RNA may have at least 95% (e.g., at least 95%, 96%, 97%, 98%, 99%, or more (e.g., 100%)) sequence identity to the nucleic acid sequence of SEQ ID NO: 133. In a further example, the inhibitory RNA may have the nucleic acid sequence of SEQ ID NO: 134.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 135. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 135. In another example, the inhibitory RNA may have at least 95% (e.g., at least 95%, 96%, 97%, 98%, 99%, or more (e.g., 100%)) sequence identity to the nucleic acid sequence of SEQ ID NO: 135. In a further example, the inhibitory RNA may have the nucleic acid sequence of SEQ ID NO: 135.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 136. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 136. In another example, the inhibitory RNA may have at least 95% (e.g., at least 95%, 96%, 97%, 98%, 99%, or more (e.g., 100%)) sequence identity to the nucleic acid sequence of SEQ ID NO: 136. In a further example, the inhibitory RNA may have the nucleic acid sequence of SEQ ID NO: 136.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 137. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 137. In another example, the inhibitory RNA may have at least 95% (e.g., at least 95%, 96%, 97%, 98%, 99%, or more (e.g., 100%)) sequence identity to the nucleic acid sequence of SEQ ID NO: 137. In a further example, the inhibitory RNA may have the nucleic acid sequence of SEQ ID NO: 137.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 138. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 138. In another example, the inhibitory RNA may have at least 95% (e.g., at least 95%, 96%, 97%, 98%, 99%, or more (e.g., 100%)) sequence identity to the nucleic acid sequence of SEQ ID NO: 138. In a further example, the inhibitory RNA may have the nucleic acid sequence of SEQ ID NO: 138.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 258. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 258. In another example, the inhibitory RNA may have at least 95% (e.g., at least 95%, 96%, 97%, 98%, 99%, or more (e.g., 100%)) sequence identity to the nucleic acid sequence of SEQ ID NO: 258. In a further example, the inhibitory RNA may have the nucleic acid sequence of SEQ ID NO: 258.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 259. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 259. In another example, the inhibitory RNA may have at least 95% (e.g., at least 95%, 96%, 97%, 98%, 99%, or more (e.g., 100%)) sequence identity to the nucleic acid sequence of SEQ ID NO: 259. In a further example, the inhibitory RNA may have the nucleic acid sequence of SEQ ID NO: 259.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 260. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 260. In another example, the inhibitory RNA may have at least 95% (e.g., at least 95%, 96%, 97%, 98%, 99%, or more (e.g., 100%)) sequence identity to the nucleic acid sequence of SEQ ID NO: 260. In a further example, the inhibitory RNA may have the nucleic acid sequence of SEQ ID NO: 260.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 261. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 261. In another example, the inhibitory RNA may have at least 95% (e.g., at least 95%, 96%, 97%, 98%, 99%, or more (e.g., 100%)) sequence identity to the nucleic acid sequence of SEQ ID NO: 261. In a further example, the inhibitory RNA may have the nucleic acid sequence of SEQ ID NO: 261.

An inhibitory RNA sequence of the disclosure may have at least 85% (e.g., at least 85%, 90%, 95%, 96%, 97%, 98%, 99%, or more (e.g., 100%)) sequence identity to the nucleic acid sequence of SEQ ID NO: 256. For example, the inhibitory RNA may have at least 90% (e.g., at least 90%, 95%, 96%, 97%, 98%, 99%, or more (e.g., 100%)) sequence identity to the nucleic acid sequence of SEQ ID NO: 256. In another example, the inhibitory RNA may have at least 95% (e.g., at least 95%, 96%, 97%, 98%, 99%, or more (e.g., 100%)) sequence identity to the nucleic acid sequence of SEQ ID NO: 256. In a further example, the inhibitory RNA may have the nucleic acid sequence of SEQ ID NO: 256.

Inhibitory Polynucleotides with Wobble Base Pairs

The disclosure further features inhibitory RNA agents having one or more wobble base pairs. The four main wobble base pairs are guanine-uracil (G-U), hypoxanthine-uracil (I-U), hypoxanthine-adenine (I-A), and hypoxanthine-cytosine (I-C), in which hypoxanthine represents the nucleoside inosine. The G-U wobble base pair has been shown to exhibit a similar thermodynamic stability to that of G-C, A-T and A-U (Saxena et al, 2003, J Biol Chem, 278 (45): 44312-9).

Accordingly, the disclosure provides an inhibitory RNA agent having a nucleotide sequence that has at least 85% (e.g., at least 85%, 90%, 95%, 96%, 97%, 98%, 99%, or more (e.g., 100%)) sequence identity to the complement of a target region of any one of SEQ ID NOs: 164-174 (e.g., the inhibitory RNA may have at least 85% (e.g., at least 86%, 87%, 88%, 89%, 90%, 91%, 92%, 93%, 94%, 95%, 96%, 97%, 98%, 99%, or more (e.g., 100%)) sequence identity to the antisense strand of a Grik2 gene sequence). In particular, an inhibitory RNA agent of the disclosure may have 1, 2 or 3 nucleotides that are not complementary to the corresponding aligned human Grik2 mRNA transcript (e.g., any one of SEQ ID NOs: 164-174). As such, an inhibitory RNA agent of the disclosure may have a nucleotide sequence that is at least 85% (e.g., at least 85%, 90%, 95%, 96%, 97%, 98%, 99%, or more (e.g., 100%)), at least 86% (e.g., at least 86%, 90%, 95%, 96%, 97%, 98%, 99%, or more (e.g., 100%)), at least 87% (e.g., at least 87%, 90%, 95%, 96%, 97%, 98%, 99%, or more (e.g., 100%)), at least 88% (e.g., at least 88%, 90%, 95%, 96%, 97%, 98%, 99%, or more (e.g., 100%)), at least 89% (e.g., at least 89%, 90%, 95%, 96%, 97%, 98%, 99%, or more (e.g., 100%)) or at least 90% (e.g., at least 90%, 95%, 96%, 97%, 98%, 99%, or more (e.g., 100%)) identical to the complement of a target region of any one of SEQ ID NOs: 164-174. The nucleotides that are not 100% identical to the complementary sequence of the aligned Grik2 mRNA sequence may be a wobble nucleotide.

The probability of off-target effects mediated by antisense RNAs designed against a particular region on a Grik2 transcript may be measured using any number of publicly available algorithms. For example, the online tool siSPOTR (“siRNA Sequence Probability-of-Off-Targeting Reduction”, which is available at world-wide-web.sispotr.icts.uiowa.edu/sispotr/index.html_, can be used).

The inhibitory RNA agents disclosed herein target an mRNA encoding a GluK2 protein (e.g., GluK2 protein including any one of SEQ ID NOs: 151-163, or GluK2 protein including at least amino acids 1 to 509 of SEQ ID NO: 151). The mRNA encoding a GluK2 protein may include a polynucleotide encoding polypeptide that contains one or more amino acid substitutions, such as one or more conservative amino acid substitutions (e.g., 1, 2, 3, 4, 5, 6, 7, 8, 9, or 10 or more amino acid substitutions, such as 1, 2, 3, 4, 5, 6, 7, 8, 9, or 10 or more conservative amino acid substitutions), relative to a polypeptide having the sequence of any one of SEQ ID NOs: 151-163.

### Grik2 Proteins and Polynucleotides Encoding the Same

The Grik2 inhibitory RNA agents disclosed herein may be designed by using the sequence of the Grik2 mRNA as a starting point by using, e.g., bioinformatic tools. Grik2 mRNA sequences may be found in NCBI Gene ID NO: 2898. In another example, a polynucleotide sequence encoding SEQ ID NO: 151, a polynucleotide sequence encoding contiguous amino acids 1 to 509 of SEQ ID NO: 151, or a polynucleotide sequence encoding the amino acid sequence of any one of SEQ ID NO: 151 (UniProtKB Q13002-1), SEQ ID NO: 152 (UniProtKB Q13002-2), SEQ ID NO: 153 (UniProtKB Q13002-3), SEQ ID NO: 154 (UniProtKB Q13002-4), SEQ ID NO: 155 (UniProtKB Q13002-5), SEQ ID NO: 156 (UniProtKB Q13002-6), SEQ ID NO: 157 (UniProtKB Q13002-7), SEQ ID NO: 158 (NCBI Accession No.: NP_001104738.2), SEQ ID NO: 159 (NCBI Accession No.: NP_034479.3), SEQ ID NO: 160 (NCBI Accession No.: NP_034479.3), SEQ ID NO: 161 (NCBI Accession No.: XP_014992481.1), SEQ ID NO: 162 (NCBI Accession No.: XP_014992483.1), and SEQ ID NO: 163 (NCBI Accession No.: NP_062182.1) can be used as a basis for designing nucleic acids that target an mRNA encoding GluK2 protein. Polynucleotide sequences encoding a GluK2 receptor may be selected from any one of SEQ ID NOs: 164-174.

The GluK2 polypeptide may have an amino acid sequence of SEQ ID NO: 151 or may be a variant thereof with at least 85% (e.g., at least 85%, 90%, 95%, 96%, 97%, 98%, 99%, or more (e.g., 100%)) sequence identity to the amino acid sequence of SEQ ID NO: 151, which is shown below (UniProt Q13002-1; GRIK2_HUMAN Glutamate receptor ionotropic, kainate 2):

(SEQ ID NO: 151)
MKIIFPILSNPVFRRTVKLLLCLLWIGYSQGTTHVLRFGGIFEYVESGPM
GAEELAFRFAVNTINRNRTLLPNTTLTYDTQKINLYDSFEASKKACDQLS
LGVAAIFGPSHSSSANAVQSICNALGVPHIQTRWKHQVSDNKDSFYVSLY
PDFSSLSRAILDLVQFFKWKTVTVVYDDSTGLIRLQELIKAPSRYNLRLK
IRQLPADTKDAKPLLKEMKRGKEFHVIFDCSHEMAAGILKQALAMGMMTE
YYHYIFTTLDLFALDVEPYRYSGVNMTGFRILNTENTQVSSIIEKWSMER
LQAPPKPDSGLLDGFMTTDAALMYDAVHVVSVAVQQFPQMTVSSLQCNRH
KPWRFGTRFMSLIKEAHWEGLTGRITFNKTNGLRTDFDLDVISLKEEGLE
KIGTWDPASGLNMTESQKGKPANITDSLSNRSLIVTTILEEPYVLFKKSD
KPLYGNDRFEGYCIDLLRELSTILGFTYEIRLVEDGKYGAQDDANGQWNG
MVRELIDHKADLAVAPLAITYVREKVIDFSKPFMTLGISILYRKPNGTNP
GVFSFLNPLSPDIWMYILLAYLGVSCVLFVIARFSPYEWYNPHPCNPDSD
VVENNFTLLNSFWFGVGALMQQGSELMPKALSTRIVGGIWWFFTLIIISS
YTANLAAFLTVERMESPIDSADDLAKQTKIEYGAVEDGATMTFFKKSKIS
TYDKMWAFMSSRRQSVLVKSNEEGIQRVLTSDYAFLMESTTIEFVTQRNC
NLTQIGGLIDSKGYGVGTPMGSPYRDKITIAILQLQEEGKLHMMKEKWWR
GNGCPEEESKEASALGVQNIGGIFIVLAAGLVLSVFVAVGEFLYKSKKNA
QLEKRSFCSAMVEELRMSLKCQRRLKHKPQAPVIVKTEEVINMHTFNDRR
LPGKETMA

The GluK2 polypeptide may have an amino acid sequence of SEQ ID NO: 152 or may be a variant thereof with at least 85% (e.g., at least 85%, 90%, 95%, 96%, 97%, 98%, 99%, or more (e.g., 100%)) sequence identity to the amino acid sequence of SEQ ID NO: 152, which is shown below (UniProt Q13002-2; GRIK2_HUMAN Isoform 2 of Glutamate receptor ionotropic, kainate 2):

(SEQ ID NO: 152)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKADLAVAPLAITYVREKVIDFSKPFMTLGISI
LYRKPNGTNPGVFSFLNPLSPDIWMYILLAYLGVSCVLFVIARFS
PYEWYNPHPCNPDSDVVENNFTLLNSFWFGVGALMQQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNCNLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKE
SSIWLVPPYHPDTV

The GluK2 polypeptide may have an amino acid sequence of SEQ ID NO: 153 or may be a variant thereof with at least 85% (e.g., at least 85%, 90%, 95%, 96%, 97%, 98%, 99%, or more (e.g., 100%)) sequence identity to the amino acid sequence of SEQ ID NO: 153, which is shown below (UniProt Q13002-3; GRIK2_HUMAN Isoform 3 of Glutamate receptor ionotropic, kainate 2):

(SEQ ID NO: 153)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKADLAVAPLAITYVREKVIDFSKPFMTLGISI
LYRKPNGTNPGVFSFLNPLSPDIWMYILLAYLGVSCVLFVIARF

The GluK2 polypeptide may have an amino acid sequence of SEQ ID NO: 154 or may be a variant thereof with at least 85% (e.g., at least 85%, 90%, 95%, 96%, 97%, 98%, 99%, or more (e.g., 100%)) sequence identity to the amino acid sequence of SEQ ID NO: 154, which is shown below (UniProt Q13002-4; GRIK2_HUMAN Isoform 4 of Glutamate receptor ionotropic, kainate 2):

(SEQ ID NO: 154)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKADLAVAPLAITYVREKVIDFSKPFMTLGISI
LYRKPNGSELMPKALSTRIVGGIWWFFTLIIISSYTANLAAFLTV
ERMESPIDSADDLAKQTKIEYGAVEDGATMTFFKKSKISTYDKMW
AFMSSRRQSVLVKSNEEGIQRVLTSDYAFLMESTTIEFVTQRNCN
LTQIGGLIDSKGYGVGTPMGSPYRDKITIAILQLQEEGKLHMMKE
KWWRGNGCPEEESKEASALGVQNIGGIFIVLAAGLVLSVFVAVGE
FLYKSKKNAQLEKRSFCSAMVEELRMSLKCQRRLKHKPQAPVIVK
TEEVINMHTFNDRRLPGKETMA

The GluK2 polypeptide may have an amino acid sequence of SEQ ID NO: 155 or may be a variant thereof with at least 85% (e.g., at least 85%, 90%, 95%, 96%, 97%, 98%, 99%, or more (e.g., 100%)) sequence identity to the amino acid sequence of SEQ ID NO: 155, which is shown below (UniProt Q13002-5; GRIK2_HUMAN Isoform 5 of Glutamate receptor ionotropic, kainate 2):

(SEQ ID NO: 155)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKADLAVAPLAITYVREKVIDFSKPFMTLGISI
LYRKPNGTNPGVFSFLNPLSPDIWMYILLAYLGVSCVLFVIARFS
PYEWYNPHPCNPDSDVVENNFTLLNSFWFGVGALMQQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNONLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKR
AKTKLPQDYVFLPILESVSISTVLSSSPSSSSLSSCS

The GluK2 polypeptide may have an amino acid sequence of SEQ ID NO: 156 or may be a variant thereof with at least 85% (e.g., at least 85%, 90%, 95%, 96%, 97%, 98%, 99%, or more (e.g., 100%)) sequence identity to the amino acid sequence of SEQ ID NO: 156, which is shown below (UniProt Q13002-6; GRIK2_HUMAN Isoform 6 of Glutamate receptor ionotropic, kainate 2):

(SEQ ID NO: 156)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKSKISTYDKMWAFMSSRRQSVLVKSNEEGIQR
VLTSDYAFLMESTTIEFVTQRNCNLTQIGGLIDSKGYGVGTPMGS
PYRDKITIAILQLQEEGKLHMMKEKWWRGNGCPEEESKEASALGV
QNIGGIFIVLAAGLVLSVFVAVGEFLYKSKKNAQLEKESSIWLVP
PYHPDTV

The GluK2 polypeptide may have an amino acid sequence of SEQ ID NO: 157 or may be a variant thereof with at least 85% (e.g., at least 85%, 90%, 95%, 96%, 97%, 98%, 99%, or more (e.g., 100%)) sequence identity to the amino acid sequence of SEQ ID NO: 157, which is shown below (UniProt Q13002-7; GRIK2_HUMAN Isoform 7 of Glutamate receptor ionotropic, kainate 2):

(SEQ ID NO: 157)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKSVLVKSNEEGIQRVLTSDYAFLMESTTIEFV
TQRNCNLTQIGGLIDSKGYGVGTPMGSPYRDKITIAILQLQEEGK
LHMMKEKWWRGNGCPEEESKEASALGVQNIGGIFIVLAAGLVLSV
FVAVGEFLYKSKKNAQLEKRAKTKLPQDYVFLPILESVSISTVLS
SSPSSSSLSSCS

The GluK2 polypeptide may have an amino acid sequence of SEQ ID NO: 158 or may be a variant thereof with at least 85% (e.g., at least 85%, 90%, 95%, 96%, 97%, 98%, 99%, or more (e.g., 100%)) sequence identity to the amino acid sequence of SEQ ID NO: 158, which is shown below (NP_001104738.2; GRIK2_MOUSE Isoform 1 precursor of Glutamate receptor ionotropic, kainate 2):

(SEQ ID NO: 158)
MKIISPVLSNLVFSRSIKVLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPSSGLNMTESQKGKPANITDSLSNRSLIVTTILEEPYVLFKKSD
KPLYGNDRFEGYCIDLLRELSTILGFTYEIRLVEDGKYGAQDDVN
GQWNGMVRELIDHKADLAVAPLAITYVREKVIDFSKPFMTLGISI
LYRKPNGTNPGVFSFLNPLSPDIWMYILLAYLGVSCVLFVIARFS
PYEWYNPHPCNPDSDVVENNFTLLNSFWFGVGALMQQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNCNLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKR
SFCSAMVEELRMSLKCQRRLKHKPQAPVIVKTEEVINMHTFNDRR
LPGKETMA

The GluK2 polypeptide may have an amino acid sequence of SEQ ID NO: 159 or may be a variant thereof with at least 85% (e.g., at least 85%, 90%, 95%, 96%, 97%, 98%, 99%, or more (e.g., 100%)) sequence identity to the amino acid sequence of SEQ ID NO: 159, which is shown below (NP_034479.3; GRIK2_MOUSE Isoform 2 precursor of Glutamate receptor ionotropic, kainate 2):

(SEQ ID NO: 159)
MKIISPVLSNLVFSRSIKVLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPSSGLNMTESQKGKPANITDSLSNRSLIVTTILEEPYVLFKKSD
KPLYGNDRFEGYCIDLLRELSTILGFTYEIRLVEDGKYGAQDDVN
GQWNGMVRELIDHKADLAVAPLAITYVREKVIDFSKPFMTLGISI
LYRKPNGTNPGVFSFLNPLSPDIWMYILLAYLGVSCVLFVIARFS
PYEWYNPHPCNPDSDVVENNFTLLNSFWFGVGALMQQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNONLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKE
SSIWLVPPYHPDTV

The GluK2 polypeptide may have an amino acid sequence of SEQ ID NO: 160 or may be a variant thereof with at least 85% (e.g., at least 85%, 90%, 95%, 96%, 97%, 98%, 99%, or more (e.g., 100%)) sequence identity to the amino acid sequence of SEQ ID NO: 160, which is shown below (NP_001345795.2; GRIK2_MOUSE Isoform 1 precursor of Glutamate receptor ionotropic, kainate 2):

(SEQ ID NO: 160)
MKIISPVLSNLVFSRSIKVLLCLLWIGYSQGTTHVLRFGGIFEYVE
SGPMGAEELAFRFAVNTINRNRTLLPNTTLTYDTQKINLYDSFEA
SKKACDQLSLGVAAIFGPSHSSSANAVQSICNALGVPHIQTRWKH
QVSDNKDSFYVSLYPDFSSLSRAILDLVQFFKWKTVTVVYDDSTG
LIRLQELIKAPSRYNLRLKIRQLPADTKDAKPLLKEMKRGKEFHV
IFDCSHEMAAGILKQALAMGMMTEYYHYIFTTLDLFALDVEPYRY
SGVNMTGFRILNTENTQVSSIIEKWSMERLQAPPKPDSGLLDGFM
TTDAALMYDAVHVVSVAVQQFPQMTVSSLQCNRHKPWRFGTRFMS
LIKEAHWEGLTGRITFNKTNGLRTDFDLDVISLKEEGLEKIGTWD
PSSGLNMTESQKGKPANITDSLSNRSLIVTTILEEPYVLFKKSDK
PLYGNDRFEGYCIDLLRELSTILGFTYEIRLVEDGKYGAQDDVNG
QWNGMVRELIDHKADLAVAPLAITYVREKVIDFSKPFMTLGISIL
YRKPNGTNPGVFSFLNPLSPDIWMYILLAYLGVSCVLFVIARFSP
YEWYNPHPCNPDSDVVENNFTLLNSFWFGVGALMQQGSELMPKAL
STRIVGGIWWFFTLIIISSYTANLAAFLTVERMESPIDSADDLAK
QTKIEYGAVEDGATMTFFKKSKISTYDKMWAFMSSRRQSVLVKSN
EEGIQRVLTSDYAFLMESTTIEFVTQRNCNLTQIGGLIDSKGYGV
GTPMGSPYRDKITIAILQLQEEGKLHMMKEKWWRGNGCPEEESKE
ASALGVQNIGGIFIVLAAGLVLSVFVAVGEFLYKSKKNAQLEKRS
FCSAMVEELRMSLKCQRRLKHKPQAPVIVKTEEVINMHTFNDRRL
PGKETMA

The GluK2 polypeptide may have an amino acid sequence of SEQ ID NO: 161 or may be a variant thereof with at least 85% (e.g., at least 85%, 90%, 95%, 96%, 97%, 98%, 99%, or more (e.g., 100%)) sequence identity to the amino acid sequence of SEQ ID NO: 161, which is shown below (XP_014992481.1; GRIK2_RHESUS MACAQUE Isoform X1, Glutamate receptor ionotropic, kainate 2):

(SEQ ID NO: 161)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKADLAVAPLAITYVREKVIDFSKPFMTLGISI
LYRKPNGTNPGVFSFLNPLSPDIWMYILLAYLGVSCVLFVIARFS
PYEWYNPHPCNPDSDVVENNFTLLNSFWFGVGALMQQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNONLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKR
SFCSAMVEELRMSLKCQRRLKHKPQAPVIVKTEEVINMHTFNDRR
LPGKETMA

The GluK2 polypeptide may have an amino acid sequence of SEQ ID NO: 162 or may be a variant thereof with at least 85% (e.g., at least 85%, 90%, 95%, 96%, 97%, 98%, 99%, or more (e.g., 100%)) sequence identity to the amino acid sequence of SEQ ID NO: 162, which is shown below (XP_014992483.1; GRIK2_RHESUS MACAQUE Isoform X1, Glutamate receptor ionotropic, kainate 2):

(SEQ ID NO: 162)
MKIIFPILSNPVFRRTVKLLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDAN
GQWNGMVRELIDHKADLAVAPLAITYVREKVIDFSKPFMTLGISI
LYRKPNGTNPGVFSFLNPLSPDIWMYILLAYLGVSCVLFVIARFS
PYEWYNPHPCNPDSDVVENNFTLLNSFWFGVGALMQQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNONLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKR
SFCSAMVEELRMSLKCQRRLKHKPQAPVIVKTEEVINMHTFNDRR
LPGKETMA

The GluK2 polypeptide may have an amino acid sequence of SEQ ID NO: 163 or may be a variant thereof with at least 85% (e.g., at least 85%, 90%, 95%, 96%, 97%, 98%, 99%, or more (e.g., 100%)) sequence identity to the amino acid sequence of SEQ ID NO: 163, which is shown below (NP_062182.1; GRIK2_RAT precursor of Glutamate receptor ionotropic, kainate 2):

(SEQ ID NO: 163)
MKIISPVLSNLVFSRSIKVLLCLLWIGYSQGTTHVLRFGGIFEYV
ESGPMGAEELAFRFAVNTINRNRTLLPNTTLTYDTQKINLYDSFE
ASKKACDQLSLGVAAIFGPSHSSSANAVQSICNALGVPHIQTRWK
HQVSDNKDSFYVSLYPDFSSLSRAILDLVQFFKWKTVTVVYDDST
GLIRLQELIKAPSRYNLRLKIRQLPADTKDAKPLLKEMKRGKEFH
VIFDCSHEMAAGILKQALAMGMMTEYYHYIFTTLDLFALDVEPYR
YSGVNMTGFRILNTENTQVSSIIEKWSMERLQAPPKPDSGLLDGF
MTTDAALMYDAVHVVSVAVQQFPQMTVSSLQCNRHKPWRFGTRFM
SLIKEAHWEGLTGRITFNKTNGLRTDFDLDVISLKEEGLEKIGTW
DPASGLNMTESQKGKPANITDSLSNRSLIVTTILEEPYVLFKKSD
KPLYGNDRFEGYCIDLLRELSTILGFTYEIRLVEDGKYGAQDDVN
GQWNGMVRELIDHKADLAVAPLAITYVREKVIDFSKPFMTLGISI
LYRKPNGTNPGVFSFLNPLSPDIWMYVLLACLGVSCVLFVIARFS
PYEWYNPHPCNPDSDVVENNFTLLNSFWFGVGALMRQGSELMPKA
LSTRIVGGIWWFFTLIIISSYTANLAAFLTVERMESPIDSADDLA
KQTKIEYGAVEDGATMTFFKKSKISTYDKMWAFMSSRRQSVLVKS
NEEGIQRVLTSDYAFLMESTTIEFVTQRNONLTQIGGLIDSKGYG
VGTPMGSPYRDKITIAILQLQEEGKLHMMKEKWWRGNGCPEEESK
EASALGVQNIGGIFIVLAAGLVLSVFVAVGEFLYKSKKNAQLEKR
SFCSAMVEELRMSLKCQRRLKHKPQAPVIVKTEEVINMHTFNDRR
LPGKETMA

The Grik2 mRNA may be a polynucleotide containing 5′ and a 3′ untranslated regions (UTR) and having a nucleic acid sequence of SEQ ID NO: 164 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 164 (RefSeq NM_021956.1:4592 Homo sapiens glutamate ionotropic receptor kainate type subunit 2 (GRIK2), transcript variant 1, mRNA), as is shown in Table 10.

The Grik2 mRNA may be a polynucleotide having a nucleic acid sequence of SEQ ID NO: 165 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 165 (RefSeq NM_021956.4:294-3020 Homo sapiens glutamate ionotropic receptor kainate type subunit 2 (GRIK2), transcript variant 1, mRNA), as is shown in Table 10.

Additionally or alternatively, the Grik2 mRNA may be a polynucleotide having a nucleic acid sequence of SEQ ID NO: 166 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 166 (RefSeq NM_175768.3:294-2903 Homo sapiens glutamate ionotropic receptor kainate type subunit 2 (GRIK2), transcript variant 2, mRNA), as is shown in Table 10.

Additionally or alternatively, the Grik2 mRNA may be a polynucleotide having a nucleic acid sequence of SEQ ID NO: 167 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 167 (RefSeq NM_001166247.1:294-2972 Homo sapiens glutamate ionotropic receptor kainate type subunit 2 (GRIK2), transcript variant 3, mRNA), as is shown in Table 10.

Additionally or alternatively, the Grik2 mRNA may be a polynucleotide having a nucleic acid sequence of SEQ ID NO: 168 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 168 (RefSeq NM 001111268.2 Mus musculus glutamate ionotropic receptor kainate type subunit 2 (GRIK2), transcript variant 4, mRNA), as is shown below.

Additionally or alternatively, the Grik2 mRNA may be a polynucleotide having a nucleic acid sequence of SEQ ID NO: 169 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 169 (RefSeq NM_010349.4 Mus musculus glutamate ionotropic receptor kainate type subunit 2 (GRIK2), transcript variant 5, mRNA), as is shown in Table 10.

Additionally or alternatively, the Grik2 mRNA may be a polynucleotide having a nucleic acid sequence of SEQ ID NO: 170 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 170 (RefSeq NM_001358866 Mus musculus glutamate ionotropic receptor kainate type subunit 2 (GRIK2), transcript variant 6, mRNA), as is shown in Table 10.

Additionally or alternatively, the Grik2 mRNA may be a polynucleotide having a nucleic acid sequence of SEQ ID NO: 171 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 171 (RefSeq XM 015136995.2 Macaca mulatta glutamate ionotropic receptor kainate type subunit 2 (GRIK2), transcript variant 7, mRNA), as is shown in Table 10.

Additionally or alternatively, the Grik2 mRNA may be a polynucleotide having a nucleic acid sequence of SEQ ID NO: 172 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 172 (RefSeq XM 015136997.2 Macaca mulatta glutamate ionotropic receptor kainate type subunit 2 (GRIK2), transcript variant X1, mRNA), as is shown in Table 10.

Additionally or alternatively, the Grik2 mRNA may be a polynucleotide having a nucleic acid sequence of SEQ ID NO: 173 or may be a variant thereof having at least 85% (e.g., at least 85%, 90%, 95%, 96%, 97%, 98%, 99%, or more (e.g., 100%)) sequence identity to the nucleic acid sequence of SEQ ID NO: 173 (RefSeq NM_019309.2 Rattus norvegicus glutamate ionotropic receptor kainate type subunit 2 (GRIK2), mRNA), as is shown in Table 10.

Additionally or alternatively, the Grik2 mRNA includes a polynucleotide corresponding to the mature GluK2 peptide coding sequence and having a nucleic acid sequence of SEQ ID NO: 174 or a variant thereof having at least 85% (e.g., at least 85%, 90%, 95%, 96%, 97%, 98%, 99%, or more (e.g., 100%)) sequence identity to the nucleic acid sequence of SEQ ID NO: 174, as is shown in Table 10.

According to the disclosed methods and compositions, the Grik2 mRNA may include a 5′ UTR, such as, e.g., a 5′ UTR encoded by a polynucleotide having the nucleic acid sequence of SEQ ID NO: 175 or a variant thereof having at least 85% (e.g., at least 85%, 90%, 95%, 96%, 97%, 98%, 99%, or more (e.g., 100%)) sequence identity to the nucleic acid sequence of SEQ ID NO: 175, as is shown in Table 10.

The Grik2 mRNA may also include a 3′ UTR, such as a 3′ UTR encoded by a polynucleotide having the nucleic acid sequence of SEQ ID NO: 176 or a variant thereof having at least 85% (e.g., at least 85%, 90%, 95%, 96%, 97%, 98%, 99%, or more (e.g., 100%)) sequence identity to the nucleic acid sequence of SEQ ID NO: 176, as is shown in Table 10.

Additionally, the Grik2 mRNA may include a polynucleotide encoding the Grik2 signal peptide sequence, such as, e.g., a signal peptide sequence encoded by the nucleic acid sequence of SEQ ID NO: 177 or a variant thereof having at least 85% (e.g., at least 85%, 90%, 95%, 96%, 97%, 98%, 99%, or more (e.g., 100%)) sequence identity to the nucleic acid sequence of SEQ ID NO: 177, as is shown in Table 10.

Additionally, the inhibitory polynucleotides of the disclosure are capable of binding within any one of exons 1-16 of the Grik2 transcript (corresponding to SEQ ID NOs: 177-193), which are described in Table 10, below.

TABLE 10
cDNA sequences encoding target Grik2 mRNA sequences

| | Organismal | | SEQ |
|---|---|---|---|
| Description | Source | RefSeq | ID NO |
| Grik2 mRNA, variant 1; | Homo sapiens | NM_021956.1:4592 | 164 |
| Includes a 5′ UTR and a 3′ UTR | | | |
| Grik2 mRNA, variant 1; (CDS only) | Homo sapiens | NM_021956.294-3020 | 165 |
| Corresponds to nt positions 294-3020 | | | |
| of SEQ ID NO: 116. | | | |
| Grik2 mRNA, variant 2 | Homo sapiens | NM_175768.3:294-2903 | 166 |
| Grik2 mRNA, variant 3 | Homo sapiens | NM_001166247.1:294-2972 | 167 |
| Grik2 mRNA, variant 4 | Mus musculus | NM_001111268 | 168 |
| Grik2 mRNA, variant 5 | Mus musculus | NM_010349 | 169 |
| Grik2 mRNA, variant 6 | Mus musculus | NM_001358866 | 170 |
| Grik2 mRNA, variant 7 | Macaca mulatta | XM_015136995.2 | 171 |
| Grik2 mRNA, variant 8 | Macaca mulatta | XM_015136997.2 | 172 |
| Grik2 mRNA, variant 9 | Rattus norvegicus | NM_019309.2 | 173 |
| Grik2 mRNA encoding mature GluK2 | Homo sapiens | NM_021956.1:4592 | 174 |
| peptide CDS | | | |
| Grik2 mRNA, 5′ UTR | Homo sapiens | NM_021956.1:4592 | 175 |
| Grik2 mRNA, 3′ UTR | Homo sapiens | NM_021956.1:4592 | 176 |
| Grik2 mRNA, signal peptide sequence | Homo sapiens | NM_021956.1:4592 | 177 |
| Grik2 mRNA, exon 1; | Homo sapiens | NM_021956.1:4592 | 178 |
| nt 1-408 of SEQ ID NO: 151 | | | |
| Grik2 mRNA, exon 2; | Homo sapiens | NM_021956.1:4592 | 179 |
| nt 409-576 of SEQ ID NO: 151 | | | |
| Grik2 mRNA, exon 3; | Homo sapiens | NM_021956.1 :4592 | 180 |
| nt 577-834 of SEQ ID NO: 151 | | | |
| Grik2 mRNA, exon 4; | Homo sapiens | NM_021956.1:4592 | 181 |
| nt 835-1016 of SEQ ID NO: 151 | | | |
| Grik2 mRNA, exon 5; | Homo sapiens | NM_021956.1:4592 | 182 |
| nt 1017-1070 of SEQ ID NO: 151 | | | |
| Grik2 mRNA, exon 6; | Homo sapiens | NM_021956.1:4592 | 183 |
| nt 1071-1244 of SEQ ID NO: 151 | | | |
| Grik2 mRNA, exon 7; | Homo sapiens | NM_021956.1:4592 | 184 |
| nt 1245-1388 of SEQ ID NO: 151 | | | |
| Grik2 mRNA, exon 8; | Homo sapiens | NM_021956.1:4592 | 185 |
| nt 1389-1496 of SEQ ID NO: 151 | | | |
| Grik2 mRNA, exon 9; | Homo sapiens | NM_021956.1:4592 | 186 |
| nt 1497-1610 of SEQ ID NO: 151 | | | |
| Grik2 mRNA, exon 10; | Homo sapiens | NM_021956.1:4592 | 187 |
| nt 1611-1817 of SEQ ID NO: 151 | | | |
| Grik2 mRNA, exon 11; | Homo sapiens | NM_021956.1:4592 | 188 |
| nt 1818-2041 of SEQ ID NO: 151 | | | |
| Grik2 mRNA, exon 12; | Homo sapiens | NM_021956.1:4592 | 189 |
| nt 2042-2160 of SEQ ID NO: 151 | | | |
| Grik2 mRNA, exon 13; | Homo sapiens | NM_021956.1:4592 | 190 |
| nt 2161-2378 of SEQ ID NO: 151 | | | |
| Grik2 mRNA, exon 14; | Homo sapiens | NM_021956.1:4592 | 191 |
| nt 2379-2604 of SEQ ID NO: 151 | | | |
| Grik2 mRNA, exon 15; | Homo sapiens | NM_021956.1:4592 | 192 |
| nt 2605-2855 of SEQ ID NO: 151 | | | |
| Grik2 mRNA, exon 16; | Homo sapiens | NM_021956.1:4592 | 193 |
| nt 2856-4592 of SEQ ID NO: 151 | | | |

### Nucleic Acid Vectors

Effective intracellular concentrations of a nucleic acid agent disclosed herein can be achieved via the stable expression of a polynucleotide encoding the agent (e.g., by integration into the nuclear or mitochondrial genome of a mammalian cell). The nucleic acid is an inhibitory RNAs (e.g., inhibitory RNA agents disclosed herein) targeting the Grik2 mRNA. In order to introduce such exogenous nucleic acids into a mammalian cell, the polynucleotide sequence for the agent can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

The agents disclosed herein can also be introduced into a mammalian cell by targeting a vector containing a polynucleotide encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using conventional and routine methods of the art.

In addition to achieving high rates of transcription and translation, stable expression of an exogenous polynucleotide in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that encodes a Grik2-targeting inhibitory RNA agent as well as, e.g., additional sequence elements used for the expression of these agents and/or the integration of these polynucleotide sequences into the genome of a mammalian cell. Certain vectors that can be used include plasmids that contain regulatory sequences, such as promoter and enhancer regions, which direct gene transcription. Other useful vectors contain polynucleotide sequences that enhance the rate of translation of these genes or improve the stability or nuclear export of the mRNA that results from gene transcription. These sequence elements include, e.g., 5′ and 3′ UTR regions, an IRES, and polyadenylation signal sequence site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin.

### Regulatory Sequences

The inhibitory RNA agents disclosed herein may be expressed at sufficiently high levels to elicit a therapeutic benefit. Accordingly, polynucleotide expression may be mediated by a promoter sequence capable of driving robust expression of the disclosed inhibitory RNA agents. According to the methods and compositions disclosed herein, the promoter may be a heterologous promoter. The term “heterologous promoter”, as used herein, refers to a promoter that is not found to be operatively linked to a given encoding sequence in nature. Useful heterologous control sequences generally include those derived from sequences encoding mammalian or viral genes.

Both heterologous promoters and other control elements, such as CNS-specific and inducible promoters, enhancers, and the like, can be used. A promoter may be derived in its entirety from a native gene (e.g., a Grik2 gone) or may be composed of different elements derived from different naturally-occurring promoters. Alternatively, the promoter may include a synthetic polynucleotide sequence. Different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known in the art.

In mammalian systems, three kinds of promoters exist and are candidates for construction of the expression vectors: (i) Pol I promoters that control transcription of large ribosomal RNAs; (ii) Pol Il promoters that control the transcription of mRNAs (that are translated into protein), small nuclear RNAs (snRNAs), and endogenous microRNAs (e.g., from introns of pre-mRNA): (iii) and Pol III promoters that uniquely transcribe small non-coding RNAs. Each has advantages and constraints to consider when designing the construct for expression of the RNAs in vivo. For example, Pol III promoters are useful for synthesizing inhibitory RNA agents (e.g., siRNA, shRNA, miRNA, or shmiRNA) from a DNA template in vivo. For greater control over tissue specific expression, Pol Il promoters can be used (e.g., for transcription of miRNAs). When a Pol Il promoter is used, translation initiation signals may be omitted so that the RNAs function as siRNA, shRNA or miRNAs and are not translated into peptides in vivo.

Polynucleotides suitable for use with the compositions and methods described herein also include those that encode an inhibitory RNA agent targeting Grik2 mRNA under control of a mammalian regulatory sequence, such as, e.g., a promoter sequence and, optionally, an enhancer sequence. Exemplary promoters that are useful for the expression of the disclosed inhibitory RNA agents in mammalian cells include cell-type specific promoters. For example, neuron-specific expression of Grik2 inhibitory RNA agents can be conferred using neuronal-specific promoters, such as, e.g., a human synapsin 1 (hSyn) promotor or Ca²⁺/calmodulin-dependent protein kinase II (CaMKII) promoter. Variants of the hSyn and CaMKII promoters have been previously described in Hioki et al. Gene Therapy 14:872-82 (2007) and Sauerwald et al. J. Biol. Chem. 265 (25): 14932-7 (1990), the disclosures of which are hereby incorporated by reference as they relate to specific hSyn and CaMKII promoter sequences. A constitutive promoter containing cytomegalovirus enhancer (e.g., CAG or CBA), U6, H1, or 7SK promoter may also be used instead. The sequences for these promoters are known in the art (sequences for these promoters are also disclosed in, e.g., WO 2022/011262, which is incorporated herein by reference).

In a particular example, the expression vectors of the disclosure include a SYN promoter (e.g., such as a human SYN promoter (hSyn), e.g., any one of SEQ ID NOs: 194-198 or a variant thereof having at least 85% (e.g., at least 85%, 90%, 95%, 96%, 97%, 98%, 99%, or more (e.g., 100%)) sequence identity to the nucleic acid sequence of any one of SEQ ID NOs: 194-198). In another example, the expression vectors of the disclosure include a CAMKII promoter (e.g., any one of SEQ ID NOs: 199-204 or a variant thereof having at least 85% (e.g., at least 85%, 90%, 95%, 96%, 97%, 98%, 99%, or more (e.g., 100%)) sequence identity to the nucleic acid sequence of any one of SEQ ID NOs: 99-204).

Exemplary promoter sequences suitable for use with the expression vectors (e.g., plasmid or viral vector, such as, e.g., an AAV or a lentiviral vector) are provided in Table 11 below.

TABLE 11
Exemplary neuron-specific promoter sequences

| | SEQ | | GenBank |
|---|---|---|---|
| Promoter | ID NO | Nucleotide sequence | RefSeq |
| Syn | 194 | CTGCAGAGGGCCCTGCGTATGAGTGCAAGTGG | M55301.1 |
| (short_1; | | GTTTTAGGACCAGGATGAGGCGGGGTGGGGGT | |
| H. sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGC | |
| Syn | 195 | CTGCAGAGGGCCCTGCGTATGAGTGCAAGTGG | M55301.1 |
| (short 2; | | GTTTTAGGACCAGGATGAGGCGGGGTGGGGGT | |
| H. sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGCCAATTGCAGCGGAGGAGTCGTGTC | |
| | | GTGCCTGAGAGCGCAG | |
| Syn | 196 | CTGCAGAGGGCCCTGCGTATGAGTGCAAGTGG | M55301.1 |
| (short_2.5; | | GTTTTAGGACCAGGATGAGGCGGGGTGGGGGT | |
| H. sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGCCAATTGCAGCGGAGGAGTCGTGTC | |
| | | GTGCCTGAGAGCGCAGGGCGCGCC | |
| Syn | 197 | CTGCAGAGGGCCCTGCGTATGAGTGCAAGTGG | M55301.1 |
| (short_3; | | GTTTTAGGACCAGGATGAGGGGGGGGGGGGT | |
| H. sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCG | |
| Syn | 198 | CTGCAGAGGGCCCTGCGTATGAGTGCAAGTGG | M55301.1 |
| (long_1; | | GTTTTAGGACCAGGATGAGGCGGGGTGGGGGT | |
| H. sapiens) | | GCCTACCTGACGACCGACCCCGACCCACTGGAC | |
| | | AAGCACCCAACCCCCATTCCCCAAATTGCGCAT | |
| | | CCCCTATCAGAGAGGGGGAGGGGAAACAGGAT | |
| | | GCGGCGAGGCGCGTGCGCACTGCCAGCTTCAG | |
| | | CACCGCGGACAGTGCCTTCGCCCCCGCCTGGC | |
| | | GGCGCGCGCCACCGCCGCCTCAGCACTGAAGG | |
| | | CGCGCTGACGTCACTCGCCGGTCCCCCGCAAAC | |
| | | TCCCCTTCCCGGCCACCTTGGTCGCGTCCGCGC | |
| | | CGCCGCCGGCCCAGCCGGACCGCACCACGCGA | |
| | | GGCGCGAGATAGGGGGGCACGGGCGCGACCAT | |
| | | CTGCGCTGCGGCGCCGGCGACTCAGCGCTGCC | |
| | | TCAGTCTGCGGTGGGCAGCGGAGGAGTCGTGT | |
| | | CGTGCCTGAGAGCGCAGGGCGCGCC | |
| CaMKII_1 | 199 | CATTATGGCCTTAGGTCACTTCATCTCCATGGGG | AJ222796.1 |
| | | TTCTTCTTCTGATTTTCTAGAAAATGAGATGGGG | |
| | | GTGCAGAGAGCTTCCTCAGTGACCTGCCCAGGG | |
| | | TCACATCAGAAATGTCAGAGCTAGAACTTGAACT | |
| | | CAGATTACTAATCTTAAATTCCATGCCTTGGGGG | |
| | | CATGCAAGTACGATATACAGAAGGAGTGAACTCA | |
| | | TTAGGGCAGATGACCAATGAGTTTAGGAAAGAA | |
| | | GAGTCCAGGGCAGGGTACATCTACACCACCCGC | |
| | | CCAGCCCTGGGTGAGTCCAGCCACGTTCACCTC | |
| | | ATTATAGTTGCCTCTCTCCAGTCCTACCTTGACG | |
| | | GGAAGCACAAGCAGAAACTGGGACAGGAGCCC | |
| | | CAGGAGACCAAATCTTCATGGTCCCTCTGGGAG | |
| | | GATGGGTGGGGAGAGCTGTGGCAGAGGCCTCA | |
| | | GGAGGGGCCCTGCTGCTCAGTGGTGACAGATA | |
| | | GGGGTGAGAAAGCAGACAGAGTCATTCCGTCAG | |
| | | CATTCTGGGTCTGTTTGGTACTTCTTCTCACGCT | |
| | | AAGGTGGCGGTGTGATATGCACAATGGCTAAAA | |
| | | AGCAGGGAGAGCTGGAAAGAAACAAGGACAGA | |
| | | GACAGAGGCCAAGTCAACCAGACCAATTCCCAG | |
| | | AGGAAGCAAAGAAACCATTACAGAGACTACAAG | |
| | | GGGGAAGGGAAGGAGAGATGAATTAGCTTCCCC | |
| | | TGTAAACCTTAGAACCCAGCTGTTGCCAGGGCA | |
| | | ACGGGGCAATACCTGTCTCTTCAGAGGAGATGA | |
| | | AGTTGCCAGGGTAACTACATCCTGTCTTTCTCAA | |
| | | GGACCATCCCAGAATGTGGCACCCACTAGCCGT | |
| | | TACCATAGCAACTGCCTCTTTGCCCCACTTAATC | |
| | | CCATCCCGTCTGTTAAAAGGGCCCTATAGTTGGA | |
| | | GGTGGGGGAGGTAGGAAGAGCGATGATCACTT | |
| | | GTGGACTAAGTTTGTTCGCATCCCCTTCTCCAAC | |
| | | CCCCTCAGTACATCACCCTGGGGGAACAGGGTC | |
| | | CACTTGCTCCTGGGCCCACACAGTCCTGCAGTA | |
| | | TTGTGTATATAAGGCCAGGGCAAAGAGGAGCAG | |
| | | GTTTTAAAGTGAAAGGCAGGCAGGTGTTGGGGA | |
| | | GGCAGTTACCGGGGCAACGGGAACAGGGCGTT | |
| | | TCGGAGGTGGTTGCCATGGGGACCTGGATGCTG | |
| | | ACGAAGGCTCGCGAGGCTGTGAGCAGCCACAG | |
| | | TGCCCTGCTCAGAAGCCCCAAGCTCGTCAGTCA | |
| | | AGCCGGTTCTCCGTTTGCACTCAGGAGCACGGG | |
| | | CAGGCGAGTGGCCCCTAGTTCTGGGGGCAGC | |
| CaMKII | 200 | GATGCTGACGAAGGCTCGCGAGGCTGTGAGCA | NM_171825 |
| (α; H. sapiens) | | GCCACAGTGCCCTGCTCAGAAGCCCCGG | |
| CaMKII | 201 | GTCTCCCGCGCCCGCGCCCGTGTCGCCGCCGT | NM_172084 |
| (β1; H. sapiens) | | GCCCGCGAGCGGGAGCCGGAGTCGCCGC | |
| CaMKII | 202 | CGTGTGCAGATGCAGGGCGCCGGTGCCCTGCG | NM 172084 |
| (β2; H. sapiens) | | GGTGCGGGTGCAGGAGCAGCGTGTGCAG | |
| CaMKII | 203 | CCCCACGCCACCCTTTCTGGTCATCTCCCCTCC | NM_172115 |
| (δ; H. sapiens) | | CGCCCCGCCCCTGCGCACACTCCCTCG | |
| CaMKII | 204 | TCTCCCCGGTAAAGTCTCGCGGTGCTGCCGGGC | NM 172171 |
| (γ; H. sapiens) | | TCAGCCCCGTCTCCTCCTCTTGCTCCC | |

In a particular example, a viral vector of the disclosure incorporates a neuron-specific promoter sequence. In a particular example, the neuron-specific promoter is a human Syn (hSyn) promoter, such as, a human Syn promoter having a nucleic acid sequence of any one of SEQ ID NOs: 194-198 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of any one of SEQ ID NOs: 194-198.

In another example, the neuron-specific promoter is a CaMKII promoter sequence, such as a CaMKII promoter sequence of any one of SEQ ID NOs: 199-204 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of any one of SEQ ID NOs: 199-204.

Additional CaMKII promoters may include the human alpha CaMKII promoter sequence described in Wang et al. (Mol. Biol. Rep. 35 (1): 37-44, 2007), the disclosure of which is incorporated in its entirety herein as it relates to the CaMKII promoter sequence.

Once a polynucleotide encoding the disclosed inhibitory RNA agent has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promotor, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode Grik2-targeting inhibitory RNA agents and additionally include a mammalian enhancer sequence. Many enhancer sequences are now known from mammalian genes, and examples are enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a polynucleotide encoding an antisense construct of the disclosure, for example, at a position 5′ or 3′ to this gene. In a particular orientation, the enhancer is positioned at the 5′ side of the promoter, which in turn is located 5′ relative to the polynucleotide encoding an inhibitory RNA agent of the disclosure. Non-limiting examples of enhancer sequences are provided in Table 12 below.

Additional regulatory elements that may be included in polynucleotides for use in the compositions and methods described herein are intron sequences. Intron sequences are non-protein-coding RNA sequences found in pre-mRNA which are removed during RNA splicing to produce the mature mRNA product. Intronic sequences are important for the regulation of gene expression in that they may be further processed to produce other non-coding RNA molecules. Alternative splicing, nonsense-mediated decay, and mRNA export are biological processes that have been shown to be regulated by intronic sequences. Intronic sequences may also facilitate the expression of a transgene through intron-mediated enhancement. Non-limiting examples of intron sequences are provided in Table 12 below.

Further regulatory elements that may be used in conjunction with the vectors of the disclosure include inverted terminal repeat (ITR) sequences. ITR sequences are found, e.g., in AAV genomes at the 5′ and 3′ ends, each typically containing about 145 base pairs. AAV ITR sequences are particularly important for AAV genome multiplication by facilitating complementary strand synthesis once an AAV vector is incorporated into a cell. Moreover, ITRs have been shown to be critical for integration of the AAV genome into the genome of the host cell and encapsidation of the AAV genome. Non-limiting examples of ITR sequences are provided in Table 12 below. 5

Additional regulatory elements suitable for incorporation into the vectors of the disclosure include polyadenylation sequences (i.e., polyA sequences). PolyA sequences are RNA tails containing a stretch of adenine bases. These sequences are appended to the 3′ end of an RNA molecule to produce a mature mRNA transcript. Several biological processes related to mRNA processing and transport are modulated by polyA sequences, including nuclear export, translation, 10 and stability. In mammalian cells, shortening of the polyA tails results in increased likelihood of mRNA degradation. Non-limiting examples of a polyA sequence are provided in Table 12, below.

TABLE 12
Exemplary regulatory sequences

| | SEQ | |
|---|---|---|
| Regulatory element | ID NO | Nucleotide sequence |
| Chimeric Intron | 205 | GTAAGTATCAAGGTTACAAGACAGGTTTAAGGAGACCAATAG |
| | | AAACTGGGCTTGTCGAGACAGAGAAGACTCTTGCGTTTCTG |
| | | ATAGGCACCTATTGGTCTTACTGACATCCACTTTGCCTTTCT |
| | | CTCCACAG |
| VH4 Intron | 206 | GTGAGTATCTCAGGGATCCAGACATGGGGATATGGGAGGTG |
| | | CCTCTGATCCCAGGGCTCACTGTGGGTCTCTCTGTTCACAG |
| CMV Enhancer | 207 | CGTTACATAACTTACGGTAAATGGCCCGCCTGGCTGACCGC |
| | | CCAACGACCCCCGCCCATTGACGTCAATAATGACGTATGTT |
| | | CCCATAGTAACGCCAATAGGGACTTTCCATTGACGTCAATGG |
| | | GTGGAGTATTTACGGTAAACTGCCCACTTGGCAGTACATCAA |
| | | GTGTATCATATGCCAAGTACGCCCCCTATTGACGTCAATGAC |
| | | GGTAAATGGCCCGCCTGGCATTATGCCCAGTACATGACCTT |
| | | ATGGGACTTTCCTACTTGGCAGTACATCTACGTATTAGTCAT |
| | | CGCTATTACCATG |
| AAV 5′-ITR | 208 | CTGCGCGCTCGCTCGCTCACTGAGGCCGCCCGGGCAAAGC |
| | | CCGGGCGTCGGGCGACCTTTGGTCGCCCGGCCTCAGTGAG |
| | | CGAGCGAGCGCGCAGAGAGGGAGTGGCCAACTCCATCACT |
| | | AGGGGTTCCT |
| Modified AAV 5′-ITR | 209 | CTGCGCGCTCGCTCGCTCACTGAGGCCGCCCGGGCAAAGC |
| (Deleted D-sequence for | | CCGGGCGTCGGGCGACCTTTGGTCGCCCGGCCTCAGTGAG |
| self-complimentary | | CGAGCGAGCGCGCAGAGAGGGAGTGG |
| AAV) | | |
| AAV 3′-ITR 1 | 210 | GAACCCCTAGTGATGGAGTTGGCCACTCCCTCTCTGCGCGC |
| | | TCGCTCGCTCACTGAGGCCGCCCGGGCAAAGCCCGGGCGT |
| | | CGGGCGACCTTTGGTCGCCCGGCCTCAGTGAGCGAGCGAG |
| | | CGCGCAGAGAGGGAGTGGCCAA |
| AAV 3′-ITR 2 | 212 | AGGAACCCCTAGTGATGGAGTTGGCCACTCCCTCTCTGCGC |
| | | GCTCGCTCGCTCACTGAGGCCGGGCGACCAAAGGTCGCCC |
| | | GACGCCCGGGCTTTGCCCGGGGGGCCTCAGTGAGCGAGCG |
| | | AGCGCGCAG |
| Modified AAV 3′-ITR | 211 | TTGGCCACTCCCTCTCTGCGCGCTCGCTCGCTCACTGAGGC |
| (Deleted D-sequence for | | CGGGCGACCAAAGGTCGCCCGACGCCCGGGCTTTGCCCGG |
| self-complimentary | | GCGGCCTCAGTGAGCGAGCGAGCGCGCAG |
| AAV) | | |
| Rabbit β-globin (RBG) | 213 | ATAAAGGAAATTTATTTTCATTGCAATAGTGTGTTGGAATTTT |
| PolyA signal #1 | | TTGTGTCTCTCA |
| RBG PolyA #2 | 214 | GATCTTTTTCCCTCTGCCAAAAATTATGGGGACATCATGAAG |
| | | CCCCTTGAGCATCTGACTTCTGGCTAATAAAGGAAATTTATT |
| | | TTCATTGCAATAGTGTGTTGGAATTTTTTGTGTCTCTCACTCG |
| RBG PolyA #3 | 215 | GATCCGATCTTTTTCCCTCTGCCAAAAATTATGGGGACATCA |
| | | TGAAGCCCCTTGAGCATCTGACTTCTGGCTAATAAAGGAAAT |
| | | TTATTTTCATTGCAATAGTGTGTTGGAATTTTTTGTGTCTCTC |
| | | ACTCG |
| Bovine growth hormone | 216 | TAGCAGGCATGCTGGGGAG |
| (BGH) PolyA | | |

In other examples, a viral vector of the disclosure incorporates one or more regulatory sequence elements capable of facilitating the expression an antisense construct of the disclosure. In one example, the regulatory sequence element is an intron sequence. For example, an intron sequence suitable for inclusion into the vector of the disclosure may be a chimeric intron such as a chimeric intron having a nucleic acid sequence of SEQ ID NO: 205 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of SEQ ID NO: 205.

In another example, the intron sequence is an immunoglobulin heavy-chain-variable 4 (VH4) intron, such as a VH4 sequence having a nucleic acid sequence of SEQ ID NO: 206 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of SEQ ID NO: 206.

In some embodiments, from 5′ to 3′, the vector includes: (a) a first promoter sequence; (b) an intron sequence; (c) a polynucleotide comprising a stem-loop sequence; (d) optionally, a second promoter sequence; and (e) optionally, a polynucleotide comprising a stem-loop sequence.

In another example, the regulatory sequence element is an enhancer sequence. For example, the enhancer sequence may be a CMV enhancer, such as a CMV enhancer having a nucleic acid sequence of SEQ ID NO: 207 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of SEQ ID NO: 207.

In some embodiments, from 5′ to 3′, the vector includes: (a) an enhancer sequence; (b) a first promoter sequence; (c) an intron sequence; (d) a polynucleotide comprising a stem-loop sequence; (e) optionally, a second promoter sequence; and (f) optionally, a second polynucleotide comprising a stem-loop sequence.

In another example, the regulatory sequence element is an ITR sequence, such as, e.g., an AAV ITR sequence. For example, the ITR sequence may be an AAV 5′ ITR sequence, such as an AAV 5′ ITR sequence having a nucleic acid sequence of SEQ ID NO: 208 or SEQ ID NO: 209 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of SEQ ID NO: 208 or SEQ ID NO: 209.

In another example, the ITR sequence is an AAV 3′ ITR sequence, such as an AAV 3′ ITR sequence having a nucleic acid sequence of any one of SEQ ID NOs: 210-212 or a variant thereof having at least 70% (e.g., at least 70%, 75%, 80%, 85%, 90%, 91%, 92%, 93%, 94%, 95%, 96%, 97%, 98%, 99% or more (e.g., 100%)) sequence identity to the nucleic acid sequence of any one of SEQ ID NOs: 210-212.

In some embodiments, the vector includes, from 5′ to 3′: (a) a 5′ ITR sequence: (b) optionally, an enhancer sequence; (c) a first promoter sequence; (d) optionally, an intron sequence; (e) a polynucleotide comprising a stem-loop sequence; (f) optionally, a second promoter sequence; (g) optionally, a polynucleotide comprising a stem-loop sequence; and (h) a 3′ ITR sequence.

In another example, the regulatory sequence element is a polyadenylation signal sequence (i.e., a polyA tail). For example, the polyadenylation signal sequence suitable for use with the vectors disclosed herein include a rabbit β-globin (RBG) polyadenylation signal sequence, such as a RBG polyadenylation signal sequence having a nucleic acid sequence of any one of SEQ ID NOs: 213-215 or a variant thereof having at least 70% (e.g., at least 71%, 75%, 80%, 85%, 90%, 91%, 92%, 93%, 94%, 95%, 96%, 97%, 98%, 99% or more (e.g., 100%)) sequence identity to the nucleic acid sequence of any one of SEQ ID NOs: 213-215. Another polyadenylation signal sequence that can be used in conjunction with the disclosed compositions and methods is a bovine growth hormone (BGH) polyadenylation signal sequence, such as a BGH polyadenylation signal sequence of SEQ ID NO: 216 or a variant thereof having at least 70% (e.g., at least 71%, 75%, 80%, 85%, 90%, 91%, 92%, 93%, 94%, 95%, 96%, 97%, 98%, 99% or more (e.g., 100%)) sequence identity to the nucleic acid sequence of SEQ ID NO: 216. In some embodiments, from 5′ to 3′, the vector includes: (a) a 5′ ITR sequence; (b) optionally, an enhancer sequence; (c) a first promoter sequence; (d) optionally, an intron sequence; (e) a polynucleotide comprising a stem-loop sequence; (f) optionally, a second promoter sequence; (g) optionally, a polynucleotide comprising a stem-loop sequence; (h) a polyadenylation signal sequence, such as a RBG polyadenylation signal sequence; and (i) a 3′ ITR sequence.

### Viral Vectors

Viral genomes provide a rich source of vectors that can be used for the efficient delivery of exogenous polynucleotides into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a parvovirus (e.g., adeno-associated viruses (AAV)), retrovirus (e.g., Retroviridae family viral vector), adenovirus (e.g., Ad5, Ad26, Ad34, Ad35, and Ad48), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, murine mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### AAV Vectors

Nucleic acids of the compositions described herein may be incorporated into an AAV vector and/or virion in order to facilitate their introduction into a cell, e.g., in connection with the methods disclosed herein. AAV vectors can be used in the central nervous system, and appropriate promoters and serotypes are discussed in, e.g., Pignataro et al., J Neural Transm 125 (3): 575-89 (2017), the disclosure of which is incorporated herein by reference as it pertains to promoters and AAV serotypes useful in CNS gene therapy. rAAV vectors useful in the compositions and methods described herein are recombinant nucleic acid constructs that include (1) a heterologous sequence to be expressed (e.g., a polynucleotide encoding a Grik2 mRNA-targeting inhibitory RNA agent) and (2) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

Examples of AAVs that can be used as a vector for incorporating a nucleic acid agent described herein (e.g., an inhibitory RNA sequence (e.g., any one of SEQ ID NOs: 1-19, 34-62, 97-108, 133-147, 226-229, and 238-241)) include, e.g., AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2, and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10 and rh74. For targeting cells located in or delivered to the central nervous system, AAV2, AAV9, and AAV10 may be particularly useful. Construction and use of AAV vectors and AAV proteins of different serotypes are described, for example, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbort et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype pseudotyped with a capsid gene derived from a serotype other than the given serotype (AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16). For example, the AAV may include a pseudotyped recombinant AAV (rAAV) vector, such as, e.g., an rAAV2/8 or rAAV2/9 vector. Methods for producing and using pseudotyped rAAV are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

The rAAV used in the compositions and methods of the disclosure may include a capsid protein from an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV. PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV-TT, AAVDJ8, or a derivative, modification, or pseudotype thereof, such as, e.g., a capsid protein that is at least 80% or more identical, e.g., 85%, 85%, 87%, 88%, 89%, 90%, 91%, 92%, 93%, 94%, 95%, 96%, 97%, 98%, 99%, 99.5%, or more, i.e., up to 100% identical, to e.g., vp1, vp2 and/or vp3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B. AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8 or AAV.HSC16.

The AAV vector, which can be used in the methods described herein, may be an Anc80 or Anc80L65 vector, as described in Zinn et al., 2015:1056-1068, which is incorporated by reference in its entirety. The AAV vector may include one of the following amino acid insertions: LGETTRP (SEQ ID NO: 14 of '956, '517, '282, or '323) or LALGETTRP (SEQ ID NO: 15 of '956, '517, '282, or '323), as described in U.S. Pat. Nos. 9,193,956; 9,458,517; and 9,587,282 and US patent application publication no. 2016/0376323, each of which is incorporated herein by reference in its entirety. Alternatively, AAV vector used in the methods described herein may be an AAV.7m8, as described in U.S. Pat. Nos. 9,193,956; 9,458,517; and 9,587,282 and US patent application publication no. 2016/0376323, each of which is incorporated herein by reference in its entirety. Further still, the AAV vector used in the methods described herein may be any AAV disclosed in U.S. Pat. No. 9,585,971, such as an AAV.PHP.B vector. Another AAV vector used in methods described herein may be any vector disclosed in Chan et al. (Nat Neurosci. 20 (8): 1172-1179, 2017), such as an AAV.PHP.eB, which comprises an AAV9 capsid protein having a peptide inserted between amino acid positions 588 and 589 and modifications A587D/588G. Furthermore, the AAV vector used in the methods described herein may be any AAV disclosed in U.S. Pat. No. 9,840,719 and WO 2015/013313, such as an AAV. Rh74 or RHM4-1 vector, each of which is incorporated heroin by reference in its entirety. Additionally, the AAV vector used in the methods described herein may be any AAV disclosed in WO 2014/172669, such as AAV rh.74, which is incorporated herein by reference in its entirety. The AAV vector used in the methods described herein may also be an AAV2/5 vector, as described in Georgiadis et al., 2016, Gene Therapy 23:857-862 and Georgiadis et al., 2018, Gene Therapy 25:450, each of which is incorporated by reference in its entirety. In further examples, the AAV vector used in the methods described herein may be any AAV disclosed in WO 2017/070491, such as an AAV2tYF vector, which is incorporated herein by reference in its entirety. Additionally, AAV vector used in the methods described herein may be an AAVLK03 or AAV3B vector, as described in Puzzo et al., 2017, Sci. Transl. Med. 29 (9): 418, which is incorporated by reference in its entirety. In additional examples, the AAV vector used in the methods described herein may be any AAV disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

Furthermore, the AAV vector used in the methods described herein may be an AAV vector disclosed in any of the following patents and patent applications, each of which is incorporated herein by reference in its entirety: United States Patent Nos. 7,282, 199; 7,906, 111; 8,524,446; 8,999,678; 8,628,966; 8,927,514; 8,734,809; U.S. Pat. Nos. 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799: PCT/EP2015/053335. The rAAV vector may have a capsid protein at least 80% or more identical, e.g., 85%, 85%, 87%, 88%, 89%, 90%, 91%, 92%, 93%, 94%, 95%, 96%, 97%, 98%, 99%, 99.5%, or more (e.g., 100%) to the vp1, vp2 and/or vp3 amino acid sequence of an AAV capsid disclosed in any of the following patents and patent applications, each of which is incorporated herein by reference in its entirety: United States Patent Nos. 7,282, 199; 7,906, 111; 8,524,446; 8,999,678; 8,628,966; 8,927,514; 8,734,809; U.S. Pat. Nos. 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

Additionally, the rAAV vector may have a capsid protein disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689, (see, e.g., SEQ ID NOs: 5-38 of '689) WO2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 2015/191508 (see, e.g., SEQ ID NOs: 80-294 of '058), and U.S. Appl. Publ. No. 20150023924 (see, e.g., SEQ ID NOs: 1, 5-10 of '924), the contents of each of which is herein incorporated by reference in its entirety, such as, e.g., an rAAV vector having a capsid protein that is at least 80% or more identical, e.g., 85%, 85%, 87%, 88%, 89%, 90%, 91%, 92%, 93%, 94%, 95%, 96%, 97%, 98%, 99%, 99.5%, or more (e.g., 100%) to the vp1, vp2 and/or vp3 amino acid sequence of an AAV capsid disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689 (see, e.g., SEQ ID NOs: 5-38 of '689) WO2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 2015/191508 (see, e.g., SEQ ID NOs: 80-294 of '508), and U.S. Appl. Publ. No. 20150023924 (see, e.g., SEQ ID NOs: 1, 5-10 of '924).

Nucleic acid sequences of AAV-based viral vectors and methods of making recombinant AAV and AAV capsids are taught, for example, in United States Patent Nos. 7,282, 199; 7,906, 111; 8,524,446; 8,999,678; 8,628,966; 8,927,514; 8,734,809; U.S. Pat. Nos. 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

Accordingly, the rAAV vector may include a capsid containing a capsid protein from two or more AAV capsid serotypes, such as, e.g., AAV serotypes selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16.

A single-stranded AAV (ssAAV) vector can be used in conjunction with the disclosed methods and compositions. Alternatively, a self-complementary AAV vector (scAAV) can be used (see, e.g., Wu, 2007, Human Gene Therapy, 18 (2): 171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

A recombinant AAV vector with a tropism for cells in the central nervous system, including but not limited to neurons and/or glial cells, can be used for delivering a polynucleotide agent (e.g., an inhibitory RNA agent) of the disclosure. Such vectors can include non-replicating “rAAV” vectors, particularly those bearing an AAV5, AAV9, or AAVrh10 capsid. AAV variant capsids can be used, including but not limited to those described by Wilson in U.S. Pat. No. 7,906,111, which is incorporated by reference herein in its entirety, with AAV/hu.31 and AAV/hu.32 being particularly preferred, as well as AAV variant capsids described by Chatterjee in U.S. Pat. Nos. 8,628,966, 8,927,514 and Smith et al., 2014, Mol Ther 22:1625-1634, each of which is incorporated by reference herein in its entirety. Furthermore, the AAV-TT vector disclosed by Tordo et al. (Brain 141:2014-31, 2018; incorporated herein by reference in its entirety), which incorporates amino acid sequences that are conserved among natural AAV2 isolates, may also be used in conjunction with the compositions and methods of the disclosure. AAV-TT variant capsids exhibit enhanced neurotropism and robust distribution throughout the CNS compared to AAV2, AAV9, and AAVrh10. Similarly, the AAV-DJ8 vector disclosed in Hammond et al. (PLOS ONE 12 (2): e0188830, 2017; incorporated by reference herein in its entirety) exhibits superior neurotropism and may be suitable for use with the compositions and methods of the disclosure.

In a particular example, the disclosure features AAV9 vectors, including an artificial genome including (i) an expression cassette containing the polynucleotide encoding an inhibitory RNA sequence (e.g., any one of SEQ ID NOs: 1-19, 34-62, 97-108, 133-147, 226-229, and 238-241,) under the control of regulatory elements and flanked by ITRs; and (ii) a viral capsid that has the amino acid sequence of the AAV9 capsid protein or is at least 95%, 96%, 97%, 98%, 99% or 99.9% identical to the amino acid sequence of the AAV9 capsid protein while retaining the biological function of the AAV9 capsid. The encoded AAV9 capsid may have the sequence of SEQ ID NO: 116 set forth in U.S. Pat. No. 7,906,111 which is incorporated by reference heroin in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV9 capsid.

Also provided herein are AAVrh10 vectors including an artificial genome including (i) an expression cassette containing the polynucleotide under the control of regulatory elements and flanked by ITRs; and (ii) a viral capsid that has the amino acid sequence of the AAVrh10 capsid protein or is at least 95%, 96%, 97%, 98%, 99% or 99.9% identical to the amino acid sequence of the AAVrh10 capsid protein while retaining the biological function of the AAVrh10capsid. The encoded AAVrh10 capsid may have the sequence of SEQ ID NO: 81 set forth in U.S. Pat. No. 9,790,427 which is incorporated by reference herein in its entirety, with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAVrh10 capsid.

Gene regulatory elements may be selected to be functional in a mammalian cell (e.g., a neuron). The resulting construct which contains the operatively linked components is flanked by (5′ and 3′) functional AAV ITR sequences. Particular examples include vectors derived from AAV serotypes having tropism for and high transduction efficiencies in cells of the mammalian CNS, particularly neurons. A review and comparison of transduction efficiencies of different serotypes is provided in this patent application. In certain examples, AAV2, AAV5, AAV9 and AAVrh10 based vectors direct long-term expression of polynucleotides in CNS, for example, by transducing neurons and/or glial cells.

The AAV expression vector which harbors the polynucleotide of interest (e.g., a polynucleotide encoding an inhibitory RNA agent described herein) flanked by AAV ITRs can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Patents Nos. 5,173, 414 and 5,139, 941; International Publications Nos. WO 92/01070 (published 23 Jan. 1992) and WO 93/03769 (published 4 Mar. 1993). Alternatively, AAV ITRs can be excised from the viral genome or from an AAV vector containing the same and fused to the 5′ and 3′ ends of a selected nucleic acid construct that is present in another vector using standard nucleic acid ligation techniques. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ relative to one or more selected nucleic acid sequences. Preferred codons for expression of the chimeric gene sequence in mammalian CNS cells can be used, and in certain cases, codon optimization of the polynucleotide can be performed by well-known methods.

The complete chimeric sequence is assembled from overlapping polynucleotides prepared by standard methods. In order to produce AAV virions, an AAV expression vector is introduced into a suitable host cell using known techniques, such as by transfection. A number of transfection techniques are generally known in the art. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

For instance, a particular viral vector of the disclosure may include, in addition to a nucleic acid sequence of the disclosure (e.g., any one of SEQ ID NOs: 1-19, 34-62, 97-108, 133-147, 226-229, and 238-241), the backbone of AAV vector plasmid with ITR derived from an AAV2 virus, a promoter such as, e.g., a neuronal promoter such as the hSyn promoter and/or CaMKII promoter, with or without the wild-type or mutant form of the WPRE, and a rabbit beta-globin polyA sequence (see Table 11 and Table 12). If desired, the hSyn promoter and the CaMKII promoter could be replaced with a constitutive promoter containing cytomegalovirus enhancer (e.g., CAG or CBA), U6, H1, or 7SK promoter in the constructs described herein.

The disclosure further relates to an rAAV including (i) an expression cassette containing a polynucleotide under the control of regulatory elements and flanked by ITRs, and (ii) an AAV capsid, wherein the polynucleotide encodes an inhibitory RNA (e.g., an ASO, such as, e.g., siRNA, shRNA, miRNA, or shmiRNA, and, in particular, an inhibitory RNA having a nucleic acid sequence of any one of SEQ ID NOs: 1-19, 34-62, 97-108, 133-147, 226-229, and 238-241, or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, and 238-241) that specifically binds to at least a portion or region of a Grik2 mRNA (e.g., any one of the portions or regions of a Grik2 mRNA described in SEQ ID NOs: 164-193) and that inhibits (e.g., knocks down) expression of GluK2 protein in a cell (e.g., a neuron).

The AAV vector may include, e.g., an inhibitory RNA (e.g., siRNA, shRNA, miRNA, or shmiRNA) sequence that that binds to the Grik2 mRNA, and a hSyn promoter. For example, the AAV vector may contain nucleic acid sequence of any one of SEQ ID NOs: 1-19, 34-62, 97-108, 133-147, 226-229, and 238-241, or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, and 238-241, and an hSyn promotor (e.g., hSyn promotor having a nucleic acid sequence of any one of SEQ ID NO: 194-198 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NO: 194-198).

Alternatively, the AAV vector may include an inhibitory RNA (e.g., siRNA, shRNA, miRNA, or shmiRNA) sequence that binds to and inhibits the expression of the Grik2 mRNA, and a CaMKII promoter. For example, the disclosed AAV vector may include a nucleic acid sequence of any one of SEQ ID NOs: 1-19, 34-62, 97-108, 133-147, 226-229, 238-241, and 258 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, 238-241, and 258 and an CaMKII promoter (e.g., CaMKII promoter having a nucleic acid sequence of any one of any one of SEQ ID NOs: 199-204 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 199-204). In some embodiments, the AAV vector may include a nucleic acid sequence of any one of SEQ ID NOs: 4, 19, 34, 135, 141, 147, and 258, or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 4, 19, 34, 135, 141, 147, and 258.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the polynucleotide. An overview of optimization strategies for packaging and transducing LVs is provided in Delenda, The Journal of Gene Medicine 6: S125 (2004), the disclosure of which is incorporated herein by reference.

The use of lentivirus-based gone transfer techniques relies on the in vitro production of recombinant lentiviral particles carrying a highly deleted viral genome in which the polynucleotide of interest is accommodated. In particular, the recombinant lentivirus are recovered through the in trans co-expression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rov Responsive Element (RRE), 3′-splice sito (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). Optionally, one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of polynucleotide expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polynucleotides are useful. The vector used in the methods and compositions described herein may include multiple promoters that permit expression more than one polynucleotide. Other elements that permit expression of multiple polynucleotides identified in the future are useful and may be utilized in the vectors suitable for use with the compositions and methods described herein. The vector used in the methods and compositions described herein may, be a clinical grade vector.

Accordingly, retroviral vectors may be employed in conjunction with the disclosed methods and compositions. Retroviruses may be chosen as gene delivery vectors due to their ability to integrate their genes into the host genome, transferring a large amount of foreign genetic material, infecting a broad spectrum of species and cell types and for being packaged in special cell-lines. In order to construct a retroviral vector, a nucleic acid encoding a gene of interest is inserted into the viral genome in the place of specific viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (e.g., by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gone transfer. Retroviral vectors are able to infect a broad variety of cell types.

Additionally, lentiviral vectors may be employed in combination with the methods and compositions disclosed herein. Accordingly, an object of the disclosure relates to a lentiviral vector including an inhibitory RNA (e.g., siRNA, shRNA, miRNA, or shmiRNA) sequence (e.g., any one of the inhibitory RNA sequences described in SEQ ID NOs: 1-19, 34-62, 97-108, 133-147, 226-229, 238-241, and 258) that binds to and inhibits the expression of the Grik2 mRNA.

Accordingly, the lentiviral vector may include the nucleic acid sequence of any one of SEQ ID NOs: 1-19, 34-62, 97-108, 133-147, 226-229, 238-241, and 258, or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, 238-241, and 258. The lentiviral vector may include an inhibitory RNA sequence (e.g., siRNA, shRNA, miRNA, or shmiRNA) that binds to and inhibits the expression of the Grik2 mRNA, and a hSyn promoter.

The lentiviral vector may include, e.g., an inhibitory RNA (e.g., siRNA, shRNA, miRNA, or shmiRNA) sequence that that binds to the Grik2 mRNA, and a hSyn promoter. For example, the lentiviral vector may contain nucleic acid sequence of any one of SEQ ID NOs: 1-19, 34-62, 97-108, 133-147, 226-229, 238-241, and 258-260 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, 238-241, and 258-260 and an hSyn promoter (e.g., hSyn promotor having a nucleic acid sequence of any one of SEQ ID NOs: 194-198 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 194-198).

Alternatively, the lentiviral vector may include an inhibitory RNA (e.g., siRNA, shRNA, miRNA, or shmiRNA) sequence that binds to and inhibits the expression of the Grik2 mRNA, and a CaMKII promoter. For example, the disclosed lentiviral vector may include a nucleic acid sequence of any one of SEQ ID NOs: 1-19, 34-62, 97-108, 133-147, 226-229, 238-241, and 258-260 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, 238-241, and 258-260 and an CaMKII promoter (e.g., CaMKII promoter having a nucleic acid sequence of any one of any one of SEQ ID NOs: 199-204 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 199-204).

Lentiviruses are complex retroviruses, which, in addition to the common rotroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV1, HIV2) and the Simian Immunodeficiency Virus (SIV). Lentiviral vectors have been generated by multiply attenuating the HIV virulence genes, for example, the genes onv, vif, vpr, vpu and nef are deleted making the vector biologically safe. Lentiviral vectors are known in the art, see, e.g., U.S. Pat. Nos. 6,013,516 and 5,994,136, both of which are incorporated herein by reference. In general, the vectors are plasmid-based or virus-based and are configured to carry the essential sequences for incorporating foreign nucleic acid and for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging proteins, namely gag, pol and env, as well as rev and tat is described in U.S. Pat. No. 5,994,136, incorporated herein by reference. This publication provides a first vector that can provide a nucleic acid encoding a viral gag and a pol gene and second vector that can provide a nucleic acid encoding a viral env to produce a packaging cell. Introducing a vector providing a heterologous gene into said packaging cell yields a producer cell which releases infectious viral particles carrying the foreign gene of interest. The env may be an amphotropic envelope protein which allows transduction of cells of human and other species. Typically, the nucleic acid molecule or the vector of the disclosure include “control sequences,” which refers collectively to promoter sequences, polyadenylation signal sequences, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

Nucleic acid vectors that exhibit tropism for specific target cells, such as, e.g., hippocampal neurons, e.g., DGCs, may be used to deliver the inhibitory polynucleotides described herein.

### Viral Regulatory Elements

Viral regulatory elements are components of delivery vehicles used to introduce nucleic acid molecules into a host cell. Viral regulatory elements are optionally retroviral regulatory elements. For example, the viral regulatory elements may be the LTR and gag sequences from HSC1 or MSCV.

The retroviral regulatory elements may be from lentiviruses or they may be heterologous sequences identified from other genomic regions. As other viral regulatory elements become known, these may be used with the methods and compositions described herein.

### Viral Vectors Encoding Grik2 Inhibitory Polynucleotides

The disclosure relates a nucleic acid vector for delivery of a heterologous polynucleotide, wherein the polynucleotide encodes an inhibitory RNA agent (e.g., siRNA, shRNA, miRNA, or shmiRNA) construct that specifically binds Grik2 mRNA and inhibits expression of GluK2 protein in a cell. Accordingly, an object of the disclosure provides a vector including an inhibitory polynucleotide sequence that is fully or substantially complementary to at least a region or portion of the Grik2 mRNA (e.g., any one of the regions or portions of a Grik2 mRNA selected from any one of SEQ ID NOs: 164-193, or variants thereof having at least 85% (e.g., at least 85%, 90%, 95%, 96%, 97%, 98%, 99%, or more (e.g., 100%)) sequence identity to the nucleic acid sequence of any one of SEQ ID NOs: 164-193. The vector of the disclosure may include any variant of the inhibitory polynucleotide sequence that is fully or substantially complementary to one or more regions of the Grik2 mRNA. Additionally, the vector of the disclosure may include any variant of the inhibitory polynucleotide sequence is fully or substantially complementary to a Grik2 mRNA encoding any variant of the GluK2 protein.

Accordingly, the DNA encoding double stranded RNA of interest is incorporated into a gone cassette, e.g., an expression cassette in which transcription of the DNA is controlled by a promoter and/or other regulatory elements. The DNA is incorporated into such expression cassettes of the vector expressing a Grik2 inhibitory RNA of interest (e.g., any one of SEQ ID NOs: 1-19, 34-62, 97-108, 133-147, 208-229, 238-241, 250-251, and 256-261) and are encapsidated by the viral vector of interest for delivery to target cells. The viral vectors of the disclosure thus encode any antisense RNA that hybridizes to any Grik2 mRNA transcript isoform (e.g., any one of SEQ ID NOs: 164-174). The viral vectors encode, e.g., any one of the inhibitory polynucleotides listed in Tables 2, 4, 6, and 8.

Vectors of the disclosure deliver polynucleotides encoding an inhibitory RNA that recognizes or binds to at least a portion or region of a Grik2 mRNA (e.g., any one of the regions or portions of Grik2 mRNA described in SEQ ID NOs: 164-193 or a variant thereof having at least 85% (e.g., at least 85%, 90%, 95%, 96%, 97%, 98%, 99%, or more (e.g., 100%)) sequence identity to the nucleic acid sequence of any one of SEQ ID NOs: 164-193). The heterologous polynucleotide encoding the inhibitory RNA agent may be part of a larger construct or scaffold that ensures the processing of such an inhibitory RNA within a cell (e.g., a mammalian cell, such as, e.g., a human cell, such as, e.g., a neuronal cell, such as, e.g., a DGC, or a glutamatergic pyramidal neuron). The polynucleotide encoding any one of the siRNAs listed in Tables 2-9 may include a precursor or a portion of a microRNA gene (e.g., E-miR-30, E-miR-218-1, or E-miR-124-3, among others), such as, e.g., a 5′ flanking sequence, a 3′ flanking sequence, or loop sequence of a microRNA gene. In some embodiments, the polynucleotide encoding any one of the siRNAs listed in Tables 2-9 may include a precursor or a portion of one or more microRNA genes (e.g., E-miR-30, E-miR-218-1, or E-miR-124-3). In some embodiments, the polynucleotide may include a precursor or a portion of two or more microRNA genes (e.g., E-miR-30, E-miR-218-1, or E-miR-124-3). In preferred embodiments, the polynucleotide may include a precursor or a portion of E-miR-30 and E-miR-218-1.

Accordingly, an object of the disclosure relates to an expression vector including a heterologous polynucleotide and containing from 5 ′to 3′, e.g., a promoter (e.g., any one of the promoters described in Table 11), optionally an intron (e.g., any one of the introns described in Table 12), a nucleotide sequence encoding an inhibitory RNA agent that inhibits Grik2 mRNA expression (e.g., inhibitory RNA agent having a nucleic acid sequence of any one of SEQ ID NOs: 1-19, 34-62, 97-108, 133-147, 208-229, 238-241, 250-251, and 256-261 or a variant thereof having at least 85% (e.g., at least 85%, 90%, 95%, 96%, 97%, 98%, 99%, or more (e.g., 100%)) sequence identity to the nucleic acid sequence of any one of SEQ ID NOs: 1-19, 34-62, 97-108, 133-147, 208-229, 238-241, 250-251, and 256-261), and a polyA sequence (e.g., any one of the polyA sequences described in Table 12). The expression vector may also include, from 5′ inverted terminal repeat (ITR) to 3′ ITR, a 5′ ITR (e.g., any one of the 5′ or 3′ ITR sequences described in Table 12), a promotor, optionally an intron, a nucleotide sequence encoding an inhibitory RNA that inhibits Grik2 mRNA expression, a polyA sequence, and a 3′ ITR. The expression vector may further contain spacer and/or linker sequences adjoined to any of the foregoing vector elements.

In particular examples, the expression vector or polynucleotide may include a nucleotide sequence that encodes a stem and a loop which form a stem-loop structure, wherein the loop includes a nucleotide sequence encoding any one of the inhibitory RNA agents listed in Tables 2-9. For example, the expression vector or polynucleotide may include a nucleic acid sequence that encodes a loop region, wherein the loop region may be derived in whole or in part from a wild type microRNA sequence gene (e.g., E-miR-30, E-miR-218-1, or E-miR-124-3, among others) or be completely artificial. In a particular example, the loop region may be an E-miR-30a loop sequence. In some embodiments, the expression vector or polynucleotide may include a nucleic acid sequence that encodes two loop regions, wherein the loop regions may be derived in whole or in part from a wild type microRNA sequences (e.g., E-miR-30, E-miR-218-1, or E-miR-124-3). In a particular embodiment, the loop regions may include an E-miR-30 loop region and an E-miR-218-1 loop region.

Furthermore, the one or more stem-loop structures may include a guide sequence (e.g., an antisense RNA sequence, such as, e.g., any one of SEQ ID NOs: 16-30, 63-79, 109-120, 139-144, 230-233, and 242-245) and a passenger sequence (e.g., any one of SEQ ID NOs: 31-45, 80-96, 121-132, 145-150, 234-237, and 246-249) that is complimentary to all or part of the guide sequence. For example, the passenger sequence may be complementary to all of the nucleotides of the guide sequence except for 10, 9, 8, 7, 6, 5, 4, 3, 2, or 1 nucleotide(s) of the guide sequence or the passenger sequence may be complementary to any one of SEQ ID NOs: 16-30, 63-79, 109-120, 139-144, 230-233, and 242-245. In a particular example, one stem-loop structure may include a guide sequence of SEQ ID NO: 19 and a passenger sequence of SEQ ID NO: 34, and a second stem-loop structure may include a guide sequence of SEQ ID NO: 141 and a passenger sequence of SEQ IS NO: 147. Any sequence variants of these four sequences (SEQ ID NOs: 19, 34, 141, and 147) having at least 85% (e.g., at least 90%, 95%, 96%, 97%, 98%, 99%, or more (e.g., 100%)) sequence identity to any one of SEQ ID NOs: 19, 34, 141, and 147 may be included in the stem-loop structures. In some embodiments, the one or more stem-loop structures may include a sequence with at least 85% (e.g., at least 90%, 95%, 99%, or more (e.g., 100%)) sequence identity to the nucleic acid sequence of any one of SEQ ID NOs: 4, 135, and 258.

Pre-miRNA or pri-miRNA scaffolds include guide (i.e., antisense) sequences of the disclosure. A pri-miRNA scaffold includes a pre-miRNA scaffold, and pri-miRNA may be 50-800 nucleotides in length (e.g., 50-800, 75-700, 100-600, 150-500, 200-400, or 250-300 nucleotides). In particular examples, the pri-mRNA may be 50-100 nucleotides (e.g., between 50-60, 60-70, 70-80, 80-90, or 90-100 nucleotides), 100-200 nucleotides (e.g., between 110-120, 120-130, 130-140, 140-150, 150-160, 160-170, 170-180, 180-190, or 190-200 nucleotides), 200-300 nucleotides (e.g., between 200-210, 210-220, 220-230, 230-240, 240-250, 250-260, 260-270, 270-280, 280-290, or 290-300 nucleotides), 300-400 nucleotides (e.g., between 300-310, 310-320, 320-330, 330-340, 340-350, 350-360, 360-370, 370-380, 380-390, or 390-400 nucleotides), 400-500 nucleotides (e.g., between 400-410, 410-420, 420-430, 430-440, 440-450, 450-460, 460-470, 470-480, 480-490, or 490-500 nucleotides), 500-600 nucleotides (e.g., between 500-510, 510-520, 520-530, 530-540, 540-550, 550-560, 560-570, 570-580, 580-590, or 590-600 nucleotides), 600-700 nucleotides (e.g., between 600-610, 610-620, 620-630, 630-640, 640-650, 650-660, 660-670, 670-680, 680-690, or 690-700 nucleotides), or 700-800 nucleotides (e.g., between 700-710, 710-720, 720-730, 730-740, 740-750, 750-760, 760-770, 770-780, 780-790, or 790-800 nucleotides). These engineered scaffolds allow processing of the pre-miRNA into a double stranded RNA comprising a guide strand and a passenger strand. As such, pre-miRNA includes a 5′ arm including the sequence encoding a guide (i.e., antisense sequence) RNA, a loop sequence usually derived from a wild-type miRNA (e.g., E-miR-30, E-miR-218-1, or E-miR-124-3, among others) and a 3′ arm including a sequence encoding a passenger (i.e., sense sequence) strand which is fully or substantially complementary to the guide strand. Pre-miRNA “stem-loop” structures are generally longer than 50 nucleotides, e.g., 50-150 nucleotides (e.g., 50-60, 60-70, 70-80, 80-90, 90-100, 100-110, 110-120, 120-130, 130-140, or 140-150 nucleotides), 50-110 nucleotides (e.g., 50-60, 60-70, 70-80, 80-90, 90-100, 100-110 nucleotides), or 50-80 nucleotides (e.g., 50-60, 60-70, 70-80 nucleotides) in length. Pri-miRNA further includes 5′ flanking and 3′ flanking sequences, flanking the 5′ and 3′ arms, respectively. Flanking sequences are not necessarily contiguous with other sequences (the arm region or the guide sequence), are unstructured, unpaired regions, and may also be derived, in whole or in part, from one or more wild-type pri-miRNA scaffolds (e.g., pri-miRNA scaffolds derived, in whole or in part, from one or more of E-miR-30, E-miR-218-1, or E-miR-124-3, among others). Flanking sequences are each at least 4 nucleotides in length, or up to 300 nucleotides or more in length (e.g., 4-300, 10-275, 20-250, 30-225, 40-200, 50-175, 60-150, 70-125, 80-100, or 90-95 nucleotides). Spacer sequences may be present as intervening between the aforementioned sequence structures, and in most instances provide linking polynucleotides, e.g., 1-30 nucleotides (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides), to provide flexibility without interfering with functionality to the overall pre-miRNA structure. The spacer may be derived from a naturally occurring linking group from a naturally occurring RNA, a portion of a naturally occurring linking group, a poly-A or poly-U, or a random sequence of nucleotides, so long as the spacer does not interfere with the processing of the double stranded RNA, nor does the spacer interfere with the binding/interaction of the guide RNA with the target mRNA sequence.

According to the methods and compositions disclosed herein, the expression vector or polynucleotide including a nucleotide sequence may further encode (i) a 5′ stem-loop arm including a guide (e.g., antisense) strand and, optionally, a 5′ spacer sequence; and (ii) a 3′ stem-loop arm including a passenger (e.g., sense) strand and optionally a 3′ spacer sequence. In another example, the expression vector or polynucleotide including a nucleotide sequence may further encode (i) a 5′ stem-loop arm including a passenger strand and, optionally, a 5′ spacer sequence; and (ii) a 3′ stem-loop arm including a guide strand and optionally a 3′ spacer sequence. In another example, a uridine wobble base is present at the 5′ end of the guide strand. In a further example, the expression vector or polynucleotide includes a leading 5′ flanking region upstream of the guide sequence and the flanking region may be of any length and may be derived in whole or in part from wild type microRNA sequence, may bo heterologous or derived from a miRNA of different origin from the other flanking regions or the loop, or may be completely artificial. A 3′ flanking region may mirror the 5′ flanking region in size and origin and the 3′ flanking region may be downstream (i.e., 3′) of the guide sequence. In yet another example, one or both of the 5′ flanking sequence and the 3′ flanking sequences are absent.

The expression vector or polynucleotide may include a nucleotide sequence that further encodes a first flanking region (e.g., any one of the 5′ flanking regions described in Table 8), said first flanking region includes a 5′ flanking sequence and, optionally, a 5′ spacer sequence. In a particular example, the first flanking region is located upstream (i.e., 5′) to said passenger strand. In another example, the expression vector or polynucleotide including a nucleotide sequence encodes a second flanking region (e.g., any one of the 3′ flanking regions described in Table 8), said second flanking region includes a 3′ flanking sequence and, optionally, a 3′ spacer sequence. In a particular example, the first flanking region is located 5′ to the guide strand.

According to the methods and compositions disclosed herein, the expression vector or polynucleotide may include a nucleic acid sequence having at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 1-34, 135-147, 226-229, or 256. In a particular example, the expression vector or polynucleotide may include a nucleic acid sequence having at least 85% identity to SEQ ID NO: 256. In some embodiments, the expression vector or polynucleotide may include the nucleic acid sequence of SEQ ID NO: 256. In another particular example, the expression vector or polynucleotide may include a nucleic acid sequence having at least 85% (e.g., at least 86%, 87%, 88%, 89%, 90%, 91%, 92%, 93%, 94%, 95%, 96%, 97%, 98%, 99%, or more (e.g., 100%)) identity to one or more of SEQ ID NOs: 1-34 and 135-147. In another example, the expression vector or polynucleotide may include a nucleic acid sequence at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 46-62. In another example, the expression vector or polynucleotide may include a nucleic acid sequence at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 97-108. In yet another example, the expression vector or polynucleotide may include a nucleic acid sequence at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 133-138.

In another example, the expression vector or polynucleotide includes a nucleotide sequence that encodes:
(a) a stem-loop sequence including, from 5′ to 3′:
   (i) a 5′ stem-loop arm including a passenger nucleotide sequence which is complementary or substantially complementary to the guide nucleotide sequence (such as a passenger sequence having a nucleic acid sequence with at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 31-45, 80-96, 121-132, 145-150, 234-237, and 246-249);
   (ii) a microRNA loop region, in which the loop region includes a microRNA loop sequence (e.g., a E-miR-30a, miR-218-1, or E-miR-124-3 loop sequence (e.g., a microRNA loop sequence having a nucleic acid selected from any one of SEQ ID NOs: 4-225);
   (iii) a 3′ stem-loop arm including a guide nucleotide sequence (such as a guide sequence having a nucleic acid sequence with at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 16-30, 63-79, 109-120, 139-144, 230-233, and 242-245);
(b) a 5′ flanking region (e.g., any one of the 5′ flanking regions described in Table 13) located 5′ to the passenger strand; and
(c) a 3′ flanking region (e.g., any one of the 3′ flanking regions described in Table 13) located 3′ to the guide strand, in which the second flanking region includes a 3′ flanking sequence and, optionally, a 3′ spacer sequence. In some embodiments, the expression vector or polynucleotide includes a nucleic acid sequence that encodes: (a) a stem-loop sequence including, from 5′ to 3′: (i) a 5′ stem-loop arm including a passenger nucleotide sequence which is complementary or substantially complementary to the guide nucleotide sequence (such as a passenger sequence having a nucleic acid sequence with at least 85% (e.g., at least 86%, 87%, 88%, 89%, 90%, 91%, 92%, 93%, 94%, 95%, 96%, 97%, 98%, 99%, or more (e.g., 100%)) identity to any one of SEQ ID NOs: 34 and 147); (ii) a microRNA loop region, in which the loop region includes a microRNA loop sequence (e.g., an E-miR-30a or an E-miR-218-1 loop sequence (e.g., a microRNA loop sequence having a nucleic acid selected from any one of SEQ ID NOs: 4 or 135); (iii) a 3′ stem-loop arm including a guide nucleotide sequence (such as a guide sequence having a nucleic acid sequence with at least 85% (e.g., at least 86%, 87%, 88%, 89%, 90%, 91%, 92%, 93%, 94%, 95%, 96%, 97%, 98%, 99%, or more (e.g., 100%)) sequence identity to any one of SEQ ID NOs: 19 and 141); (b) a 5′ flanking region (e.g., any one of the 5′ flanking regions described in Table 13) located 5′ to the passenger strand; and
   (c) a 3′ flanking region (e.g., any one of the 3′ flanking regions described in Table 13) located 3′ to the guide strand, in which the second flanking region includes a 3′ flanking sequence and, optionally, a 3′ spacer sequence. In some embodiments, the expression vector or polynucleotide includes a nucleic acid sequence that includes two stem-loop sequences, in which (a) one stem-loop sequence contains a guide sequence of SEQ ID NO: 19 or a variant thereof with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 19, a microRNA loop region of SEQ ID NO: 4 or a variant thereof with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO:4, and a passenger sequence of SEQ ID NO: 34 or a variant thereof with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO:34, and (b) a second stem-loop sequence containing a guide sequence of SEQ ID NO: 141 or a variant thereof with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO:141, a microRNA loop region of SEQ ID NO: 135 or a variant thereof with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 135, and a passenger sequence of SEQ ID NO: 147 or a variant thereof with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 147. In preferred embodiments, the expression vector or polynucleotide includes a nucleic acid sequence that includes two stem-loop sequences, in which (a) one stem-loop sequence contains the guide sequence of SEQ ID NO: 19, the microRNA loop region of SEQ ID NO: 4, and the passenger sequence of SEQ ID NO: 34, and (b) the second stem-loop sequence contains the guide sequence of SEQ ID NO: 141, the microRNA loop region of SEQ ID NO: 135, and the passenger sequence of SEQ ID NO: 147. In some embodiments, the expression vector or polynucleotide includes a nucleotide sequence with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 256. In some embodiments, the expression vector or polynucleotide has the nucleic acid sequence of SEQ ID NO: 256.

In another example, the expression vector or polynucleotide includes a nucleotide sequence that encodes:
(a) a stem-loop sequence including, from 5′ to 3′:
   (i) a 5′ stem-loop arm including a guide nucleotide sequence (such as a guide sequence having a nucleic acid sequence with at least 85% (e.g., at least 86%, 87%, 88%, 89%, 90%, 91%, 92%, 93%, 94%, 95%, 96%, 97%, 98%, 99%, or more (e.g., 100%)) sequence identity to any one of SEQ ID NOs: 16-30, 63-79, 109-120, 139-144, 230-233, and 242-245);
   (ii) a microRNA loop region, in which the loop region includes a microRNA loop sequence (e.g., a E-miR-30a, miR-218-1, or E-miR-124-3 loop sequence (e.g., a microRNA loop sequence having a nucleic acid selected from any one of SEQ ID NOs: 219, 222, or 225);
   (iii) a 3′ stem-loop arm including a passenger nucleotide sequence which is complementary or substantially complementary to the guide nucleotide sequence (such as a passenger sequence having a nucleic acid sequence with at least 85% (e.g., at least 86%, 87%, 88%, 89%, 90%, 91%, 92%, 93%, 94%, 95%, 96%, 97%, 98%, 99%, or more (e.g., 100%)) sequence identity to any one of SEQ ID NOs: 31-45, 80-96, 121-132, 145-150, 234-237, and 246-249);
(b) a 5′ flanking region (e.g., any one of the 5′ flanking regions described in Table 13) located 5′ to the guide strand; and
(c) a 3′ flanking region (e.g., any one of the 3′ flanking regions described in Table 13) located 3′ to the passenger strand, in which the second flanking region includes a 3′ flanking sequence and, optionally, a 3′ spacer sequence.

The length of the aforementioned guide strand and passenger strand may be between 19-50 (e.g., 19, 20, 21, 22, 23, 24, 25, 26-30, 31-35, 36-40, 41-45, or 46-50) nucleotides in length. In a particular example, the length of the guide strand is 19 nucleotides. In another example, the length of the guide strand is 20 nucleotides. In another example, the length of the guide strand is 21 nucleotides. In another example, the length of the guide strand is 22 nucleotides. In another example, the length of the guide strand is 23 nucleotides. In another example, the length of the guide strand is 24 nucleotides. In another example, the length of the guide strand is 25 nucleotides. In another example, the length of the guide strand is 26-30 nucleotides. In another example, the length of the guide strand is 31-35 nucleotides. In another example, the length of the guide strand is 36-40 nucleotides. In another example, the length of the guide strand is 41-45 nucleotides. In another example, the length of the guide strand is 46-50 nucleotides. In a particular example, the length of the passenger strand is 19 nucleotides. In another example, the length of the passenger strand is 20 nucleotides. In another example, the length of the passenger strand is 21 nucleotides. In another example, the length of the passenger strand is 22 nucleotides. In another example, the length of the passenger strand is 23 nucleotides. In another example, the length of the passenger strand is 24 nucleotides. In another example, the length of the passenger strand is 25 nucleotides. In another example, the length of the passenger strand is 26-30 nucleotides. In another example, the length of the passenger strand is 31-35 nucleotides. In another example, the length of the passenger strand is 36-40 nucleotides. In another example, the length of the passenger strand is 41-45 nucleotides. In another example, the length of the passenger strand is 46-50 nucleotides.

The length of the guide and passenger sequence may vary based on the miRNA scaffold into which the guide and passenger strands are incorporated. When a given guide is adapted into a miRNA scaffold, the length of the guide can be extended to accommodate the natural structure and processing of a given miRNA scaffold. For example, guide sequences produced by the E-miR-30 scaffold are typically 22 nucleotides long. For most scaffolds, the guide sequences are extended at the 3′ end to be additionally complementary to the target mRNA sequence, but in some cases may involve modifying the 5′ start site of the guide, depending on the sequence of the miRNA scaffold.

In certain cases, it may be desirable to modify miRNA guide and passenger strand expression levels and/or processing patterns to improve or modify targeting capacity of a given construct. As such, within a given miRNA framework/scaffold, the location of the guide and passenger strand may be exchanged; this may be in the context of a design including a stuffer sequence (e.g., SEQ ID NO: 250 or SEQ ID NO: 251), or may be in the context of a design without a stuffer. This may additionally be in the context of a dual construct, or a concatenated construct. In order to accommodate this change, the sequence of the guide and/or passenger strand may be modified from the template “parental” design. Alternatively, modifications may be made to the guide and/or passenger strand sequence in order to affect changes in guide and passenger strand expression and/or processing patterns.

In a particular example, the vector or polynucleotide includes a E-miR-30a sequence, in which the 5′ flanking region includes a nucleotide sequence at least 90% (e.g., at least 90%, 91%, 92%, 93%, 94%, 95%, 96%, 97%, 98%, 99%, or more (e.g., 100%)) identical to SEQ ID NO: 217 (see Table 13).

In some embodiments, the vector or polynucleotide includes a E-miR-30a sequence, in which the 3′ flanking region includes a nucleotide sequence at least 90% (e.g., at least 90%, 91%, 92%, 93%, 94%, 95%, 96%, 97%, 98%, 99%, or more (e.g., 100%)) identical to SEQ ID NO: 218 (see Table 13).

In another example, the vector or polynucleotide includes a E-miR-30a structure in which the loop region includes the nucleotide sequence of SEQ ID NO: 219, or a sequence at least 90% (e.g., at least 90%, 91%, 92%, 93%, 94%, 95%, 96%, 97%, 98%, 99%, or more (e.g., 100%)) identical to SEQ ID NO: 219 (see Table 13).

In a particular example, the vector or polynucleotide includes a miR-218-1 sequence, in which the 5′ flanking region includes a nucleotide sequence at least 90% (e.g., at least 90%, 91%, 92%, 93%, 94%, 95%, 96%, 97%, 98%, 99%, or more (e.g., 100%)) identical to SEQ ID NO: 220 (see Table 13).

In some embodiments, the vector or polynucleotide includes a miR-218-1 sequence, in which the 3′ flanking region includes a nucleotide sequence at least 90% (e.g., at least 90%, 91%, 92%, 93%, 94%, 95%, 96%, 97%, 98%, 99%, or more (e.g., 100%)) identical to SEQ ID NO: 221 (see Table 13).

In another example, the vector or polynucleotide includes a miR-218-1 structure in which the loop region includes the nucleotide sequence of SEQ ID NO: 222, or a sequence at least 90% (e.g., at least 90%, 91%, 92%, 93%, 94%, 95%, 96%, 97%, 98%, 99%, or more (e.g., 100%)) identical to SEQ ID NO: 222 (see Table 13).

In a particular example, the vector or polynucleotide includes a E-miR-124-3 sequence, in which the 5′ flanking region includes a nucleotide sequence at least 90% (e.g., at least 90%, 91%, 92%, 93%, 94%, 95%, 96%, 97%, 98%, 99%, or more (e.g., 100%)) identical to SEQ ID NO: 223 (see Table 13). In some embodiments, the vector or polynucleotide includes a E-miR-124-3 sequence, in which the 3′ flanking region includes a nucleotide sequence at least 90% (e.g., at least 90%, 91%, 92%, 93%, 94%, 95%, 96%, 97%, 98%, 99%, or more (e.g., 100%)) identical to SEQ ID NO: 224 (see Table 13).

In another example, the vector or polynucleotide includes a E-miR-124-3 structure in which the loop region includes the nucleotide sequence of SEQ ID NO: 225, or a sequence at least 90% (e.g., at least 90%, 91%, 92%, 93%, 94%, 95%, 96%, 97%, 98%, 99%, or more (e.g., 100%)) identical to SEQ ID NO: 225 (see Table 13).

The expression vector may be a plasmid and may include, e.g., one or more of an intron sequence (e.g., an intron sequence of SEQ ID NO: 205 or SEQ ID NO: 206 or a variant thereof having at least 85% (e.g., at least 85% 90%, 91%, 92%, 93%, 94%, 95%, 96%, 97%, 98%, 99%, or more (e.g., 100%)) sequence identity to the nucleic acid sequence of SEQ ID NO: 205 or SEQ ID NO: 206), a linker sequence, or a stuffer sequence (e.g., SEQ ID NO: 250 or SEQ ID NO: 251).

TABLE 13
MicroRNA Sequences

| | SEQ ID | |
|---|---|---|
| Description | NO | Nucleotide sequence |
| E-hsa-miR- | 217 | GTTTGAATGAGGCTTCAGTACTTTACAGAATCGTTGCCTGCACATCT |
| 30a | | TGGAAACACTTGCTGGGATTACTTCTTCAGGTTAACCCAACAGAAGG |
| 5′ flanking | | CTAAAGAAGGTATATTGCTGTTGACAGTGAGCGAC |
| region | | |
| E-hsa-miR- | 218 | GCTGCCTACTGCCTCGGACTTCAAGGGGCTACTTTAGGAGCAATTA |
| 30a | | TCTTGTTTACTAAAACTGAATACCTTGCTATCTCTTTGATACATTTTTA |
| 3′ flanking | | CAAAGCTGAATTAAAATGGTATAAATTA |
| region | | |
| E-hsa-miR- | 219 | CTGTGAAGCCACAGATGGG |
| 30a | | |
| loop region | | |
| E-hsa-miR- | 220 | ACGTTTCCAGAACGTCTGTAGCTTTTCTCCTCCTTCCCTCCATTTTCC |
| 218-1 | | TCTTGGTCTTACCTTTGGCCTAGTGGTTGGTGTAGTGATAATGTAGC |
| 5′ flanking | | GAGATTTTCTG |
| region | | |
| E-hsa-miR- | 221 | TGGAACGTCACGCAGCTTTCTACAGCATGACAAGCTGCTGAGGCTT |
| 218-1 | | AAATCAGGATTTTCCTGTCTCTTTCTACAAAATCAAAATGAAAAAAGA |
| 3′ flanking | | GGGCTTTTTAGGCATCTCCGAGATTATGTG |
| region | | |
| E-hsa-miR- | 222 | GGTTGCGAGGTATGAGTAAA |
| 218-1 | | |
| loop region | | |
| E-hsa-miR- | 223 | TCTGCCGCGGAAAGGGGAGAAGTGTGGGCTCCTCCGAGTCGGGGG |
| 124-3 | | CGGACTGGGACAGCACAGTCGGCTGAGCGCAGCGCCCCCGCCCTG |
| 5′ flanking | | CCCGCCACGCGGCGAAGACGCCTGAGCGTTCGCGCCCCTCGGGC |
| region | | GAGGACCCCACGCAAGCCCGAGCCGGTCCCGACCCTGGCCCCGAC |
| | | GCTCGCCGCCCGCCCCAGCCCTGAGGGCCCCTC |
| E-hsa-miR- | 224 | GAGAGGCGCCTCCGCCGCTCCTTTCTCATGGAAATGGCCCGCGAG |
| 124-3 | | CCCGTCCGGCCCAGCGCCCCTCCCGCGGGAGGAAGGCGAGCCCG |
| 3′ flanking | | GCCCCCGGCGGCCATTCGCGCCGCGGACAAATCCGGCGAACAATG |
| region | | CGCCCGCCCAGAGTGCGGCCCAGCTGCCGGGCCGGGGATCTGGC |
| | | CGCGGGACACAAAGGGGCCCGCACGCCTCTGGCGT |
| E-hsa-miR- | 225 | ATTTAATGTCTATACAAT |
| 124-3 | | |
| loop region | | |

Accordingly, an object of the disclosure relates to a vector including a polynucleotide having the nucleic acid sequence of any one of SEQ ID NOs: 1-19, 34-62, 97-108, 133-147, 208-229, 238-241, 250-251, and 256-261 or a variant thereof having at least 85% (at least 85%, 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 208-229, 238-241, 250-251, and 256-261. For example, the vector may include a polynucleotide having at least 90% (e.g., at least 90%, 95%, 96%, 97%, 98%, 99%, or more (e.g., 100%)) sequence identity to the nucleic acid sequence of any one of SEQ ID NOs: 1-19, 34-62, 97-108, 133-147, 208-229, 238-241, 250-251, and 256-261. In another example, the vector may include a polynucleotide having least 95% (e.g., at least 95%, 96%, 97%, 98%, 99%, or more (e.g., 100%)) sequence identity to the nucleic acid sequence of any one of SEQ ID NOs: 1-19, 34-62, 97-108, 133-147, 208-229, 238-241, 250-251, and 256-261. The vector of the disclosure may further include a polynucleotide having the nucleic acid sequence of any one of SEQ ID NOs: 1-19, 34-62, 97-108, 133-147, 208-229, 238-241, 250-251, and 256-261.

In particular, the vector may include the sequence of any one of SEQ ID NOs: 1-19, 34-62, 97-108, 133-147, 208-229, 238-241, 250-251, and 256-261 or a variant thereof having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 208-229, 238-241, 250-251, and 256-261 and a promoter (e.g., any one of the promoters listed in Table 11).

The variants discussed above may include, for instance, naturally-occurring variants due to allelic variations between individuals (e.g., polymorphisms), alternative splicing forms, etc. The term variant also includes genes sequences of the disclosure from other sources or organisms. Variants may be substantially homologous to sequences according to the disclosure. Variants of the genes of the disclosure also include nucleic acid sequences, which hybridize to a sequence as defined above (or a complementary strand thereof) under stringent hybridization conditions. Typical stringent hybridization conditions include temperatures above 30° C., above 35° C., or in excess of 42° C., and/or salinity of less than about 500 mM or less than 200 mM. Hybridization conditions may be adjusted by, e.g., modifying the temperature, salinity and/or the concentration of other reagents such as SDS, SSC, etc.

The disclosure further provides non-viral vectors (e.g., a plasmid containing a polynucleotide encoding a Grik2-targeting inhibitory RNA agent disclosed herein) for the delivery of heterologous polynucleotides to target cells of interest. In other cases, the viral vector of the disclosure may be an AAV vector adenoviral, a retroviral, a lentiviral, or a herpesvirus vector.

One or more expression cassettes may be employed. Each expression cassette may include at least a promoter sequence (e.g., a neuronal cell promoter) operably linked to a sequence encoding the RNA of interest. Each expression cassette may consist of additional regulatory elements, spacers, introns, UTRs, polyadenylation site, and the like. The expression cassette can be polycistronic with respect to the polynucleotides encoding e.g., two or more inhibitory RNA agents. The expression cassette may further include a promoter, a nucleic acid encoding one or more inhibitory RNA agents of interest, and a polyA sequence. In a particular example, the expression cassette includes 5′-promoter sequence, a polynucleotide sequence encoding a first inhibitory RNA agent of interest (e.g., any one of SEQ ID NOs: 16-30, 63-79, 109-120, 139-144, 230-233, and 242-245), a sequence encoding a second inhibitory RNA agent of interest (e.g., any one of SEQ ID NOs: 16-30, 63-79, 109-120, 139-144, 230-233, and 242-245), and a polyA sequence-3′. In a preferred embodiment, the expression cassette includes a sequence with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 256. In some embodiments, the expression cassette has the sequence of SEQ ID NO: 256. In a preferred embodiment, the expression cassette includes a sequence with at least 85% (e.g., at least 86%, 87%, 88%, 89%, 90%, 91%, 92%, 93%, 94%, 95%, 96%, 97%, 98%, 99%, or more (e.g., 100%)) sequence identity to SEQ ID NO: 257. In some embodiments, the expression cassette has the sequence of SEQ ID NO: 257.

The viral vector may further include a nucleic acid sequence encoding an antibiotic resistance gene such as the genes of resistance AmpR, kanamycin, hygromycin B, geneticin, blasticidin S, gentamycin, carbenicillin, chloramphenicol, nourseothricin, or puromycin.

### Exemplary Expression Cassettes

The disclosure provides expression cassettes that, when incorporated into an expression vector (e.g., a plasmid or viral vector (e.g., AAV or lentiviral vector)), promote the expression of a heterologous polynucleotide encoding an inhibitory RNA agent (e.g., inhibitory RNA agent having a nucleic acid sequence of any one of SEQ ID NOs: 16-30, 63-79, 109-120, 139-144, 230-233, and 242-245) that hybridizes to and inhibits the expression of a Grik2 mRNA. Generally, an expression cassette incorporated into a nucleic acid vector will include a heterologous polynucleotide containing a heterologous gene regulatory sequence (e.g., a promoter (e.g., any one of the promoters described in Table 11) and, optionally, an enhancer sequence (e.g., an enhancer sequence described in Table 12)), a 5′ flanking sequence (e.g., a 5′ flanking sequence described in Table 13), a stem-loop sequence containing a stem-loop 5′ arm, a loop sequence (e.g., a microRNA loop sequence described in Table 13), a stem-loop 3′ arm, a 3′ flanking sequence (e.g., a 3′ flanking sequence described in Table 13), optionally, a Woodchuck Hepatitis Posttranscriptional Regulatory Element (WRPE), and a polyA sequence (e.g., SEQ ID NOs: 213-216). In the case of an AAV vector, the expression cassette may be flanked on its 5′ and 3′ ends by a 5′ ITR and a 3′ ITR sequence (e.g., any one of the 5′ or 3′ ITR sequences described in Table 12), respectively. Typically, AAV2 ITR sequences are contemplated for use in conjunction with the methods and compositions disclosed herein, however, ITR sequences from other AAV serotypes disclosed herein may also be employed (see section “AAV Vectors” above). The general architecture of the construct includes at least the following elements oriented in a 5′ to 3′ direction:
(i) A 5′ ITR sequence (for AAV vectors only; see Table 12);
(ii) a promoter sequence (e.g., any one of the promoter sequences listed in Table 11);
(iii) a 5′ flanking sequence (e.g., see Table 13);
(iv) a stem-loop sequence having at least 85% (e.g., at least 86%, 87%, 88%, 89%, 90%, 91%, 92%, 93%, 94%, 95%, 96%, 97%, 98%, 99%, or more (e.g., 100%)) sequence identity to the nucleic acid sequence of any one of SEQ ID NOs: 1-19, 34-62, 97-108, 133-147, 226-229, and 238-241;
(v) optionally, a WPRE sequence;
(vi) a polyA sequence (e.g., see Table 12); and
(vii) a 3′ ITR sequence (for AAV vectors only; see Table 12).

In a particular example, the passenger sequence that is substantially complementary to the inhibitory RNA sequence of any one of SEQ ID NOs: 16-30, 63-79, 109-120, 139-144, 230-233, and 242-245 has no more than 7 (e.g., no more than 7, 6, 5, 4, 3, 2, or 1) mismatched nucleotides (i.e., mismatches) relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 6 (e.g., no more than 6, 5, 4, 3, 2, or 1) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 5 (e.g., no more than 5, 4, 3, 2, or 1) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 4 (e.g., no more than 4, 3, 2, or 1) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 3 (e.g., no more than 3, 2, or 1) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 2 (e.g., no more than 2 or 1) mismatches relative to the inhibitory RNA sequence of any one of SEQ ID NOs: 16-30, 63-79, 109-120, 139-144, 230-233, and 242-245. In yet another example, the passenger sequence that is substantially complementary to the inhibitory RNA sequence of any one of SEQ ID NOs: 16-30, 63-79, 109-120, 139-144, 230-233, and 242-245 has no more than 1 mismatch relative to the inhibitory RNA sequence of any one of SEQ ID NOs: 16-30, 63-79, 109-120, 139-144, 230-233, and 242-245.

In another example, the passenger sequence that is substantially complementary to the inhibitory RNA sequence of any one of SEQ ID NOs: 16-30, 63-79, 109-120, 139-144, 230-233, and 242-245 has no more than 10 (e.g., no more than 10, 9, 8, 7, or 6) mismatched nucleotides (i.e., mismatches) relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 9 (e.g., no more than 9, 8, 7, or 6) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 8 (e.g., no more than 8, 7, or 6) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 7 (e.g., no more than 7 or 6) mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 6 mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 5 mismatches relative to the inhibitory RNA sequence of any one of SEQ ID NOS: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 4 mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 3 mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 2 mismatches relative to the inhibitory RNA sequence of any one of SEQ ID NOs: 16-30, 63-79, 109-120, 139-144, 230-233, and 242-245. In another example, the passenger sequence that is substantially complementary to the inhibitory RNA sequence of any one of SEQ ID NOs: 16-30, 63-79, 109-120, 139-144, 230-233, and 242-245 has no more than 1 mismatch relative to the inhibitory RNA sequence of any one of SEQ ID NOs: 16-30, 63-79, 109-120, 139-144, 230-233, and 242-245.

Multi-Gene miRNA Cassettes

The flank/stem-loop/flank construct (e.g., pri-miRNA) may be treated as a single miRNA “cassette” and can be concatenated (e.g., provided in a multi-gene arrangement driven by one or more promoters). More than one (e.g., at least 2, 3, 4, 5, 6, 7, 8, 9, 10, or more) pre-miR stem-loop sequence may be embedded in arbitrary polynucleotide sequences of a longer transcript (such as, e.g., an intron) or between endogenous microRNA flanking sequences (5′ and 3′ to each stem-loop, such as -5p and -3p sequences). Each pre-miR stem-loop sequence may be expressed under the control of a dedicated promoter (e.g., as a multi-gene construct with separate promoter sequences, each of which independently regulates the expression of an individual pre-miR stem-loop sequence; i.e., each promoter functions independent of the other to produce individual microRNAs). It has been shown that flanking sequences that can provide at least a 5-bp-extended stem were sufficient for the processing of the stem-loop (Sun, et al. BioTechniques. 41:59-63, July 2006, incorporated herein by reference). Spacer sequences may be positioned between the 3′ flanking sequence of a first miRNA expression cassette and the 5′ flanking sequence of a second miRNA expression cassette. Spacer sequences may be derived from coding or noncoding (e.g., intron) sequences and are of various lengths, but are not considered part of the stem-loop-flank sequence (Rousset, F. et al., Molecular Therapy: Nucleic Acids, 14:352-63, 2019, incorporated herein by reference.)

An exemplary expression cassette may include a nucleotide sequence containing: (a) a first polynucleotide encoding a first miRNA sequence containing a guide RNA sequence that hybridizes to a Grik2 mRNA; and (b) a second polynucleotide encoding a second miRNA sequence containing a guide RNA sequence that hybridizes to a Grik2 mRNA. For example, the expression cassette may include, from 5′ to 3′: (a) a first 5′ flanking region located 5′ to a guide strand, said first flanking region that includes a first 5′ flanking sequence (e.g., see Table 13) sequence identity thereto): (b) a first stem-loop structure that includes a polynucleotide of any one of SEQ ID NOs: 1-19, 34-62, 97-108, 133-147, 226-229, and 238-241; (c) a first 3′ flanking region (e.g., see Table 13) located 3′ to said passenger strand and a 3′ spacer sequence; (d) a second 5′ flanking region (e.g., see Table 13) located 5′ to a guide strand; (e) a second stem-loop structure that includes a polynucleotide of any one of SEQ ID NOs: 1-15, 46-62, 97-108, 133-138, 226-229, and 238-214; (f) a second 3′ flanking region that includes a 3′ flanking sequence (e.g., see Table 13) located 3′ to the passenger strand. In some embodiments, the expression cassette may include, from 5′ to 3′: (a) a first 5′ flanking region located 5′ to a guide strand, said first flanking region that includes a first 5′ flanking sequence (e.g., see Table 13) sequence identity thereto); (b) a first stem-loop structure that includes a polynucleotide of any one or more SEQ ID NOs: 4, 19 and 34; (c) a first 3′ flanking region (e.g., see Table 13) located 3′ to said passenger strand and a 3′ spacer sequence; (d) a second 5′ flanking region (e.g., see Table 13) located 5′ to a guide strand; (e) a second stem-loop structure that includes a polynucleotide of any one or more of SEQ ID NOs: 135, 141, and 147; (f) a second 3′ flanking region that includes a 3′ flanking sequence (e.g., see Table 13) located 3′ to the passenger strand. In some embodiments, the sequences of the first and second stem-loop structures are reversed such that the first stem-loop structure includes a polynucleotide of any one of SEQ ID NOs: 135, 141, and 147, and the second stem-loop structure includes a polynucleotide of any one of SEQ ID NOs: 4, 19, and 34. The first 5′ flanking sequence, first 3′ flanking sequence, the second 5′ flanking sequence, and the second 3′ flanking sequence may be selected from Table 13.

A polycistronic or multi-gene rAAV expression construct may include a transgene made up of sequential (e.g., contiguous or non-contiguous) miRNA-encoding polynucleotides X₁, such as (X₁)ₙ. The X₁ polynucleotide includes any one of the guide-passenger pairs listed in Tables 3, 5, 7, and 9, any one of the 5′ and 3′ flanking sequences listed in Table 13, and any one of the loop sequences listed in Table 13. The polycistronic transgene having the formula, (X₁)ₙ, is under control of a single promoter positioned at the 5′ end of the transgene such that the promoter and transgene have the formula, promoter-(X₁)ₙ, where n is an integer from 1-10 (e.g., 1, 2, 3, 4, 5, 6, 7, 8, 9, or 10). The multi-gene construct can further include a stuffer sequence (e.g., SEQ ID NO: 250 or SEQ ID NO: 251) at the 3′ end of the construct to improve manufacturing efficiency of the vectors.

### Dual-miRNA Dual-Promoter Expression Cassettes

A multi-gene expression cassette containing more than one (e.g., at least 2, 3, 4, 5, 6, 7, 8, 9, 10, or more) pre-miR stem-loop sequence may include more than one promoter sequence to regulate the expression of each individual pre-miR stem-loop sequence, such that each individual pre-miR stem-loop sequence is operably linked to a dedicated promoter sequence. In such cases, the expression construct features a structure of formula, (promoter-X1) n, where X1 is a polynucleotide of any one of SEQ ID NOs: 1-19, 34-62, 97-108, 133-147, 226-229, and 238-241, and n is an integer from 1-10 (e.g., 1, 2, 3, 4, 5, 6, 7, 8, 9, or 10). Additional regulatory elements, such as enhancer sequences, terminator sequences, polyadenylation signal sequences, introns, and/or sequences, capable of forming secondary structures, such as any one of the regulatory elements disclosed herein, may be operably linked to the 5′ end and/or the 3′ end of the promoter-X1 structure.

In a particular example, the dual-miRNA expression cassette includes two pre-miR stem-loop sequences, each under control of an individual promoter sequence (e.g., a promoter sequence disclosed herein). The two promoters in the dual-miRNA cassette may be identical promoters or different promoters.

In a specific example, the dual-miRNA expression cassette includes a nucleotide sequence comprising, from 5′ to 3′: (a) a first promoter sequence (e.g., any one of the promoter sequences disclosed herein, e.g., Table 11, e.g., an hSyn promoter or CaMKII promoter; (b) a first 5′ flanking region located 5′ to a first stem-loop sequence (e.g., see Table 13); (c) a first stem-loop sequence that includes a polynucleotide of any one of SEQ ID NOs: 1-19, 34-62, 97-108, 133-147, 226-229, and 238-241; (d) a first 3′ flanking region located 3′ to the first guide nucleotide sequence (e.g., see Table 13); (e) a second promoter sequence (e.g., see Table 11), or a variant thereof with at least 85% (at least 86%, 87%, 88%, 89%, 90%, 91%, 92%, 93%, 94%, 95%, 96%, 97%, 98%, 99%, or more (e.g., 100%)) sequence identity thereto); (f) a second 5′ flanking region located 5′ to a second stem-loop sequence (e.g., see Table 13); (g) a second stem-loop sequence that includes a polynucleotide of any one of SEQ ID NOs: 1-19, 34-62, 97-108, 133-147, 226-229, and 238-241; and (h) a second 3′ flanking region located 3′ to the polynucleotide (e.g., see Table 13). If desired, the hSyn promoter and the CaMKII promoter could be replaced with a constitutive promoter containing cytomegalovirus enhancer (e.g., CAG or CBA), U6, H1, or 7SK promoter.

In another example, the first promotor is a SYN promotor (e.g., see Table 11) and the second promoter is a CAMKII promoter (e.g., see Table 11).

Sequence identity for the sequences described in the aforementioned dual-miRNA expression cassettes may be determined over a range of 10-1500 (e.g., 20-1400, 30-1300, 40-1200, 50-1100, 60-1000, 70-900, 80-800, 90-700, 100-600, 200-500, or 300-400) nucleotides. For example, sequence identity to the aforementioned dual-miRNA expression cassettes may be determined over nucleotides. In another example, sequence identity to the aforementioned dual-miRNA expression cassettes is determined over 20 nucleotides. In another example, sequence identity to the aforementioned dual-miRNA expression cassettes is determined over 30 nucleotides. In another example, sequence identity to the aforementioned dual-miRNA expression cassettes is determined over 40 nucleotides. In another example, sequence identity to the aforementioned dual-miRNA expression cassettes is determined over 50 nucleotides. In another example, sequence identity to the aforementioned dual-miRNA expression cassettes is determined over 60 nucleotides. In another example, sequence identity to the aforementioned dual-miRNA expression cassettes is determined over 70 nucleotides. In another example, sequence identity to the aforementioned dual-miRNA expression cassettes is determined over 80 nucleotides. In another example, sequence identity to the aforementioned dual-miRNA expression cassettes is determined over 90 nucleotides. In another example, sequence identity to the aforementioned dual-miRNA expression cassettes is determined over 100 nucleotides. In another example, sequence identity to the aforementioned dual-miRNA expression cassettes is determined over 150 nucleotides. In another example, sequence identity to the aforementioned dual-miRNA expression cassettes is determined over 200 nucleotides. In another example, sequence identity to the aforementioned dual-miRNA expression cassettes is determined over 250 nucleotides. In another example, sequence identity to the aforementioned dual-miRNA expression cassettes is determined over 300 nucleotides. In another example, sequence identity to the aforementioned dual-miRNA expression cassettes is determined over 350 nucleotides. In another example, sequence identity to the aforementioned dual-miRNA expression cassettes is determined over 400 nucleotides. In another example, sequence identity to the aforementioned dual-miRNA expression cassettes is determined over 450 nucleotides. In another example, sequence identity to the aforementioned dual-miRNA expression cassettes is determined over 500 nucleotides. In another example, sequence identity to the aforementioned dual-miRNA expression cassettes is determined over 550 nucleotides. In another example, sequence identity to the aforementioned dual-miRNA expression cassettes is determined over 600 nucleotides. In another example, sequence identity to the aforementioned dual-miRNA expression cassettes is determined over 650 nucleotides. In another example, sequence identity to the aforementioned dual-miRNA expression cassettes is determined over 700 nucleotides. In another example, sequence identity to the aforementioned dual-miRNA expression cassettes is determined over 750 nucleotides. In another example, sequence identity to the aforementioned dual-miRNA expression cassettes is determined over 800 nucleotides. In another example, sequence identity to the aforementioned dual-miRNA expression cassettes is determined over 850 nucleotides. In another example, sequence identity to the aforementioned dual-miRNA expression cassettes is determined over 900 nucleotides. In another example, sequence identity to the aforementioned dual-miRNA expression cassettes is determined over 950 nucleotides. In another example, sequence identity to the aforementioned dual-miRNA expression cassettes is determined over 1000 nucleotides. In another example, sequence identity to the aforementioned dual-miRNA expression cassettes is determined over 1100 nucleotides. In another example, sequence identity to the aforementioned dual-miRNA expression cassettes is determined over 1200 nucleotides. In another example, sequence identity to the aforementioned dual-miRNA expression cassettes is determined over 1300 nucleotides. In another example, sequence identity to the aforementioned dual-miRNA expression cassettes is determined over 1400 nucleotides. In yet another example, sequence identity to the aforementioned dual-miRNA expression cassettes is determined over 1500 nucleotides.

The dual-miRNA expression cassettes described above may include a promotor that is a Synapsin promoter and/or Calcium/Calmodulin Dependent Protein Kinase Il promoter.

MicroRNA loop sequences suitable for use in conjunction with the dual-miRNA expression cassette disclosed herein may be a E-miR-30, miR-218-1, or E-miR-124-3 loop sequence.

Dual-miRNA expression cassettes of the disclosure may also incorporate a 5′-ITR (e.g., see Table 12) on the 5′ end of the expression cassette and a 3′-ITR (e.g., see Table 12) on the 3′ end of the expression cassette.

Moreover, the dual-miRNA expression constructs disclosed herein may include a first polyadenylation (polyA) signal operably linked between the 3′ end of the first 3′ flanking region and the 5′ end of the second promoter and/or a second polyA signal operably linked between the second 3′ end of the second 3′ flanking region and the 3′ ITR. The first polyA signal and the second polyA signal may be identical (e.g., both are RBG or BGH polyA signals) or different (e.g., the first polyA signal is RBG polyA and second polyA signal is BGH polyA; or the first polyA signal is BGH polyA and the second polyA signal is RBG polyA).

Improvements in the Manufacture of AAV Vectors Containing Multiple miRNA Sequences

Preparation of AAV vectors using plasmids encoding a single or dual miRNA expression cassette (e.g., expression cassettes disclosed herein) may potentially be hampered by improper AAV genome packaging. First, pri-miRNA sequences are short (<200 bases) and, depending on promoter length, design of a transgene cassette with a single promoter controlling expression of a single miRNA may result in an AAV genome that is significantly shorter than the maximum packaging capacity of AAV (˜4.8 kb). It is therefore possible that a single capsid may be loaded with more than one vector genome if the anticipated full genome length is <2.4 kb (half the packaging capacity of AAV). This can be mediated by polymerase read-through without proper endonuclease nicking that allows for the production of AAV genome dimers (or trimers) that can then be packaged into the AAV capsid if they are of appropriate length. This subsequently introduces significant heterogeneity into the population of AAV vector particles, which renders manufacturing and characterization of a drug product significantly more difficult.

Second, shRNA- and miRNA-based transgenes inherently have significant secondary structure due to the inclusion of the hairpin. It has been shown that these internal secondary structures within an AAV genome can function as a “false” ITR during AAV genome replication and packaging, resulting in truncation events and a heterogeneous population of AAV vector particles containing a mixture of full and partial vector genomes.

We have discovered that padding of an AAV genome having sizes below the AAV packaging capacity with additional sequences (e.g., additional pre-miRNA stem-loop sequences, a second promoter sequence, stuffer sequences (e.g., SEQ ID NO: 250 or SEQ ID NO: 251), using self-complementary AAV vectors, etc.) substantially improves AAV packaging by avoiding incorporation of copies of partial (i.e., truncated) AAV genomes and prevents or reduces multiple-packaging events. Therefore, the constructs described herein avoid improper packaging of AAV genomes by incorporating the aforementioned sequences into an AAV expression cassette to increase vector size to a value closer to the maximal AAV packaging capacity. In some embodiments, the vector comprises one or more (e.g., 1, 2, or more) stuffer sequences. In some embodiments, the one or more stuffer sequences are positioned at the 3′ end of the expression cassette. In some embodiments, the one or more stuffer sequences have at least 85% (e.g., at least 86%, 90%, 95%, 96%, 97%, 98%, 99%, or more (e.g., 100%)) sequence identity to the nucleic acid sequence of SEQ ID NO: 250. In some embodiments, the one or more stuffer sequences have at least 90% (e.g., at least 91%, 95%, 96%, 97%, 98%, 99%, or more (e.g., 100%)) sequence identity to the nucleic acid sequence of SEQ ID NO: 250. In some embodiments, the one or more stuffer sequences have at least 95% (e.g., at least 96%, 97%, 98%, 99%, or more (e.g., 100%)) sequence identity to the nucleic acid sequence of SEQ ID NO: 250. In some embodiments, the one or more stuffer sequences have at least 99% sequence identity to the nucleic acid sequence of SEQ ID NO: 250. In some embodiments, the one or more stuffer sequences have the nucleic acid sequence of SEQ ID NO: 250. In some embodiments, the one or more stuffer sequences have at least 85% (e.g., at least 86%, 90%, 95%, 96%, 97%, 98%, 99%, or more (e.g., 100%)) sequence identity to the nucleic acid sequence of SEQ ID NO: 251. In some embodiments, the one or more stuffer sequences have at least 90% (e.g., at least 91%, 95%, 96%, 97%, 98%, 99%, or more (e.g., 100%)) sequence identity to the nucleic acid sequence of SEQ ID NO: 251. In some embodiments, the one or more stuffer sequences have at least 95% (e.g., at least 96%, 97%, 98%, 99%, or more (e.g., 100%)) sequence identity to the nucleic acid sequence of SEQ ID NO: 251. In some embodiments, the one or more stuffer sequences have at least 99% sequence identity to the nucleic acid sequence of SEQ ID NO: 251. In some embodiments, the one or more stuffer sequences have the nucleic acid sequence of SEQ ID NO: 251.

For example, there may be cases where a construct expressing ono miRNA under control of a single promoter or two miRNAs from two separate promoters (dual construct approach) may not perform in vivo as predicted by in vitro/ex vivo/in silico evaluation. In these cases, the following strategies can be implemented to establish a genomic length that produces a homogeneous, full-length, singly packaged population of AAV vector particles.

First, if expression of a single miRNA “guide” is desired, a stuffer sequence (e.g., SEQ ID NO: 250 or SEQ ID NO: 251) may be added to increase the total length of the AAV vector genome without disrupting the promoter or miR cassette itself. This stuffer may be added downstream of the transgene cassette (3′ of polyA sequence). In some embodiments, the AAV vector includes a sequence with at least 85% (e.g., at least 86%, 87%, 88%, 89%, 90%, 95%, 99%, or more (e.g., 100%)) sequence identity to any one of SEQ ID NOs: 252-257. In a preferred embodiment, the AAV vector includes the sequence of any one of SEQ ID NOs: 252-257. The length and content of this stuffer may be changed while retaining its ability to improve AAV packaging homogeneity. Additionally, this stuffer may be placed upstream (5′) of the promoter. Stuffer sequences may be utilized in the context of an scAAV vector or a single-stranded (ss) AAV vector.

Second, if more than one miRNA is to be expressed but a single promotor strategy is selected, the vector can be prepared by concatemerization of multiple miRNA cassettes. While concatemerization of multiple miRNA cassettes (e.g., up to 5 miRNA cassettes) using the same scaffold can result in recombination between homologous sequences within the vector genome, concatemerization of miRNA cassettes using different scaffolds with non-homologous flanking and loop sequences can ameliorate this packaging concern. If the inclusion of additional miRNA cassettes does not result in a vector genome of appropriate length, a stuffer sequence may be incorporated as described above to increase length to improve packaging efficiency. In some embodiments, the AAV vector includes a sequence with at least 85% (e.g., at least 86%, 87%, 88%, 89%, 90%, 95%, 99%, or more (e.g., 100%)) sequence identity to any one of SEQ ID NOs: 256-257. In a preferred embodiment, the AAV vector includes the sequence of any one of SEQ ID NOs: 256-257.

### Insect Expression Vectors

Inhibitory polynucleotides described herein may also be encoded in a suitable insect expression vector (e.g., a viral vector such as a baculovirus viral vector) and expressed in an insect expression system. In some embodiments, an inhibitory polynucleotide(s) described herein can be incorporated into a nucleic acid vector (e.g., a baculovirus vector or a baculovirus-based vector, a retroviral vector, or other viral vector), transfected into an insect cell line (e.g., Sf9 cells), and cultured under conditions that permit expression of the polynucleotide(s). For example, the insect cell line (e.g., Sf9 cells) can be transfected transiently or stably with an inhibitory polynucleotide(s) of the disclosure.

Baculovirus delivery vectors and modified host cells corresponding thereto are commercially available, for example, pAcGP67, pAcSECG2TA, pVL1392, pVL1393, pAcGHLT, pAcAB4; pBAC-3, pBAC6, pBACgus-6, pBACsurf-1, pPolh-FLAG, and pPolh-MAT. Methods for baculovirus and insect cell expression systems are well known in the art, as described in Summers and Smith, Texas Agricultural Experiment Station Bulletin No. 1555 (1987), and Luckow and Summers, Bio/Technology 6:47 (1988), incorporated herein by reference. It will be understood by those skilled in the art that the expression system is not limited to the baculovirus expression system. What is important is that the expression system permits expression of an inhibitory polynucleotide of the disclosure. For example, other suitable insect expression systems include an Entomopox virus system (insect mammavirus) and a cytoplasmic polyhedrosis virus (CPV) system.

### Bacterial Expression Vectors

Standard bacterial vectors include, e.g., bacteriophages X and M13, as well as plasmids, such as pBR322, pSKF, and pET23D. In some embodiments, an inhibitory polynucleotide(s) described herein or a nucleic acid vector encoding the same may be incorporated into a plasmid or alternative bacterial expression vector and cultured under conditions that permit expression of the polynucleotide(s). Suitable cloning and expression vectors for use with bacterial host cells have been described by Pouwels et al. (Cloning Vectors: A Laboratory manual, Elsevier, New York, 1985). Exemplary bacterial strains may include Escherichia coli, Bacillus subtilis, Salmonella typhimurium, or any bacterial strain capable of expressing heterologous polynucleotides. Elements that are typically included in bacterial expression vectors include replicons, a gene encoding antibiotic resistance to permit selection of bacteria that harbor recombinant plasmids, and unique restriction sites in nonessential regions of the plasmid to allow insertion of recombinant sequences.

### Pharmaceutical Compositions

The inhibitory polynucleotides described herein or nucleic acid vectors encoding the same may be formulated into pharmaceutical compositions for administration to a mammalian (e.g., a human) subject in a biologically compatible form suitable for administration in vivo.

The compositions disclosed herein may be formulated in any suitable vehicle for delivery to a subject (e.g., a human). For instance, they may be formulated in a pharmaceutically acceptable suspension, dispersion, solution, or emulsion. Suitable mediums include saline and liposomal preparations. More specifically, pharmaceutically acceptable carriers may include sterile aqueous of non-aqueous solutions, suspensions, and emulsions. Recombinant human album (rAlbumin Human NF RECOMBUMIN® Prime) may also be used as a stabilizer with an AAV vector (Albumedix, Nottingham UK). Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles suitable for intravenous administration include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

The compositions described herein may be used in the form of the free base, in the form of salts, solvates, and as prodrugs. All forms are within the methods described herein. In accordance with the methods of the disclosure, the described compounds or salts, solvates, or prodrugs thereof may be administered to a patient in a variety of forms depending on the selected route of administration.

Accordingly, the compositions described herein may be formulated for administration, for example, by oral, parenteral, intrathecal, intracerebroventricular, intraparenchymal, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, intracerebroventricular, intraparenchymal, rectal, and topical modes of administration. Parenteral administration may be by continuous infusion over a selected period of time.

Solutions of an agent described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include sterile aqueous solutions or dispersions and sterile powders for the extemporaneous preparation of sterile injectable solutions or dispersions. In all cases the form must be sterile and must be fluid to the extent that may be easily administered via syringe. Local, regional, or systemic administration also may be appropriate. A composition described herein may advantageously be contacted by administering an injection or multiple injections to the target site, spaced for example, at approximately, 1 cm intervals.

The compositions described herein may be administered to an animal, e.g., a human, alone or in combination with pharmaceutically acceptable carriers, as noted herein, the proportion of which is determined by the solubility and chemical nature of the compound, chosen route of administration, and standard pharmaceutical practice.

Accordingly, the disclosure provides a pharmaceutical composition containing an inhibitory RNA agent disclosed herein (e.g., siRNA, shRNA, miRNA, or shmiRNA). In particular, the disclosure provides a composition including a vector including an inhibitory RNA agent of the disclosure. In a particular example, the disclosure provides a pharmaceutical composition containing a vector (e.g., lentiviral or AAV vector) including an inhibitory RNA of the disclosure operably linked to a promoter, as is disclosed herein. The pharmaceutical composition may include an AAV vector including (a) a viral capsid; and (b) an artificial polynucleotide including an expression cassette flanked by AAV ITRs, wherein the expression cassette includes a polynucleotide encoding an inhibitory polynucleotide that binds to and inhibits the expression of a Grik2 mRNA, operably linked to one or more regulatory sequences that control expression of the polynucleotide in CNS cells.

The inhibitory RNA agents disclosed herein may be combined with pharmaceutically acceptable excipients, and, optionally, sustained-release matrices, such as, e.g., biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions disclosed heroin may be formulated for intracerebral (e.g., intraparenchymal or intracerebroventricular), intramuscular, intravenous, transdermal, local, oral, sublingual, subcutaneous, or rectal administration.

The active component of the composition (e.g., an inhibitory RNA agent targeting Grik2), alone or in combination with another therapeutic agent, can be administered in a unit administration form as a mixture with conventional pharmaceutical supports to subjects in need thereof. Suitable unit administration forms include oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal, intracerebral, stereotactic, and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions, formulations including sesame oil, peanut oil, or aqueous propylene glycol, and sterile powders for the extemporaneous preparation of sterile injectable solutions or dispersions. In all cases, the form must be sterile and must be fluid to the extent that easy syringability is facilitated. It must be stable under the conditions of manufacture and storage and must be preserved against the contaminating action of microorganisms, such as bacteria and fungi. Solutions including compounds of the disclosure as free base or pharmacologically acceptable salts can be prepared in water suitably mixed with a surfactant, such as hydroxypropyl cellulose.

Dispersions can also be prepared in glycerol, liquid polyethylene glycols, and mixtures thereof and in oils. Under ordinary conditions of storage and use, these preparations may contain a preservative to prevent the growth of microorganisms. The inhibitory polynucleotide agents disclosed herein can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids like acetic, oxalic, tartaric, mandolic, and the like. Salts formed with the free carboxyl groups can also be derived from inorganic bases such as, e.g., sodium, potassium, ammonium, calcium, or ferric hydroxides, and such organic bases as isopropylamine, trimethylamine, histidine, procaine, and the like. The carrier can also be a solvent or dispersion medium containing, e.g., water, ethanol, polyol (for example, glycerol, propylene glycol, and liquid polyethylene glycol, and the like), suitable mixtures thereof, and vegetables oils. The proper fluidity can be maintained by the use of a coating (e.g., lecithin), by the maintenance of the required particle size in the case of dispersion, and by the use of surfactants.

The prevention of the action of microorganisms can be brought about by various antibacterial and antifungal agents such as, e.g., parabens, chlorobutanol, phenol, sorbic acid, thimerosal, and the like. In some cases, it will be pharmaceutical compositions of the disclosure may include isotonic agents such as, e.g., sugars or sodium chloride.

Prolonged absorption of the injectable compositions can be brought about by the use of agents delaying absorption such as, e.g., e.g., aluminum monostearate and gelatin. Sterile injectable solutions are prepared by incorporating the active agents in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required additional ingredients from those disclosed herein. In the case of sterile powders for the preparation of sterile injectable solutions, the preferred methods of preparation may be vacuum-drying and freeze-drying techniques which yield a powder of the active ingredient plus any additional desired ingredient from a previously sterile-filtered solution thereof. Upon formulation, solutions will be administered in a manner compatible with the dosage requirement and in such amount as is therapeutically effective.

The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. Sterile aqueous media which can be employed are well-known in the art. For example, one dosage could be dissolved in 1 mL of isotonic NaCl solution and added to 1000 mL of hypodermoclysis fluid or injected at the proposed site of infusion. Some variation in dosage will necessarily occur depending on the condition of the subject being treated. The practitioner responsible for administration can, in any event, using appropriate patient information and art-recognized methods, determine the appropriate dose for the individual subject.

### Methods of Diagnosis

A subject (e.g., a human subject) may, e.g., using methods well-known in the art, be diagnosed as having epilepsy (e.g., TLE), and, thus, identified as in need of treatment using the compositions and methods disclosed herein. For example, diagnosis of an epilepsy in a subject may be guided by neurophysiological testing to identify the epileptogenic focus and the severity of epileptiform activity in the brain of the subject. Exemplary neurophysiological testing methods well-known in the art include electroencephalography (EEG), magnetoencephalography (MEG), functional MRI (fMRI), single-photon emission computed tomography (SPECT), and positron emission tomography (PET). EEG and MEG provide a continuous measure of cortical function with high temporal resolution and facilitate the detection of interictal (period between seizures) epileptiform discharges, which may be indicative of a positive diagnosis of an epileptic condition in the subject. Comparison of brain activity in the subject relative to a norm appropriate for the subject's age, medical history, and lifestyle (e.g., a reference population, such as, e.g., non-epileptic patient population) may be done to determine the diagnosis with respect to an epilepsy in the subject.

The subject may be diagnosed as having any one of a number of epileptic conditions including, but not limited to a TLE (e.g., mTLE or lTLE), benign Rolandic epilepsy, a frontal lobe epilepsy, infantile spasms, a juvenile myoclonic epilepsy, a juvenile absence epilepsy, a childhood absence epilepsy (pyknolepsy), a hot water epilepsy, Lennox-Gastaut syndrome, Landau-Kleffner syndrome, Dravet syndrome, a progressive myoclonus epilepsy, a reflex epilepsy, Rasmussen's syndrome, a limbic epilepsy, status epilepticus, an abdominal epilepsy, massive bilateral myoclonus, a catamenial epilepsy, Jacksonian seizure disorder, Lafora disease, and a photosensitive epilepsy. In cases where the epileptic condition is TLE, the TLE may be characterized by characterized by focal or generalized seizures.

The type of epilepsy that a patient can be diagnosed on the basis of localizing the epileptogenic focus to a particular brain region (e.g., mesial temporal lobe, lateral frontal lobe, frontal lobe, etc.) using the disclosed methods. Electrophysiological signature of epileptic brain activity may also be used to identify the particular type or subtype of epilepsy in a subject. For example, the presence of fast (250-600 Hz) sharp wave ripples (SPW-Rs) in cortical regions (e.g., hippocampus or cerebral cortex) may be indicative of a positive diagnosis of a TLE in the subject. In another example, Lennox-Gastaut syndrome is often characterized by the presence of fast-run electrographic oscillations (10-15 Hz) recorded across the neocortex and thalamus. Furthermore, video monitoring of a subject in an inpatient facility may be indicative of a diagnosis of epilepsy in the patient if the patient appears to overtly exhibit behavioral manifestations of epileptic seizures, such as, e.g., generalized convulsions, temporary absence (decreased levels of consciousness for periods lasting˜10 seconds), tonus, myoclonus, loss of bowel or bladder control, biting of the tongue, fatigue, headache, difficulty speaking, abnormal behavior (e.g., motionless staring or automatic movements of hands or mouth), psychosis, and/or localized weakness. Self-reported symptoms from the subject being diagnosed for epilepsy may also be indicative of a positive diagnosis. Such self-reported symptoms may include sensations of déjà vu or jamais vu, auras, amnesia, a spontaneous and unprovoked fear and anxiety, nausea, auditory, visual, olfactory, gustatory, or tactile hallucinations, visual distortions (e.g., macropsia or micropsia), dissociation or derealization, synesthesia, dysphoric or euphoric feelings, fear, anger, or ineffable sensations.

### Pharmaceutical Uses

Disclosed herein are methods for the treatment of an epilepsy (e.g., TLE) in a subject diagnosed with or at risk of developing an epileptic condition by administration of the compositions described above (e.g., an inhibitory RNA agent or a nucleic acid vector encoding the same). Upon administration, the inhibitory RNA agents of the disclosure are capable of binding to and inhibiting the expression of a Grik2 mRNA. The targeting of Grik2 by an inhibitory RNA agent disclosed herein may be manifested by a decrease in the levels of Grik2 mRNA expressed by a first cell or group of cells (e.g., neuronal cells; such cells may be present, for example, in the subject or in a sample derived from a subject) in which Grik2 is transcribed and which has or have been treated (e.g., by contacting the cell or cells with an inhibitory polynucleotide of the disclosure, or by administering an inhibitory polynucleotide of the disclosure to a subject in which the cells are or were present). In a particular example, the expression of Grik2 is decreased in the first cell or group of cells, as compared to a second cell or group of cells substantially identical to the first cell or group of cells but which has not or have not been so treated (control cell(s) not treated with an inhibitory polynucleotide or not treated with an inhibitory polynucleotide targeted to the gone of interest). The degree of decrease in the levels of mRNA of a gene of interest (e.g., Grik2) may be expressed in terms of:

$$\frac{( mRNA in control cells ) - ( mRNA in treated cells )}{( mRNA in control cells )} \times 100 \%$$

A change in the levels of expression of a gene (e.g., a Grik2 gene) may be assessed in terms of a reduction of a parameter that is functionally linked to the expression of the gene of interest, e.g., protein expression of the gene of interest or signaling downstream of the protein. A change in the levels of expression of the gone of interest may be determined in any cell expressing the gene of interest, either endogenous or heterologous from an expression construct, and by any assay known in the art.

A change in the level of expression of Grik2 may be manifested by a decrease in the level of the GluK2 protein that is expressed by a cell or group of cells (e.g., the level of GluK2 protein expressed in a sample derived from a subject). As is explained above, for the assessment of Grik2 mRNA suppression, the change in the level of GluK2 protein expression in a treated cell or group of cells may similarly be expressed as a percentage of the level of protein in a control cell or group of cells.

A control cell or group of cells that may be used to assess the change in the expression of the Grik2 gene includes a cell or group of cells that has not yet been contacted with an inhibitory polynucleotide of the disclosure. For example, the control cell or group of cells may be derived from an individual subject (e.g., a human or animal subject) prior to treatment of the subject with an inhibitory polynucleotide.

The level of Grik2 mRNA expressed by a cell or group of cells may be determined using any method known in the art for assessing mRNA expression. For example, the level of expression Grik2 mRNA in a sample may be determined by detecting a transcribed polynucleotide, or portion thereof, e.g., mRNA. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating mRNA may be detected using methods the described in PCT Publication WO2012/177906, the entire contents of which are hereby incorporated herein by reference. The level of expression of the gene of interest may also be determined using a nucleic acid probe. The term “probe,” as used herein, refers to any molecule that is capable of selectively binding to a specific sequence, e.g., to an mRNA. Probes can be synthesized using well-known and conventional methods of the art or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. The mRNA may be immobilized on a solid surface and contacted with a probe, for example by running the isolated mRNA on an agarose gel and transferring the mRNA from the gel to a membrane, such as nitrocellulose. The probe(s) may also be immobilized on a solid surface and the mRNA is contacted with the probe(s), for example, in an AFFYMETRIX gone chip array. Known mRNA detection methods in the art may be adapted for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatolli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques wellknown in the art. These detection schemes are especially useful for the detection of nucleic acid molecules if such molecules are present in very low numbers. In particular aspects of the disclosure, the level of expression of a gene of interest is determined by quantitative fluorogenic RT-PCR (i.e., the TAQMAN™ System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads, or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

The level of mRNA expression may also be assessed using branched DNA (bDNA) assays or real time PCR (qPCR). The use of this PCR method is described and exemplified in the Examples presented herein. Such methods can also be used for the detection of nucleic acids of the gene of interest.

Furthermore, the level of GluK2 protein produced by the expression the Grik2 gene may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

Accordingly, the aforementioned assays for measuring Grik2 mRNA or GluK2 protein expression may be used to identify a subject (e.g., a subject suffering from an epilepsy, such as, e.g., TLE) as being in need of therapeutic treatment with one or more inhibitory RNA agents disclosed herein (e.g., any one of the inhibitory RNA agents described in Table 2) or a nucleic acid vector encoding the same. For example, a patient identified as having TLE may exhibit an epileptogenic focus within the temporal lobe of one hemisphere of the brain that causes uncontrollable (e.g., treatment-resistant, e.g., chronic) seizures. As is discussed herein, such an epileptogenic focus may result from, e.g., aberrant sprouting of recurrent dentate granule cell mossy fibers and abnormal (i.e., increased) expression of Grik2 at the recurrent synapses formed by said mossy fibers. Using the assays described above, a determination can be made as to whether the subject would benefit from a therapy using one or more of the Grik2 inhibitory RNA agents disclosed herein, e.g., by performing a small biopsy on brain tissue collected from the hippocampus of the epileptogenic hemisphere and from the same region in the healthy hemisphere. A showing that the epileptogenic hemisphere exhibits higher (e.g., by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more (e.g., 100%)) levels of expression of Grik2 mRNA or GluK2 protein as compared to the unaffected hemisphere would indicate that the patient may benefit from therapy using the methods and compositions disclosed herein. In the case that the subject with TLE presents with an epileptogenic focus in both brain hemispheres, Grik2 mRNA or GluK2 protein levels could be compared between hippocampal tissue obtained from one or more hemispheres from the TLE-afflicted subject and hippocampal tissue from the same hemisphere(s) of a healthy control subject (e.g., from post-mortem tissue of a subject without TLE) using the assays disclosed above. A showing that the epileptogenic hemisphere(s) of the TLE-afflicted subject exhibits higher (e.g., by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more (e.g., 100%)) expression of Grik2 mRNA or GluK2 protein as compared to the same hemisphere(s) of a healthy subject would indicate that the TLE-afflicted subject would benefit from therapeutic treatment with the disclosed compositions and methods. Grik2 mRNA levels or GluK2 protein levels in the neuronal cells of a subject suspected to be in need of treatment can also be compared to standard or reference levels of these analytes that are known to indicate a disease state.

In addition, the assays described above may be utilized to determine whether a subject (e.g., a subject suffering from an epilepsy, such as, e.g., TLE) has responded to treatment using the compositions and methods disclosed herein. For example, as is discussed above, hippocampal brain tissue from an epileptogenic brain hemisphere(s) can be obtained from the TLE-afflicted subject by way of a small biopsy prior to treatment with the compositions and methods disclosed herein and expression of Grik2 mRNA or GluK2 protein may be assessed using the aforementioned assays. The subject may then be administered treatment according to the methods and compositions disclosed herein. Subsequent to the recovery of the patient following treatment (e.g., 1, 5, 10, 15, 30, 60, 90, or more days after treatment) with the disclosed methods and compositions, a second biopsy may be performed over the same brain regions assessed prior to treatment and levels of Grik2 mRNA or GluK2 protein may again be assessed. A showing that the TLE-afflicted subject exhibits lower (e.g., by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more (e.g., 100%)) levels of expression of Grik2 mRNA or GluK2 protein would indicate that the subject was responsive to treatment. Alternatively, Grik2 mRNA or GluK2 protein levels may be compared with respect to expression of the same from one or more healthy control subjects. A showing that Grik2 mRNA or GluK2 protein levels in the TLE-afflicted patient after treatment are statistically indistinguishable from levels of the same in one or more healthy control subjects would indicate that the patient is responsive to treatment. Grik2 mRNA levels or GluK2 protein levels in the neuronal cells of a treated subject can also be compared to standard or reference levels of these analytes that are known to indicate the absence of a disease state.

### Methods of Treatment

A subject with epilepsy (e.g., TLE) may be treated using the compositions and methods described herein. The compositions (e.g., an inhibitory RNA agent-containing composition or a vector containing the same) may be administered as a preventative treatment to a subject in need thereof (e.g., a subject diagnosed as having or being at risk of having an epilepsy (e.g., TLE). A subject at risk of developing an epilepsy may show early symptoms of an epilepsy or may not yet be symptomatic when treatment is administered.

### Routes of Administration

The compositions disclosed herein may be administered to a subject (e.g., a subject identified as having TLE) using standard methods. For example, the compositions disclosed herein can be administered by any of a number of different routes including, e.g., systemic administration. Non-limiting examples of systemic administration include enteral (e.g., oral) or parenteral (e.g., intravenous, intra-arterial, transmucosal, intraperitoneal, epicutaneous, intramucosal (e.g., intranasal or sublingual), intramuscular, or transdermal) administration. Additional routes of administration may include intradermal, subcutaneous, and percutaneous injection.

The compositions disclosed herein may also be administered using methods suitable for local delivery of inhibitory RNA agents or nucleic acid vectors encoding the same. Non-limiting examples of local administration include epicutaneous (e.g., topical), intra-articular, and inhalational routes. In particular, the disclosed compositions may be locally administered to brain tissue (e.g., neural cells, such as e.g., neurons and/or astroglia)) of the subject.

In particular, the inhibitory RNA agents and nucleic acid vectors encoding the same may be administered locally to brain tissue of the subject, such as brain tissue determined to exhibit increased epileptiform activity. Local administration to the brain generally includes any method suitable for delivery of an inhibitory RNA agent or a nucleic vector encoding the same to brain cells (e.g., neural cells), such that at least a portion of cells of a selected, synaptically connected cell population is contacted with the composition. Vectors may be delivered to any cells of the CNS, including neurons, astroglia, or both. Generally, the vector is delivered to the cells of the CNS, including, e.g., cells of the spinal cord, brainstem (medulla, pons, and midbrain), cerebellum, diencephalon (e.g., thalamus and hypothalamus), telencephalon (corpus striatum, cerebral cortex (e.g., cortical regions in the occipital, temporal, parietal, or frontal lobes), or combinations thereof, or any suitable subpopulation of cells therein. Further sites for delivery include the red nucleus, amygdala, entorhinal cortex, and neurons in ventrolateral or anterior nuclei of the thalamus.

The vectors of the disclosure may be delivered by way of stereotactic injections or microinjections directly into the parenchyma or ventricles of the CNS. In a particular example, the vectors of the disclosure may be delivered directly to one or more epileptic foci in the brain of the subject. For example, the subject may be administered a vector of the disclosure by means of a stereotactic injection directly into one or both hemispheres of the allocortex (e.g., hippocampus) or neocortex (e.g., frontal lobe). In a particular example, the subject is administered a vector of the disclosure by means of a stereotactic injection directly into one or both hemispheres of the hippocampus. Alternatively, the vectors of the disclosure may be administered by intravenous injection, for example in the context of vectors that exhibit tropism for CNS tissues, including but not limited to AAV5, AAV9, or AAVrh10.

To deliver a vector of the disclosure specifically to a particular region and to a particular population of CNS cells, the vector may be administered by stereotaxic microinjection. For example, subjects may have a stereotactic frame base surgically fixed in place (screwed into the skull). The brain with a stereotactic frame base (e.g., MRI compatible stereotactic frame base with fiducial markings) is imaged using high resolution MRI. The MRI images are then transferred to a computer which runs stereotactic software. A series of coronal, sagittal and axial images are used to determine the target injection site and trajectory of the cannula or injection needle used for injecting a composition of the disclosure into the brain. The software directly translates the trajectory into three-dimensional coordinates appropriate for the stereotactic frame. Holes are drilled above the entry site and the stereotactic apparatus is positioned with the injection noodle implanted at the given depth. The composition (such as a composition disclosed herein) may be injected at the target sites. In the case that the composition includes an integrating vector, rather than producing viral particles, the spread of the vector is minor and mainly a function of passive diffusion from the site of injection. The degree of diffusion may be controlled by adjusting the ratio of vector to fluid carrier.

Additional routes of administration may also include local application of the vector under direct visualization, e.g., superficial cortical application, or other non-stereotactic application. The vector may be delivered intrathecally (e.g., directly into the cisterna magna), in the ventricles (e.g., using intracerebroventricular (ICV) injection) or by intravenous injection.

In one example, the method of the disclosure includes intracerebral or intracerebroventricular administration through stereotaxic injections. However, other known delivery methods may also be adapted in accordance with the disclosure. For example, for a more widespread distribution of the composition across the CNS, it may be injected into the cerebrospinal fluid, e.g., by lumbar puncture. To direct the composition to the peripheral nervous system, it may be injected into the spinal cord, one or more peripheral ganglia, or under the skin (subcutaneously or intramuscularly) of the body part of interest. In certain situations, the composition can be administered via an intravascular approach. For example, the composition can be administered intra-arterially (carotid) in situations where the blood-brain barrier is disturbed or not disturbed. Moreover, for more global delivery, the composition can be administered during the “opening” of the blood-brain barrier achieved by infusion of hypertonic solutions including mannitol.

The most suitable route for administration in any given case will depend on the particular composition administered, the subject, the particular epilepsy being treated, pharmaceutical formulation methods, administration methods (e.g., administration time and administration route), the subject's age, body weight, sex, severity of the diseases being treated, the subject's diet, and the subject's excretion rate.

### Combination Therapy

The compositions disclosed heroin may be administered to a subject in need thereof (e.g., a human subject) to treat an epilepsy (e.g., a TLE) in combination with one or more additional therapeutic modalities (e.g., 1, 2, 3, or more additional therapeutic modalities), including other therapeutic agents or physical interventions (e.g., rehabilitation therapy or surgical intervention). The two or more agents can be administered at the same time (e.g., administration of all agents occurs within 15 minutes, 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be performed by any appropriate route including, but not limited to, oral routes, intravenous routes, intramuscular routes, local routes, and direct absorption through mucous membrane tissues. The therapeutic agents can be administered by the same route or by different routes. For example, a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In cases in which the subject is diagnosed as having or at risk of developing an epilepsy (e.g., a TLE), the second therapeutic agent may include one or more antiepileptic drug (AED) including, but not limited to valproate, lamotrigine, ethosuximide, topiramate, lacosamide, levetiracetam, clobazam, stiripentol, benzodiazepine, phenytoin, carbamazepine, primidone, phenobarbital, gabapentin, pregabalin, tiagabine, zonisamide, felbamate, and/or vigabatrin. In some cases, the second therapeutic modality may be surgical intervention, such as, e.g., surgical resection of the epileptogenic brain region (e.g., a temporal lobe resection) using methods well-known in the art, such as, e.g., radiosurgery (e.g., gamma knife or laser ablation). Additional therapeutic modalities that can be administered together with the methods and compositions of the disclosure include vagus nerve stimulation, deep brain stimulation, transcranial magnetic stimulation, and ketogenic diet.

In particular examples, the subject may be administered an immune suppressant, including a regimen of corticosteroid alone, or tacrolimus or rapamycin (sirolimus), e.g., in combination with mycophenolic acid or in combination with a corticosteroid such as prednisolone and/or methylprednisolone. Other immune suppression regimens well known in the art can be employed in conjunction with the methods and compositions of the disclosure. Such immune suppression treatments may be administered before, after, or concomitantly with administration of the inhibitory nucleic acid molecule(s) (e.g., inhibitor RNA agent(s)) described herein.

### Dosing

Subjects that can be treated as described herein are subjects diagnosed as having or at risk of developing an epilepsy (e.g., a TLE). A subject that can be treated using the disclosed methods and compositions include, e.g., a subject who has had one or more previous therapeutic interventions related to the treatment of epilepsy or a subject who has had no previous therapeutic intervention for treatment of an epilepsy.

The inhibitory RNA agent of the disclosure may be administered in an amount and for a time effective to result in one or more of (e.g., 2 or more, 3 or more, 4 or more of): (a) decrease the level of Grik2 mRNA and/or GluK2 protein in a cell of the subject, (b) delayed onset of the disorder, (c) increased survival of subject, (d) increased progression free survival of a subject, (c) recovery or change in GluK2 protein function, (f) reduce risk of seizure recurrence; (g) reduction of excitotoxicity and associated neuronal cell death in the CNS; (h) restoration of a physiological excitation-inhibition balance in the affected region of the CNS (e.g., the hippocampus); and/or (i) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation).

Accordingly, the disclosure relates to a method for treating an epilepsy (e.g., TLE) in a subject in need thereof, in which the method includes administering an effective amount of a vector including an inhibitory polynucleotide encoding an inhibitory RNA (e.g., an ASO, such as, e.g., siRNA, shRNA, miRNA, or shmiRNA) that binds specifically to Grik2 mRNA and inhibits expression of GluK2 protein in the subject. In particular, the invention provides a method of treating an epilepsy in a subject in need thereof including administering to the subject a therapeutically effective amount of an inhibitory RNA agent disclosed herein or a nucleic acid vector encoding the same.

The epilepsy to be treated utilizing the disclosed methods and compositions may be TLE (e.g., mTLE or lTLE), benign Rolandic epilepsy, a frontal lobe epilepsy, infantile spasms, a juvenile myoclonic epilepsy, a juvenile absence epilepsy, a childhood absence epilepsy (pyknolepsy), a hot water epilepsy, Lennox-Gastaut syndrome, Landau-Kleffner syndrome, Dravet syndrome, a progressive myoclonus epilepsy, a reflex epilepsy, Rasmussen's syndrome, a limbic epilepsy, status epilepticus, an abdominal epilepsy, massive bilateral myoclonus, a catamenial epilepsy, Jacksonian seizure disorder, Lafora disease, and a photosensitive epilepsy. For example, a subject may be diagnosed with a TLE (e.g., mTLE or lTLE), such as, a TLE characterized by focal or generalized seizures. In some cases, the epilepsy may be a chronic epilepsy, such as, e.g., an epilepsy that is refractory to treatment (i.e., a pharmaco-resistant epilepsy, such as a pharmaco-resistant TLE).

For the treatment of epilepsy and to ameliorate the symptoms of seizures and epileptiform discharges as is discussed herein, a useful polynucleotide may be deployed by a vector which encodes a functional RNA, e.g., siRNA, shRNA, miRNA, or shmiRNA, that inhibits the expression of Grik2 mRNA.

The disclosed compositions can be administered in amounts determined to be appropriate by those of skill in the art. In some cases, the rAAV is administered at a dose of 10⁵, 10⁶, 10⁷, 10⁸, 10⁹, 10¹⁰, 10¹¹, 10¹², 10¹³, 10¹⁴, or 10¹⁵ genome copies (GC) per subject. In some embodiments the rAAV is administered at a dose of 10⁵, 10⁶, 10⁷, 10⁸, 10⁹, 10¹⁰, 10¹¹, 10¹², 10¹³, or 10¹⁴ GC/kg (total weight of the subject).

In some cases, 1×10¹² to 5×10¹⁴ GC are administered. In some cases, a flat dose of 1×10¹² to 5×10¹⁴ GC is administered to a pediatric patient or an adult patient.

In some cases, dosages are measured by the number of GC administered to the cerebrospinal fluid (CSF) of the patient (e.g., injected intrathecally, e.g., via suboccipital puncture or lumbar puncture) per gram of the patient's brain mass. In some cases, 10⁵, 10⁶, 10⁷, 10⁸, 10⁹, 10⁹, 10¹⁰, 10¹¹, 10¹², 10¹³, 10¹⁴, or 10¹⁵ genome copies per gram of patient's brain mass are administered.

In some cases, 1×10⁵ genome copies per gram of patient's brain mass are administered. In some cases, 1×10⁶ genome copies per gram of patient's brain mass are administered. In some cases, 1×10⁷ genome copies per gram of patient's brain mass are administered. In some cases, 1×10⁸ genome copies per gram of patient's brain mass are administered. In some cases, 1×10⁹ genome copies per gram of patient's brain mass are administered. In some cases, 1×10¹⁰ genome copies per gram of patient's brain mass are administered. In some cases, 5×10¹⁰ genome copies per gram of patient's brain mass are administered. In some cases, 1×10⁹ to 1×10¹¹ genome copies per gram of patient's brain mass are administered. In some cases, 1×10⁹ to 5×10¹⁰ genome copies per gram of patient's brain mass are administered. In some cases, 2×10⁹ to 9×10¹⁰ genome copies per gram of patient's brain mass are administered. In some cases, 5×10⁹ to 1×10¹¹ genome copies per gram of patient's brain mass are administered. In other cases, 1×10¹⁰ to 5×10¹⁰ genome copies per gram of patient's brain mass are administered. In other embodiments, 1×10¹⁰ to 9×10¹⁰ genome copies per gram of patient's brain mass are administered. The patient's (subject's) brain weight estimation is obtained from an MRI brain volume determination, which is converted to brain mass and utilized to calculate a precise dose of drug administered. Brain weights may also be estimated based on age range, using a published database.

Optionally, the disclosed agents may be administered as part of a pharmaceutically acceptable composition suitable for delivery to a subject, as is described herein. The disclosed agents are included within these compositions in amounts sufficient to provide a desired dosage and/or elicit a therapeutically beneficial effect, as can be readily determined by those of skill in the art.

The disclosed compositions described herein may be administered in an amount (e.g., an effective amount) and for a time sufficient to treat the subject or to effect one of the outcomes described above (e.g., a reduction in one or more symptoms of disease in the subject). The disclosed compositions may be administered once or more than once. The disclosed compositions may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months or more following administration of a composition of the disclosure depending on the composition and the route of administration used for treatment. Methods of evaluating treatment efficacy are disclosed herein (see, e.g., “Pharmaceutical Uses”). Depending on the outcome of the evaluation, treatment may be continued or ceased, treatment frequency or dosage may change, or the patient may be treated with a different disclosed composition. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated. For example, a subject diagnosed with TLE and treated with a composition disclosed herein may be given one or more (e.g., 1, 2, 3, 4, 5, 6, 7, 8, 9, 10, or more) additional treatments if initial or subsequent rounds of treatment do not elicit a therapeutic benefit (e.g., reduction of any one of the symptoms disclosed herein or a reduction in the levels of Grik2 mRNA or GluK2 protein levels in the afflicted brain region of the subject).

### Kits

The disclosure also provides kits that include a composition disclosed herein that inhibits the expression of a Grik2 gone in a subject (e.g., an inhibitory RNA targeting a Grik2 mRNA) for use in the prevention or treatment of an epilepsy (e.g., a TLE, such as treatment-refractory TLE). The kits can optionally include an agent or device for delivering the composition to the subject. In other examples, the kits may include one or more sterile applicators, such as syringes or needles. Further, the kits may optionally include other agents, e.g., anesthetics or antibiotics. The kit can also include a package insert that instructs a user of the kit, such as a physician, to perform the methods disclosed herein.

### Examples

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1: Design and Synthesis of Inhibitory Polynucleotides Targeting a Grik2 mRNA

Grik2-targeting inhibitory polynucleotide sequences were designed based on their predicted complementarity to a human Grik2 mRNA, e.g., a Grik2 mRNA sequence of SEQ ID NO: 164 and synthesized according to methods known in the art. The inhibitory polynucleotides contain a stem-loop sequence that includes an antisense guide strand sequence that specifically hybridizes to a region of the Grik2 mRNA and a sense passenger strand sequence that is substantially complementary to the guide strand. The anti-Grik2 stem-loop sequences can be transfected into human neuroblastoma cell line SHSY5Y or murine N2A neuronal cell line. Total RNA can be extracted and small RNA sequencing is performed to quantify the level of guide and passenger strands and to identify Drosha and Dicer cleavage sites. To measure the Grik2 knockdown efficacy, the constructs can be co-transfected with human Grik2 cDNA into HEK293 cells, mRNA can be extracted and real time quantitative polymerase chain reaction (RT-qPCR) for Grik2 mRNA can be performed. In addition, the synthetic stem-loop RNAs can be transfected into SHSY5Y cells followed by mRNA extraction and Grik2 RT-qPCR.

### Example 2: Treatment of an Epilepsy in a Human Subject by Administration of a Viral Vector Encoding One or More Inhibitory Polyribonucleotides Targeting a Grik2 mRNA

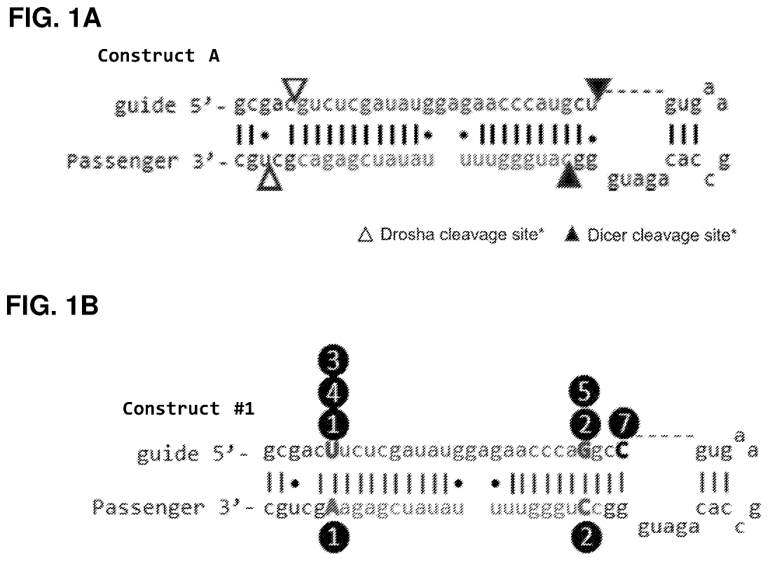

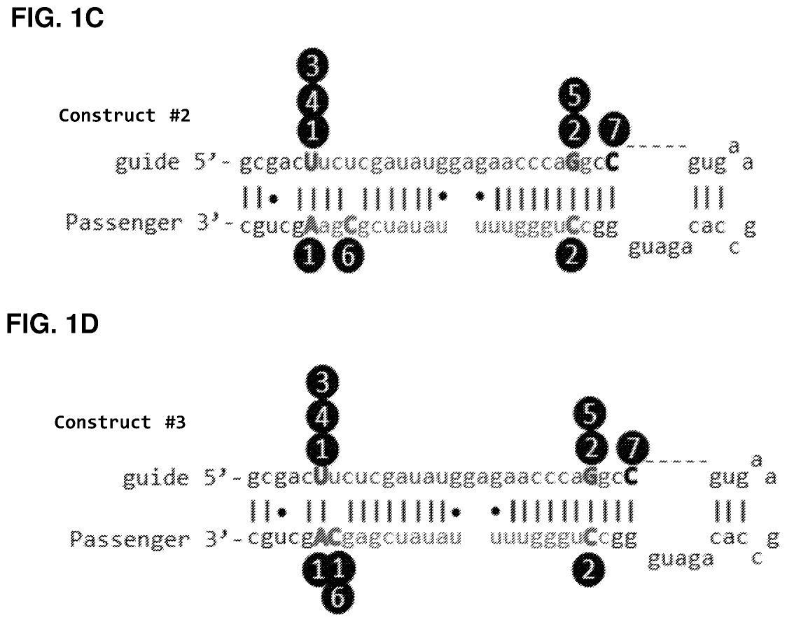

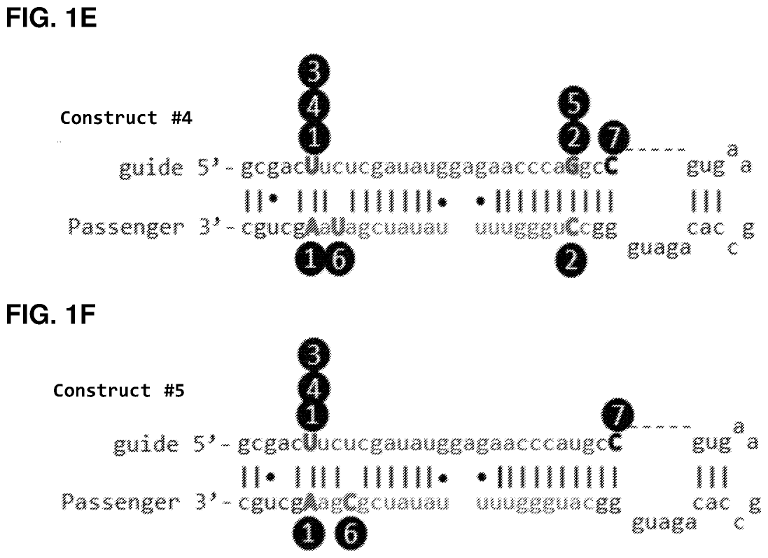

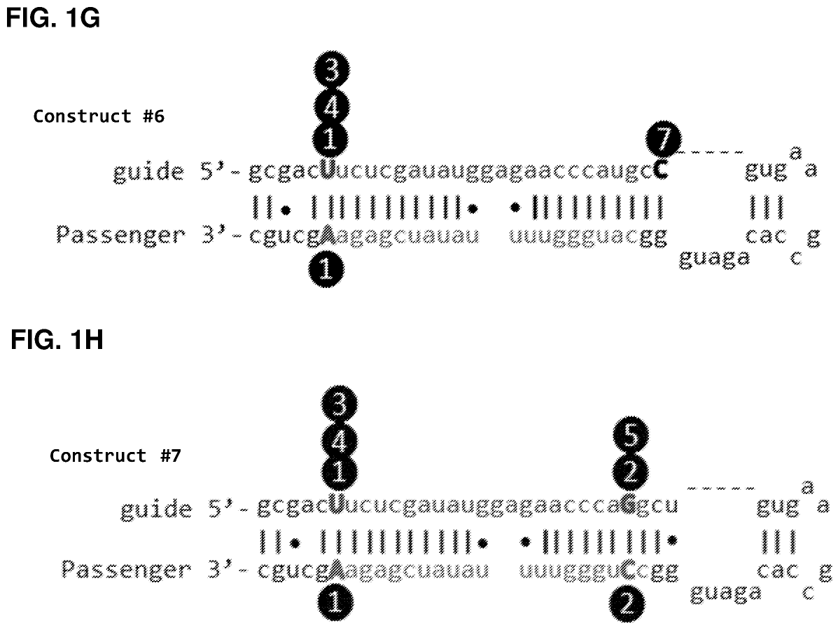

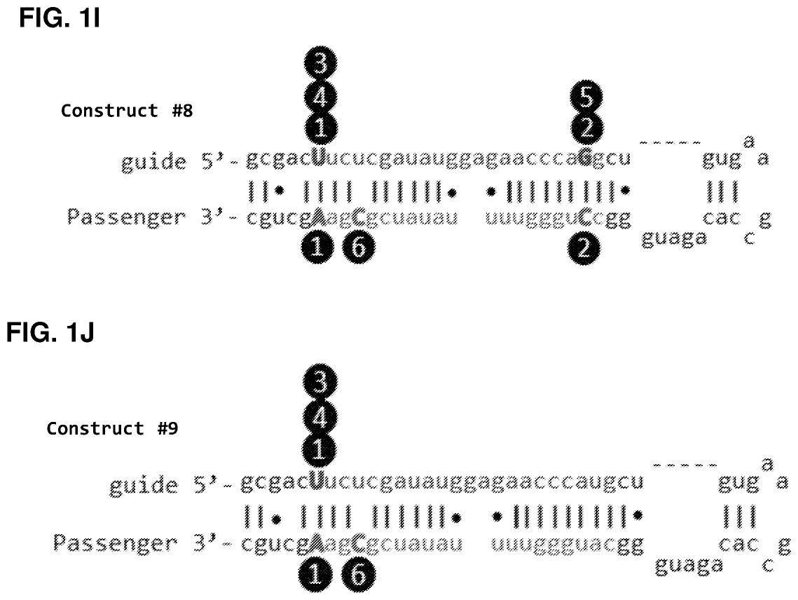

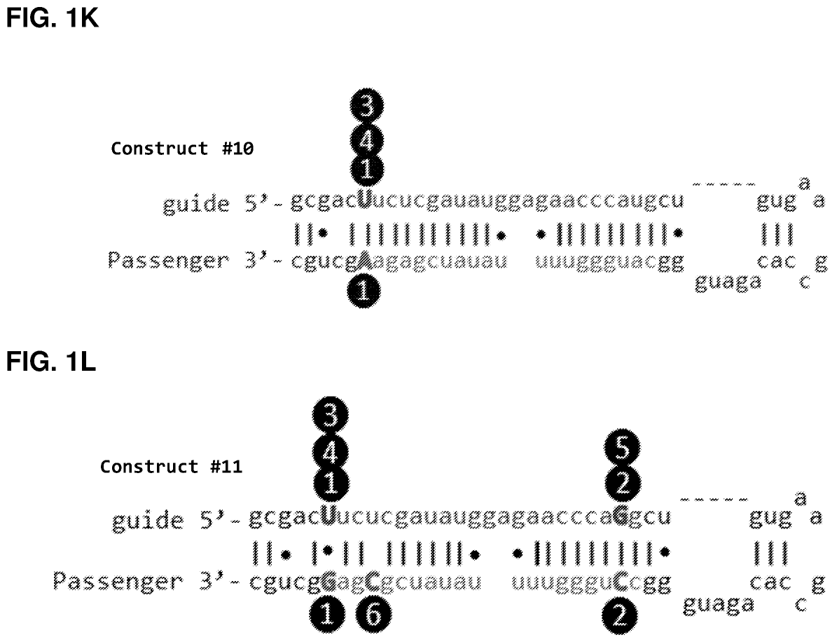

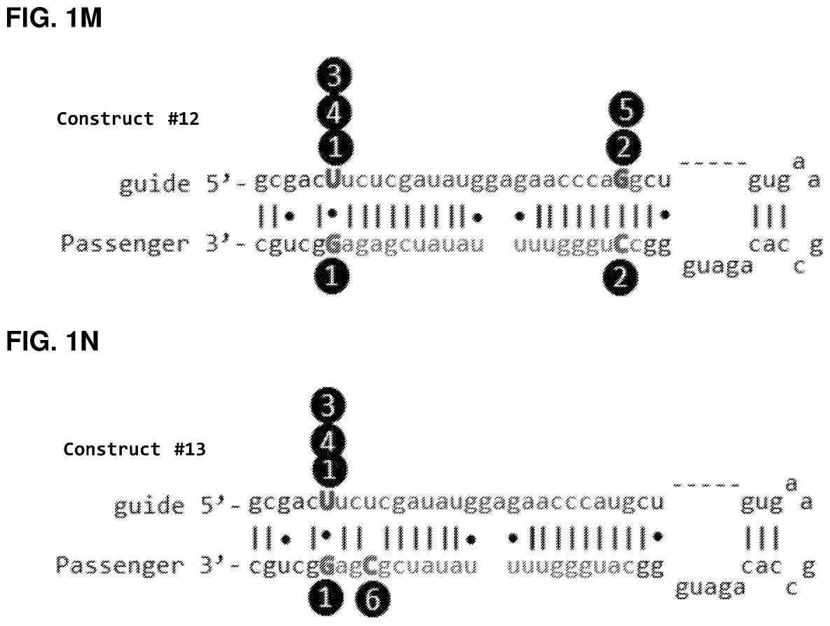

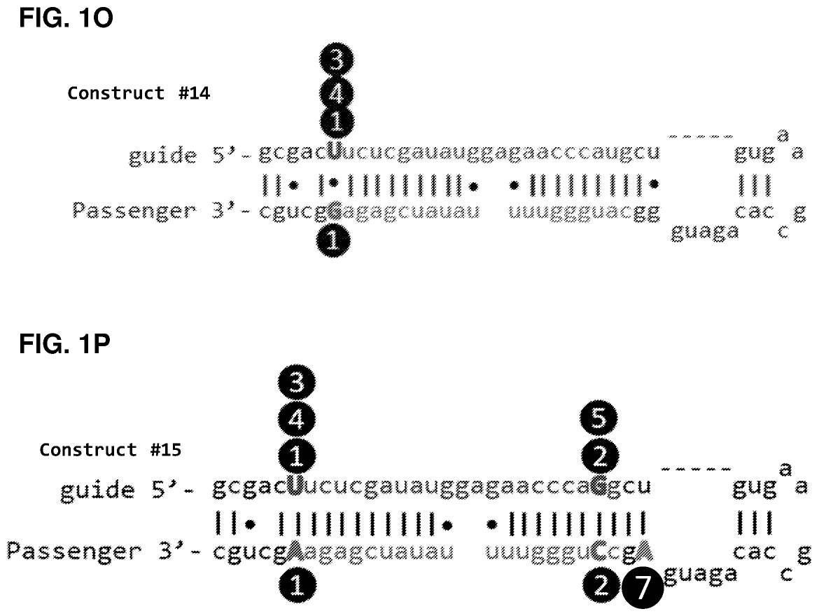

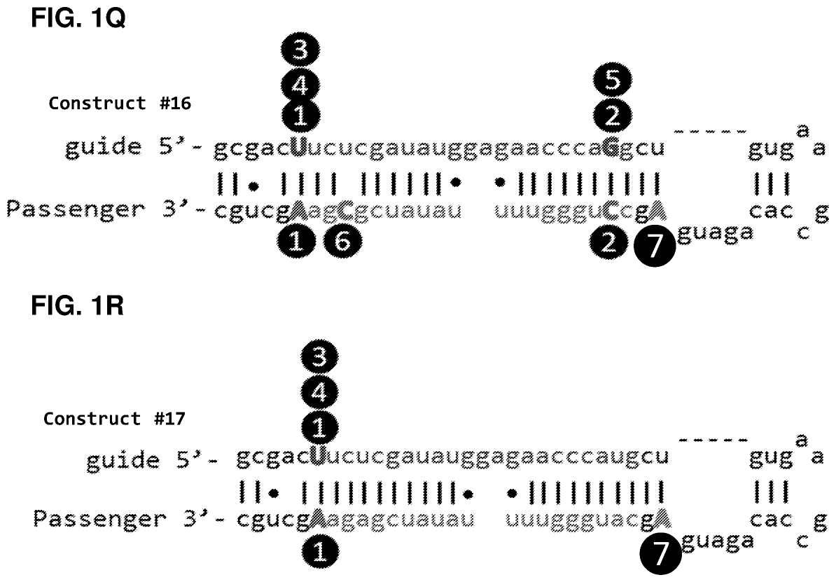

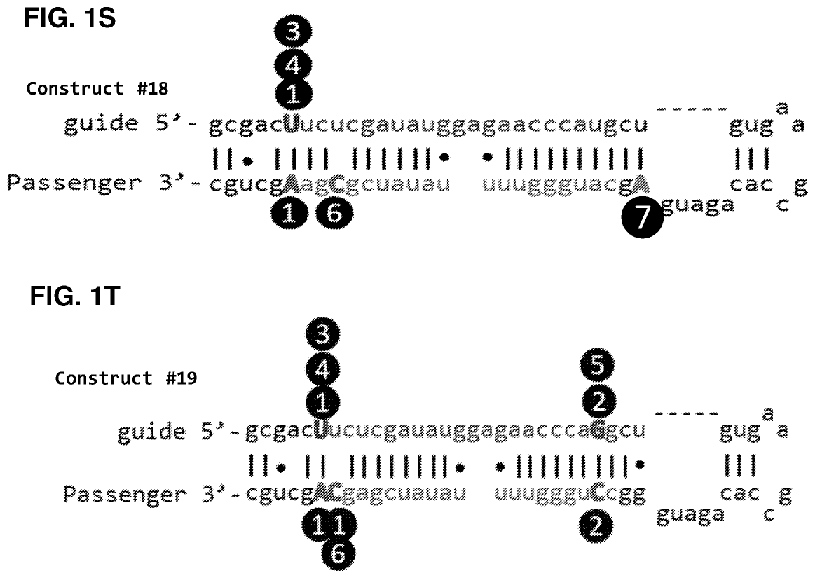

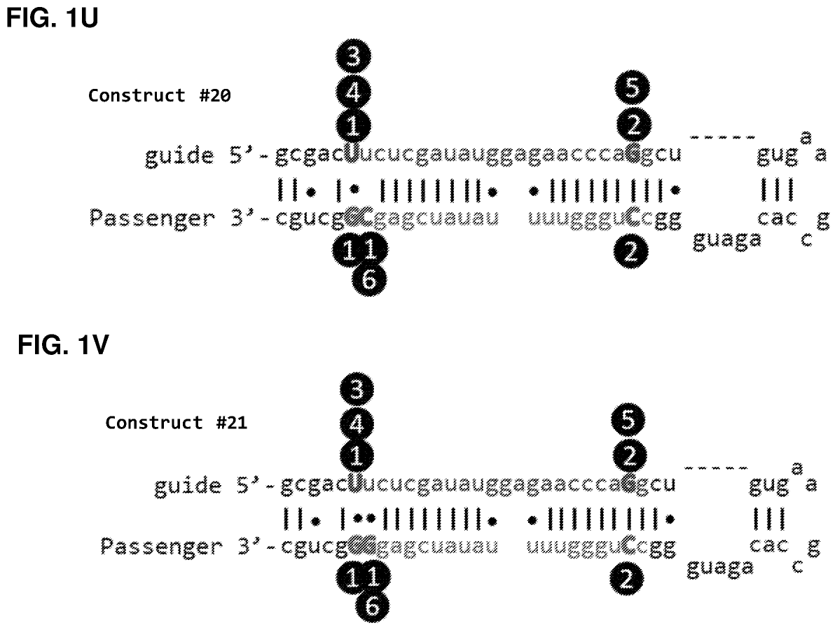

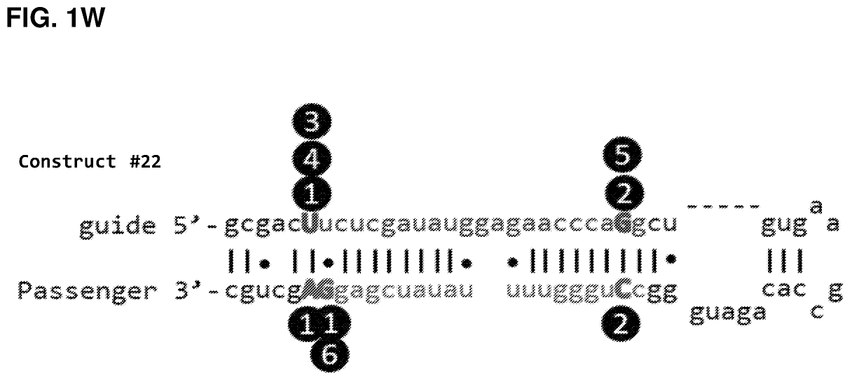

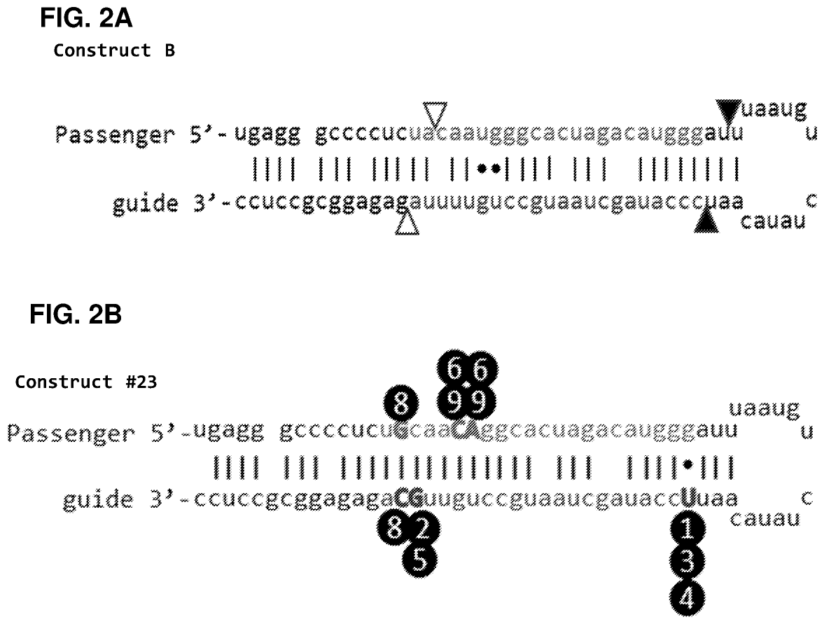

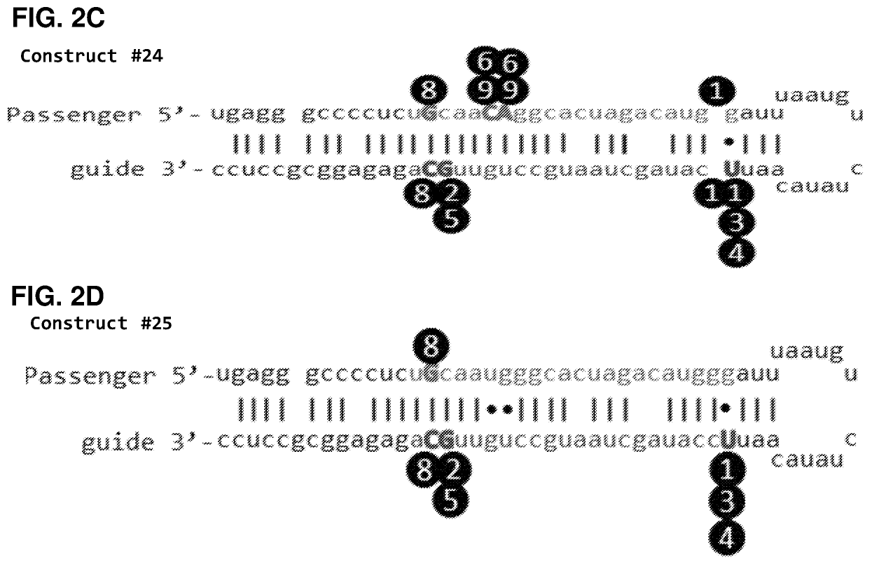

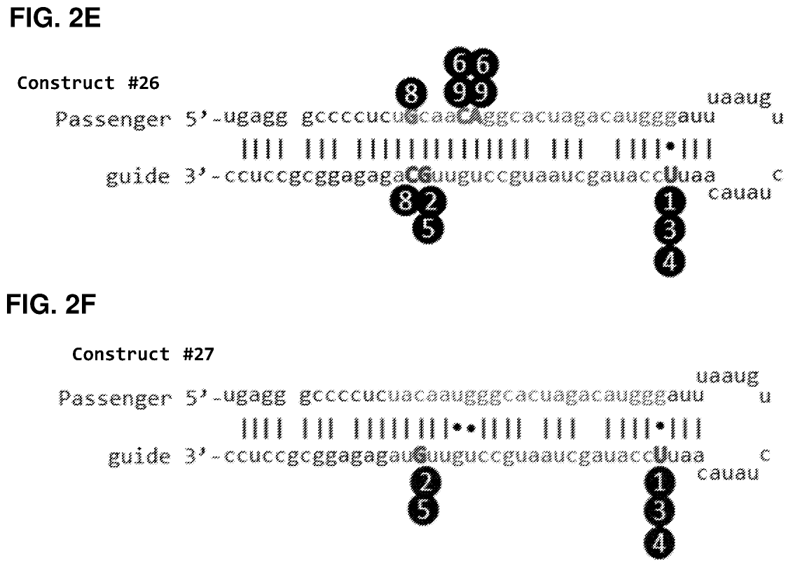

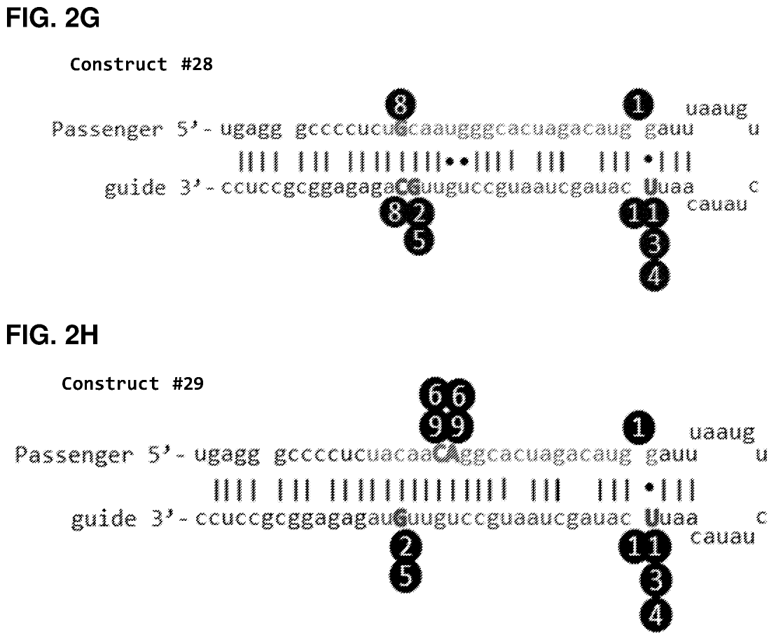

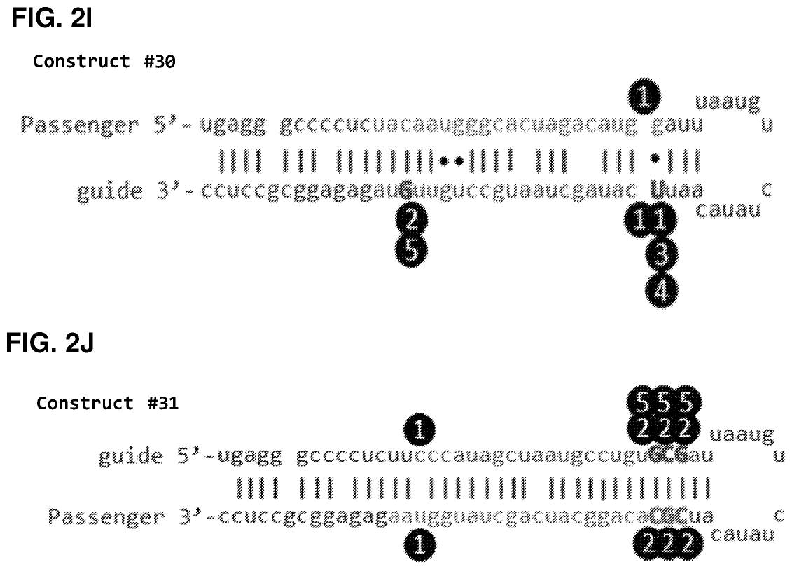

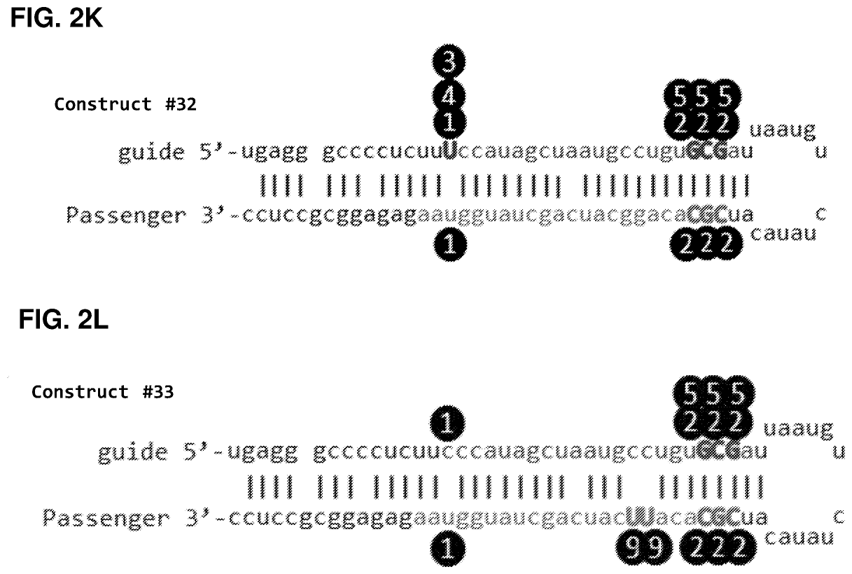

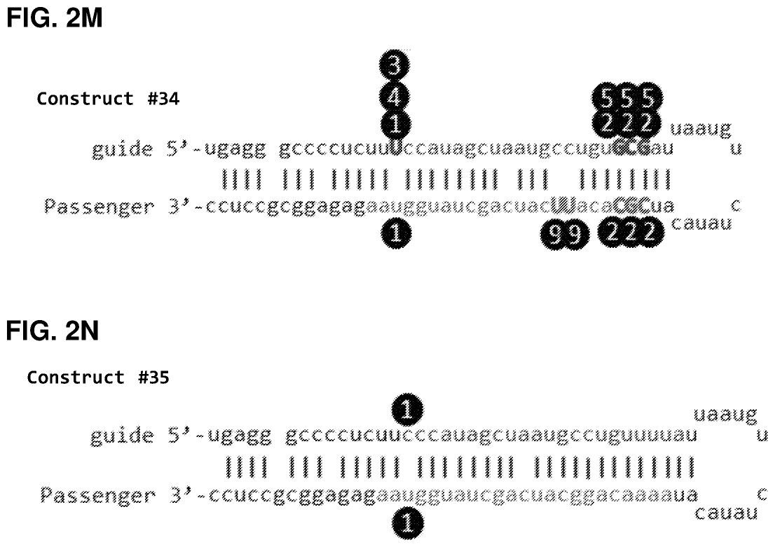

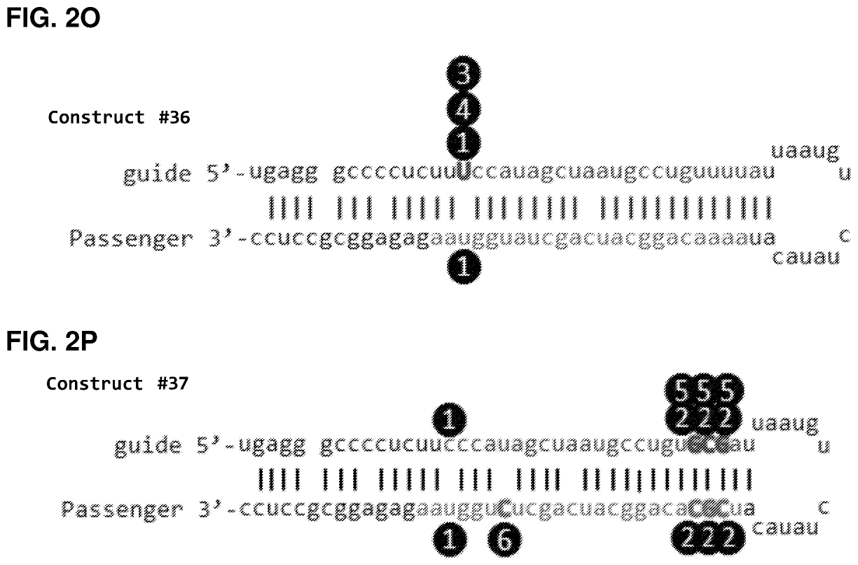

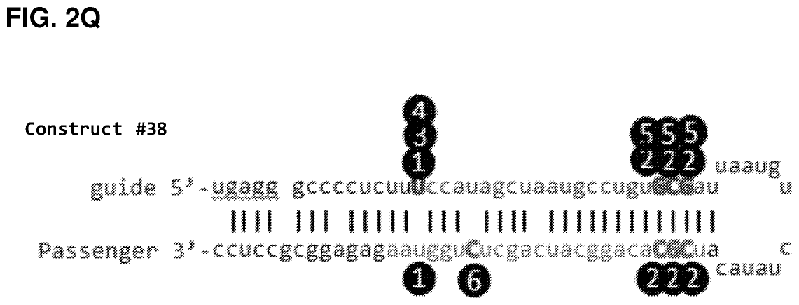

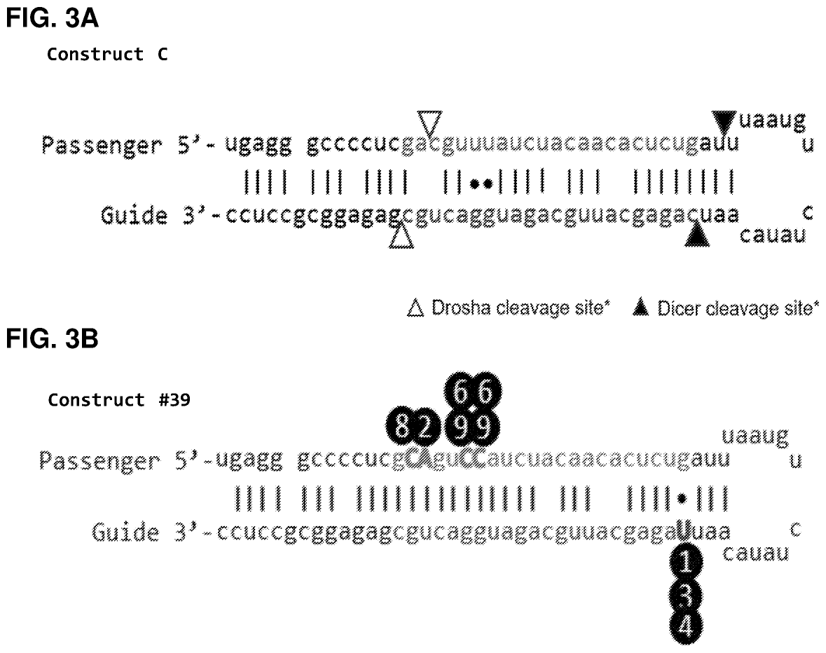

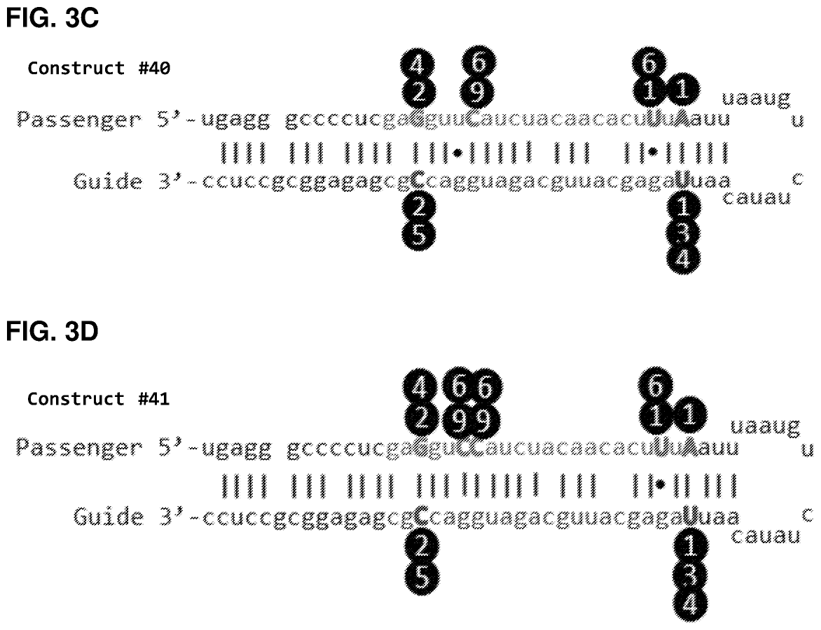

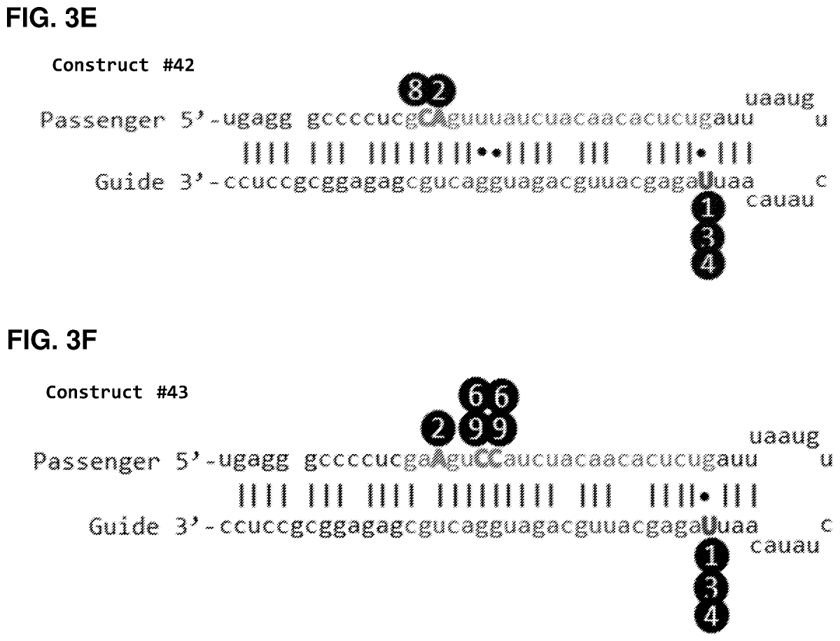

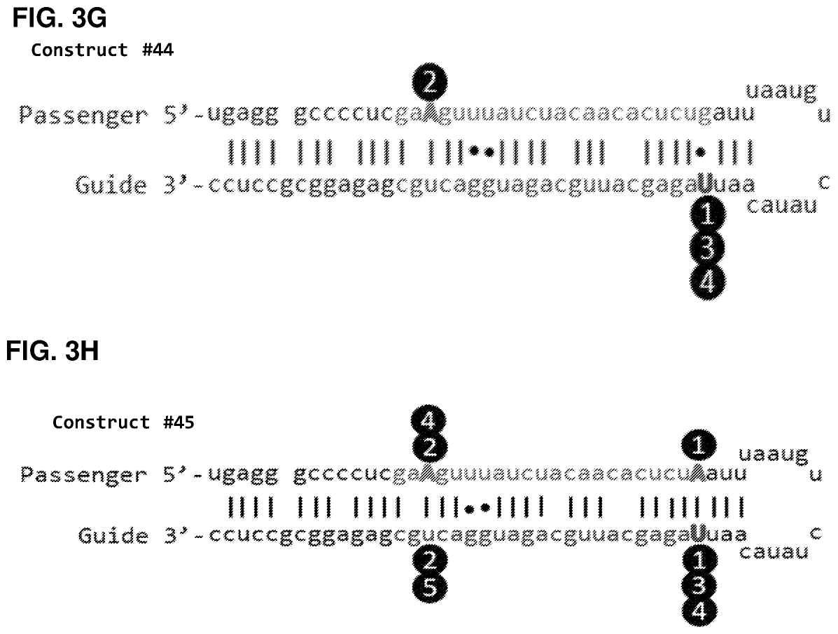

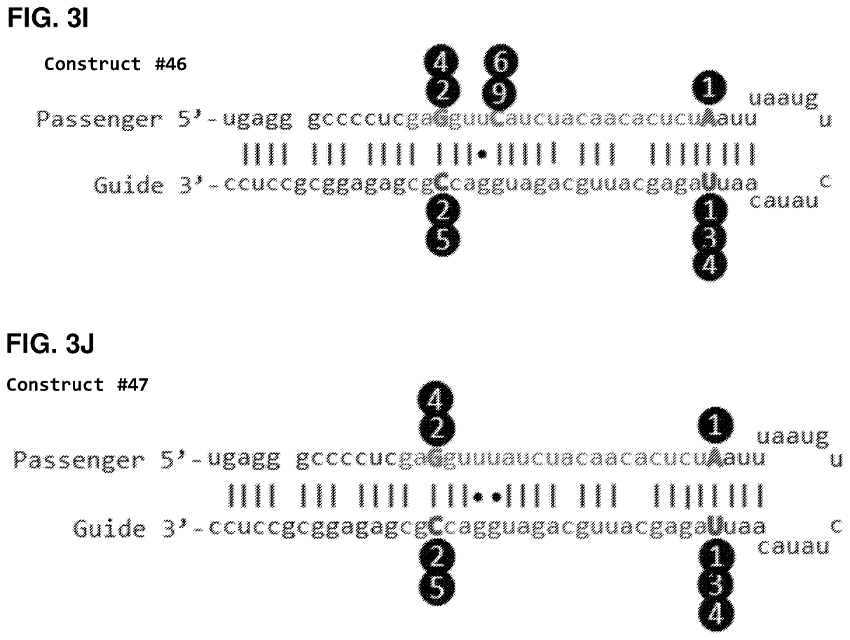

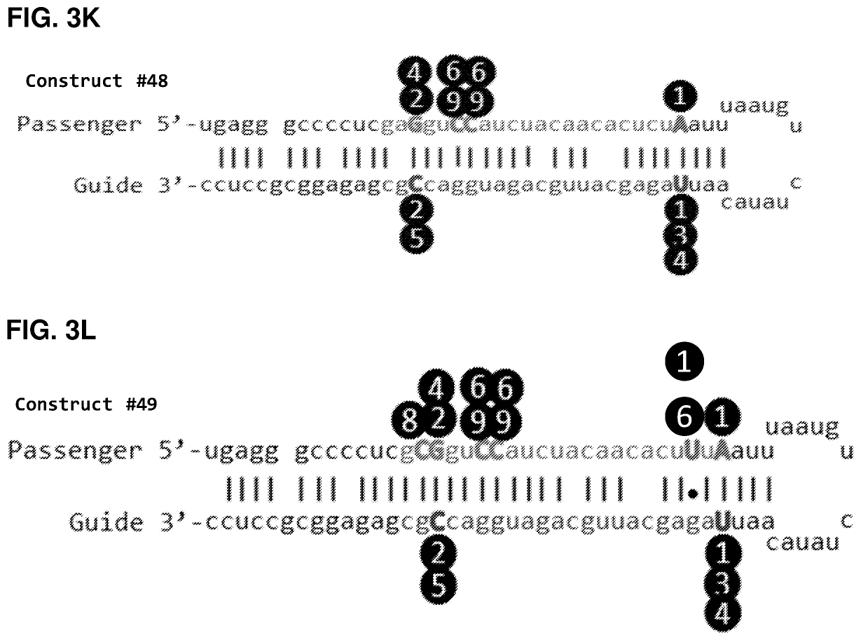

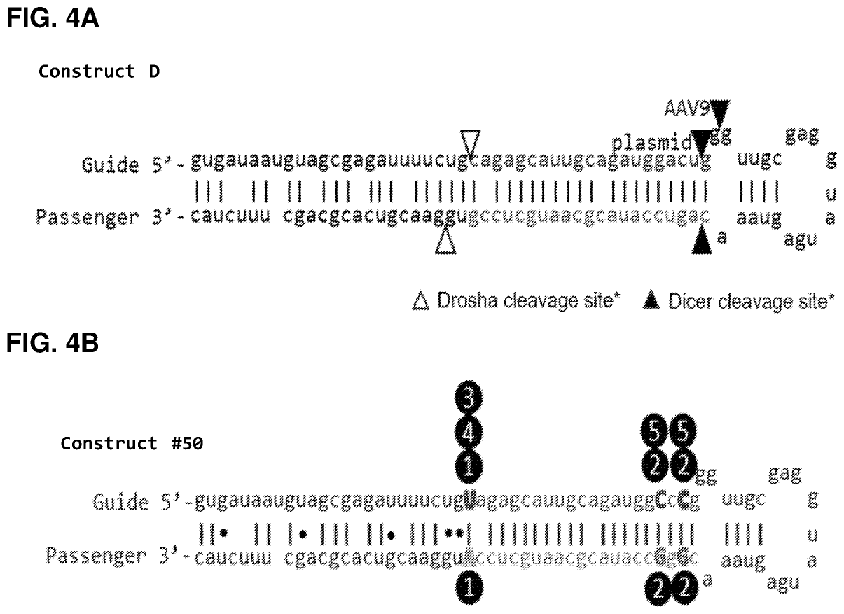

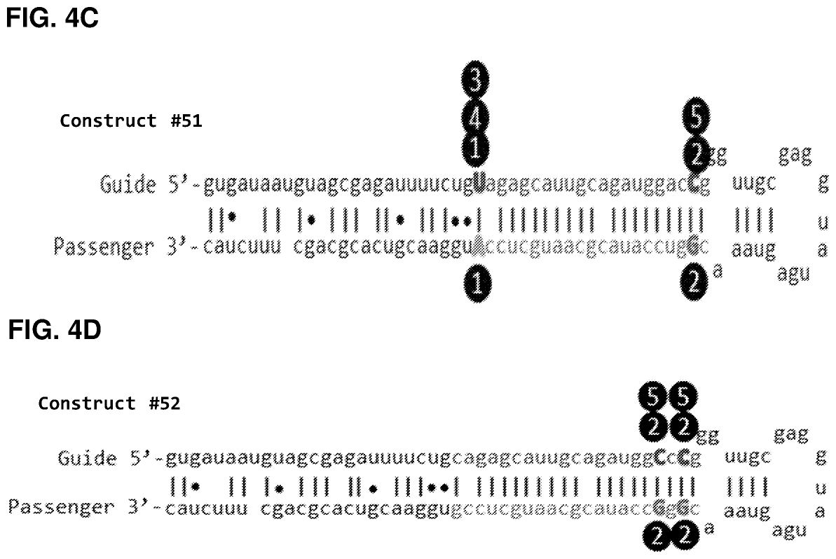

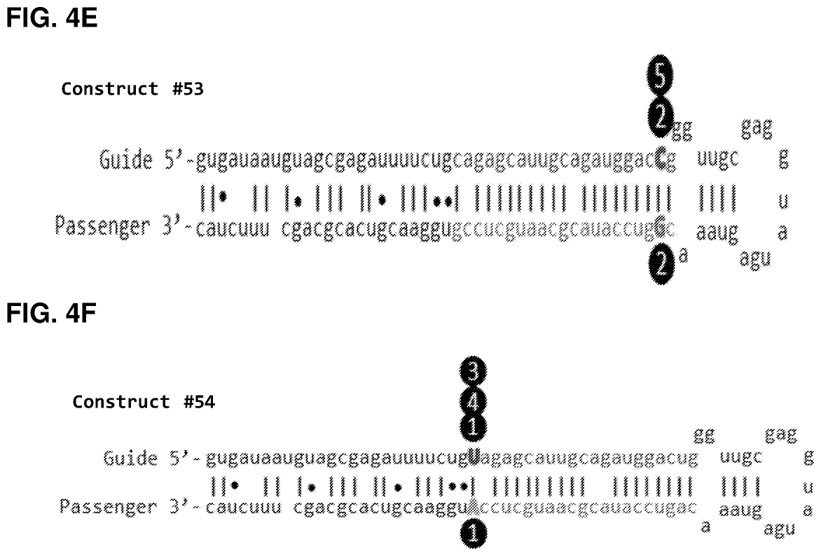

A subject, such as a human subject (e.g., a pediatric or adult subject) diagnosed as having an epilepsy (e.g., a TLE, such as, e.g., mTLE or lTLE), can be treated with a composition described herein to reduce one of more epilepsy symptoms including, but not limited to one or more of (e.g., 2 or more, 3 or more, 4 or more of): (a) risk of seizure recurrence; (b) reduction of excitotoxicity and associated neuronal cell death in the CNS; (c) restoration of a physiological excitation-inhibition balance in the affected region of the CNS; (d) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation), and (e) pathological sprouting of recurrent mossy fibers of dentate gyrus granule cells in the hippocampus. The method of treatment can optionally include diagnosing or identifying the subject as a candidate for treatment with a composition of the disclosure before administration. The composition can include an inhibitory polynucleotide (e.g., a polynucleotide encoding an inhibitory RNA sequence of the disclosure) targeting a Grik2 mRNA or a nucleic acid vector (e.g., a viral vector, such as an AAV vector, e.g., an AAV vector having any one of the serotypes selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV. RHM4-1, AAV.hu37, AAV.Anc80, AAV.Anc80L65, AAV.7m8, AAV.PHP.B, AAV. PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a lentiviral vector) containing a polynucleotide encoding the same (e.g., an AAV9 vector). Exemplary inhibitory polynucleotides may have no less than 85% (e.g., at least 85%, 86%, 87%, 88%, 89%, 90%, 91%, 92%, 93%, 94%, 95%, 96%, 97%, 98%, 99%, or more (e.g., 100%)) sequence identity to any one of the nucleic acid sequences of SEQ ID NOs: 1-19, 34-62, 97-108, 133-147, 226-229, and 238-241, or they may have the sequence of one or more of SEQ ID NOs: 1-19, 34-62, 97-108, 133-147, 226-229, and 238-241, (see also FIGS. 1B-1W, FIGS. 2B-2Q, FIGS. 3B-3L, FIGS. 4B-4F, FIGS. 5B-5E, and FIGS. 6A-6B for representative construct diagrams). Furthermore, the viral vector (e.g., AAV vector) may be incorporate an expression cassette containing the inhibitory polynucleotide and regulatory sequences that facilitate heterologous expression of the polynucleotide, such as, e.g., a promoter sequence (see Tables 11 and 12).

The subject can be administered the composition by any suitable means, including, e.g., intravenous, intraperitoneal, subcutaneous, or transdermal administration, or by way of administration directly to the central nervous system of the animal (e.g., stereotactic, intraparenchymal, intrathecal, or intracerebroventricular injection). The composition can be administered in a therapeutically effective amount, such as at a dose of 10⁵, 10⁶, 10⁷, 10⁸, 10⁹, 10¹⁰, 10¹¹, 10¹², 10¹³, 10¹⁴, or 10¹⁵ genome copies (GC) per subject, at a dose of 10⁶, 10⁶, 10⁷, 10⁸, 10⁹, 10¹⁰, 10¹¹, 10¹², 10¹³, or 10¹⁴ GC/kg (total weight of the subject), at a dose of 10⁵, 10⁶, 10⁷, 10⁸, 10⁹, 10¹⁰, 10¹¹, 10¹², 10¹³, 10¹⁴, or 10¹⁵ GC per gram of patient's brain mass are administered. The subject's brain weight estimation is obtained from an MRI brain volume determination, which is converted to brain mass and utilized to calculate a precise dose of drug administered. Brain weights may also be estimated based on ago range, using a published database. The agent can be administered bimonthly, once a month, once every two weeks, or at least once a week or more (e.g., 1, 2, 3, 4, 5, 6, or 7 times a week or more). The composition may be administered in combination with a second therapeutic modality, such as a second therapeutic agent (e.g., an anti-epileptic drug), surgical intervention (e.g., surgical resection, radiosurgery, gamma knife, or laser ablation), vagus nerve stimulation, deep brain stimulation, or transcranial magnetic stimulation.

The composition can be administered to the subject in an amount sufficient to decrease one or more of (e.g., 2 or more, 3 or more, 4 or more of): (a) risk of seizure recurrence; (b) reduction of excitotoxicity and associated neuronal cell death in the CNS; (c) restoration of a physiological excitation-inhibition balance in the affected region of the CNS; (d) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation), and (e) pathological sprouting of recurrent mossy fibers of dentate gyrus granule cells in the hippocampus by 10% or more (e.g., 10%, 20%, 30%, 40%, 50%, 60%, 70%, 80%, 90%, 95% or more). The above-listed symptoms of epilepsy may be assessed using standard methods, such as neurological examination, electroencephalogram, magneto encephalogram, CT scan, PET scan, fMRI scan, videography, and visual observation. Measures of epilepsy symptoms from before and after administration of the composition can be compared to evaluate the efficacy of the treatment. A finding of a reduction in the symptoms of epilepsy described above indicates that the composition has successfully treated the epilepsy in the subject.

The numerals in black circles shown in FIGS. 1B-1W, FIGS. 2B-2Q, FIGS. 3B-3L, FIGS. 4B-4F correspond to distinct rationales for the design of the disclosed anti-Grik2 constructs. Accordingly, each of the numeral-designated rationales are as follows:

[symbol] RISC assembly favors the miRNA strand that has a 5′ end with a greater propensity to fray or to be liberated from the duplex (Schwarz et al., Cell 115:199-208 (2003); Khvorova et al., Cell 115:209-16 (2003); and Medley et al., Wiley Interdiscip. Rev. RNA 12: e1627 (2021)). The constructs disclosed herein favor guide selection by RISC, via destabilizing base pairing at the 5′ end of the guide strand (introduction of a U-A pair, U-G wobble pair, or mismatch, or delete one G-C pair).

[symbol] RISC assembly favors the miRNA strand that has a 5′ end with a greater propensity to fray or to be liberated from the duplex. The constructs disclosed herein disfavor passenger selection by RISC via tightening of base-pairing at the 5′ end of the passenger strand by introduction of a G-C pair at the 5′ end of the passenger strand.

[symbol] The 5′ terminal nucleotide of a small guide RNA is anchored in the phosphate-binding pocket of Argonaute (AGO) proteins and unavailable for base pairing with its target RNA (Medley et al., Wiley Interdiscip. Rev. RNA 12: e1627 (2021); Ma et al., Nature 434:666-70 (2005); Parker et al., Nature 434:663-6 (2005); and Ghildiyal et al., RNA 16:43-56 (2010)).

[symbol] 5′-U is the most preferred nucleotide for AGO2 association (Seitz et al., Silence 2:4 (2011); Frank et al., Nature 465:818-22 (2010); De et al., Mol. Cell 50:344-55 (2013); and Czech et al., Nat. Rev. Genet. 12:19-31 (2011)). Introduction of U as the first (5′) nucleotide of the guide strand, and introduction of G or C as the first (5′) nucleotide for the passenger strand facilitates AGO2 association.

[symbol] Introduction of 3′ mismatches between guide and its target mRNA leads to more potent silencing of abundant mRNAs in mammalian cells (De et al., Mol. Cell 50:344-55 (2013); Bofill-De Ros Methods 103:157-66 (2016); and Amarzguioui et al., Nucleic Acids Res. 31:589-95 (2003)). While extensive complementarity in the seed region (guide nucleotide 2-8 (g2-g8)) and the middle region of guide strand are crucial for AGO2-mediated mRNA target cleavage, base paring at the 3′ end is not required. Indeed, mismatches at position g18, g19, g20, g21 with target mRNA attenuate the release of the guide RNA from AGO2, an unloading activity mediated by target mRNA.

[symbol] Mismatch in the seed region (guide strand nucleotide positions 2-8) between guide strand and passenger strand promote unwinding of passenger strand during RISC loading, thereby promoting RISC maturation (Ghildiyal et al., RNA 16:43-56 (2010); Tomari et al., Cell 130:299-308 (2007); Matranga et al., Cell 123:607-20 (2005); and Kawamata et al., Nat. Struct. Mol. Biol. 16:953-60 (2009)). The constructs disclosed herein promote guide-RISC complex maturation by introduction of a mismatch in the seed region of the guide strand, or reduce or prevent passenger-RISC complex maturation by conversion of a mismatch to a pairing nucleotide in the corresponding region of the passenger strand (passenger strand nucleotide positions 2-8).

[symbol] Introduction of G-C or U-A base pairing to replace a U-G wobble at a junction of the stem region and the loop region facilitates cleavage by Dicer protein (Liu et al., Cell 173:1191-203 (2018)).

[symbol] Introduction of G-C base pairing in the flanking region adjacent to the stem-loop structure (i.e., Drosha cleavage site) facilitates higher precision of cleavage by Drosha.

[symbol] Introduction of paired or mismatched base-pairs in the stem region by mimicking the scaffold structure of E-miR-124.

### Example 3: In Vitro Knockdown Efficacy of Grik2 mRNA Using Synthetic Inhibitory Polynucleotides

In order to determine the in vitro efficacy of knockdown using the inhibitory polynucleotides of the disclosure, stem-loop RNAs were synthesized by Integrated DNA Technologies, and reconstituted in Nuclease Free Duplex Buffer. Intramolecular annealed oligonucleotides were obtained after incubation at 95° C. for 2 minutes and followed by gradual cooling to room temperature. The siRNA negative control (siNegative, Cat. #AM4621) and two positive control siRNAs targeting Grik2 (siPositive-1 and siPositive-2, Cat. #4392420 and 4392420) were obtained from Life Technologies. Transfection of SH-SY5Y cells with annealed stem-loop RNA oligonucleotides or siRNAs at 10 nM using LIPOFECTAMINE™ RNAIMAX Transfection Reagent was carried out according to the manufacturer's protocol. A transfection mixture without the RNA (RNAIMAX only) served as a further transfection control. Transfections were carried out in quadruplicate. Cells were further cultured at 37° C., 5% CO₂ until 72 hours post-transfection before being subjected to cell lysis and qRT-PCR. The expression levels of Grik2 mRNA were normalized to actin (ACTB). The relative Grik2 expression levels were obtained by comparison with siNegative control that was set to 100%.

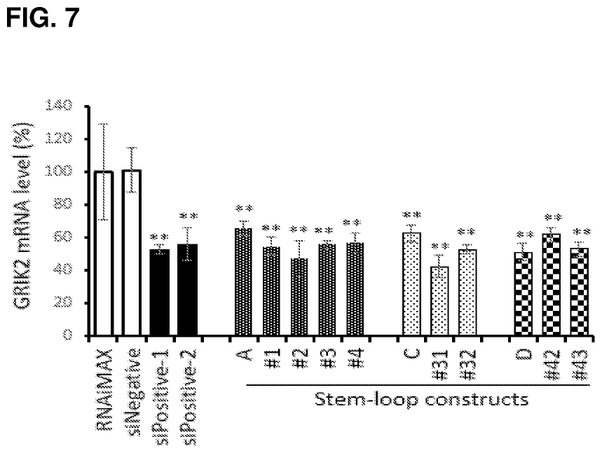

In comparison with the negative control and transfection reagent alone (RNAiMAX), all tested stem-loop RNA oligonucleotides as well as the positive controls showed Grik2 mRNA knockdown in SH-SY5Y cells (FIG. 7). Statistically significant knockdown rates (between 42% and 69% of remaining mRNA expression) were obtained at 72 hours after transfection. Grik2 knockdown efficacy was maintained by the tested constructs as compared with the miRNA Constructs A, C, and D.

### Example 4: RNA Sequencing Indicates Favorable microRNA Processing Properties in Mouse Neurons

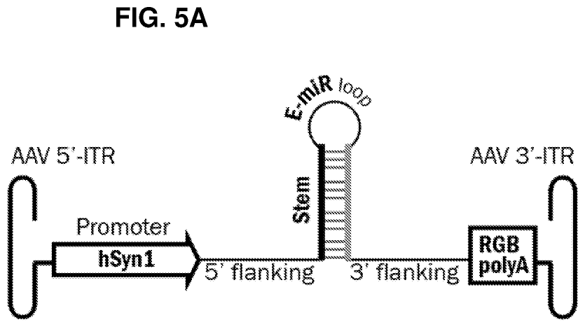

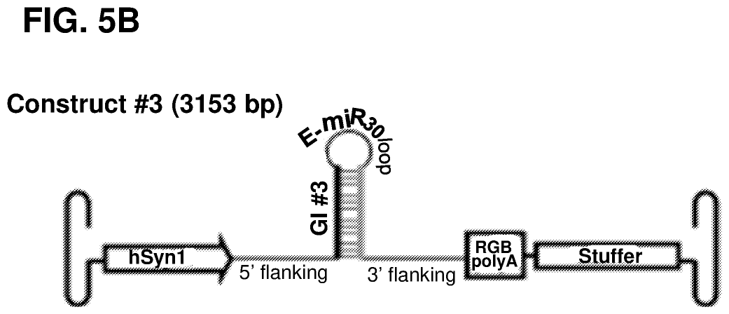

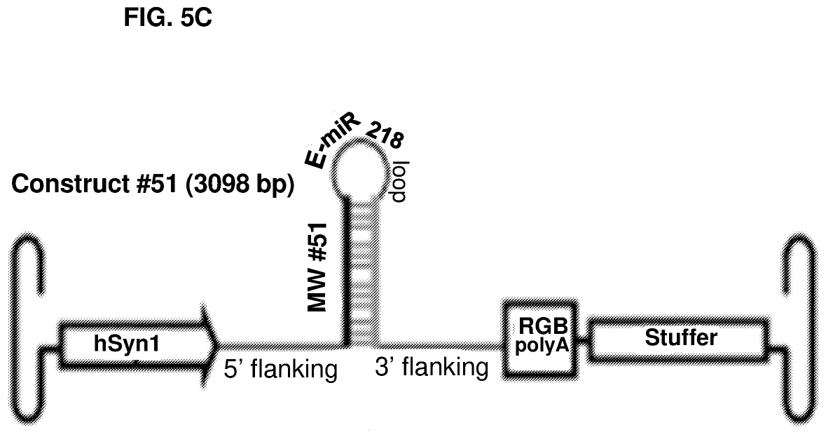

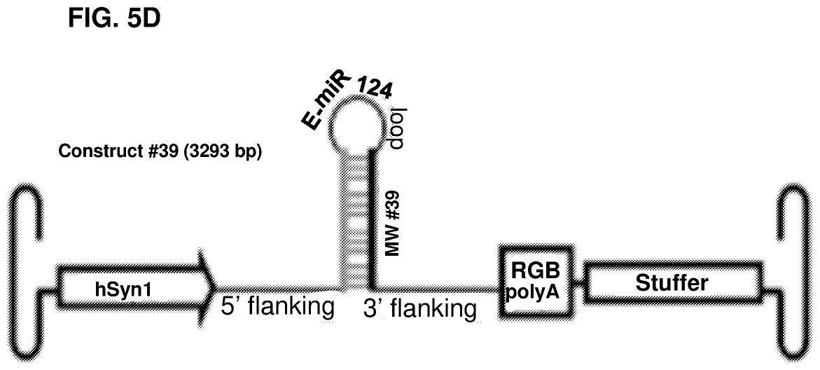

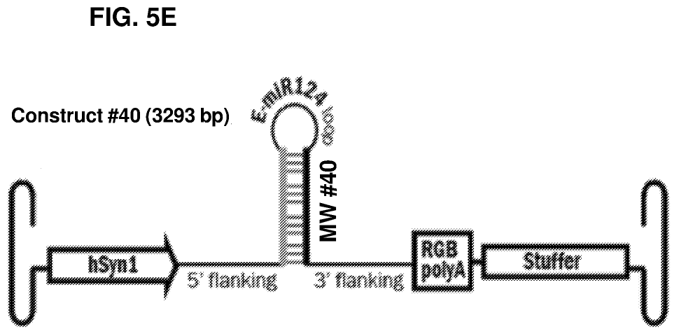

Stem-loop sequences were embedded in endogenous microRNA scaffolds (i.e., E-miR-30, E-miRNA-124-3, or E-miR-218-1) and subcloned under regulatory control of the neuron-specific promotor hSyn1 in cis plasmids encoding the AAV genome (pro-AAV) (FIG. 5A-5E). The plasmids were transfected into N2A mouse neuron cells. Mouse N2A neuroblast cells were seeded in 24-well plate at 9.0E4 cells/well and after 24 hours the cells were transfected with 250 ng plasmids and 1.2 μl Lipofectamine 3000/well. After 48 hours, RNA enriched for small RNAs was isolated using the Qiagen miRNeasy mini kit and was subjected to library preparation and small RNA sequencing. The microRNA sequencing library was generated using NEXTFLEX® small RNA kit from Perkin Elmer/BIOO. Sequencing was performed on the MiSeq platform.

SH-SY5Y cells were transfected by electroporation. The total RNA was extracted by Trizol and the microRNA sequencing library was generated using NEBNEXT® kit from New England Biolabs (NEB). Sequencing was performed on HiSeq in Novogene.

The rationally designed miRNAs were expressed and processed in mouse N2A neurons after plasmid transfection. Compared with the parental sequences (Constructs A, C, and D), the rationally designed microRNA constructs exhibited an improved guide-to-passenger (G:P) ratio (see Table 14). Specifically, G: P ratio 0.8 for Construct A was increased to a ratio of between 11 and 27 for four of the rationally designed constructs (Constructs #1-4) that have one or more modifications as compared to Construct A. Benefiting from the passenger decrease as well as potentially miRNA maturation improvement, the guide strand level (guide strand count percentage in total miRNA pool) was increased from 0.25% to 1.3-2.6% for Constructs #1-4. The G: P ratio of 1 and guide level of 2.8% for Construct C were increased to above 100 and 5%-21%, respectively, in Constructs #39, #40 and #41. Constructs #50 and #51 exhibited an improved G:P ratio of above 2000 relative to a G: P ratio of 1 for Construct D. Finally, the Construct #51 guide level increased to 5.1%.

Similar results were observed using the human neuroblastoma cell line SH-SY5Y. The G/P ratio of GI in both the original construct and the redesigned constructs was higher than that observed in N2A cells. This may be due to differences in biogenesis/degradation homeostasis in the two cell systems, or may be due to the different kits used for sequencing library preparation.

TABLE 14
Improvement of guide/passenger ratio

| | | N2A cells | | | | SH-SY5Y cells* | |
|---|---|---|---|---|---|---|---|
| Parental | | | | Increase | Increase | | Increase |
| guide, | | G:P | Guide | of G:P | of guide | | of G:P |
| scaffold | Construct | ratio | level | (fold) | (fold) | G:P | (fold) |
| GI | Construct A | 0.8 | 0.25% | | | 30.3 | |
| E-mir30a | SEQ ID NO: 1 | | | | | | |
| | Construct #1 | 10 | 2.66% | 12.5 | 10.6 | 782.5 | 25.8 |
| | (SEQ ID NO: 2) | | | | | | |
| | Construct #2 | 13 | 1.98% | 16.2 | 7.9 | 941.6 | 31.1 |
| | (SEQ ID NO: 3) | | | | | | |
| | Construct #3 | 23 | 2.10% | 28.8 | 8.4 | 912.2 | 30.1 |
| | (SEQ ID NO: 4) | | | | | | |
| | Construct #4 | 24 | 1.33% | 30 | 5.3 | 616.9 | 20.4 |
| | (SEQ ID NO: 5) | | | | | | |
| MW, | Construct D | 5.8 | 0.93% | | | 34 | |
| E-mir218-1 | (SEQ ID NO: 133) | | | | | | |
| | Construct #50 | 529 | 0.42% | 91 | 0.45 | 217 | 6.4 |
| | (SEQ ID NO: 134) | | | | | | |
| | Construct #51 | 1863 | 5.11% | 321 | 5.5 | 755 | 22.2 |
| | (SEQ ID NO: 135) | | | | | | |
| MW, | Construct C | 1 | 2.83% | | | 0.25 | |
| E-mir124-3 | (SEQ ID NO: 97) | | | | | | |
| | Construct #39 | 121 | 19.92% | 121 | 7.0 | 1572 | 6288 |
| | (SEQ ID NO: 98) | | | | | | |
| | Construct #40 | 12312 | 4.97% | 12312 | 1.8 | NP** | |
| | (SEQ ID NO: 99) | | | | | | |
| | Construct #41 | 535 | 21.3% | 535 | 7.5 | 8296 | 33184 |
| | (SEQ ID NO: 100) | | | | | | |
| G9, | Construct B | 0.01 | 0.30% | | | 0.0015 | |
| E-mir124-3 | (SEQ ID NO: 80) | | | | | | |
| | Construct #23 | 0.72 | 0.39% | 72 | 1.3 | 0.016 | 10.7 |
| | (SEQ ID NO: 81) | | | | | | |
| | Construct #24 | 0.12 | 0.21% | 12 | 0.7 | 0.0078 | 5.2 |
| | (SEQ ID NO: 82) | | | | | | |
| | Construct #31 | — | — | — | — | 0.0657 | 43.8 |
| | (SEQ ID NO: 89) | | | | | | |

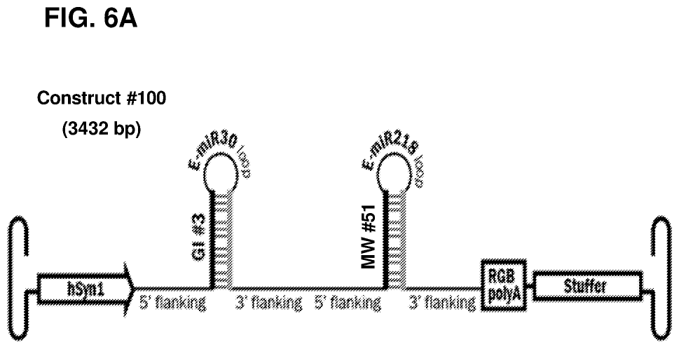

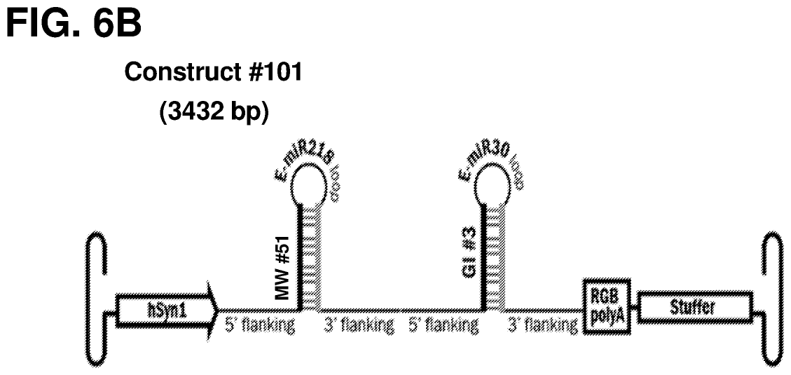

Two redesign constructs, #3 (GI, E-miR-30a) and #51 (MW, E-miR218-1), were selected and concatenated in two different orders in two constructs #100 (FIG. 6A, SEQ ID NO: 256) and #101 (FIG. 6B, SEQ ID NO: 257). A strong cluster effect was observed in both concatemer constructs, with MW boosted and GI suppressed compared to the single constructs #102 (FIG. 5B) and #103 (FIG. 5C) in N2A cells. Nevertheless, the total production of GI and MW produced by either concatemer was higher than that separately produced in the single constructs carrying the stuffer sequence (Table 15).

TABLE 15
Sequencing analysis for the concatemer construct of the redesigns in N2A cells

| Construct (in plasmid) | GI G:P | GI %* | MW G:P | MW %* |
|---|---|---|---|---|
| #102 (#3 and stuffer, SEQ ID: 252)) | 37.4 | 0.7% | | |
| #103 (#51 and stuffer, SEQ ID: 253) | | | 375.0 | 0.8% |
| #100 (#3 + #51 and stuffer, SEQ ID: 256) | 26.8 | 0.1% | 581.0 | 2.4% |
| #101 (#51 + #3 and stuffer, SEQ ID: 257) | 61.8 | 0.4% | 690.0 | 6.9% |

*guide counts/total miRNA pool

42 additional redesigns were generated from the original four constructs (construct A (Table 16), construct B (Table 17), construct C (Table 18) and construct D (Table 19)). To reduce the number of transfection and samples for sequencing, co-transfection of 2-3 plasmids was carried out in N2A cells (e.g., a co-transfection of the constructs #7, #42, and #27, in Table 16, 17, and 18) followed by small RNA sequencing. Most of these redesigns exhibited improved guide/passenger ratio relative to the original constructs. In particular, construct B containing the G9 guide, which exhibited a guide/passenger ratio below 0.01, was dramatically improved by the changes resulting in construct #36, which exhibited a guide/passenger ratio of 90.5 (Table 17). Four redesigns encoding GI in the scaffold of hsa-mir-30a exhibited improved guide/passenger ratios above 100 (Table 16). Due to co-transfection with the plasmids in the samples, the guide % in the total miRNA pool might be underestimated for each redesign constructs.

TABLE 16
Redesigns for GI in hsa-mir-30a scaffold from construct A

| | | | N. of plasmids in |
|---|---|---|---|
| Construct | Guide %* | G/P ratio | co-transfection |
| #3 | 0.9% | 26.6 | 1 |
| #3 | 1.8% | 22.4 | 1 |
| #5 | 0.89% | 85.2 | 3 |
| #6 | 0.89% | 16.8 | 3 |
| #7 | 1.1% | 25.8 | 3 |
| #8 | 0.61% | 55 | 3 |
| #9 | 0.72% | 78 | 3 |
| #10 | 3.2% | 50 | 3 |
| #11 | 0.26% | No passenger | 3 |
| #12 | 1.2% | 105 | 3 |
| #13 | 0.32% | No passenger | 3 |
| #14 | 1.0% | 22.6 | 3 |
| #15 | 0.89% | 22.6 | 2 |
| #16 | 0.38% | 49.1 | 3 |
| #17 | 0.9% | 34.8 | 1 |
| #18 | 0.5% | 104 | 1 |

*guide counts/total miRNA pool
The constructs #19, #20, #21, and #22 have close redesign features as #11 and #13 in the table, and were not experimentally tested in the plasmid transfection and small RNA sequencing analysis.

TABLE 17
Redesigns for G9 in hsa-mir-124-3 scaffold from construct B

| | | | N. of plasmids in |
|---|---|---|---|
| Construct | Guide %* | G/P ratio | co-transfection |
| B | 0.24% | 0.0038 | 1 |
| #25 | 1.3% | 1.6 | 3 |
| #26 | 1.0% | 0.64 | 3 |
| #27 | 1.0% | 1.1 | 3 |
| #28 | 0.44% | 0.2 | 3 |
| #29 | 0.49% | 0.3 | 3 |
| #30 | 0.48% | 0.16 | 3 |
| #35 | 0.22% | 3.7 | 3 |
| #36 | 0.65% | 90.5 | 2 |

*guide counts/total miRNA pool

TABLE 18
Redesigns for MW in hsa-mir-124-3 scaffold from construct C

| | | | N. of plasmids in |
|---|---|---|---|
| Construct | Guide %* | G/P ratio | co-transfection |
| C | 1.7% | 0.63 | 1 |
| #39 | 17.9% | 99 | 1 |
| #40 | 11.7% | No passenger | 1 |
| #42 | 14.3% | 838 | 3 |
| #43 | 10.3% | 75 | 3 |
| #44 | 11.5% | 782 | 3 |
| #45 | 13.1% | 666 | 3 |
| #46 | 17.6% | 271 | 3 |
| #47 | 12.4% | 1947 | 3 |
| #48 | 9.7% | 60 | 3 |
| #49 | 7.5% | 393 | 3 |

*guide counts/total miRNA pool

TABLE 19
Redesigns for MW in hsa-mir-218-1 scaffold from construct D

| | | | N. of plasmids in |
|---|---|---|---|
| Construct | Guide %* | G/P ratio | co-transfection |
| #51 | 3.1% | 1192 | 1 |
| #51 | 8.5% | 899 | 1 |
| #52 | 0.06% | 44 | 3 |
| #53 | 0.6% | 139 | 3 |
| #54 | 4.4% | 884 | 3 |

*guide counts/total miRNA pool

### Example 5: GLUK2 Protein is Significantly Reduced in Mouse Cortical Neural Cells after Treatment with AAV9 Vectors Encoding Redesigned Concatemers

For protein collection, dissociated cortical neurons from P0-P1 C57BI6/J mouse were prepared and neurons were seeded in six-well plates at a concentration of 5.5e+5 cells per well. Two or three days after plating (day-in vivo, DIV2-3), half of the medium was removed, and viruses were added with MOI 7.5E+4. At DIV 13, mouse neuronal cultures were lysed and the lysate was used for SDS PAGE and immunoblotting. For immunostaining, the following antibodies were applied: rabbit anti-GluK2/3 (clone NL9 04-921; Merck-Millipore) and mouse anti-β-actin (A5316; Sigma) were used as primary and an appropriate 800 nm fluorophore-conjugated secondary antibody produced in goat (IRDye 800 goat anti-mouse Li-COR 926-32210 or IRDye 800 goat anti-rabbit Li-COR 926-32211) was used as secondary antibody. Target proteins were detected by reading at 800 nm on Li-COR. Analysis was performed with the Empiria studio software. For quantification, the intensity of the fluorescent signal of each lane was normalized by beta-actin expression and then by the control condition.

For RNA collection and miRNA quantification by stem-loop RT-qPCR, mouse cortical cultures were rinsed in ice-cold PBS at DIV 13, and then scraped into 700 μl of Qiazol (QIAGEN). Total RNA were extracted using the miRNeasy mini kit (QIAGEN 217004). Total RNA contents and quality of sample were assessed by NANODROP™ One Spectrophotometer (Thermofisher). 20 ng of total RNA or synthetic oligo RNAs (103-109 copies) were reverse-transcribed into cDNA with TaqMan microRNA reverse transcription kit and reverse transcription primers specific for GI or MW sequences, followed with qPCR by using TAQMAN™ fast universal qPCR master mix and qPCR primers/probe specific to GI or MW. The copies of GI or MW in samples were calculated from the standard curves established with synthetic GI or MW oligo RNAs.

### Results

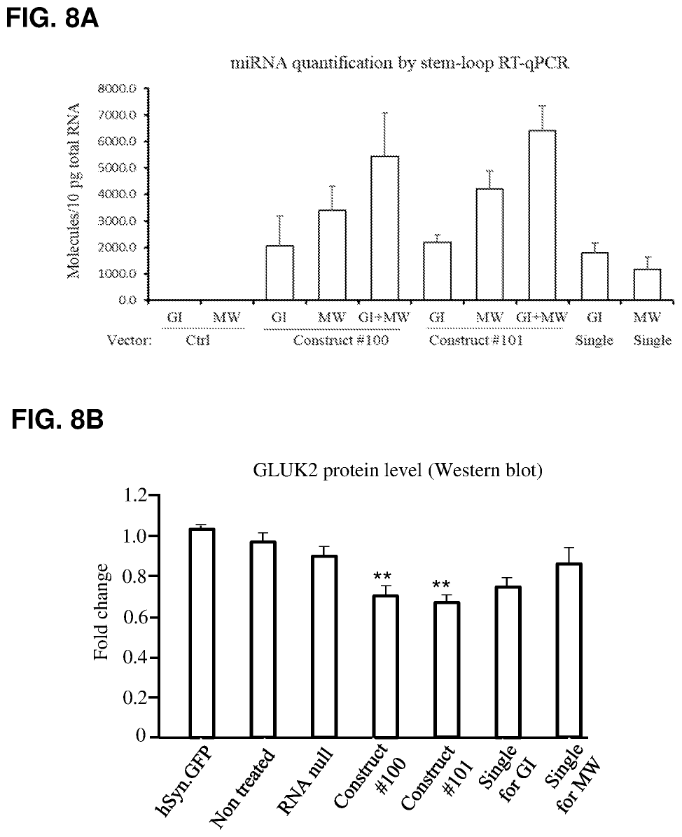

Compared to the single construct vectors (#102, SEQ ID NO: 252; #103, SEQ ID NO: 253), the concatemer vectors #100 and #101 (SEQ ID NOs: 256 and 257, respectively) produced both GI and MW and thus higher level of therapeutic miRNAs in MCN cells (FIG. 8A). A cluster effect might exist as MW expression seems boosted when concatenated with GI in the same transcript. The negative control AAV9 RNA null vector (Ctrl) carries the stuffer sequence in the vector genome but not containing any miRNA construct. Consistently, significant GLUK2 lowering was observed in the MCN samples treated with the concatemer vectors #100 and #101 but not in the single construct vectors (FIG. 8B).

### Example 6: Grik2 Transcripts are Significantly Reduced in Human GlutaNeuron Cells after Treatment with AAV9 Vectors Encoding Redesigned Concatemers

The iPSC induced ICELL® GlutaNeurons (Fujifilm Cat.R1034) were thawed and the cells were seeded with a density of 2E+6 cells/well in PLO/Matrigel-coated 6-well plate. After 24 hours, the AAV9 vector #100 or RNA null vector #106 (SEQ ID NO: 262) was added to the cells at an MOI of 3E+5 in 2 mL fresh media (n=4 per condition). The cells were harvested 11 days post-transduction by adding Qiazol lysis buffer with addition of spike-in RNA oligo (3 μL of 0.1 nM nine oligo mix per 50,000 cells). Total RNA was extracted and miRNAs were enriched by following the protocol for separating large RNA and small RNA/miRNA-enriched fractions using Qiagen miRNeasy kit. The microRNA sequencing library was generated using NEXTFLEX® small RNA kit from Perkin Elmer/BIOO. Sequencing was performed on the HiSeq platform (Genewiz). The RNA-seq library was prepared and sequenced as well.

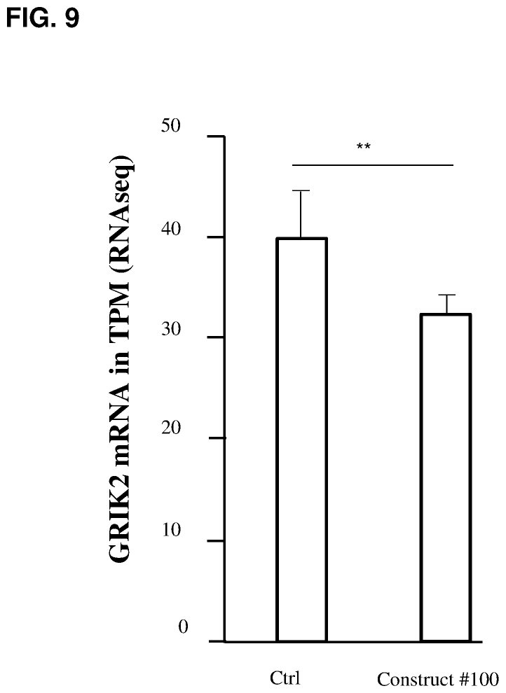

The small RNA sequencing analysis showed that synthetic miRNAs GI and MW were processed at a physiologically active level with high guide/passenger ratio in Construct #100-treated iPSC-derived GlutaNeuron cells (Table 20). With 50% transduction efficiency, Grik2 transcripts were reduced 18.5% in #100-treated iPSC-derived GlutaNeuron cells (n=4) (FIG. 9).

TABLE 20
Small RNA sequencing for iPSC-derived GlutaNeuron cells transduced with #100

| #100 sample | 1 | 2 | 3 | 4 | average |
|---|---|---|---|---|---|
| GI guide counts | 2613 | 42656 | 15518 | 117694 | |
| MW guide counts | 9538 | 156031 | 49625 | 358452 | |
| GI guides/cell | 423.9 | 477.7 | 536.9 | 567.1 | 501.4 |
| MW guides/cell | 1547.2 | 1747.5 | 1717.1 | 1727.1 | 1684.7 |
| total guides/cell | 1971.1 | 2225.2 | 2254.0 | 2294.2 | 2186.1 |
| Guide/passenger ratio -GI | 522.6 | 370.9 | 316.7 | 476.5 | 421.7 |
| Guide/passenger ratio -MW | 733.7 | 1608.6 | 1378.5 | 1525.3 | 1311.5 |

### Example 7: Seizure Activity was Suppressed in Human Hippocampal Organotypic Slices Transduced with the Concatemer Vector

Human organotypic slices were individually transferred to a recording chamber maintained at 30-32° C. and continuously perfused (2-3 mL/min) with oxygenated (95% 02 and 5% CO2) ACSF in the presence of 5 μM gabazine (Sigma-Aldrich); ACSF contained (in mM): NaCl (126.0), KCl (3.5), NaH₂PO₄ (1.2), NaHCO₃ (26), MgCl₂ (1.3), CaCl₂) (2.0), and glucose (10), pH˜7.4 (Sigma-Aldrich). Local field potential (LFP) recordings were made with a monopolar Nichrome wire placed in the granule cell layer of the dentate gyrus. DAM-80 amplifier was used for recording (low filter, 0.1 Hz; highpass filter, 3 KHz; World Precision Instruments, Sarasota, FL); data were digitized (20 kHz) with a Digidata 1440A (Molecular Devices) to a computer, and acquired using Clampex 10.1 software (PClamp, Molecular Devices). Signals were analyzed off-line using Clampfit 9.2 (PClamp) and MiniAnalysis 6.0.1 (Synaptosoft, Decatur, GA).

### Results

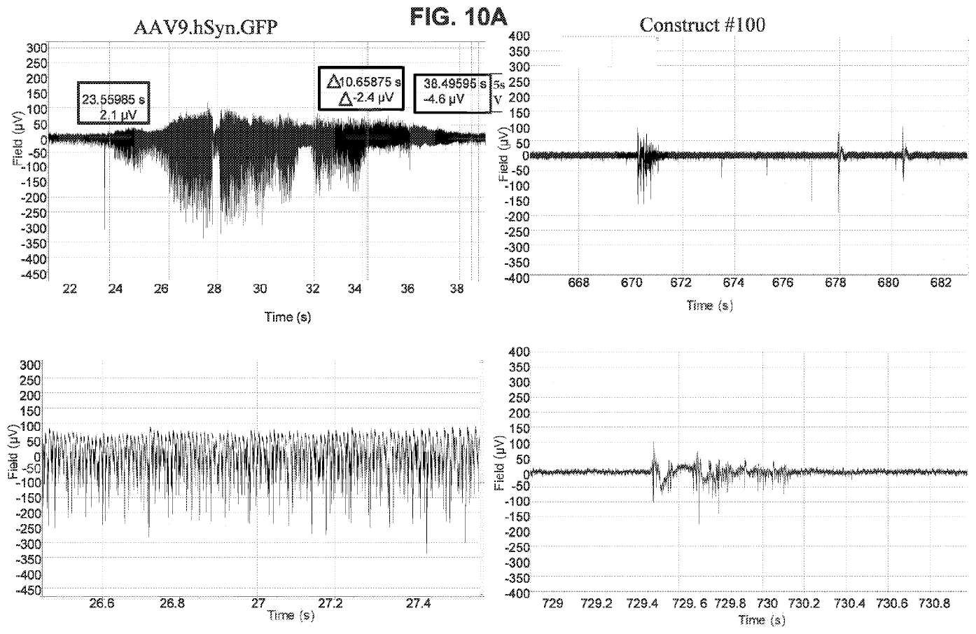

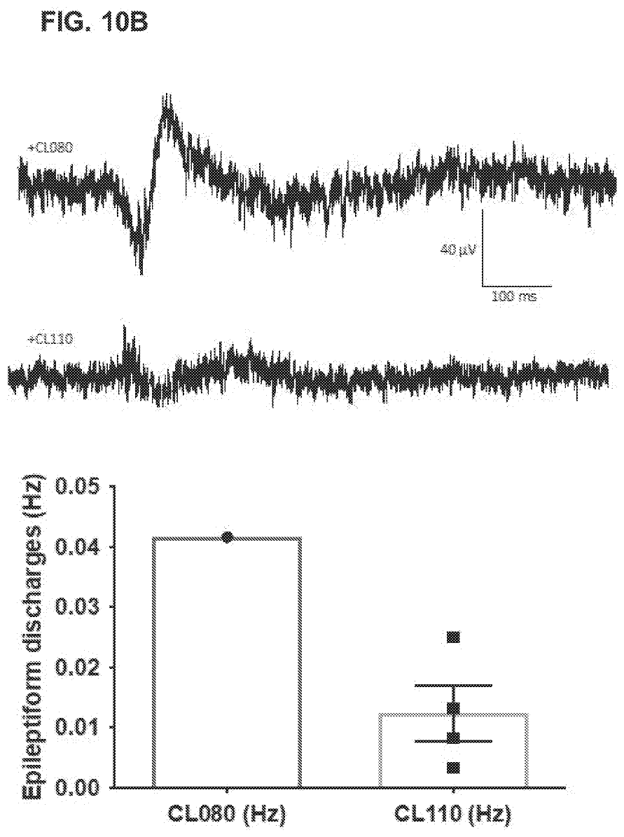

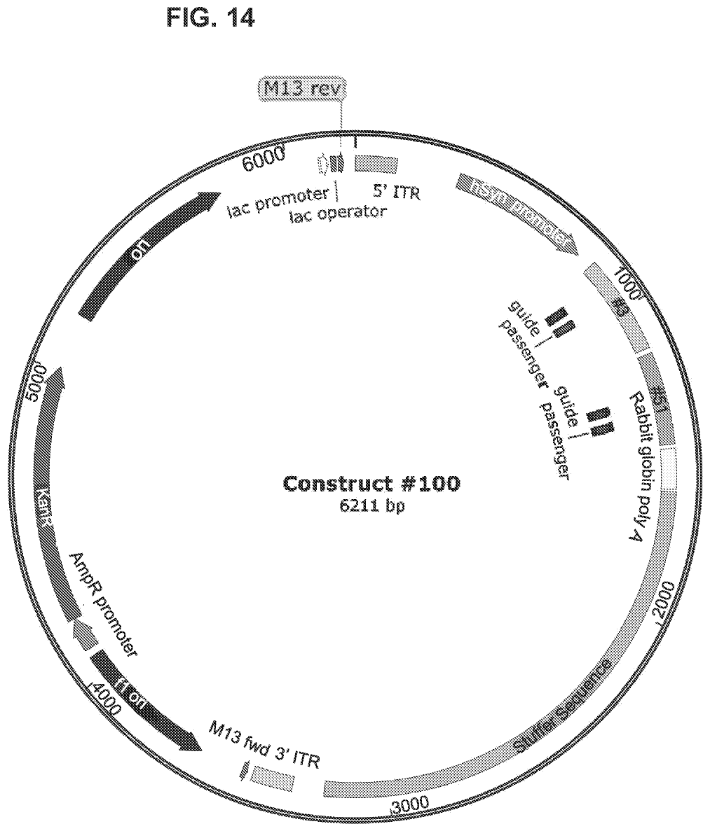

The concatemer vector #100 (the vector map is depicted in FIG. 14) suppressed spontaneous seizures from TLE hippocampus ex-vivo under hyperexcitable condition in presence of 4-AP/gabazine (FIG. 10A) and under physiological condition (FIG. 10B).

### Example 8: Physiological and Behavioral Studies in Pilocarpine Epileptic Mouse Model Indicates

### Amelioration in Symptoms of Epilepsy

### AAV Administration

Male Swiss mice that had previously been rendered epileptic by systemic injection of pilocarpine at least 2 months were placed in a stereotaxic frame. Four holes were drilled to bilaterally inject AAV9 into the dorsal and ventral dentate gyrus of the hippocampus. A defined volume of a solution containing AAV was slowly infused at the rate of 0.2 μl/minute (1.0 μL of 2.5e8 GC/injection site, two injection sites/hemisphere, thus 5e8 GC/hemisphere and 1e9/brain).

### Hyperlocomotion

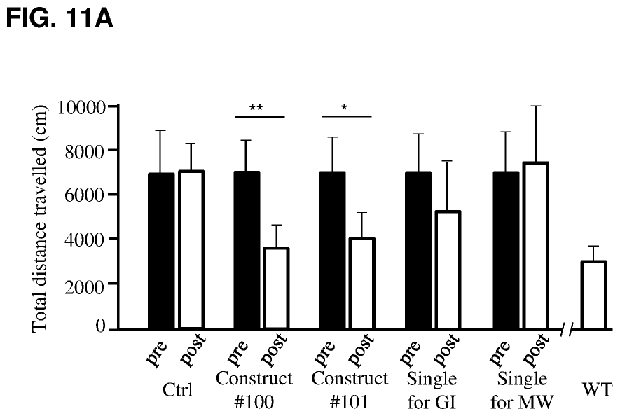

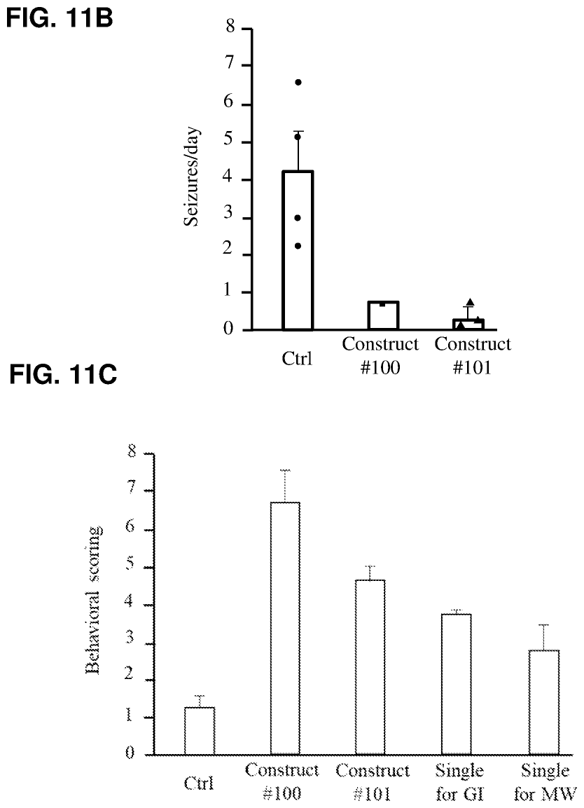

The locomotion of epileptic mice (>2 months after SE) was evaluated 1 week before and 2 weeks after AAV injection (FIG. 11A). The locomotion of non-epileptic mice (wild type Swiss male mico, 18-21 weeks old) were also evaluated. Mice were transferred to the behaviour analysis room 1 day prior experiments for habituation to the environment; the mice were kept at room temperature (20-22° C.) in a 9:00-18:00 light/dark cycle with ad libitum access to food and water. All materials that have been in contact with the animal tested were washed with acetic acid thereafter in order to prevent olfactory cues. First, spontaneous exploration behaviour was tested with the open field test (Müller et al., 2009). Briefly, the mice were placed into the center of a 50×50×50 cm blue polyvinyl chloride box for 10 min, and the trajectories were recorded with a video camera connected to a tracking software EthoVision Color (Noldus, The Netherlands); the speed and the total distance covered by the mico during 10 min exploration were analysed.

### EEG Electrode Implantation and Recording

Treated mice are implanted with one depth wire electrode 3 weeks after AAV injection. Surgeries are performed under isoflurane anaesthesia. The electrodes are placed stereotactically into the dentate gyrus (DG) (Paxinos and Watson coordinates from bregma: AP-2.55 mm, ML+1.65 mm, DV-2.25 mm). An additional screw is placed over the cerebellum, serving as ground electrode. The electrode and the screw are secured on the skull with dental cement. During the recovery, the animals will be given 5 mg/kg s.c. carprofen (RIMADYL®) 24 and 48 hours later.

EEG (amplified (1000X), filtered at 0.16-97 Hz pass, acquired at 500 Hz) was monitored using a telemetric system (Data Sciences International, St. Paul, MN) for 5 days, 24 h per day. Intrahippocampal EEG traces represent the difference in potential between the electrode inserted into the DG and an electrode positioned above the cerebellum.

### Small RNA Sequencing Analysis

After four weeks, the animals were sacrificed, hippocampi were resected and snap-frozen. After lysis total RNA were extracted from same mouse hippocampal tissue by using ALLPREP® DNA/RNA mini kit (Qiagen, REF #80204). 400 ng total RNA with addition of a spike-ins RNA oligo mix (4500 molecules/10 pg total RNA) was taken for library preparation with the NEXTFLEX® Small RNA-seq v3 Kit with UDIs for Illumina (NOVA-5132-22) in GenomeScan. Possible adapter dimers were removed using a Ampure bead size selection. Clustering and DNA sequencing using the NovaSeq6000 was performed according to manufacturer's protocols.

### Results

Construct #100 and Construct #101 were effective in reducing hyperlocomotion phenotype in the pilocarpine model in vivo at the tested dose of 1e9 GC/brain (FIG. 11A). All other constructs, including the RNA null control construct (#106) and two single constructs, did not reduce the hyperlocomotion after treatment. EEG assessment further demonstrated that Construct #100 and Construct #101 are effective in reducing the number of average seizures per day versus control (FIG. 11B). Based on these results, the concatemer design found in Construct #100 and Construct #101 with one promoter driving expression of two miRNAs appears to be more effective than the single designs found in Construct #102 and Construct #103 with one promoter driving expression of one miRNA.

Five animal behaviors (nesting, shaking, hairs, handling, and locomotion) were scored independently by three operators. Compared to the pilocarpine-induced epileptic mice treated with RNA null control vector #106, the behaviors of the mice treated with Grik2-targeting vectors were improved, with Construct #100 showing most dramatic effect (FIG. 11C).

The total number of copies of GI and MW that were expressed and processed from the Construct #100 AAV9 vector bilaterally injected in hippocampi of a mouse was measured by small RNA sequencing analysis. Per 10 pg of total RNA (average amount per cell), 392.2 copies and 573.1 copies of GI, 1331.3 copies and 2138.8 copies of MW, were determined in two hippocampi, respectively (Table 21). The molecules of the miRNAs are in the physiological active range. The guide/passenger ratio for both GI and MW was above 100, indicating that the passenger strands were less than 5 copies/10 pg and was below the physiologically active level.

TABLE 21
Small RNA sequencing for two hippocampi
treated with the concatemer #100

| | right side | left side |
|---|---|---|
| GI guide copies/cell | 392.2 | 573.1 |
| MW guide copies/cell | 1331.3 | 2138.8 |
| Total miRNA copies/cell | 1723.5 | 2711.9 |
| Guide/passenger ratio - GI | 435.4 | 292.7 |
| Guide/passenger ratio - MW | 369.5 | 241.0 |

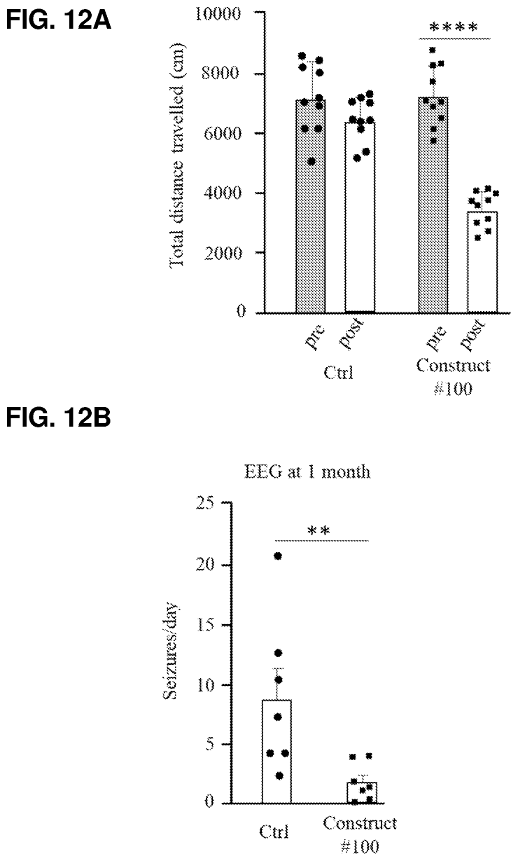

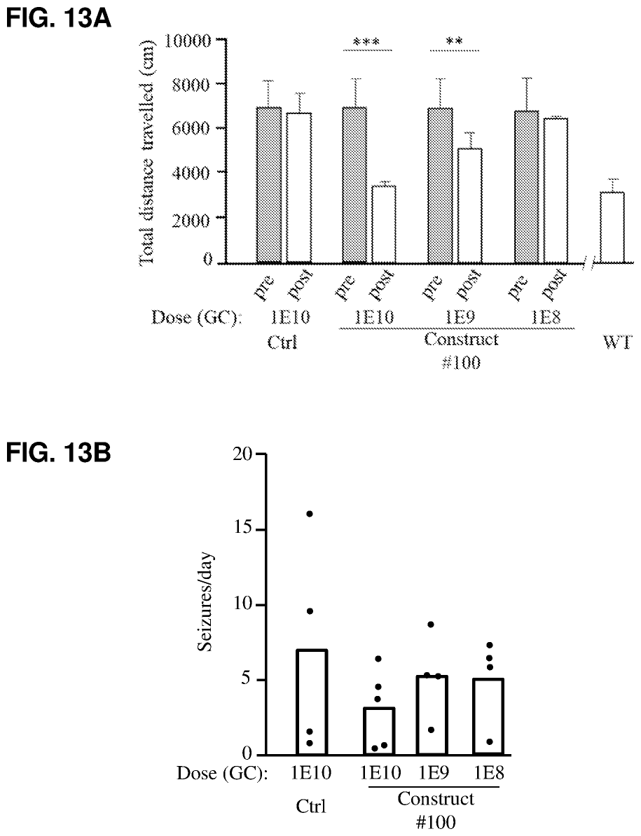

To further assess the efficaciousness of these microRNA constructs in reducing epileptic symptoms in the brains of pilocarpine epileptic mice, the injection dose was amended to 2.5×10⁹ GC/injection site, with 2 injection sites per hemisphere, resulting in 5×10⁹ GC/hemisphere and 1×10¹⁰ GC/brain after a dose response study was performed (FIGS. 12A and 12B). Again, Construct #100 proved effective in reducing both hyperlocomotion and reducing seizure activity in vivo. In an independent dose-response study, dose dependent reduction of hyperlocomotion phenotype (FIG. 13A) and seizures (FIG. 13B) was observed in pilocarpine mice treated with the vector #100 at the highest doses 1E+9 and 1E+10 per brain vs the RNA null control vector.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

### REFERENCES

Throughout this application, various references describe the state of the art to which this disclosure pertains. The disclosures of these references are hereby incorporated by reference into the disclosure:
Bahn S., Volk B., Wisden W. (1994). Kainate receptor gene expression in the developing rat brain. J. Neurosci. 14 5525-5547. 10.1523/JNEUROSCI. 14-09-05525.1994.
Boudreau Ryan L., Rodríguez-Lebron Edgardo, Davidson Beverly L., RNAi medicine for the brain: progresses and challenges, Human Molecular Genetics, Volume 20, Issue R1, 15 Apr. 2011, Pages R21-R27
Bouvier G., Larsen R. S., Rodríguez-Moreno A., Paulsen O., Sjostrom P. J. (2018). Towards resolving the presynaptic NMDA receptor debate. Curr. Opin. Neurobiol. 51 1-7. 10.1016/j.conb.2017.12.020
Crépel V, Mulle C (2015) Physiopathology of kainate receptors in epilepsy. Curr Opin Pharmacol 20:83-88; doi: 10.1016/j.coph.2014.11.012. Epub 2014 Dec. 13.
Englot, D J., et al (2013) Seizure outcomes after resective surgery for extra-temporal lobe epilepsy in pediatric patients: A systematic review. J. Neurosurgery. 12 (2): 97-201
Fritsch B., Reis J., Gasior M., Kaminski R. M., Michael A., Rogawski M. A. (2014). Role of GluK1 kainate receptors in seizures, epileptic discharges, and epileptogenesis. J. Neurosci. 34 5765-5775. 10.1523/JNEUROSCI.5307-13.2014
Gabriel S, Njunting M, Pomper J K, Merschhemke M, Sanabria ERG, Eilers A, Kivi A, Zeller M, Meencke H-J, Cavalheiro E a, Heinemann U, Lehmann T-N(2004) Stimulus and potassium-induced epileptiform activity in the human dentate gyrus from patients with and without hippocampal sclerosis. J Neurosci 24:10416-10430.
Gruber A., Lorenz R., Bernhart S. H., Neubock R., Hofacker I. L (2008). The Vienna RNA Websuite. Nucleic Acids Research. 36: W70-4
Hardy J. (2010). Genetic analysis of pathways to Parkinson disease. Neuron, 68 (2), 201-206. doi: 10.1016/j.neuron.2010.10.014
Molyan Z., Lancaster B., Whoal H. V. (2004). Metabotropic regulation of intrinsic excitability by synaptic activation of kainate receptors. J. Neurosci. 24 4530-4534. 10.1523/JNEUROSCI.5356-03.2004
Melyan Z., Wheal H. V., Lancaster B. (2002). Metabotropic-mediated kainate receptor regulation of isAHP and excitability in pyramidal cells. Neuron 34 107-114. 10.1016/S0896-6273 (02) 00624-4
Mulle C., Sailer A., Perez-Otaño I., Dickinson-Anson H., Castillo P. E., Bureau I., et al. (1998). Altered synaptic physiology and reduced susceptibility to kainate-induced seizures in GluR6-deficient mice. Nature 392 601-605.
Peret A, Christie L a., Ouedraogo D W, Gorlewicz A, Epsztein J, Mulle C, Crépel V (2014) Contribution of Aberrant GluK2-Containing Kainato Receptors to Chronic Seizures in Temporal Lobe Epilepsy. Cell Rep 8:347-354.
Reiner A, Arant RJ, and Isacoff EY (2012) Assembly Stoichiometry of the GluK2/GluK5 Kainate Receptor Complex. Cell Rep 1:234-240.
Represa A, Le Gall La Salle G, Ben-Ari Y (1989a) Hippocampal plasticity in the kindling model of epilepsy in rats. Neurosci Lett 99:345-350.
Represa A, Robain O, Tremblay E, Ben-Ari Y (1989b) Hippocampal plasticity in childhood epilepsy. Neurosci Lett 99:351-355.
Rodríguez-Moreno A., Herreras O., Lerma J. (1997). Kainate receptors presynaptically downregulate GABAergic inhibition in the rat hippocampus. Neuron 19 893-901. 10.1016/S0896-6273 (00) 80970-8.
Rodríguez-Moreno A., Sihra T. S. (2007a). Kainate receptors with a metabotropic modus operandi. Trends Neurosci. 30 630-637.
Rodríguez-Moreno A., Sihra T. S. (2007b). Metabotropic actions of kainate receptors in the CNS. J. Neurochem. 103 2121-2135.
Sapru Mohan K., Yates Jonathan W., Hogan Shea, Jiang Lixin, Halter Jeremy, Bohn Martha C. (2006). Silencing of human a-synuclein in vitro and in rat brain using lentiviral-mediated RNAi. Neurology. 198:382-390
Smolders I., Bortolotto Z. A., Clarke V. R., Warre R., Khan G. M., O'Neill M. J., et al. (2002). Antagonists of GLU (K5)-containing kainate receptors prevent pilocarpine-induced limbic seizures. Nat. Neurosci. 5 796-804. 10.1038/nn88
Sutula T, Cascino G, Cavazos J, Parada I, Ramirez L (1989) Mossy fiber synaptic reorganization in the epileptic human temporal lobe. Ann Neurol 26:321-330.
Tauck D L, Nadler J V (1985) Evidence of functional mossy fiber sprouting in hippocampal formation of kainic acid-treated rats. J Neurosci 5:1016-1022
Valbuena S., Lerma J. (2016). Non-canonical signaling, the hidden life of ligand-gated ion channels. Neuron 92 316-329. 10.1016/j.neuron.2016.10.016
Wang L., Bai J., and Hu Y. (2007) Identification of the RA Response Element and Transcriptional Silencer in Human alphaCaMKII Promoter. Mol. Biol. Rep. 35 (1): 37-44
Zinn, E., Pacouret, S., Khaychuk, V., Turunen, H. T., Carvalho, L. S., Andres-Mateos, E., . . . . Vandenberghe, L. H. (2015). In Silico Reconstruction of the Viral Evolutionary Lineage Yields a Potent Gene Therapy Vector. Cell reports, 12 (6), 1056-1068. doi: 10.1016/j.celrep.2015.07.019

1. An isolated polynucleotide that specifically binds a Grik2 mRNA comprising a stem-loop region comprising a 5′ arm (5p), a loop region, and a 3′ arm (3p), wherein the stem-loop region comprises a guide strand sequence and a passenger strand sequence, and the guide strand sequence and passenger strand sequence comprises:
(a) a uracil (U)-adenine (A) base pair or a U-guanine (G) base pair at the 5′ end of the guide strand;
(b) a cytosine (C)-G pair at the 5′ end of the passenger strand;
(c) a U at the 5′ end of the guide strand sequence;
(d) a mismatch in a seed region between the guide strand and passenger strand sequences; and/or
(e) a C-G base pair or U-A base pair to replace a U-G wobble at a junction of the stem region and the loop region of the polynucleotide.

2.-6. (canceled)

7. The polynucleotide of claim 1, wherein;
(i) the stem-loop region is a polynucleotide having at least 85% sequence identity to the nucleic acid sequence of SEQ ID NO: 2 or an RNA thereof, wherein the guide strand sequence has the nucleic acid sequence of SEQ ID NO: 17 or an RNA thereof, and/or the passenger strand sequence has the nucleic acid sequence of SEQ ID NO: 32 or an RNA thereof;
(ii) the stem-loop region is a polynucleotide having at least 85% sequence identity to the nucleic acid sequence of SEQ ID NO: 3 or an RNA thereof, wherein the guide strand sequence has the nucleic acid sequence of SEQ ID NO: 18 or an RNA thereof, and/or the passenger strand sequence has the nucleic acid sequence of SEQ ID NO: 33 or an RNA thereof;
(iii) the stem-loop region is a polynucleotide having at least 85% sequence identity to the nucleic acid sequence of SEQ ID NO: 4 or an RNA thereof, wherein the guide strand sequence has the nucleic acid sequence of SEQ ID NO: 19 or an RNA thereof, and/or the passenger strand sequence has the nucleic acid sequence of SEQ ID NO: 34 or an RNA thereof;
(iv) the stem-loop region is a polynucleotide having at least 85% sequence identity to the nucleic acid sequence of SEQ ID NO: 5 or an RNA thereof, wherein the guide strand sequence has the nucleic acid sequence of SEQ ID NO: 20 or an RNA thereof, and/or the passenger strand sequence has the nucleic acid sequence of SEQ ID NO: 35 or an RNA thereof;
(v) the stem-loop region is a polynucleotide having at least 85% sequence identity to the nucleic acid sequence of SEQ ID NO: 6 or an RNA thereof, wherein the guide strand sequence has the nucleic acid sequence of SEQ ID NO: 21 or an RNA thereof, and/or the passenger strand sequence has the nucleic acid sequence of SEQ ID NO: 36 or an RNA thereof;
(vi) the stem-loop region is a polynucleotide having at least 85% sequence identity to the nucleic acid sequence of SEQ ID NO: 7 or an RNA thereof, wherein the guide strand sequence has the nucleic acid sequence of SEQ ID NO: 22 or an RNA thereof, and/or the passenger strand sequence has the nucleic acid sequence of SEQ ID NO: 37 or an RNA thereof;
(vii) the stem-loop region is a polynucleotide having at least 85% sequence identity to the nucleic acid sequence of SEQ ID NO: 8 or an RNA thereof, wherein the guide strand sequence has the nucleic acid sequence of SEQ ID NO: 23 or an RNA thereof, and/or the passenger strand sequence has the nucleic acid sequence of SEQ ID NO: 38 or an RNA thereof;
(viii) the stem-loop region is a polynucleotide having at least 85% sequence identity to the nucleic acid sequence of SEQ ID NO: 9 or an RNA thereof, wherein the guide strand sequence has the nucleic acid sequence of SEQ ID NO: 24 or an RNA thereof, and/or the passenger strand sequence has the nucleic acid sequence of SEQ ID NO: 39 or an RNA thereof;
(ix) the stem-loop region is a polynucleotide having at least 85% sequence identity to the nucleic acid sequence of SEQ ID NO: 10 or an RNA thereof, wherein the guide strand sequence has the nucleic acid sequence of SEQ ID NO: 25 or an RNA thereof, and/or the passenger strand sequence has the nucleic acid sequence of SEQ ID NO: 40 or an RNA thereof;
(x) the stem-loop region is a polynucleotide having at least 85% sequence identity to the nucleic acid sequence of SEQ ID NO: 11 or an RNA thereof, wherein the guide strand sequence has the nucleic acid sequence of SEQ ID NO: 26 or an RNA thereof, and/or the passenger strand sequence has the nucleic acid sequence of SEQ ID NO: 41 or an RNA thereof;
(xi) the stem-loop region is a polynucleotide having at least 85% sequence identity to the nucleic acid sequence of SEQ ID NO: 12 or an RNA thereof, wherein the guide strand sequence has the nucleic acid sequence of SEQ ID NO: 27 or an RNA thereof, and/or the passenger strand sequence has the nucleic acid sequence of SEQ ID NO: 42 or an RNA thereof;
(xii) the stem-loop region is a polynucleotide having at least 85% sequence identity to the nucleic acid sequence of SEQ ID NO: 13 or an RNA thereof, wherein the guide strand sequence has the nucleic acid sequence of SEQ ID NO: 28 or an RNA thereof, and/or the passenger strand sequence has the nucleic acid sequence of SEQ ID NO: 43 or an RNA thereof;
(xiii) the stem-loop region is a polynucleotide having at least 85% sequence identity to the nucleic acid sequence of SEQ ID NO: 14 or an RNA thereof, wherein the guide strand sequence has the nucleic acid sequence of SEQ ID NO: 29 or an RNA thereof, and/or the passenger strand sequence has the nucleic acid sequence of SEQ ID NO: 44 or an RNA thereof;
(xiv) the stem-loop region is a polynucleotide having at least 85% sequence identity to the nucleic acid sequence of SEQ ID NO: 15 or an RNA thereof, wherein the guide strand sequence has the nucleic acid sequence of SEQ ID NO: 30 or an RNA thereof, and/or the passenger strand sequence has the nucleic acid sequence of SEQ ID NO: 45 or an RNA thereof;
(xv) the stem-loop region is a polynucleotide having at least 85% sequence identity to the nucleic acid sequence of SEQ ID NO: 226 or an RNA thereof, wherein the guide strand sequence has the nucleic acid sequence of SEQ ID NO: 230 or an RNA thereof, and/or the passenger strand sequence has the nucleic acid sequence of SEQ ID NO: 234 or an RNA thereof;
(xvi) the stem-loop region is a polynucleotide having at least 85% sequence identity to the nucleic acid sequence of SEQ ID NO: 227 or an RNA thereof, wherein the guide strand sequence has the nucleic acid sequence of SEQ ID NO: 231 or an RNA thereof, and/or the passenger strand sequence has the nucleic acid sequence of SEQ ID NO: 235 or an RNA thereof;
(xvii) the stem-loop region is a polynucleotide having at least 85% sequence identity to the nucleic acid sequence of SEQ ID NO: 228 or an RNA thereof, wherein the guide strand sequence has the nucleic acid sequence of SEQ ID NO: 232 or an RNA thereof, and/or the passenger strand sequence has the nucleic acid sequence of SEQ ID NO: 236 or an RNA thereof;
(xviii) the stem-loop region is a polynucleotide having at least 85% sequence identity to the nucleic acid sequence of SEQ ID NO: 229 or an RNA thereof, wherein the guide strand sequence has the nucleic acid sequence of SEQ ID NO: 233 or an RNA thereof, and/or the passenger strand sequence has the nucleic acid sequence of SEQ ID NO: 237 or an RNA thereof;
(xix) the stem-loop region is a polynucleotide having at least 85% sequence identity to the nucleic acid sequence of SEQ ID NO: 238 or an RNA thereof, wherein the guide strand sequence has the nucleic acid sequence of SEQ ID NO: 242 or an RNA thereof, and/or the passenger strand sequence has the nucleic acid sequence of SEQ ID NO: 246 or an RNA thereof;
(xx) the stem-loop region is a polynucleotide having at least 85% sequence identity to the nucleic acid sequence of SEQ ID NO: 239 or an RNA thereof, wherein the guide strand sequence has the nucleic acid sequence of SEQ ID NO: 243 or an RNA thereof, and/or the passenger strand sequence has the nucleic acid sequence of SEQ ID NO: 247 or an RNA thereof;
(xxi) the stem-loop region is a polynucleotide having at least 85% sequence identity to the nucleic acid sequence of SEQ ID NO: 240 or an RNA thereof, wherein the guide strand sequence has the nucleic acid sequence of SEQ ID NO: 244 or an RNA thereof, and/or the passenger strand sequence has the nucleic acid sequence of SEQ ID NO: 248 or an RNA thereof;
(xxii) the stem-loop region is a polynucleotide having at least 85% sequence identity to the nucleic acid sequence of SEQ ID NO: 241 or an RNA thereof, wherein the guide strand sequence has the nucleic acid sequence of SEQ ID NO: 245 or an RNA thereof, and/or the passenger strand sequence has the nucleic acid sequence of SEQ ID NO: 249 or an RNA thereof;
(xxiii) the stem-loop region is a polynucleotide having at least 85% sequence identity to the nucleic acid sequence of SEQ ID NO: 47 or an RNA thereof, wherein the guide strand sequence has the nucleic acid sequence of SEQ ID NO: 64 or an RNA thereof, and/or the passenger strand sequence has the nucleic acid sequence of SEQ ID NO: 81 or an RNA thereof;
(xxiv) the stem-loop region is a polynucleotide having at least 85% sequence identity to the nucleic acid sequence of SEQ ID NO: 48 or an RNA thereof, wherein the guide strand sequence has the nucleic acid sequence of SEQ ID NO: 65 or an RNA thereof, and/or the passenger strand sequence has the nucleic acid sequence of SEQ ID NO: 82 or an RNA thereof;
(xxv) the stem-loop region is a polynucleotide having at least 85% sequence identity to the nucleic acid sequence of SEQ ID NO: 49 or an RNA thereof, wherein the guide strand sequence has the nucleic acid sequence of SEQ ID NO: 66 or an RNA thereof, and/or the passenger strand sequence has the nucleic acid sequence of SEQ ID NO: 83 or an RNA thereof;
(xxvi) the stem-loop region is a polynucleotide having at least 85% sequence identity to the nucleic acid sequence of SEQ ID NO: 50 or an RNA thereof, wherein the guide strand sequence has the nucleic acid sequence of SEQ ID NO: 67 or an RNA thereof, and/or the passenger strand sequence has the nucleic acid sequence of SEQ ID NO: 84 or an RNA thereof;
(xxvii) the stem-loop region is a polynucleotide having at least 85% sequence identity to the nucleic acid sequence of SEQ ID NO: 51 or an RNA thereof, wherein the guide strand sequence has the nucleic acid sequence of SEQ ID NO: 68 or an RNA thereof, and/or the passenger strand sequence has the nucleic acid sequence of SEQ ID NO: 85 or an RNA thereof;
(xxviii) the stem-loop region is a polynucleotide having at least 85% sequence identity to the nucleic acid sequence of SEQ ID NO: 52 or an RNA thereof, wherein the guide strand sequence has the nucleic acid sequence of SEQ ID NO: 69 or an RNA thereof, and/or the passenger strand sequence has the nucleic acid sequence of SEQ ID NO: 86 or an RNA thereof;
(xxix) the stem-loop region is a polynucleotide having at least 85% sequence identity to the nucleic acid sequence of SEQ ID NO: 53 or an RNA thereof, wherein the guide strand sequence has the nucleic acid sequence of SEQ ID NO: 70 or an RNA thereof, and/or the passenger strand sequence has the nucleic acid sequence of SEQ ID NO: 87 or an RNA thereof;
(xxx) the stem-loop region is a polynucleotide having at least 85% sequence identity to the nucleic acid sequence of SEQ ID NO: 54 or an RNA thereof, wherein the guide strand sequence has the nucleic acid sequence of SEQ ID NO: 71 or an RNA thereof, and/or the passenger strand sequence has the nucleic acid sequence of SEQ ID NO: 88 or an RNA thereof;
(xxxi) the stem-loop region is a polynucleotide having at least 85% sequence identity to the nucleic acid sequence of SEQ ID NO: 55 or an RNA thereof, wherein the guide strand sequence has the nucleic acid sequence of SEQ ID NO: 72 or an RNA thereof, and/or the passenger strand sequence has the nucleic acid sequence of SEQ ID NO: 89 or an RNA thereof;
(xxxii) the stem-loop region is a polynucleotide having at least 85% sequence identity to the nucleic acid sequence of SEQ ID NO: 56 or an RNA thereof, wherein the guide strand sequence has the nucleic acid sequence of SEQ ID NO: 73 or an RNA thereof, and/or the passenger strand sequence has the nucleic acid sequence of SEQ ID NO: 90 or an RNA thereof;
(xxxiii) the stem-loop region is a polynucleotide having at least 85% sequence identity to the nucleic acid sequence of SEQ ID NO: 57 or an RNA thereof, wherein the guide strand sequence has the nucleic acid sequence of SEQ ID NO: 74 or an RNA thereof, and/or the passenger strand sequence has the nucleic acid sequence of SEQ ID NO: 91 or an RNA thereof;
(xxxiv) the stem-loop region is a polynucleotide having at least 85% sequence identity to the nucleic acid sequence of SEQ ID NO: 58 or an RNA thereof, wherein the guide strand sequence has the nucleic acid sequence of SEQ ID NO: 75 or an RNA thereof, and/or the passenger strand sequence has the nucleic acid sequence of SEQ ID NO: 92 or an RNA thereof;
(xxxv) the stem-loop region is a polynucleotide having at least 85% sequence identity to the nucleic acid sequence of SEQ ID NO: 59 or an RNA thereof, wherein the guide strand sequence has the nucleic acid sequence of SEQ ID NO: 76 or an RNA thereof, and/or the passenger strand sequence has the nucleic acid sequence of SEQ ID NO: 93 or an RNA thereof;
(xxxvi) the stem-loop region is a polynucleotide having at least 85% sequence identity to the nucleic acid sequence of SEQ ID NO: 60 or an RNA thereof, wherein the guide strand sequence has the nucleic acid sequence of SEQ ID NO: 77 or an RNA thereof, and/or the passenger strand sequence has the nucleic acid sequence of SEQ ID NO: 94 or an RNA thereof;
(xxxvii) the stem-loop region is a polynucleotide having at least 85% sequence identity to the nucleic acid sequence of SEQ ID NO: 61 or an RNA thereof, wherein the guide strand sequence has the nucleic acid sequence of SEQ ID NO: 78 or an RNA thereof, and/or the passenger strand sequence has the nucleic acid sequence of SEQ ID NO: 95 or an RNA thereof;
(xxxviii) the stem-loop region is a polynucleotide having at least 85% sequence identity to the nucleic acid sequence of SEQ ID NO: 62 or an RNA thereof, wherein the guide strand sequence has the nucleic acid sequence of SEQ ID NO: 79 or an RNA thereof, and/or the passenger strand sequence has the nucleic acid sequence of SEQ ID NO: 96 or an RNA thereof;
(xxxix) the stem-loop region is a polynucleotide having at least 85% sequence identity to the nucleic acid sequence of SEQ ID NO: 98 or an RNA thereof, wherein the guide strand sequence has the nucleic acid sequence of SEQ ID NO: 110 or an RNA thereof, and/or the passenger strand sequence has the nucleic acid sequence of SEQ ID NO: 122 or an RNA thereof;
(xl) the stem-loop region is a polynucleotide having at least 85% sequence identity to the nucleic acid sequence of SEQ ID NO: 99 or an RNA thereof, wherein the guide strand sequence has the nucleic acid sequence of SEQ ID NO: 111 or an RNA thereof, and/or the passenger strand sequence has the nucleic acid sequence of SEQ ID NO: 123 or an RNA thereof;
(xli) the stem-loop region is a polynucleotide having at least 85% sequence identity to the nucleic acid sequence of SEQ ID NO: 100 or an RNA thereof, wherein the guide strand sequence has the nucleic acid sequence of SEQ ID NO: 112 or an RNA thereof, and/or the passenger strand sequence has the nucleic acid sequence of SEQ ID NO: 124 or an RNA thereof;
(xlii) the stem-loop region is a polynucleotide having at least 85% sequence identity to the nucleic acid sequence of SEQ ID NO: 101 or an RNA thereof, wherein the guide strand sequence has the nucleic acid sequence of SEQ ID NO: 113 or an RNA thereof, and/or the passenger strand sequence has the nucleic acid sequence of SEQ ID NO: 125 or an RNA thereof;
(xliii) the stem-loop region is a polynucleotide having at least 85% sequence identity to the nucleic acid sequence of SEQ ID NO: 102 or an RNA thereof, wherein the guide strand sequence has the nucleic acid sequence of SEQ ID NO: 114 or an RNA thereof, and/or the passenger strand sequence has the nucleic acid sequence of SEQ ID NO: 126 or an RNA thereof;
(xliv) the stem-loop region is a polynucleotide having at least 85% sequence identity to the nucleic acid sequence of SEQ ID NO: 103 or an RNA thereof, wherein the guide strand sequence has the nucleic acid sequence of SEQ ID NO: 115 or an RNA thereof, and/or the passenger strand sequence has the nucleic acid sequence of SEQ ID NO: 127 or an RNA thereof;
(xlv) the stem-loop region is a polynucleotide having at least 85% sequence identity to the nucleic acid sequence of SEQ ID NO: 104 or an RNA thereof, wherein the guide strand sequence has the nucleic acid sequence of SEQ ID NO: 116 or an RNA thereof, and/or the passenger strand sequence has the nucleic acid sequence of SEQ ID NO: 128 or an RNA thereof;
(xlvi) the stem-loop region is a polynucleotide having at least 85% sequence identity to the nucleic acid sequence of SEQ ID NO: 105 or an RNA thereof, wherein the guide strand sequence has the nucleic acid sequence of SEQ ID NO: 117 or an RNA thereof, and/or the passenger strand sequence has the nucleic acid sequence of SEQ ID NO: 129 or an RNA thereof;
(xlvii) the stem-loop region is a polynucleotide having at least 85% sequence identity to the nucleic acid sequence of SEQ ID NO: 106 or an RNA thereof, wherein the guide strand sequence has the nucleic acid sequence of SEQ ID NO: 118 or an RNA thereof, and/or the passenger strand sequence has the nucleic acid sequence of SEQ ID NO: 130 or an RNA thereof;
(xlviii) the stem-loop region is a polynucleotide having at least 85% sequence identity to the nucleic acid sequence of SEQ ID NO: 107 or an RNA thereof, wherein the guide strand sequence has the nucleic acid sequence of SEQ ID NO: 119 or an RNA thereof, and/or the passenger strand sequence has the nucleic acid sequence of SEQ ID NO: 131 or an RNA thereof;
(xlix) the stem-loop region is a polynucleotide having at least 85% sequence identity to the nucleic acid sequence of SEQ ID NO: 108 or an RNA thereof, wherein the guide strand sequence has the nucleic acid sequence of SEQ ID NO: 120 or an RNA thereof, and/or the passenger strand sequence has the nucleic acid sequence of SEQ ID NO: 132 or an RNA thereof;
(I) the stem-loop region is a polynucleotide having at least 85% sequence identity to the nucleic acid sequence of SEQ ID NO: 134 or an RNA thereof, wherein the guide strand sequence has the nucleic acid sequence of SEQ ID NO: 140 or an RNA thereof, and/or the passenger strand sequence has the nucleic acid sequence of SEQ ID NO: 146 or an RNA thereof;
(li) the stem-loop region is a polynucleotide having at least 85% sequence identity to the nucleic acid sequence of SEQ ID NO: 135 or an RNA thereof, wherein the guide strand sequence has the nucleic acid sequence of SEQ ID NO: 141 or an RNA thereof, and/or the passenger strand sequence has the nucleic acid sequence of SEQ ID NO: 147 or an RNA thereof;
(lii) the stem-loop region is a polynucleotide having at least 85% sequence identity to the nucleic acid sequence of SEQ ID NO: 136 or an RNA thereof, wherein the guide strand sequence has the nucleic acid sequence of SEQ ID NO: 142 or an RNA thereof, and/or the passenger strand sequence has the nucleic acid sequence of SEQ ID NO: 148 or an RNA thereof;
(liii) the stem-loop region is a polynucleotide having at least 85% sequence identity to the nucleic acid sequence of SEQ ID NO: 137 or an RNA thereof, wherein the guide strand sequence has the nucleic acid sequence of SEQ ID NO: 143 or an RNA thereof, and/or the passenger strand sequence has the nucleic acid sequence of SEQ ID NO: 149 or an RNA thereof; and/or
(liv) the stem-loop region is a polynucleotide having at least 85% sequence identity to the nucleic acid sequence of SEQ ID NO: 138 or an RNA thereof, wherein the guide strand sequence has the nucleic acid sequence of SEQ ID NO: 144 or an RNA thereof, and/or the passenger strand sequence has the nucleic acid sequence of SEQ ID NO: 150 or an RNA thereof.

8.-168. (canceled)

169. The polynucleotide of claim 1, wherein the polynucleotide comprises an antisense oligonucleotide (ASO), wherein the ASO comprises a short interfering RNA (siRNA), a short hairpin RNA (shRNA), a microRNA (miRNA), or a short hairpin-adapted miRNA (shmiRNA).

170. (canceled)

171. The polynucleotide of claim 1, wherein the polynucleotide comprises 19, 20, or 21 nucleotides.

172.-174. (canceled)

175. The polynucleotide of claim 1, wherein;
(i) the Grik2 mRNA is encoded by a nucleic acid sequence of SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, or SEQ ID NO: 174; and/or
(ii) the polynucleotide is capable of reducing a level of GluK2 protein in a cell contacted with the polynucleotide by at least 10% relative to a cell that is not contacted with the polynucleotide.

176.-177. (canceled)

178. The polynucleotide of claim 175, wherein the cell is a human cell and/or a neuron, wherein the neuron is a hippocampal neuron.

179.-180. (canceled)

181. The polynucleotide of claim 178, wherein the hippocampal neuron is a dentate granule cell (DGC) or a glutamatergic pyramidal neuron.

182. A vector comprising the polynucleotide of claim 1, wherein the vector is:
(i) replication defective;
(ii) an expression vector; and/or
(iii) a mammalian, insect, bacterial, or viral vector.

183.-197. (canceled)

198. An expression cassette comprising:
I)
a polynucleotide comprising a stem-loop sequence having at least 85% sequence identity to a nucleic acid sequence selected from the group consisting of SEQ ID NOs: 1-15, 46-62, 97-108, 133-138, 226-229, 238-241, and 256-261; or
II)
from 5′ to 3′;
(a) a promoter sequence;
(b) a first polynucleotide comprising a stem-loop sequence having at least 85% sequence identity to a nucleic acid sequence selected from the group consisting of SEQ ID NOs: 1-19, 34-62, 97-108, 133-147, 226-229, or 238-241; and
(c) a second polynucleotide comprising a stem-loop sequence having at least 85% sequence identity to a nucleic acid sequence selected from the group consisting of SEQ ID NOs: 1-19, 34-62, 97-108, 133-147, 226-229 or 238-241.

199. The expression cassette of claim 198, wherein the polynucleotide of I) or each of the first polynucleotide and the second polynucleotide of II) independently comprises a passenger sequence which is complementary or substantially complementary to a guide sequence, wherein the passenger sequence is located 5′ or 3′ relative to a guide sequence, and wherein the expression cassette further comprises:
(i) a 5′ flanking region located 5′ relative to a guide sequence;
(ii) a 3′ flanking region located 3′ relative to the guide sequence; and
(iii) a loop region located between the guide sequence and the passenger sequence, wherein the loop region comprises a microRNA loop sequence, wherein the microRNA loop sequence is a E-miR-30, miR-218-1, or E-miR-124-3 sequence.

200.-203. (canceled)

204. The expression cassette of claim 199, wherein:
(i) the 5′ flanking region comprises a polynucleotide having at least 85% sequence identity to the nucleic acid sequence of any one of SEQ ID NOs: 217, 220, or 223;
(ii) the 3′ flanking region comprises a polynucleotide having at least 85% sequence identity to the nucleic acid sequence of any one of SEQ ID NOs: 218, 221, or 224; and
(iii) the microRNA loop sequence comprises a polynucleotide having at least 85% sequence identity to the nucleic acid sequence of any one of SEQ ID NOs: 219, 222, or 225.

205.-207. (canceled)

208. The expression cassette of claim 198, further comprising:
(i) a 5′-inverted terminal repeat (ITR) sequence on the 5′ end of said expression cassette;
(ii) a 3′-ITR sequence on the 3′ end of said expression cassette;
(iii) an enhancer sequence;
(iv) an intron sequence;
(v) one or more polyadenylation signal sequences, wherein the one or more polyadenylation signal sequences is a rabbit beta-globin (RBG) polyadenylation signal sequence or a bovine growth hormone (BGH) polyadenylation signal sequence; and/or
(vi) the promoter is selected from the group consisting of an hSyn promoter or CaMKII promoter.

209. (canceled)

210. The expression cassette of claim 208 or 209, wherein:
the 5′-ITR sequence comprises a polynucleotide having at least 85% sequence identity to the nucleic acid sequence of SEQ ID NO: 208 or SEQ ID NO: 209;
the 3′-ITR sequence comprises a polynucleotide having at least 85% sequence identity to the nucleic acid sequence of any one of SEQ ID NOs: 210-212;
the enhancer sequence comprises a polynucleotide having at least 85% sequence identity to the nucleic acid sequence of SEQ ID NO: 207;
the intron sequence comprises a polynucleotide having at least 85% sequence identity to the nucleic acid sequence of SEQ ID NO: 205 or SEQ ID NO: 206; and/or
wherein the RBG polyadenylation signal sequence comprises a polynucleotide having at least 85% sequence identity to the nucleic acid sequence of any one of SEQ ID NOs: 213-215, or the BGH polyadenylation signal sequence comprises a polynucleotide having at least 85% sequence identity to the nucleic acid sequence of SEQ ID NO: 216.

211.-219. (canceled)

220. The expression cassette of claim 198, wherein the expression cassette is incorporated into a vector.

221. The expression cassette of claim 198, wherein the expression cassette comprises at least 80% sequence identity to the sequence of SEQ ID NO: 256.

222. The expression cassette of claim 221, wherein the expression cassette comprises at least 85%, 86%, 87%, 88%, 89%, 90%, 91%, 92%, 93%, 94%, 95%, 96%, 97%, 98%, 99%, or 100% sequence identity to the sequence of SEQ ID NO: 256.

223. A method of inhibiting Grik2 expression in a cell comprising contacting the cell with at least one polynucleotide of claim 1.

224. The method of claim 223, wherein the polynucleotide specifically hybridizes to a Grik2 mRNA and inhibits or reduces the expression of Grik2 in the cell, wherein the method reduces a level of GluK2 protein in the cell by at least 10%, at least 10%, at least at least 15%, at least 20%, at least 25%, at least 30%, at least 35%, at least 40%, at least 45%, at least 50%, at least 55%, at least 60%, at least 65%, at least 70%, or at least 75%.

225.-226. (canceled)

227. The method of claim 223, wherein the cell is a human cell and/or a neuron, wherein the neuron is a hippocampal neuron.

228.-229. (canceled)

230. The method of claim 227, wherein the hippocampal neuron is a DGC or a glutamatergic pyramidal neuron.

231. (canceled)

232. A method of treating or ameliorating a disorder in a subject in need thereof comprising administering to the subject at least one polynucleotide of claim 1, wherein the subject is a human.

233. (canceled)

234. The method of claim 232, wherein the disorder is an epilepsy.

235. The method of claim 234, wherein the epilepsy is a temporal lobe epilepsy (TLE), chronic epilepsy, and/or a refractory epilepsy.

236. (canceled)

237. The method of claim 235, wherein the TLE is a lateral TLE (lTLE) or a mesial TLE (mTLE).

238.-239. (canceled)

240. A pharmaceutical composition comprising the polynucleotide of claim 1, and a pharmaceutically acceptable carrier, diluent, or excipient.

241. A kit comprising the pharmaceutical composition of claim 240, and a package insert, wherein the package insert comprises instructions for use of the pharmaceutical composition in a method of treatment.

242. (canceled)

243. The polynucleotide of claim 1, wherein the stem-loop region comprises a polynucleotide having at least 85%, 86%, 87%, 88%, 89%, 90%, 91%, 92%, 93%, 94%, 95%, 96%, 97%, 98%, 99%, or 100% sequence identity to SEQ ID NO: 4 or 135, or an RNA thereof.

244. The expression cassette of claim 198, wherein the polynucleotide of I) or each of the first polynucleotide and the second polynucleotide of II) independently comprise a polynucleotide having at least 85%, 86%, 87%, 88%, 89%, 90%, 91%, 92%, 93%, 94%, 95%, 96%, 97%, 98%, 99%, or 100% sequence identity to SEQ ID NO: 4 or 135.


SEQUENCE LISTING (262 sequences)

SEQ ID NO: 1 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaatta

SEQ ID NO: 2 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagaagctgcctactgcctcggacttcaaggggctactttaggagcaattatcttgtttactaaaactgaataccttgctatctctttgatacatttttacaaagctgaattaaaatggtataaatta

SEQ ID NO: 3 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgcgaagctgcctactgcctcggacttcaaggggctactttaggagcaattatcttgtttactaaaactgaataccttgctatctctttgatacatttttacaaagctgaattaaaatggtataaatta

SEQ ID NO: 4 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaatta

SEQ ID NO: 5 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgataagctgcctactgcctcggacttcaaggggctactttaggagcaattatcttgtttactaaaactgaataccttgctatctctttgatacatttttacaaagctgaattaaaatggtataaatta

SEQ ID NO: 6 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgccgtgaagccacagatgggcatgggttttatatcgcgaagctgcctactgcctcggacttcaaggggctactttaggagcaattatcttgtttactaaaactgaataccttgctatctctttgatacatttttacaaagctgaattaaaatggtataaatta

SEQ ID NO: 7 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgccgtgaagccacagatgggcatgggttttatatcgagaagctgcctactgcctcggacttcaaggggctactttaggagcaattatcttgtttactaaaactgaataccttgctatctctttgatacatttttacaaagctgaattaaaatggtataaatta

SEQ ID NO: 8 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgagaagctgcctactgcctcggacttcaaggggctactttaggagcaattatcttgtttactaaaactgaataccttgctatctctttgatacatttttacaaagctgaattaaaatggtataaatta

SEQ ID NO: 9 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgcgaagctgcctactgcctcggacttcaaggggctactttaggagcaattatcttgtttactaaaactgaataccttgctatctctttgatacatttttacaaagctgaattaaaatggtataaatta

SEQ ID NO: 10 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgctgtgaagccacagatgggcatgggttttatatcgcgaagctgcctactgcctcggacttcaaggggctactttaggagcaattatcttgtttactaaaactgaataccttgctatctctttgatacatttttacaaagctgaattaaaatggtataaatta

SEQ ID NO: 11 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgctgtgaagccacagatgggcctgggttttatatcgcgaggctgcctactgcctcggacttcaaggggctactttaggagcaattatcttgtttactaaaactgaataccttgctatctctttgatacatttttacaaagctgaattaaaatggtataaatta

SEQ ID NO: 12 - DNA, 313 bp, Artificial Sequence; identical to SEQ ID NO: 11
  Synthetic Construct

SEQ ID NO: 13 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgagaggctgcctactgcctcggacttcaaggggctactttaggagcaattatcttgtttactaaaactgaataccttgctatctctttgatacatttttacaaagctgaattaaaatggtataaatta

SEQ ID NO: 14 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgctgtgaagccacagatgggcatgggttttatatcgcgaggctgcctactgcctcggacttcaaggggctactttaggagcaattatcttgtttactaaaactgaataccttgctatctctttgatacatttttacaaagctgaattaaaatggtataaatta

SEQ ID NO: 15 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgctgtgaagccacagatgggcatgggttttatatcgagaggctgcctactgcctcggacttcaaggggctactttaggagcaattatcttgtttactaaaactgaataccttgctatctctttgatacatttttacaaagctgaattaaaatggtataaatta

SEQ ID NO: 16 - DNA, 22 bp, Artificial Sequence
  Synthetic Construct
gtctcgatatggagaacccatg

SEQ ID NO: 17 - DNA, 22 bp, Artificial Sequence
  Synthetic Construct
ttctcgatatggagaacccagg

SEQ ID NO: 18 - DNA, 22 bp, Artificial Sequence
  Synthetic Construct
ttctcgatatggagaacccagg

SEQ ID NO: 19 - DNA, 22 bp, Artificial Sequence
  Synthetic Construct
ttctcgatatggagaacccagg

SEQ ID NO: 20 - DNA, 22 bp, Artificial Sequence
  Synthetic Construct
ttctcgatatggagaacccagg

SEQ ID NO: 21 - DNA, 22 bp, Artificial Sequence
  Synthetic Construct
ttctcgatatggagaacccatg

SEQ ID NO: 22 - DNA, 22 bp, Artificial Sequence
  Synthetic Construct
ttctcgatatggagaacccatg

SEQ ID NO: 23 - DNA, 22 bp, Artificial Sequence
  Synthetic Construct
ttctcgatatggagaacccagg

SEQ ID NO: 24 - DNA, 22 bp, Artificial Sequence
  Synthetic Construct
ttctcgatatggagaacccagg

SEQ ID NO: 25 - DNA, 22 bp, Artificial Sequence
  Synthetic Construct
ttctcgatatggagaacccatg

SEQ ID NO: 26 - DNA, 22 bp, Artificial Sequence
  Synthetic Construct
ttctcgatatggagaacccatg

SEQ ID NO: 27 - DNA, 22 bp, Artificial Sequence
  Synthetic Construct
ttctcgatatggagaacccagg

SEQ ID NO: 28 - DNA, 22 bp, Artificial Sequence
  Synthetic Construct
ttctcgatatggagaacccagg

SEQ ID NO: 29 - DNA, 22 bp, Artificial Sequence
  Synthetic Construct
ttctcgatatggagaacccatg

SEQ ID NO: 30 - DNA, 22 bp, Artificial Sequence
  Synthetic Construct
ttctcgatatggagaacccatg

SEQ ID NO: 31 - DNA, 20 bp, Artificial Sequence
  Synthetic Construct
catgggttttatatcgagac

SEQ ID NO: 32 - DNA, 20 bp, Artificial Sequence
  Synthetic Construct
cctgggttttatatcgagaa

SEQ ID NO: 33 - DNA, 20 bp, Artificial Sequence
  Synthetic Construct
cctgggttttatatcgcgaa

SEQ ID NO: 34 - DNA, 20 bp, Artificial Sequence
  Synthetic Construct
cctgggttttatatcgagca

SEQ ID NO: 35 - DNA, 20 bp, Artificial Sequence
  Synthetic Construct
cctgggttttatatcgataa

SEQ ID NO: 36 - DNA, 20 bp, Artificial Sequence
  Synthetic Construct
catgggttttatatcgcgaa

SEQ ID NO: 37 - DNA, 20 bp, Artificial Sequence
  Synthetic Construct
catgggttttatatcgagaa

SEQ ID NO: 38 - DNA, 20 bp, Artificial Sequence
  Synthetic Construct
cctgggttttatatcgagaa

SEQ ID NO: 39 - DNA, 20 bp, Artificial Sequence
  Synthetic Construct
cctgggttttatatcgcgaa

SEQ ID NO: 40 - DNA, 20 bp, Artificial Sequence
  Synthetic Construct
catgggttttatatcgcgaa

SEQ ID NO: 41 - DNA, 20 bp, Artificial Sequence
  Synthetic Construct
catgggttttatatcgagaa

SEQ ID NO: 42 - DNA, 20 bp, Artificial Sequence
  Synthetic Construct
cctgggttttatatcgcgag

SEQ ID NO: 43 - DNA, 20 bp, Artificial Sequence
  Synthetic Construct
cctgggttttatatcgagag

SEQ ID NO: 44 - DNA, 20 bp, Artificial Sequence
  Synthetic Construct
catgggttttatatcgcgag

SEQ ID NO: 45 - DNA, 20 bp, Artificial Sequence
  Synthetic Construct
catgggttttatatcgagag

SEQ ID NO: 46 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaatcccatagctaatgcctgttttagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 47 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctgcaacaggcactagacatgggatttaatgtctatacaattccatagctaatgcctgttgcagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 48 - DNA, 485 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctgcaacaggcactagacatggatttaatgtctatacaattcatagctaatgcctgttgcagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 49 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctgcaatgggcactagacatgggatttaatgtctatacaattccatagctaatgcctgttgcagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 50 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaacaggcactagacatgggatttaatgtctatacaattccatagctaatgcctgttgtagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 51 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatgggatttaatgtctatacaattccatagctaatgcctgttgtagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 52 - DNA, 485 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctgcaatgggcactagacatggatttaatgtctatacaattcatagctaatgcctgttgcagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 53 - DNA, 485 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaacaggcactagacatggatttaatgtctatacaattcatagctaatgcctgttgtagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 54 - DNA, 485 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctacaatgggcactagacatggatttaatgtctatacaattcatagctaatgcctgttgtagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 55 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcttcccatagctaatgcctgtgcgattaatgtctatacatcgcacaggcatcagctatggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 56 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctttccatagctaatgcctgtgcgattaatgtctatacatcgcacaggcatcagctatggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 57 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcttcccatagctaatgcctgtgcgattaatgtctatacatcgcacattcatcagctatggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 58 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctttccatagctaatgcctgtgcgattaatgtctatacatcgcacattcatcagctatggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 59 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcttcccatagctaatgcctgttttattaatgtctatacataaaacaggcatcagctatggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 60 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctttccatagctaatgcctgttttattaatgtctatacataaaacaggcatcagctatggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 61 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcttcccatagctaatgcctgtgcgattaatgtctatacatcgcacaggcatcagctctggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 62 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctttccatagctaatgcctgtgcgattaatgtctatacatcgcacaggcatcagctctggtaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 63 - DNA, 22 bp, Artificial Sequence
  Synthetic Construct
cccatagctaatgcctgtttta

SEQ ID NO: 64 - DNA, 22 bp, Artificial Sequence
  Synthetic Construct
tccatagctaatgcctgttgca

SEQ ID NO: 65 - DNA, 21 bp, Artificial Sequence
  Synthetic Construct
tcatagctaatgcctgttgca

SEQ ID NO: 66 - DNA, 22 bp, Artificial Sequence
  Synthetic Construct
tccatagctaatgcctgttgca

SEQ ID NO: 67 - DNA, 22 bp, Artificial Sequence
  Synthetic Construct
tccatagctaatgcctgttgta

SEQ ID NO: 68 - DNA, 22 bp, Artificial Sequence
  Synthetic Construct
tccatagctaatgcctgttgta

SEQ ID NO: 69 - DNA, 21 bp, Artificial Sequence
  Synthetic Construct
tcatagctaatgcctgttgca

SEQ ID NO: 70 - DNA, 21 bp, Artificial Sequence
  Synthetic Construct
tcatagctaatgcctgttgta

SEQ ID NO: 71 - DNA, 21 bp, Artificial Sequence
  Synthetic Construct
tcatagctaatgcctgttgta

SEQ ID NO: 72 - DNA, 22 bp, Artificial Sequence
  Synthetic Construct
cccatagctaatgcctgtgcga

SEQ ID NO: 73 - DNA, 22 bp, Artificial Sequence
  Synthetic Construct
tccatagctaatgcctgtgcga

SEQ ID NO: 74 - DNA, 22 bp, Artificial Sequence
  Synthetic Construct
cccatagctaatgcctgtgcga

SEQ ID NO: 75 - DNA, 22 bp, Artificial Sequence
  Synthetic Construct
tccatagctaatgcctgtgcga

SEQ ID NO: 76 - DNA, 22 bp, Artificial Sequence
  Synthetic Construct
cccatagctaatgcctgtttta

SEQ ID NO: 77 - DNA, 22 bp, Artificial Sequence
  Synthetic Construct
tccatagctaatgcctgtttta

SEQ ID NO: 78 - DNA, 22 bp, Artificial Sequence
  Synthetic Construct
cccatagctaatgcctgtgcga

SEQ ID NO: 79 - DNA, 22 bp, Artificial Sequence
  Synthetic Construct
tccatagctaatgcctgtgcga

SEQ ID NO: 80 - DNA, 22 bp, Artificial Sequence
  Synthetic Construct
tacaatgggcactagacatggg

SEQ ID NO: 81 - DNA, 22 bp, Artificial Sequence
  Synthetic Construct
tgcaacaggcactagacatggg

SEQ ID NO: 82 - DNA, 21 bp, Artificial Sequence
  Synthetic Construct
tgcaacaggcactagacatgg

SEQ ID NO: 83 - DNA, 22 bp, Artificial Sequence
  Synthetic Construct
tgcaatgggcactagacatggg

SEQ ID NO: 84 - DNA, 22 bp, Artificial Sequence
  Synthetic Construct
tacaacaggcactagacatggg

SEQ ID NO: 85 - DNA, 22 bp, Artificial Sequence
  Synthetic Construct
tacaatgggcactagacatggg

SEQ ID NO: 86 - DNA, 21 bp, Artificial Sequence
  Synthetic Construct
tgcaatgggcactagacatgg

SEQ ID NO: 87 - DNA, 21 bp, Artificial Sequence
  Synthetic Construct
tacaacaggcactagacatgg

SEQ ID NO: 88 - DNA, 21 bp, Artificial Sequence
  Synthetic Construct
tacaatgggcactagacatgg

SEQ ID NO: 89 - DNA, 24 bp, Artificial Sequence
  Synthetic Construct
tcgcacaggcatcagctatggtaa

SEQ ID NO: 90 - DNA, 24 bp, Artificial Sequence
  Synthetic Construct
tcgcacaggcatcagctatggtaa

SEQ ID NO: 91 - DNA, 24 bp, Artificial Sequence
  Synthetic Construct
tcgcacattcatcagctatggtaa

SEQ ID NO: 92 - DNA, 24 bp, Artificial Sequence
  Synthetic Construct
tcgcacattcatcagctatggtaa

SEQ ID NO: 93 - DNA, 24 bp, Artificial Sequence
  Synthetic Construct
taaaacaggcatcagctatggtaa

SEQ ID NO: 94 - DNA, 24 bp, Artificial Sequence
  Synthetic Construct
taaaacaggcatcagctatggtaa

SEQ ID NO: 95 - DNA, 24 bp, Artificial Sequence
  Synthetic Construct
tcgcacaggcatcagctctggtaa

SEQ ID NO: 96 - DNA, 24 bp, Artificial Sequence
  Synthetic Construct
tcgcacaggcatcagctctggtaa

SEQ ID NO: 97 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 98 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgcagtccatctacaacactctgatttaatgtctatacaattagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 99 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaggttcatctacaacactttaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 100 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaggtccatctacaacactttaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 101 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgcagtttatctacaacactctgatttaatgtctatacaattagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 102 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaagtccatctacaacactctgatttaatgtctatacaattagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 103 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaagtttatctacaacactctgatttaatgtctatacaattagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 104 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaagtttatctacaacactctaatttaatgtctatacaattagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 105 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaggttcatctacaacactctaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 106 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaggtttatctacaacactctaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 107 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaggtccatctacaacactctaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 108 - DNA, 487 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgcggtccatctacaacactttaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 109 - DNA, 22 bp, Artificial Sequence
  Synthetic Construct
cagagcattgcagatggactgc

SEQ ID NO: 110 - DNA, 22 bp, Artificial Sequence
  Synthetic Construct
tagagcattgcagatggactgc

SEQ ID NO: 111 - DNA, 22 bp, Artificial Sequence
  Synthetic Construct
tagagcattgcagatggaccgc

SEQ ID NO: 112 - DNA, 22 bp, Artificial Sequence
  Synthetic Construct
tagagcattgcagatggaccgc

SEQ ID NO: 113 - DNA, 22 bp, Artificial Sequence
  Synthetic Construct
tagagcattgcagatggactgc

SEQ ID NO: 114 - DNA, 22 bp, Artificial Sequence
  Synthetic Construct
tagagcattgcagatggactgc

SEQ ID NO: 115 - DNA, 22 bp, Artificial Sequence
  Synthetic Construct
tagagcattgcagatggactgc

SEQ ID NO: 116 - DNA, 22 bp, Artificial Sequence
  Synthetic Construct
tagagcattgcagatggactgc

SEQ ID NO: 117 - DNA, 22 bp, Artificial Sequence
  Synthetic Construct
tagagcattgcagatggaccgc

SEQ ID NO: 118 - DNA, 22 bp, Artificial Sequence
  Synthetic Construct
tagagcattgcagatggaccgc

SEQ ID NO: 119 - DNA, 22 bp, Artificial Sequence
  Synthetic Construct
tagagcattgcagatggaccgc

SEQ ID NO: 120 - DNA, 22 bp, Artificial Sequence
  Synthetic Construct
tagagcattgcagatggaccgc

SEQ ID NO: 121 - DNA, 22 bp, Artificial Sequence
  Synthetic Construct
gacgtttatctacaacactctg

SEQ ID NO: 122 - DNA, 22 bp, Artificial Sequence
  Synthetic Construct
gcagtccatctacaacactctg

SEQ ID NO: 123 - DNA, 22 bp, Artificial Sequence
  Synthetic Construct
gaggttcatctacaacacttta

SEQ ID NO: 124 - DNA, 22 bp, Artificial Sequence
  Synthetic Construct
gaggtccatctacaacacttta

SEQ ID NO: 125 - DNA, 22 bp, Artificial Sequence
  Synthetic Construct
gcagtttatctacaacactctg

SEQ ID NO: 126 - DNA, 22 bp, Artificial Sequence
  Synthetic Construct
gaagtccatctacaacactctg

SEQ ID NO: 127 - DNA, 22 bp, Artificial Sequence
  Synthetic Construct
gaagtttatctacaacactctg

SEQ ID NO: 128 - DNA, 22 bp, Artificial Sequence
  Synthetic Construct
gaagtttatctacaacactcta

SEQ ID NO: 129 - DNA, 22 bp, Artificial Sequence
  Synthetic Construct
gaggttcatctacaacactcta

SEQ ID NO: 130 - DNA, 22 bp, Artificial Sequence
  Synthetic Construct
gaggtttatctacaacactcta

SEQ ID NO: 131 - DNA, 22 bp, Artificial Sequence
  Synthetic Construct
gaggtccatctacaacactcta

SEQ ID NO: 132 - DNA, 22 bp, Artificial Sequence
  Synthetic Construct
gcggtccatctacaacacttta

SEQ ID NO: 133 - DNA, 292 bp, Artificial Sequence
  Synthetic Construct
acgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 134 - DNA, 292 bp, Artificial Sequence
  Synthetic Construct
acgtttccagaacgtctgtagcttttctcctccttccctccattttcctcttggtcttacctttggcctagtggttggtgtagtgataatgtagcgagattttctgtagagcattgcagatggcccgggttgcgaggtatgagtaaacgggccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 135 - DNA, 292 bp, Artificial Sequence
  Synthetic Construct
acgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 136 - DNA, 292 bp, Artificial Sequence
  Synthetic Construct
acgtttccagaacgtctgtagcttttctcctccttccctccattttcctcttggtcttacctttggcctagtggttggtgtagtgataatgtagcgagattttctgcagagcattgcagatggcccgggttgcgaggtatgagtaaacgggccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 137 - DNA, 292 bp, Artificial Sequence
  Synthetic Construct
acgtttccagaacgtctgtagcttttctcctccttccctccattttcctcttggtcttacctttggcctagtggttggtgtagtgataatgtagcgagattttctgcagagcattgcagatggaccgggttgcgaggtatgagtaaacggtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 138 - DNA, 292 bp, Artificial Sequence
  Synthetic Construct
acgtttccagaacgtctgtagcttttctcctccttccctccattttcctcttggtcttacctttggcctagtggttggtgtagtgataatgtagcgagattttctgtagagcattgcagatggactgggttgcgaggtatgagtaaacagtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 139 - DNA, 21 bp, Artificial Sequence
  Synthetic Construct
cagagcattgcagatggactg

SEQ ID NO: 140 - DNA, 21 bp, Artificial Sequence
  Synthetic Construct
tagagcattgcagatggcccg

SEQ ID NO: 141 - DNA, 21 bp, Artificial Sequence
  Synthetic Construct
tagagcattgcagatggaccg

SEQ ID NO: 142 - DNA, 21 bp, Artificial Sequence
  Synthetic Construct
cagagcattgcagatggcccg

SEQ ID NO: 143 - DNA, 21 bp, Artificial Sequence
  Synthetic Construct
cagagcattgcagatggaccg

SEQ ID NO: 144 - DNA, 21 bp, Artificial Sequence
  Synthetic Construct
tagagcattgcagatggactg

SEQ ID NO: 145 - DNA, 21 bp, Artificial Sequence
  Synthetic Construct
cagtccatacgcaatgctccg

SEQ ID NO: 146 - DNA, 21 bp, Artificial Sequence
  Synthetic Construct
cgggccatacgcaatgctcca

SEQ ID NO: 147 - DNA, 21 bp, Artificial Sequence
  Synthetic Construct
cggtccatacgcaatgctcca

SEQ ID NO: 148 - DNA, 21 bp, Artificial Sequence
  Synthetic Construct
cgggccatacgcaatgctccg

SEQ ID NO: 149 - DNA, 21 bp, Artificial Sequence
  Synthetic Construct
cggtccatacgcaatgctccg

SEQ ID NO: 150 - DNA, 21 bp, Artificial Sequence
  Synthetic Construct
cagtccatacgcaatgctcca

SEQ ID NO: 151 - protein, 908 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 152 - protein, 869 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKESSIWLVPPYHPDTV

SEQ ID NO: 153 - protein, 584 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARF

SEQ ID NO: 154 - protein, 832 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 155 - protein, 892 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRAKTKLPQDYVFLPILESVSISTVLSSSPSSSSLSSCS

SEQ ID NO: 156 - protein, 682 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKESSIWLVPPYHPDTV

SEQ ID NO: 157 - protein, 687 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRAKTKLPQDYVFLPILESVSISTVLSSSPSSSSLSSCS

SEQ ID NO: 158 - protein, 908 aa, Mus musculus [sequence omitted]

SEQ ID NO: 159 - protein, 869 aa, Mus musculus
MKIISPVLSNLVFSRSIKVLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPSSGLNMTESQKGKPANITDSLSNRSLIVTTILEEPYVLFKKSDKPLYGNDRFEGYCIDLLRELSTILGFTYEIRLVEDGKYGAQDDVNGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKESSIWLVPPYHPDTV

SEQ ID NO: 160 - protein, 908 aa, Mus musculus [sequence omitted]

SEQ ID NO: 161 - protein, 908 aa, M. mulatta; identical to SEQ ID NO: 151

SEQ ID NO: 162 - protein, 908 aa, M. mulatta; identical to SEQ ID NO: 151

SEQ ID NO: 163 - protein, 908 aa, Rattus norvegicus
MKIISPVLSNLVFSRSIKVLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDVNGQWNGMVRELIDHKADLAVAPLAITYVREKVIDFSKPFMTLGISILYRKPNGTNPGVFSFLNPLSPDIWMYVLLACLGVSCVLFVIARFSPYEWYNPHPCNPDSDVVENNFTLLNSFWFGVGALMRQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 164 - DNA, 4592 bp, Homo sapiens
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcgtgtgggcacagaaacaccatgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 165 - DNA, 2727 bp, Homo sapiens
atgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataa

SEQ ID NO: 166 - DNA, 2610 bp, Homo sapiens
atgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaaggaatcttctatttggttagtgccaccataccatccagacactgtttag

SEQ ID NO: 167 - DNA, 2679 bp, Homo sapiens
atgaagattattttcccgattctaagtaatccagtcttcaggcgcaccgttaaactcctgctctgtttactgtggattggatattctcaaggaaccacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagagagccaagactaagttacctcaagactatgtattcctccctattttggagtcagtttccatttctacagtgttgtcatcatcaccatcttcatcatcattatcatcatgttcttaa

SEQ ID NO: 168 - DNA, 10568 bp, Homo sapiens
acttgcgctctcgctggcggctgcgtggccgaggctgggagcccgggacttcccgctgaaccgcctcctgccgcagctctgagaggactaccccagtccctaccctccctcttcaccctagccgcaggctcgcgcggctggacattgtgcttgctggatttttcccggatgctcccagactaacatggatgtcccaccatcccttgcagtggaagcttgctccttggcgcagtgagtgaagaacatgcagagactgctaatgggtttgggaagcggagactccttcctctttctgtgaccatgccgtgattgtgtttgcggccactattccacgcatccttcttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttgttgtggatcggatattcgcaaggaaccacacatgtgttaagattcggtggtatatttgaatatgtggaatctggccctatgggagctgaagaacttgcattcagatttgctgtgaatacaatcaacaggaacaggactctgctacccaataccacgttaacatatgatacacagaagatcaatctctatgacagttttgaagcatctaagaaagcttgtgatcagctgtctcttggggtggctgccatcttcggtccttcacacagttcatcagcaaatgctgttcagtccatctgcaatgctctgggggttcctcacatacagacccgctggaagcaccaggtgtcagacaataaggattccttctatgtcagtctctacccagacttctcttccctcagccgtgccatcttggatttggtgcagttttttaagtggaaaactgtcacagttgtgtatgacgacagcactggtctcattcgcttgcaagagctcatcaaagctccatcaaggtacaatcttcgacttaaaattcgtcagctgccagctgatacaaaagatgcaaagcctttgctgaaagagatgaagaggggcaaggagttccacgtgatcttcgactgcagccatgaaatggcagcaggcattttaaagcaggcattagctatgggaatgatgacagaatactaccactatatatttacgactctggacctcttcgctcttgatgtggagccctacagatacagtggcgtaaatatgacagggttcagaatactaaatacagagaatacccaagtctcctccatcatcgagaagtggtcgatggaacggttacaggcacctccaaaacctgactcaggtttgctggatggatttatgacgactgatgctgctctgatgtatgatgcagtgcacgttgtgtctgtagctgtccaacagtttccccagatgacagtcagctccttgcaatgcaatcgacacaaaccctggcgctttgggactcgcttcatgagcctaattaaagaggctcattgggaaggtcttacaggcagaattacatttaacaaaaccaatggattgcgaacagattttgatttggatgtgatcagtctcaaggaagaaggtctggagaagattgggacttgggatccatccagtggcctgaatatgacagaaagtcagaaagggaagccagcaaatattacagattcattgtctaatcgttctttgattgttaccaccattttggaagaaccatatgtcctgtttaagaagtctgacaaacctctctatgggaatgatcgatttgaaggctactgtattgatcttctacgagagttatctacaatccttggctttacatatgaaattaggcttgtggaggatgggaaatatggagcccaggatgatgtgaatggacaatggaatggaatggttcgtgagctaattgatcataaagctgaccttgcagttgctccactggctattacctatgttcgtgagaaggtcatcgacttttcaaagccgtttatgactcttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccctatgagtggtataatccacacccttgcaaccctgactcagacgtggtggaaaacaattttaccttgctaaatagtttctggtttggagttggagctctcatgcagcaaggttctgagctcatgcccaaagcactctccaccaggatagtgggaggcatttggtggtttttcacacttatcatcatttcttcgtataccgctaacctagccgcctttctgaccgtggaacgcatggagtcgcctattgactctgctgacgatttagctaagcaaaccaagatagaatatggagcagtagaggacggcgcaaccatgacgtttttcaagaaatcaaaaatctcaacgtatgataaaatgtgggcatttatgagcagcaggagacagtctgtgcttgtcaaaagcaatgaggaagggattcaacgtgtcctcacctccgattatgctttcttaatggagtcaacgaccatcgagtttgttacccagcggaactgtaacctcacgcagattggtggccttatagactccaaaggctatggtgttggcactcccatgggttctccatatcgagacaaaatcaccatagccattcttcagctgcaggaggaaggcaagctgcacatgatgaaggagaagtggtggcgaggcaatggctgcccagaggaggagagcaaagaggccagtgctctaggggtgcagaatattggtggtatcttcattgtcctggcagccggcttggtgctctcagtttttgtggcagtgggagagtttttatacaaatccaaaaaaaacgctcaattggaaaagaggtccttctgtagcgccatggtggaagaactgagaatgtctctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtcttttttttttttttcaaacaatttagcgagaatgtttcctgtggaaatatgcaacctgtgcaaaataaaatgagttacctcatgccgctgtgtctatgaactagagactcttgtgatctaagcagtttcagtgatcagacttgatttacaagcaccatggatcgacaaagttacacggggttacactgtttatcatgggttcctcccttcctttgagtgaatgttacatgaaaatgttgtggctggtttcaaatgcagtccagagagaaactgctggttccttctgaagctcaactgttgtcaggagatggaatgttggggcccaaaaggataaccaataaaaatgccataatttataaaagcaaaacaaaaagcgtgtgaaatctgcaaaaattgtagtgtcacaagaaacagtatagtcccatggtcaccaacaaaatgaggtgataatgttactagcccccaatactcagtaaaatcatcatctgaatagataatgtgttcatagaatgtggaaaaaatgtaatgcaaaacatatcagtattcaatcaaagtggaacagaaagcagaccaccatcagttattttcctttctcaatagtctgtgtcatggattgtgatatagatggcaattatctatctaattgttttcttaaaatacccatggcaaatattttaaaatgcaacttgctcccaggaacccctaccctaacctacactagaaataaaaaagccaccactggtataaagattctgatgtaaaagatatgtttttcaatccttgtcatgaattgtaaaacagggctcagtattactggttatatggaagactgaagctttcactctgacattctgatatgtcagctgaaactctccttcctcctggaaaggaccttgatggagcctgggcagattccattgataagactggggacttgtcacctatacagaactacgtgacagaactttgaggtggactgcatttaacaatagtcacaatgttaaaagaacaaaattcttgagcagttttttttttctgttttgttttaaaaaatgttcaggtttatttgtggaaatgcaagatttctatgaaaatagtttttgtatggaaatttttgtaatactttttatcaacaaaataagaacatgtgttcctgtcaggggtgtgatgtcaagcatgaacggtagtgcgtgtgcaccaccaacgtttggtgaaaactatttttatcaagaaaaaggaatcatagaagagaaatattttcaagttagatactataaaagctaggtgcactaccaccacggcttgtcacgccacacccctgagtcccacaaggcagataacatattgtaatgaacagttgtgtgtaaaatgataaaagacacagacctcttgacaacattgtgaaaacagttgagtgcacacagtttgctgtttgaatccaatgcacaaaaattttacaaaactccattaaaattatgtccattttactttcagctttggctttgatttttctcttgcatgtgtaaatgaatgtaacatggtggttttgtatagaaaatatacatcaaggggtcttaggatctcaaagttagaatcttcccaacttacagcaaaaaggaaaaggccatcctggaggtgctcctctctttctctctcccttcctctgtctctctctttgactctgtctctttgtgtctcagtctttctctatatcagtttttctctgtccctctctacctctggctctatcactctctgtctcatttacacacatacacacacacacacacacacacacacacacacgaataaagacatatacattggttttagaattagggtagctggaataaaaagaatatgattgtagagatggcaacctttatcttatctcatttgtagctggaaattgactaagttcactgtgctgcattatgttgtggaatggtaattatctactttggttcaactcatatccaatttcagaattttctgtgcattgatacttcaataatcatcagcagaggaacaaaaagggaaaagtttagaattaataattaattttagatcctaacatattaatagaaacaaactataacagttttacgttttgaaaatcaaatctgtaagattcaacttattttcctgattaattaattaattaattctaagtgtgcaattataattggaatctgacaaaaaaaaaaccccactggaaaagtttccataatgtatttcttaaaatagtaaaaattgcataatcaaattatctcaaattaattaagatttatatatgtgagcactttaaatattttatgctatgattattatcagatttcaatgattattttgttcaagctacaaatgtagctatacaaatcactgctaaagtagcagtactggtgtattagtgccaaccaagtttaaataaggaaaataatataagtattcagattattaagatgctgtttttaacaaacaaattttaaattttatgaaaatatgaatttgtaaaggaaacaaacttcattattaatattatggggaaattctgtgaatatatacaatctgacacatgtagaatttgcacattcgatgagaaactgtgtcaaaaatgatcaattgaagcaactcatttaataaaaaagaactctcactaagcaatgctttaatatgttttaaagatataattttaaacacttggaaatcttatatatgtgagtataaaaacacattaaatattcatatcttaatttacttaaaagagttttaacttcaatttattactgaatttaataatcataaacattgggtataaaattacaactttattgttaagcagccagaagagaatgaattctggtaaaatttgataatgattttattgaaaaattgaattaagttcttaaatatgtaacaaccttactagacattaactttataaatgatattaaatgtttataatatattcttttacattctaattctaattaatacttcatacatgtataagtacttaaattttccaaaacacctggtcataatatatatattaattttaattataataaaaatgctaattactttcatacataatatcaaacaatgaaaaaactctaggttggaagcatgtaagagttctcagcattttctagaggaaaaatcaaaagcaaagagaagctaatacctgttctaggcaacatcagaacctatgaattgagaggatgaatggaggctcatggacttcagctatagaaaccaacaaggatggagacaccagaggttttatacccacatttactaccactaaataatcagtatctccctgggaacaccaaagaaacacacataattaccactgaacaaccctaactctcatgggaacacaagagaaatatagtctagtttataaatgtgtaaaatgaagaatagtttattgatgggaatatttcagatattttgttgctcacttatatctacatataagtgcatatgtaataaaaattcatatcccatggcaccattcttgggtggtatagatatgaaacattgctggtttcaaaatgtttaaggattgtgtcaacttttgatgtctgtttactttgaaatatattgtaactgcagaaaagtgcatatacaaggcatatatacacagaattgctgctagttgttatagttttaaacacagttattgtgccatttaacccaggctcaatgaaagttctctgacttgctgataacattttcagggaagaaattacagtgatctttgatcatgaaatgtataattaactcatctgtgtttgactatgccaatagagtgtgtacagcattgtagagaaacagtcttgggattttgtggcagcttttaaaggtagaatcatagcaatgaaaactttggttatgcctgtcctacctaaatgtatacaaaatttgaaccattttcctccattgtgagctgatacgtgaccagatctgcagacatgtaaaattgtgaagttattcccttgtctttaggtatttgcctcaatgcaaattcaaatgtaataattttttttatttctcttaaaatatgtcagttaattatttgattttttctcaattttggtggttttatagaaatgctgaataggctcaatgggttttagatgttttatgtctattattctactctagtctataattatctggatattcattgactcctagctcaaaattagttccttggaaaatttgaagactttccactatttcctcttccccctcatctatttcccattttctcccccatttccatctctttcccccattcctcatcctttatatatatatatatatatatatatacatatatatatatatatatgtgtgtgtgtgtgtgtgtgtgtgtgtgtgtgtgtgtgtgtgtgtacttgcttttttattatcttgtctaatgttgagattgctgtggaaaacctaaaatcatgatcagatggtcacagttctttgatcatcttcttcttcttcttcttcttcttcttcttcttcttcttcttcttcttcttcttcttcttcttcttcttcttcttcttcctcttcctcctcctcctcctcttcctcctcctcttcctcttcgtcgtcttctccttctcctccttcttttccttctcttcctccacctcttcctttcttcttcttttccctcttcttcctccttctcttgtgcctcctctttttgtttttcttacttctctctttgtctctatctctatgtttctgtttctttgtctctgtctctctctctgtgtctctgtctgtcagtctctgtcggcctgtctgtctttctctctgtctcagatgtagattgatctgtatcaattgtactaacccttactagactccttaataaagctactatttttcaattttaagaaaagaccttgaaataatacttcattgtcttcctttcttcatatttcctttgttctctctaaatctgttcttatgacttgtaattacttaactttgaatcttcagtctatttacttttctatttctctatattgttattttaagtctataatctaaaatacttttttagtttctcctatctttgacattgttttaatttctgtaagttactgtttaattcctatgacatcttcacttatatcatagtagttgatcataattgttctttattcccatcattaggaaaagacattgtatattcctgttcttttctatttttttcacctgagttgaaggtattattgagataacagttgagtcatttaccctccagttactcctcccagttcttttcactttctctcccacatgtatctacttcctgtttgtctctcattcaaaaaggaaaggcctctaagagataacagcaaaatttaacaacataaaatataataacataaaacacacaagcaaaaactgtcacatcaaagttgaacaagataaaccaacaaaacaaaaagagcctcaagaaggcagaagaatcagagagcctcttgttcacttacctcagttgaattagaattggttcttatcctgtgggtagtgacccatttagaggtagaatgaccctttcttaaagttttcctaagacaatcagacaatatagatgtttacattatgatttataaatagcaaaattatagttatgaaatagaggcaaaaataattttatggttttgggtcattacaacattaagaaccatatttgagggttctagccttaggaaggttgaaaaccactgaattataaagggccttgtctccttgtgtcctctacccttttgaatctttcacatttctcacctcatcttctgtgggtgggatttagtggagacatcccactgaaagctgtgttccaaatgcatctgtttgttaaatgtcatccgtcaactcttactgttctctttatgatgatgtaaagtagaataaacatcactggcaaatttttgtttatgttaagttgtgacagaatgttcatctaacttgagtaatcctgtaggtgaatatatgtggactacttgatgaataatctgtccaagtacctccgtttggtttttacttcttgggtatattagatttgcagagaggtgtcttttaacttcattcctaatgccttgaaatcctgatcactatatttcagccaccagagtcaagaacttatcactgcaaattctgccctgtgtgtgagtgaataaagcaatttttgtcccatttattctgaaaatttgcgtttaattatatctcacattcttcatactagaatatacattttcctcttttaaaaatatgcacacctgacagactgtgtttgaaatagtgggaacaccatactctatggttccagtgtctgttttgaccatcacattactatcttctctcccaagggccaagtctaccaagtggaatatgcctttaatgctattaaccaggctggtcttatacttgtagctgtcagaggaaaagactgctcaggttttgtcacatagatgaaagtacatgacaaattactagattccaacacacttgttgaagataactgaaaccagtggttgtataatgatgggagtaacagctgacagactccaggtacaaatggcacactatgaggtagttaattggaaagacaaaggtaactatgagtttcctgtggacatgctatgtaaaagaattgctgatattactcaagtcaacacacagaataatgaaatgaggctgcttcattgttgtttgatttttttttactggtataaatgaaaaaacaaggccctcaagtgtactagtggaatcctataggtgttaagccatgacagcagaagttaaatgaatataaccaaccagcttccttgaaaacaataagtgaggaagaaaattgattggatatttgaataaacatttagataatgtaattacatgtctgcctactattctgtcgattggtttcaaactttcagaaatagaaattggaagagttagagttgaaaatactcgattcaggattcttacagaagcagagattggcactcatcttgttgttctagcagagagactgaacattgtcatcagtttaccaaatctgtgatgccacttgcctgtgtgtttgataaaaaccaacatcatagaggctccacagcttaaaatggaacctcttccactcctgccactgagctgcttaggactctgtataaataaaaacagtccttttggaaaaataaatatgtacactgtacttaaaaataaacacatgaaatttttatgtgctacgttaaaacctaactccaaaatttaaagaaacgctacaattgtccacatccattaaaagactccttgttattttatgttctcttttgtaccactattaaattgattctctattgcta

SEQ ID NO: 169 - DNA, 10655 bp, Mus musculus
acttgcgctctcgctggcggctgcgtggccgaggctgggagcccgggacttcccgctgaaccgcctcctgccgcagctctgagaggactaccccagtccctaccctccctcttcaccctagccgcaggctcgcgcggctggacattgtgcttgctggatttttcccggatgctcccagactaacatggatgtcccaccatcccttgcagtggaagcttgctccttggcgcagtgagtgaagaacatgcagagactgctaatgggtttgggaagcggagactccttcctctttctgtgaccatgccgtgattgtgtttgcggccactattccacgcatccttcttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttgttgtggatcggatattcgcaaggaaccacacatgtgttaagattcggtggtatatttgaatatgtggaatctggccctatgggagctgaagaacttgcattcagatttgctgtgaatacaatcaacaggaacaggactctgctacccaataccacgttaacatatgatacacagaagatcaatctctatgacagttttgaagcatctaagaaagcttgtgatcagctgtctcttggggtggctgccatcttcggtccttcacacagttcatcagcaaatgctgttcagtccatctgcaatgctctgggggttcctcacatacagacccgctggaagcaccaggtgtcagacaataaggattccttctatgtcagtctctacccagacttctcttccctcagccgtgccatcttggatttggtgcagttttttaagtggaaaactgtcacagttgtgtatgacgacagcactggtctcattcgcttgcaagagctcatcaaagctccatcaaggtacaatcttcgacttaaaattcgtcagctgccagctgatacaaaagatgcaaagcctttgctgaaagagatgaagaggggcaaggagttccacgtgatcttcgactgcagccatgaaatggcagcaggcattttaaagcaggcattagctatgggaatgatgacagaatactaccactatatatttacgactctggacctcttcgctcttgatgtggagccctacagatacagtggcgtaaatatgacagggttcagaatactaaatacagagaatacccaagtctcctccatcatcgagaagtggtcgatggaacggttacaggcacctccaaaacctgactcaggtttgctggatggatttatgacgactgatgctgctctgatgtatgatgcagtgcacgttgtgtctgtagctgtccaacagtttccccagatgacagtcagctccttgcaatgcaatcgacacaaaccctggcgctttgggactcgcttcatgagcctaattaaagaggctcattgggaaggtcttacaggcagaattacatttaacaaaaccaatggattgcgaacagattttgatttggatgtgatcagtctcaaggaagaaggtctggagaagattgggacttgggatccatccagtggcctgaatatgacagaaagtcagaaagggaagccagcaaatattacagattcattgtctaatcgttctttgattgttaccaccattttggaagaaccatatgtcctgtttaagaagtctgacaaacctctctatgggaatgatcgatttgaaggctactgtattgatcttctacgagagttatctacaatccttggctttacatatgaaattaggcttgtggaggatgggaaatatggagcccaggatgatgtgaatggacaatggaatggaatggttcgtgagctaattgatcataaagctgaccttgcagttgctccactggctattacctatgttcgtgagaaggtcatcgacttttcaaagccgtttatgactcttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccctatgagtggtataatccacacccttgcaaccctgactcagacgtggtggaaaacaattttaccttgctaaatagtttctggtttggagttggagctctcatgcagcaaggttctgagctcatgcccaaagcactctccaccaggatagtgggaggcatttggtggtttttcacacttatcatcatttcttcgtataccgctaacctagccgcctttctgaccgtggaacgcatggagtcgcctattgactctgctgacgatttagctaagcaaaccaagatagaatatggagcagtagaggacggcgcaaccatgacgtttttcaagaaatcaaaaatctcaacgtatgataaaatgtgggcatttatgagcagcaggagacagtctgtgcttgtcaaaagcaatgaggaagggattcaacgtgtcctcacctccgattatgctttcttaatggagtcaacgaccatcgagtttgttacccagcggaactgtaacctcacgcagattggtggccttatagactccaaaggctatggtgttggcactcccatgggttctccatatcgagacaaaatcaccatagccattcttcagctgcaggaggaaggcaagctgcacatgatgaaggagaagtggtggcgaggcaatggctgcccagaggaggagagcaaagaggccagtgctctaggggtgcagaatattggtggtatcttcattgtcctggcagccggcttggtgctctcagtttttgtggcagtgggagagtttttatacaaatccaaaaaaaacgctcaattggaaaaggaatcttctatttggttagtgccaccataccatccagacactgtttagtaaacttttgaaactttctaaaagaggtttttaatgatgaggtccttctgtagcgccatggtggaagaactgagaatgtctctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtcttttttttttttttcaaacaatttagcgagaatgtttcctgtggaaatatgcaacctgtgcaaaataaaatgagttacctcatgccgctgtgtctatgaactagagactcttgtgatctaagcagtttcagtgatcagacttgatttacaagcaccatggatcgacaaagttacacggggttacactgtttatcatgggttcctcccttcctttgagtgaatgttacatgaaaatgttgtggctggtttcaaatgcagtccagagagaaactgctggttccttctgaagctcaactgttgtcaggagatggaatgttggggcccaaaaggataaccaataaaaatgccataatttataaaagcaaaacaaaaagcgtgtgaaatctgcaaaaattgtagtgtcacaagaaacagtatagtcccatggtcaccaacaaaatgaggtgataatgttactagcccccaatactcagtaaaatcatcatctgaatagataatgtgttcatagaatgtggaaaaaatgtaatgcaaaacatatcagtattcaatcaaagtggaacagaaagcagaccaccatcagttattttcctttctcaatagtctgtgtcatggattgtgatatagatggcaattatctatctaattgttttcttaaaatacccatggcaaatattttaaaatgcaacttgctcccaggaacccctaccctaacctacactagaaataaaaaagccaccactggtataaagattctgatgtaaaagatatgtttttcaatccttgtcatgaattgtaaaacagggctcagtattactggttatatggaagactgaagctttcactctgacattctgatatgtcagctgaaactctccttcctcctggaaaggaccttgatggagcctgggcagattccattgataagactggggacttgtcacctatacagaactacgtgacagaactttgaggtggactgcatttaacaatagtcacaatgttaaaagaacaaaattcttgagcagttttttttttctgttttgttttaaaaaatgttcaggtttatttgtggaaatgcaagatttctatgaaaatagtttttgtatggaaatttttgtaatactttttatcaacaaaataagaacatgtgttcctgtcaggggtgtgatgtcaagcatgaacggtagtgcgtgtgcaccaccaacgtttggtgaaaactatttttatcaagaaaaaggaatcatagaagagaaatattttcaagttagatactataaaagctaggtgcactaccaccacggcttgtcacgccacacccctgagtcccacaaggcagataacatattgtaatgaacagttgtgtgtaaaatgataaaagacacagacctcttgacaacattgtgaaaacagttgagtgcacacagtttgctgtttgaatccaatgcacaaaaattttacaaaactccattaaaattatgtccattttactttcagctttggctttgatttttctcttgcatgtgtaaatgaatgtaacatggtggttttgtatagaaaatatacatcaaggggtcttaggatctcaaagttagaatcttcccaacttacagcaaaaaggaaaaggccatcctggaggtgctcctctctttctctctcccttcctctgtctctctctttgactctgtctctttgtgtctcagtctttctctatatcagtttttctctgtccctctctacctctggctctatcactctctgtctcatttacacacatacacacacacacacacacacacacacacacacgaataaagacatatacattggttttagaattagggtagctggaataaaaagaatatgattgtagagatggcaacctttatcttatctcatttgtagctggaaattgactaagttcactgtgctgcattatgttgtggaatggtaattatctactttggttcaactcatatccaatttcagaattttctgtgcattgatacttcaataatcatcagcagaggaacaaaaagggaaaagtttagaattaataattaattttagatcctaacatattaatagaaacaaactataacagttttacgttttgaaaatcaaatctgtaagattcaacttattttcctgattaattaattaattaattctaagtgtgcaattataattggaatctgacaaaaaaaaaaccccactggaaaagtttccataatgtatttcttaaaatagtaaaaattgcataatcaaattatctcaaattaattaagatttatatatgtgagcactttaaatattttatgctatgattattatcagatttcaatgattattttgttcaagctacaaatgtagctatacaaatcactgctaaagtagcagtactggtgtattagtgccaaccaagtttaaataaggaaaataatataagtattcagattattaagatgctgtttttaacaaacaaattttaaattttatgaaaatatgaatttgtaaaggaaacaaacttcattattaatattatggggaaattctgtgaatatatacaatctgacacatgtagaatttgcacattcgatgagaaactgtgtcaaaaatgatcaattgaagcaactcatttaataaaaaagaactctcactaagcaatgctttaatatgttttaaagatataattttaaacacttggaaatcttatatatgtgagtataaaaacacattaaatattcatatcttaatttacttaaaagagttttaacttcaatttattactgaatttaataatcataaacattgggtataaaattacaactttattgttaagcagccagaagagaatgaattctggtaaaatttgataatgattttattgaaaaattgaattaagttcttaaatatgtaacaaccttactagacattaactttataaatgatattaaatgtttataatatattcttttacattctaattctaattaatacttcatacatgtataagtacttaaattttccaaaacacctggtcataatatatatattaattttaattataataaaaatgctaattactttcatacataatatcaaacaatgaaaaaactctaggttggaagcatgtaagagttctcagcattttctagaggaaaaatcaaaagcaaagagaagctaatacctgttctaggcaacatcagaacctatgaattgagaggatgaatggaggctcatggacttcagctatagaaaccaacaaggatggagacaccagaggttttatacccacatttactaccactaaataatcagtatctccctgggaacaccaaagaaacacacataattaccactgaacaaccctaactctcatgggaacacaagagaaatatagtctagtttataaatgtgtaaaatgaagaatagtttattgatgggaatatttcagatattttgttgctcacttatatctacatataagtgcatatgtaataaaaattcatatcccatggcaccattcttgggtggtatagatatgaaacattgctggtttcaaaatgtttaaggattgtgtcaacttttgatgtctgtttactttgaaatatattgtaactgcagaaaagtgcatatacaaggcatatatacacagaattgctgctagttgttatagttttaaacacagttattgtgccatttaacccaggctcaatgaaagttctctgacttgctgataacattttcagggaagaaattacagtgatctttgatcatgaaatgtataattaactcatctgtgtttgactatgccaatagagtgtgtacagcattgtagagaaacagtcttgggattttgtggcagcttttaaaggtagaatcatagcaatgaaaactttggttatgcctgtcctacctaaatgtatacaaaatttgaaccattttcctccattgtgagctgatacgtgaccagatctgcagacatgtaaaattgtgaagttattcccttgtctttaggtatttgcctcaatgcaaattcaaatgtaataattttttttatttctcttaaaatatgtcagttaattatttgattttttctcaattttggtggttttatagaaatgctgaataggctcaatgggttttagatgttttatgtctattattctactctagtctataattatctggatattcattgactcctagctcaaaattagttccttggaaaatttgaagactttccactatttcctcttccccctcatctatttcccattttctcccccatttccatctctttcccccattcctcatcctttatatatatatatatatatatatatacatatatatatatatatatgtgtgtgtgtgtgtgtgtgtgtgtgtgtgtgtgtgtgtgtgtgtacttgcttttttattatcttgtctaatgttgagattgctgtggaaaacctaaaatcatgatcagatggtcacagttctttgatcatcttcttcttcttcttcttcttcttcttcttcttcttcttcttcttcttcttcttcttcttcttcttcttcttcttcttcctcttcctcctcctcctcctcttcctcctcctcttcctcttcgtcgtcttctccttctcctccttcttttccttctcttcctccacctcttcctttcttcttcttttccctcttcttcctccttctcttgtgcctcctctttttgtttttcttacttctctctttgtctctatctctatgtttctgtttctttgtctctgtctctctctctgtgtctctgtctgtcagtctctgtcggcctgtctgtctttctctctgtctcagatgtagattgatctgtatcaattgtactaacccttactagactccttaataaagctactatttttcaattttaagaaaagaccttgaaataatacttcattgtcttcctttcttcatatttcctttgttctctctaaatctgttcttatgacttgtaattacttaactttgaatcttcagtctatttacttttctatttctctatattgttattttaagtctataatctaaaatacttttttagtttctcctatctttgacattgttttaatttctgtaagttactgtttaattcctatgacatcttcacttatatcatagtagttgatcataattgttctttattcccatcattaggaaaagacattgtatattcctgttcttttctatttttttcacctgagttgaaggtattattgagataacagttgagtcatttaccctccagttactcctcccagttcttttcactttctctcccacatgtatctacttcctgtttgtctctcattcaaaaaggaaaggcctctaagagataacagcaaaatttaacaacataaaatataataacataaaacacacaagcaaaaactgtcacatcaaagttgaacaagataaaccaacaaaacaaaaagagcctcaagaaggcagaagaatcagagagcctcttgttcacttacctcagttgaattagaattggttcttatcctgtgggtagtgacccatttagaggtagaatgaccctttcttaaagttttcctaagacaatcagacaatatagatgtttacattatgatttataaatagcaaaattatagttatgaaatagaggcaaaaataattttatggttttgggtcattacaacattaagaaccatatttgagggttctagccttaggaaggttgaaaaccactgaattataaagggccttgtctccttgtgtcctctacccttttgaatctttcacatttctcacctcatcttctgtgggtgggatttagtggagacatcccactgaaagctgtgttccaaatgcatctgtttgttaaatgtcatccgtcaactcttactgttctctttatgatgatgtaaagtagaataaacatcactggcaaatttttgtttatgttaagttgtgacagaatgttcatctaacttgagtaatcctgtaggtgaatatatgtggactacttgatgaataatctgtccaagtacctccgtttggtttttacttcttgggtatattagatttgcagagaggtgtcttttaacttcattcctaatgccttgaaatcctgatcactatatttcagccaccagagtcaagaacttatcactgcaaattctgccctgtgtgtgagtgaataaagcaatttttgtcccatttattctgaaaatttgcgtttaattatatctcacattcttcatactagaatatacattttcctcttttaaaaatatgcacacctgacagactgtgtttgaaatagtgggaacaccatactctatggttccagtgtctgttttgaccatcacattactatcttctctcccaagggccaagtctaccaagtggaatatgcctttaatgctattaaccaggctggtcttatacttgtagctgtcagaggaaaagactgctcaggttttgtcacatagatgaaagtacatgacaaattactagattccaacacacttgttgaagataactgaaaccagtggttgtataatgatgggagtaacagctgacagactccaggtacaaatggcacactatgaggtagttaattggaaagacaaaggtaactatgagtttcctgtggacatgctatgtaaaagaattgctgatattactcaagtcaacacacagaataatgaaatgaggctgcttcattgttgtttgatttttttttactggtataaatgaaaaaacaaggccctcaagtgtactagtggaatcctataggtgttaagccatgacagcagaagttaaatgaatataaccaaccagcttccttgaaaacaataagtgaggaagaaaattgattggatatttgaataaacatttagataatgtaattacatgtctgcctactattctgtcgattggtttcaaactttcagaaatagaaattggaagagttagagttgaaaatactcgattcaggattcttacagaagcagagattggcactcatcttgttgttctagcagagagactgaacattgtcatcagtttaccaaatctgtgatgccacttgcctgtgtgtttgataaaaaccaacatcatagaggctccacagcttaaaatggaacctcttccactcctgccactgagctgcttaggactctgtataaataaaaacagtccttttggaaaaataaatatgtacactgtacttaaaaataaacacatgaaatttttatgtgctacgttaaaacctaactccaaaatttaaagaaacgctacaattgtccacatccattaaaagactccttgttattttatgttctcttttgtaccactattaaattgattctctattgcta

SEQ ID NO: 170 - DNA, 10557 bp, Mus musculus
aaacaggactaatgcttctaagatgattttggatttgggggttggaggatattagaaactaccctctggtaacgaaacgaacagattaccgtttaacatttcttcatcttcatactgctcgcgcggctggacattgtgcttgctggatttttcccggatgctcccagactaacatggatgtcccaccatcccttgcagtggaagcttgctccttggcgcagtgagtgaagaacatgcagagactgctaatgggtttgggaagcggagactccttcctctttctgtgaccatgccgtgattgtgtttgcggccactattccacgcatccttcttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttgttgtggatcggatattcgcaaggaaccacacatgtgttaagattcggtggtatatttgaatatgtggaatctggccctatgggagctgaagaacttgcattcagatttgctgtgaatacaatcaacaggaacaggactctgctacccaataccacgttaacatatgatacacagaagatcaatctctatgacagttttgaagcatctaagaaagcttgtgatcagctgtctcttggggtggctgccatcttcggtccttcacacagttcatcagcaaatgctgttcagtccatctgcaatgctctgggggttcctcacatacagacccgctggaagcaccaggtgtcagacaataaggattccttctatgtcagtctctacccagacttctcttccctcagccgtgccatcttggatttggtgcagttttttaagtggaaaactgtcacagttgtgtatgacgacagcactggtctcattcgcttgcaagagctcatcaaagctccatcaaggtacaatcttcgacttaaaattcgtcagctgccagctgatacaaaagatgcaaagcctttgctgaaagagatgaagaggggcaaggagttccacgtgatcttcgactgcagccatgaaatggcagcaggcattttaaagcaggcattagctatgggaatgatgacagaatactaccactatatatttacgactctggacctcttcgctcttgatgtggagccctacagatacagtggcgtaaatatgacagggttcagaatactaaatacagagaatacccaagtctcctccatcatcgagaagtggtcgatggaacggttacaggcacctccaaaacctgactcaggtttgctggatggatttatgacgactgatgctgctctgatgtatgatgcagtgcacgttgtgtctgtagctgtccaacagtttccccagatgacagtcagctccttgcaatgcaatcgacacaaaccctggcgctttgggactcgcttcatgagcctaattaaagaggctcattgggaaggtcttacaggcagaattacatttaacaaaaccaatggattgcgaacagattttgatttggatgtgatcagtctcaaggaagaaggtctggagaagattgggacttgggatccatccagtggcctgaatatgacagaaagtcagaaagggaagccagcaaatattacagattcattgtctaatcgttctttgattgttaccaccattttggaagaaccatatgtcctgtttaagaagtctgacaaacctctctatgggaatgatcgatttgaaggctactgtattgatcttctacgagagttatctacaatccttggctttacatatgaaattaggcttgtggaggatgggaaatatggagcccaggatgatgtgaatggacaatggaatggaatggttcgtgagctaattgatcataaagctgaccttgcagttgctccactggctattacctatgttcgtgagaaggtcatcgacttttcaaagccgtttatgactcttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccctatgagtggtataatccacacccttgcaaccctgactcagacgtggtggaaaacaattttaccttgctaaatagtttctggtttggagttggagctctcatgcagcaaggttctgagctcatgcccaaagcactctccaccaggatagtgggaggcatttggtggtttttcacacttatcatcatttcttcgtataccgctaacctagccgcctttctgaccgtggaacgcatggagtcgcctattgactctgctgacgatttagctaagcaaaccaagatagaatatggagcagtagaggacggcgcaaccatgacgtttttcaagaaatcaaaaatctcaacgtatgataaaatgtgggcatttatgagcagcaggagacagtctgtgcttgtcaaaagcaatgaggaagggattcaacgtgtcctcacctccgattatgctttcttaatggagtcaacgaccatcgagtttgttacccagcggaactgtaacctcacgcagattggtggccttatagactccaaaggctatggtgttggcactcccatgggttctccatatcgagacaaaatcaccatagccattcttcagctgcaggaggaaggcaagctgcacatgatgaaggagaagtggtggcgaggcaatggctgcccagaggaggagagcaaagaggccagtgctctaggggtgcagaatattggtggtatcttcattgtcctggcagccggcttggtgctctcagtttttgtggcagtgggagagtttttatacaaatccaaaaaaaacgctcaattggaaaagaggtccttctgtagcgccatggtggaagaactgagaatgtctctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtcttttttttttttttcaaacaatttagcgagaatgtttcctgtggaaatatgcaacctgtgcaaaataaaatgagttacctcatgccgctgtgtctatgaactagagactcttgtgatctaagcagtttcagtgatcagacttgatttacaagcaccatggatcgacaaagttacacggggttacactgtttatcatgggttcctcccttcctttgagtgaatgttacatgaaaatgttgtggctggtttcaaatgcagtccagagagaaactgctggttccttctgaagctcaactgttgtcaggagatggaatgttggggcccaaaaggataaccaataaaaatgccataatttataaaagcaaaacaaaaagcgtgtgaaatctgcaaaaattgtagtgtcacaagaaacagtatagtcccatggtcaccaacaaaatgaggtgataatgttactagcccccaatactcagtaaaatcatcatctgaatagataatgtgttcatagaatgtggaaaaaatgtaatgcaaaacatatcagtattcaatcaaagtggaacagaaagcagaccaccatcagttattttcctttctcaatagtctgtgtcatggattgtgatatagatggcaattatctatctaattgttttcttaaaatacccatggcaaatattttaaaatgcaacttgctcccaggaacccctaccctaacctacactagaaataaaaaagccaccactggtataaagattctgatgtaaaagatatgtttttcaatccttgtcatgaattgtaaaacagggctcagtattactggttatatggaagactgaagctttcactctgacattctgatatgtcagctgaaactctccttcctcctggaaaggaccttgatggagcctgggcagattccattgataagactggggacttgtcacctatacagaactacgtgacagaactttgaggtggactgcatttaacaatagtcacaatgttaaaagaacaaaattcttgagcagttttttttttctgttttgttttaaaaaatgttcaggtttatttgtggaaatgcaagatttctatgaaaatagtttttgtatggaaatttttgtaatactttttatcaacaaaataagaacatgtgttcctgtcaggggtgtgatgtcaagcatgaacggtagtgcgtgtgcaccaccaacgtttggtgaaaactatttttatcaagaaaaaggaatcatagaagagaaatattttcaagttagatactataaaagctaggtgcactaccaccacggcttgtcacgccacacccctgagtcccacaaggcagataacatattgtaatgaacagttgtgtgtaaaatgataaaagacacagacctcttgacaacattgtgaaaacagttgagtgcacacagtttgctgtttgaatccaatgcacaaaaattttacaaaactccattaaaattatgtccattttactttcagctttggctttgatttttctcttgcatgtgtaaatgaatgtaacatggtggttttgtatagaaaatatacatcaaggggtcttaggatctcaaagttagaatcttcccaacttacagcaaaaaggaaaaggccatcctggaggtgctcctctctttctctctcccttcctctgtctctctctttgactctgtctctttgtgtctcagtctttctctatatcagtttttctctgtccctctctacctctggctctatcactctctgtctcatttacacacatacacacacacacacacacacacacacacacacgaataaagacatatacattggttttagaattagggtagctggaataaaaagaatatgattgtagagatggcaacctttatcttatctcatttgtagctggaaattgactaagttcactgtgctgcattatgttgtggaatggtaattatctactttggttcaactcatatccaatttcagaattttctgtgcattgatacttcaataatcatcagcagaggaacaaaaagggaaaagtttagaattaataattaattttagatcctaacatattaatagaaacaaactataacagttttacgttttgaaaatcaaatctgtaagattcaacttattttcctgattaattaattaattaattctaagtgtgcaattataattggaatctgacaaaaaaaaaaccccactggaaaagtttccataatgtatttcttaaaatagtaaaaattgcataatcaaattatctcaaattaattaagatttatatatgtgagcactttaaatattttatgctatgattattatcagatttcaatgattattttgttcaagctacaaatgtagctatacaaatcactgctaaagtagcagtactggtgtattagtgccaaccaagtttaaataaggaaaataatataagtattcagattattaagatgctgtttttaacaaacaaattttaaattttatgaaaatatgaatttgtaaaggaaacaaacttcattattaatattatggggaaattctgtgaatatatacaatctgacacatgtagaatttgcacattcgatgagaaactgtgtcaaaaatgatcaattgaagcaactcatttaataaaaaagaactctcactaagcaatgctttaatatgttttaaagatataattttaaacacttggaaatcttatatatgtgagtataaaaacacattaaatattcatatcttaatttacttaaaagagttttaacttcaatttattactgaatttaataatcataaacattgggtataaaattacaactttattgttaagcagccagaagagaatgaattctggtaaaatttgataatgattttattgaaaaattgaattaagttcttaaatatgtaacaaccttactagacattaactttataaatgatattaaatgtttataatatattcttttacattctaattctaattaatacttcatacatgtataagtacttaaattttccaaaacacctggtcataatatatatattaattttaattataataaaaatgctaattactttcatacataatatcaaacaatgaaaaaactctaggttggaagcatgtaagagttctcagcattttctagaggaaaaatcaaaagcaaagagaagctaatacctgttctaggcaacatcagaacctatgaattgagaggatgaatggaggctcatggacttcagctatagaaaccaacaaggatggagacaccagaggttttatacccacatttactaccactaaataatcagtatctccctgggaacaccaaagaaacacacataattaccactgaacaaccctaactctcatgggaacacaagagaaatatagtctagtttataaatgtgtaaaatgaagaatagtttattgatgggaatatttcagatattttgttgctcacttatatctacatataagtgcatatgtaataaaaattcatatcccatggcaccattcttgggtggtatagatatgaaacattgctggtttcaaaatgtttaaggattgtgtcaacttttgatgtctgtttactttgaaatatattgtaactgcagaaaagtgcatatacaaggcatatatacacagaattgctgctagttgttatagttttaaacacagttattgtgccatttaacccaggctcaatgaaagttctctgacttgctgataacattttcagggaagaaattacagtgatctttgatcatgaaatgtataattaactcatctgtgtttgactatgccaatagagtgtgtacagcattgtagagaaacagtcttgggattttgtggcagcttttaaaggtagaatcatagcaatgaaaactttggttatgcctgtcctacctaaatgtatacaaaatttgaaccattttcctccattgtgagctgatacgtgaccagatctgcagacatgtaaaattgtgaagttattcccttgtctttaggtatttgcctcaatgcaaattcaaatgtaataattttttttatttctcttaaaatatgtcagttaattatttgattttttctcaattttggtggttttatagaaatgctgaataggctcaatgggttttagatgttttatgtctattattctactctagtctataattatctggatattcattgactcctagctcaaaattagttccttggaaaatttgaagactttccactatttcctcttccccctcatctatttcccattttctcccccatttccatctctttcccccattcctcatcctttatatatatatatatatatatatatacatatatatatatatatatgtgtgtgtgtgtgtgtgtgtgtgtgtgtgtgtgtgtgtgtgtgtacttgcttttttattatcttgtctaatgttgagattgctgtggaaaacctaaaatcatgatcagatggtcacagttctttgatcatcttcttcttcttcttcttcttcttcttcttcttcttcttcttcttcttcttcttcttcttcttcttcttcttcttcttcctcttcctcctcctcctcctcttcctcctcctcttcctcttcgtcgtcttctccttctcctccttcttttccttctcttcctccacctcttcctttcttcttcttttccctcttcttcctccttctcttgtgcctcctctttttgtttttcttacttctctctttgtctctatctctatgtttctgtttctttgtctctgtctctctctctgtgtctctgtctgtcagtctctgtcggcctgtctgtctttctctctgtctcagatgtagattgatctgtatcaattgtactaacccttactagactccttaataaagctactatttttcaattttaagaaaagaccttgaaataatacttcattgtcttcctttcttcatatttcctttgttctctctaaatctgttcttatgacttgtaattacttaactttgaatcttcagtctatttacttttctatttctctatattgttattttaagtctataatctaaaatacttttttagtttctcctatctttgacattgttttaatttctgtaagttactgtttaattcctatgacatcttcacttatatcatagtagttgatcataattgttctttattcccatcattaggaaaagacattgtatattcctgttcttttctatttttttcacctgagttgaaggtattattgagataacagttgagtcatttaccctccagttactcctcccagttcttttcactttctctcccacatgtatctacttcctgtttgtctctcattcaaaaaggaaaggcctctaagagataacagcaaaatttaacaacataaaatataataacataaaacacacaagcaaaaactgtcacatcaaagttgaacaagataaaccaacaaaacaaaaagagcctcaagaaggcagaagaatcagagagcctcttgttcacttacctcagttgaattagaattggttcttatcctgtgggtagtgacccatttagaggtagaatgaccctttcttaaagttttcctaagacaatcagacaatatagatgtttacattatgatttataaatagcaaaattatagttatgaaatagaggcaaaaataattttatggttttgggtcattacaacattaagaaccatatttgagggttctagccttaggaaggttgaaaaccactgaattataaagggccttgtctccttgtgtcctctacccttttgaatctttcacatttctcacctcatcttctgtgggtgggatttagtggagacatcccactgaaagctgtgttccaaatgcatctgtttgttaaatgtcatccgtcaactcttactgttctctttatgatgatgtaaagtagaataaacatcactggcaaatttttgtttatgttaagttgtgacagaatgttcatctaacttgagtaatcctgtaggtgaatatatgtggactacttgatgaataatctgtccaagtacctccgtttggtttttacttcttgggtatattagatttgcagagaggtgtcttttaacttcattcctaatgccttgaaatcctgatcactatatttcagccaccagagtcaagaacttatcactgcaaattctgccctgtgtgtgagtgaataaagcaatttttgtcccatttattctgaaaatttgcgtttaattatatctcacattcttcatactagaatatacattttcctcttttaaaaatatgcacacctgacagactgtgtttgaaatagtgggaacaccatactctatggttccagtgtctgttttgaccatcacattactatcttctctcccaagggccaagtctaccaagtggaatatgcctttaatgctattaaccaggctggtcttatacttgtagctgtcagaggaaaagactgctcaggttttgtcacatagatgaaagtacatgacaaattactagattccaacacacttgttgaagataactgaaaccagtggttgtataatgatgggagtaacagctgacagactccaggtacaaatggcacactatgaggtagttaattggaaagacaaaggtaactatgagtttcctgtggacatgctatgtaaaagaattgctgatattactcaagtcaacacacagaataatgaaatgaggctgcttcattgttgtttgatttttttttactggtataaatgaaaaaacaaggccctcaagtgtactagtggaatcctataggtgttaagccatgacagcagaagttaaatgaatataaccaaccagcttccttgaaaacaataagtgaggaagaaaattgattggatatttgaataaacatttagataatgtaattacatgtctgcctactattctgtcgattggtttcaaactttcagaaatagaaattggaagagttagagttgaaaatactcgattcaggattcttacagaagcagagattggcactcatcttgttgttctagcagagagactgaacattgtcatcagtttaccaaatctgtgatgccacttgcctgtgtgtttgataaaaaccaacatcatagaggctccacagcttaaaatggaacctcttccactcctgccactgagctgcttaggactctgtataaataaaaacagtccttttggaaaaataaatatgtacactgtacttaaaaataaacacatgaaatttttatgtgctacgttaaaacctaactccaaaatttaaagaaacgctacaattgtccacatccattaaaagactccttgttattttatgttctcttttgtaccactattaaattgattctctattgcta

SEQ ID NO: 171 - DNA, 4579 bp, M. mulatta
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctgtgaccatgccgtgatcgtgtctgcggtcaccactcgacgcatccttatttctgcccgaacccgggagccgaacgctagatcggggaagtgggtgccgtgcgtgtgggcacagaaacaccatgaagattattttccccattctaagtaatccagtcttcaggcgcaccgttaaactcctgctctgtttactgtggattggatattctcaaggaactacacatgtattaagatttggtggtatttttgaatatgtggaatctggcccaatgggagctgaggaacttgcattcagatttgctgtgaatacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaatgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtacaatcttcgactcaaaattcgtcagctacctgctgatacaaaggatgcaaaacccttactaaaagaaatgaaaagaggcaaggagtttcacgtaatctttgattgtagccatgaaatggcagcaggcattttaaaacaggcattagctatgggaatgatgacagaatactatcattacatctttaccactctggacctctttgctcttgacgttgagccgtaccgatacagtggtgttaatatgacagggttcagaatattaaatacagaaaatacccaagtttcctccatcattgaaaagtggtcaatggaacgattgcaggcacctccgaaacccgattcaggtttgctggatggatttatgacgactgatgctgctctaatgtatgatgctgtgcatgtggtgtctgtggccgtccaacagtttccccagatgacagtcagttccttgcagtgtaatcgacataaaccctggcgcttcgggactcgctttatgagtctaattaaagaggcacattgggaaggcctcacaggcagaataactttcaacaaaaccaatggcttgcgaacagattttgatttggatgtgatcagtctcaaggaagaaggtctagaaaagattggaacgtgggatccagccagtggcctgaatatgacagaaagtcaaaagggaaagccggcaaacatcacagattccttatccaatcgttctttgattgttaccaccattttggaagaaccctatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactccaaaggttatggcgttggcactcccatgggttctccatatcgagacaaaattaccatagcaattcttcagctgcaagaggaaggcaagctgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcagttggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagcgagaatgtttcctgtggaaatatgcaacctgtgcaaaataaaatgagttacctcatgccgctgtgtctatgaactagagactctgtgatctaagcaattgcaatgatcagacttgatttacaagcatcatggatcaaccaagttacacggggttacactgttaatcatgggttcctcccttcttctgagtgaatgttacatgagcattttgtggctggtttcaaatgcagtccagtgagaaattacaggttccttttgaagctcaactgttgccaggagatggaatatcgacgcccaacagggcaaccaatcaaagtgtcactaagaatacaaatatttggaatcagcaaaaactgtagtgttacagaacagtacagtcttctgaacacccagatcatagaggtgatgatgctactagcccccaactactcagtataattattgtctgaatacacagtatatgtttataggatgtgaaaaaatgtaatgcaaaacaaatttgaatcccatggcagttggaatataaagcagatgtttatcacttattttcctttttttcttttcctttttgttttttttttttttgacagtctgtgtctctgattgagatagaaatgccaatttttaaggcaataatgcttttcttaagttccctaaggcagaagatttaacatgcaactctaccatatccctttctattcccccaacaccttttctctaacctccgtatcccaaataataataataatagtaataataaaaacagttggttcagtgattctgaattaaaaggataatgttttgcaatctttaagttgtaaaaagggctgagtattggctgtgtggaagactaaagctttcattctaacattcagacatagcaatccaaaccctgttcctgctgtaaatgaacttgatggagcatgggcagatctcagtggtacgagaaaggggactggtcatctatagaaaaatctgtgagagaacttggacatggactgcatttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagagaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataactgttgagtgcacacagtttgctgtttgaatccaatgcacaaaattaaaaaaaaatcattaaaattatgttcattttactttca

SEQ ID NO: 172 - DNA, 4722 bp, M. mulatta
ccgtcttcgcctttgccgtcactcgcgctcggcgccggcggctgcgctggtcggtctgggagccggggactttccgcccgacctcctccggctgctcctccccgaggaccaccccaccccctccccacccacctcacccctagcgccaggctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctgtgaccatgccgtgatcgtgtctgcggtcaccactcgacgcatccttatttctgcccgaacccgggagccgaacgctagatcggggaagtgggtgccgtgcgtgtgggcacagaaacaccatgaagattattttccccattctaagtaatccagtcttcaggcgcaccgttaaactcctgctctgtttactgtggattggatattctcaaggaactacacatgtattaagatttggtggtatttttgaatatgtggaatctggcccaatgggagctgaggaacttgcattcagatttgctgtgaatacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaatgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtacaatcttcgactcaaaattcgtcagctacctgctgatacaaaggatgcaaaacccttactaaaagaaatgaaaagaggcaaggagtttcacgtaatctttgattgtagccatgaaatggcagcaggcattttaaaacaggcattagctatgggaatgatgacagaatactatcattacatctttaccactctggacctctttgctcttgacgttgagccgtaccgatacagtggtgttaatatgacagggttcagaatattaaatacagaaaatacccaagtttcctccatcattgaaaagtggtcaatggaacgattgcaggcacctccgaaacccgattcaggtttgctggatggatttatgacgactgatgctgctctaatgtatgatgctgtgcatgtggtgtctgtggccgtccaacagtttccccagatgacagtcagttccttgcagtgtaatcgacataaaccctggcgcttcgggactcgctttatgagtctaattaaagaggcacattgggaaggcctcacaggcagaataactttcaacaaaaccaatggcttgcgaacagattttgatttggatgtgatcagtctcaaggaagaaggtctagaaaagattggaacgtgggatccagccagtggcctgaatatgacagaaagtcaaaagggaaagccggcaaacatcacagattccttatccaatcgttctttgattgttaccaccattttggaagaaccctatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactccaaaggttatggcgttggcactcccatgggttctccatatcgagacaaaattaccatagcaattcttcagctgcaagaggaaggcaagctgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcagttggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagcgagaatgtttcctgtggaaatatgcaacctgtgcaaaataaaatgagttacctcatgccgctgtgtctatgaactagagactctgtgatctaagcaattgcaatgatcagacttgatttacaagcatcatggatcaaccaagttacacggggttacactgttaatcatgggttcctcccttcttctgagtgaatgttacatgagcattttgtggctggtttcaaatgcagtccagtgagaaattacaggttccttttgaagctcaactgttgccaggagatggaatatcgacgcccaacagggcaaccaatcaaagtgtcactaagaatacaaatatttggaatcagcaaaaactgtagtgttacagaacagtacagtcttctgaacacccagatcatagaggtgatgatgctactagcccccaactactcagtataattattgtctgaatacacagtatatgtttataggatgtgaaaaaatgtaatgcaaaacaaatttgaatcccatggcagttggaatataaagcagatgtttatcacttattttcctttttttcttttcctttttgttttttttttttttgacagtctgtgtctctgattgagatagaaatgccaatttttaaggcaataatgcttttcttaagttccctaaggcagaagatttaacatgcaactctaccatatccctttctattcccccaacaccttttctctaacctccgtatcccaaataataataataatagtaataataaaaacagttggttcagtgattctgaattaaaaggataatgttttgcaatctttaagttgtaaaaagggctgagtattggctgtgtggaagactaaagctttcattctaacattcagacatagcaatccaaaccctgttcctgctgtaaatgaacttgatggagcatgggcagatctcagtggtacgagaaaggggactggtcatctatagaaaaatctgtgagagaacttggacatggactgcatttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagagaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataactgttgagtgcacacagtttgctgtttgaatccaatgcacaaaattaaaaaaaaatcattaaaattatgttcatttta

SEQ ID NO: 173 - DNA, 3058 bp, Rattus norvegicus
ggctggacattgtgcttgctggatttttcccggatgctcccggactaacatggatgtcccaccatcccttgcagtggaagcttgctccttggcgcagtgagagtgaagaacatgcagcgactgctaatgggtttgggaagcggagactccttcctctttctgtgaccatgccgtgattgtgtctgcggccactactccacgcatcttccttctcgtccaagcccggagcctaacgctagatcggggaagtgggtgccgcgcgcgcaggcacggaaacatcatgaagattatttccccagttttaagtaatctagtcttcagtcgctccattaaagtcctgctctgcttattgtggatcggatattcgcaaggaaccacacatgtgttaagattcggtggtatatttgaatatgtggaatctggccccatgggagcagaagaacttgcattcagatttgctgtgaataccatcaacagaaacaggactttgctgcccaacaccactttaacttatgatactcagaagatcaatctctatgacagttttgaagcatctaagaaagcttgtgatcagctgtctcttggggtggctgctatcttcggtccttcacacagttcatcagccaatgctgtgcagtccatctgcaatgctctgggggttccccacatacagacccgctggaagcaccaggtgtcagacaacaaggattccttctacgtcagtctctacccagacttctcttccctgagccgcgccatcttggatttggtgcagttttttaagtggaaaactgtcacagttgtgtatgacgacagcactggtctcattcgcttgcaagagctcatcaaagctccatcgaggtacaatcttcgacttaaaattcgtcagctgccagctgataccaaagatgcaaaacctttgctgaaggagatgaaaagaggcaaggagttccacgtgatcttcgactgcagccatgagatggcagcaggcattttaaaacaggcattagctatgggaatgatgacagaatactatcactatatatttacaactctggacctctttgctcttgacgtggagccctacagatacagtggcgtaaatatgacagggttcaggatactaaatacagagaatacccaagtctcctccatcatcgaaaagtggtctatggaacggttacaggcgcctccaaaacctgactcaggtttgctggatggatttatgacgactgatgctgctctgatgtatgatgcagtgcacgttgtgtctgtggctgtccaacagtttccccagatgacagtcagctccttgcaatgcaatcgacacaaaccctggcgctttgggacccgcttcatgagtctaattaaagaggctcactgggaaggtctcacaggcagaataacatttaacaaaaccaatggattacggacagattttgatttggatgtgatcagtctcaaggaagaaggtctggagaagattggaacttgggatccagccagtggcctgaatatgacagaaagtcagaaaggaaagccagcaaatatcacagactcattgtctaatcgttctttgattgttaccaccattttggaagaaccgtatgttctgtttaagaagtctgacaaaccactctatgggaatgatcgatttgaaggctactgtattgatctcctacgagagttatctacaatccttggctttacatatgagattaggcttgtggaggatgggaaatatggagcccaggatgatgtgaacggacaatggaatggaatggttcgtgaactaatcgatcataaagctgaccttgcagttgctccactggctataacctatgttcgtgagaaggtcatcgacttttcaaagccgtttatgacacttggaataagtattttgtaccgcaagcccaatggtacaaacccaggcgtcttctccttcctgaatcctctctcccctgatatctggatgtatgttctgctggcttgcttgggtgtcagttgtgtgctctttgtcatagccaggtttagtccctatgagtggtataacccacacccttgcaaccctgactcagacgtggtggaaaacaattttaccttgctaaatagtttctggtttggagttggagctctcatgcggcaaggttctgagctcatgcccaaagcactctccaccaggatagtgggaggcatttggtggtttttcacacttatcatcatttcttcgtataccgctaacctagccgcctttctgactgtggaacgcatggagtcgcccattgactctgctgacgatttagctaagcaaaccaagatagagtatggagcagtggaggacggcgcaaccatgacgttttttaagaaatcaaaaatttcaacgtatgataaaatgtgggcgtttatgagcagcaggagacagtctgtgcttgtcaaaagcaatgaggaagggatccaacgagtcctcacctcggattatgctttcttaatggagtcaacaaccatcgagtttgttacacagcggaactgtaacctcacgcagattggcggccttatagactccaaaggctatggcgttggcactcctatgggctctccatatcgagacaaaatcaccatagcaattcttcagctgcaggaggaaggcaagctgcacatgatgaaggagaaatggtggcggggcaatggctgcccagaggaggagagcaaagaggccagtgctctgggggtgcagaatattggtggtatcttcattgtcctggcagccggcttggtgctctcagtttttgtggcagtgggagagtttttatacaaatccaaaaaaaacgctcaattggaaaagaggtccttctgtagcgctatggtggaagagctgagaatgtccctgaagtgccagcgtcggctcaaacataagccacaggccccagttattgtgaaaacagaagaagttatcaacatgcacacatttaacgacagaaggttgccaggtaaagaaaccatggcatgaagctgggaggccaatcacccaagcacaaactgtcgtctttttttttttttt

SEQ ID NO: 174 - DNA, 2631 bp, Homo sapiens
accacacatgtattaagatttggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggca

SEQ ID NO: 175 - DNA, 293 bp, Homo sapiens
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcgtgtgggcacagaaacacc

SEQ ID NO: 176 - DNA, 1572 bp, Homo sapiens
agctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 177 - DNA, 93 bp, Homo sapiens
atgaagattattttcccgattctaagtaatccagtcttcaggcgcaccgttaaactcctgctctgtttactgtggattggatattctcaagga

SEQ ID NO: 178 - DNA, 408 bp, Homo sapiens
gctcgcgcggccggacattgtgggtgtgcgtgctggatttctcccggatgctctccgactaacatggatgtcccaccattccttgcagtggaaggttgttccttggcgcagtgagtgaagaacatgcagcgattgctaatgggtttgggaagcggagactccttcctctctctatgaccatgccgtgatcgtgtctgcggtcaccactcgacgcatcctcatttctacccgaacccaggagccgaacgctagatcggggaagtgggtgccgtgcgtgtgggcacagaaacaccatgaagattattttcccgattctaagtaatccagtcttcaggcgcaccgttaaactcctgctctgtttactgtggattggatattctcaaggaaccacacatgtattaagatttg

SEQ ID NO: 179 - DNA, 168 bp, Homo sapiens
gtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaag

SEQ ID NO: 180 - DNA, 258 bp, Homo sapiens
cctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactg

SEQ ID NO: 181 - DNA, 182 bp, Homo sapiens
gtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacag

SEQ ID NO: 182 - DNA, 54 bp, Homo sapiens
gcattagctatgggaatgatgacagaatactatcattatatctttaccactctg

SEQ ID NO: 183 - DNA, 174 bp, Homo sapiens
gacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacg

SEQ ID NO: 184 - DNA, 144 bp, Homo sapiens
actgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagag

SEQ ID NO: 185 - DNA, 108 bp, Homo sapiens
gcacattgggaaggcctcacaggcagaataactttcaacaaaaccaatggcttgagaacagattttgatttggatgtgatcagtctgaaggaagaaggtctagaaaag

SEQ ID NO: 186 - DNA, 114 bp, Homo sapiens
attggaacgtgggatccagccagtggcctgaatatgacagaaagtcaaaagggaaagccagcgaacatcacagattccttatccaatcgttctttgattgttaccaccattttg

SEQ ID NO: 187 - DNA, 207 bp, Homo sapiens
gaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcat

SEQ ID NO: 188 - DNA, 224 bp, Homo sapiens
aaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccag

SEQ ID NO: 189 - DNA, 119 bp, Homo sapiens
gtttagtccttatgagtggtataatccacacccttgcaaccctgactcagacgtggtggaaaacaattttaccttgctaaatagtttctggtttggagttggagctctcatgcagcaag

SEQ ID NO: 190 - DNA, 218 bp, Homo sapiens
gttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaag

SEQ ID NO: 191 - DNA, 226 bp, Homo sapiens
aaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgg

SEQ ID NO: 192 - DNA, 251 bp, Homo sapiens
gttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaag

SEQ ID NO: 193 - DNA, 1737 bp, Homo sapiens
aggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 194 - DNA, 429 bp, Artificial Sequence
  Synthetic Construct
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgc

SEQ ID NO: 195 - DNA, 469 bp, Artificial Sequence
  Synthetic Construct
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcag

SEQ ID NO: 196 - DNA, 401 bp, Artificial Sequence
  Synthetic Construct
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcg

SEQ ID NO: 197 - DNA, 477 bp, Artificial Sequence
  Synthetic Construct
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcc

SEQ ID NO: 198 - DNA, 477 bp, Artificial Sequence
  Synthetic Construct
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcc

SEQ ID NO: 199 - DNA, 60 bp, Artificial Sequence
  Synthetic Construct
gatgctgacgaaggctcgcgaggctgtgagcagccacagtgccctgctcagaagccccgg

SEQ ID NO: 200 - DNA, 60 bp, Artificial Sequence
  Synthetic Construct
gtctcccgcgcccgcgcccgtgtcgccgccgtgcccgcgagcgggagccggagtcgccgc

SEQ ID NO: 201 - DNA, 60 bp, Artificial Sequence
  Synthetic Construct
cgtgtgcagatgcagggcgccggtgccctgcgggtgcgggtgcaggagcagcgtgtgcag

SEQ ID NO: 202 - DNA, 60 bp, Artificial Sequence
  Synthetic Construct
ccccacgccaccctttctggtcatctcccctcccgccccgcccctgcgcacactccctcg

SEQ ID NO: 203 - DNA, 60 bp, Artificial Sequence
  Synthetic Construct
tctccccggtaaagtctcgcggtgctgccgggctcagccccgtctcctcctcttgctccc

SEQ ID NO: 204 - DNA, 1289 bp, Artificial Sequence
  Synthetic Construct
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 205 - DNA, 133 bp, Artificial Sequence
  Synthetic Construct
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 206 - DNA, 82 bp, Artificial Sequence
  Synthetic Construct
gtgagtatctcagggatccagacatggggatatgggaggtgcctctgatcccagggctcactgtgggtctctctgttcacag

SEQ ID NO: 207 - DNA, 304 bp, Artificial Sequence
  Synthetic Construct
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 208 - DNA, 130 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 209 - DNA, 106 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtgg

SEQ ID NO: 210 - DNA, 143 bp, Artificial Sequence
  Synthetic Construct
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 211 - DNA, 110 bp, Artificial Sequence
  Synthetic Construct
ttggccactccctctctgcgcgctcgctcgctcactgaggccgggcgaccaaaggtcgcccgacgcccgggctttgcccgggcggcctcagtgagcgagcgagcgcgcag

SEQ ID NO: 212 - DNA, 129 bp, Artificial Sequence
  Synthetic Construct
ggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 213 - DNA, 55 bp, Oryctolagus cuniculus
ataaaggaaatttattttcattgcaatagtgtgttggaattttttgtgtctctca

SEQ ID NO: 214 - DNA, 127 bp, Oryctolagus cuniculus
gatctttttccctctgccaaaaattatggggacatcatgaagccccttgagcatctgacttctggctaataaaggaaatttattttcattgcaatagtgtgttggaattttttgtgtctctcactcg

SEQ ID NO: 215 - DNA, 132 bp, Oryctolagus cuniculus
gatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 216 - DNA, 19 bp, Bos taurus
tagcaggcatgctggggag

SEQ ID NO: 217 - DNA, 129 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgac

SEQ ID NO: 218 - DNA, 123 bp, Artificial Sequence
  Synthetic Construct
gctgcctactgcctcggacttcaaggggctactttaggagcaattatcttgtttactaaaactgaataccttgctatctctttgatacatttttacaaagctgaattaaaatggtataaatta

SEQ ID NO: 219 - DNA, 19 bp, Artificial Sequence
  Synthetic Construct
ctgtgaagccacagatggg

SEQ ID NO: 220 - DNA, 106 bp, Artificial Sequence
  Synthetic Construct
acgtttccagaacgtctgtagcttttctcctccttccctccattttcctcttggtcttacctttggcctagtggttggtgtagtgataatgtagcgagattttctg

SEQ ID NO: 221 - DNA, 124 bp, Artificial Sequence
  Synthetic Construct
tggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 222 - DNA, 20 bp, Artificial Sequence
  Synthetic Construct
ggttgcgaggtatgagtaaa

SEQ ID NO: 223 - DNA, 212 bp, Artificial Sequence
  Synthetic Construct
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctc

SEQ ID NO: 224 - DNA, 213 bp, Artificial Sequence
  Synthetic Construct
gagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 225 - DNA, 18 bp, Artificial Sequence
  Synthetic Construct
atttaatgtctatacaat

SEQ ID NO: 226 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgagcctgggttttatatcgagaagctgcctactgcctcggacttcaaggggctactttaggagcaattatcttgtttactaaaactgaataccttgctatctctttgatacatttttacaaagctgaattaaaatggtataaatta

SEQ ID NO: 227 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgagcctgggttttatatcgcgaagctgcctactgcctcggacttcaaggggctactttaggagcaattatcttgtttactaaaactgaataccttgctatctctttgatacatttttacaaagctgaattaaaatggtataaatta

SEQ ID NO: 228 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgctgtgaagccacagatgagcatgggttttatatcgagaagctgcctactgcctcggacttcaaggggctactttaggagcaattatcttgtttactaaaactgaataccttgctatctctttgatacatttttacaaagctgaattaaaatggtataaatta

SEQ ID NO: 229 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccatgctgtgaagccacagatgagcatgggttttatatcgcgaagctgcctactgcctcggacttcaaggggctactttaggagcaattatcttgtttactaaaactgaataccttgctatctctttgatacatttttacaaagctgaattaaaatggtataaatta

SEQ ID NO: 230 - DNA, 22 bp, Artificial Sequence
  Synthetic Construct
ttctcgatatggagaacccagg

SEQ ID NO: 231 - DNA, 22 bp, Artificial Sequence
  Synthetic Construct
ttctcgatatggagaacccagg

SEQ ID NO: 232 - DNA, 22 bp, Artificial Sequence
  Synthetic Construct
ttctcgatatggagaacccatg

SEQ ID NO: 233 - DNA, 22 bp, Artificial Sequence
  Synthetic Construct
ttctcgatatggagaacccatg

SEQ ID NO: 234 - DNA, 22 bp, Artificial Sequence
  Synthetic Construct
agcctgggttttatatcgagaa

SEQ ID NO: 235 - DNA, 22 bp, Artificial Sequence
  Synthetic Construct
agcctgggttttatatcgcgaa

SEQ ID NO: 236 - DNA, 22 bp, Artificial Sequence
  Synthetic Construct
agcatgggttttatatcgagaa

SEQ ID NO: 237 - DNA, 22 bp, Artificial Sequence
  Synthetic Construct
agcatgggttttatatcgcgaa

SEQ ID NO: 238 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaatta

SEQ ID NO: 239 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgagcggctgcctactgcctcggacttcaaggggctactttaggagcaattatcttgtttactaaaactgaataccttgctatctctttgatacatttttacaaagctgaattaaaatggtataaatta

SEQ ID NO: 240 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgaggggctgcctactgcctcggacttcaaggggctactttaggagcaattatcttgtttactaaaactgaataccttgctatctctttgatacatttttacaaagctgaattaaaatggtataaatta

SEQ ID NO: 241 - DNA, 313 bp, Artificial Sequence
  Synthetic Construct
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggctgtgaagccacagatgggcctgggttttatatcgaggagctgcctactgcctcggacttcaaggggctactttaggagcaattatcttgtttactaaaactgaataccttgctatctctttgatacatttttacaaagctgaattaaaatggtataaatta

SEQ ID NO: 242 - DNA, 22 bp, Artificial Sequence
  Synthetic Construct
ttctcgatatggagaacccagg

SEQ ID NO: 243 - DNA, 22 bp, Artificial Sequence
  Synthetic Construct
ttctcgatatggagaacccagg

SEQ ID NO: 244 - DNA, 22 bp, Artificial Sequence
  Synthetic Construct
ttctcgatatggagaacccagg

SEQ ID NO: 245 - DNA, 22 bp, Artificial Sequence
  Synthetic Construct
ttctcgatatggagaacccagg

SEQ ID NO: 246 - DNA, 20 bp, Artificial Sequence
  Synthetic Construct
cctgggttttatatcgagca

SEQ ID NO: 247 - DNA, 20 bp, Artificial Sequence
  Synthetic Construct
cctgggttttatatcgagcg

SEQ ID NO: 248 - DNA, 20 bp, Artificial Sequence
  Synthetic Construct
cctgggttttatatcgaggg

SEQ ID NO: 249 - DNA, 20 bp, Artificial Sequence
  Synthetic Construct
cctgggttttatatcgagga

SEQ ID NO: 250 - DNA, 1269 bp, Artificial Sequence
  Synthetic Construct
atagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattac

SEQ ID NO: 251 - DNA, 29 bp, Artificial Sequence
  Synthetic Construct
cgagtttaattggtttatagaactcttca

SEQ ID NO: 252 - DNA, 3121 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 253 - DNA, 3066 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 254 - DNA, 3261 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgcagtccatctacaacactctgatttaatgtctatacaattagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 255 - DNA, 3261 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgaggttcatctacaacactttaatttaatgtctatacaattagagcattgcagatggaccgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 256 - DNA, 3432 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 257 - DNA, 3432 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgtcacgggatccgaattcgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattactcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 258 - DNA, 622 bp, Homo sapiens
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 259 - DNA, 1142 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 260 - DNA, 1282 bp, Homo sapiens
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 261 - DNA, 1830 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 262 - DNA, 3103 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacgacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggttgatctgaagcaattcgttgagtctgccttcgcccttgcccactctctcacctcctccccccggctctgactgactttactcccacaggtgagcggcggatgcccttctcctctggctgtaattagtcttggtttacttgtttcttttctgtggctgttgaaagccttgaggggctctggagggccctttgtgtgggggagtgcttgggggtgttgttgtgtgtgtgttggggagtctttgtgctcttctgcctgtgctgtgagtctgtggttgttgggctttgtgtctctcagtgtgttaggggagttgctggggtgtgcccttgtgtgggggggctgtaggggaacaaaggctgttgtgggtgtgtgttgggggggtgagcagggggtgtgggttttgttggctgcaaccccccctgcacccccctccctagttgctgagcatgcctgctttggtgtgggctcttatgggttggttgggcttcttgctggtgggggtggtgcaggtgggggtgctggtgggtgggctccttggctgggagggcttggggaggggttgtgcccctgagtctgtgctgttaggttgtagctcagccattgccttttttgtagagggtcagggacttcctttgtcccaaatctgtgtgagctaaatctgggaggtctctcaccccctctagtggttgggtaagtgtgtgtctgcaggaaggaaagggtgggagggccttttgtttcttctcttccccttctccctctccagccttgggctgtcttgggggatgctgcctttggggggatgggcagggtgggtttgcttctggttgtgactgtgctctagagcctctgctaacctgttctgccttcttctttttcctacagctcctgggcaattgctggttattgtgctgtctctattttggcaaagaattctctgaatttcgaccacccataatacccattaccctggtagatatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaaagtagctagcgcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag